(19) United States
(12) Patent Application Publication
(10) Pub. No.: US 2006/0009431 A1
(43) Pub. Date: Jan. 12, 2006

(54) Nitrosated and nitrosylated compounds, compositions and methods use

(75) Inventors: Richard A. Earl, Westford, MA (US); David S. Garvey, Dover, MA (US); Ricky D. Gaston, Malden, MA (US); Chia-En Lin, Concord, MA (US); Ramani R. Ranatunge, Lexington, MA (US); Stewart K. Richardson, Tolland, CT (US); Cheri A. Stevenson, Haverhill, MA (US)

Correspondence Address: EDWARD D GRIEFF;HALE & DORR LLP, 1455 PENNSYLVANIA AVE, NW, WASHINGTON, DC, 20004, US

(73) Assignee: NitroMed, Inc., Lexington, MA (US)

(21) Appl. No.: 11/221,901

(22) Filed: Sep. 9, 2005

Related U.S. Application Data
(63) Continuation of application No. PCT/US04/07943, filed on Mar. 15, 2004.
(60) Provisional application No. 60/453,963, filed on Mar. 13, 2003.
(60) Provisional application No. 60/482,134, filed on Jun. 25, 2003.

(51) Int. Cl. A61K 31/56; A61K 31/21

(52) U.S. Cl. 514/171; 514/509

(57) ABSTRACT

The invention describes novel nitrosated and/or nitrosylated compounds of the invention, and pharmaceutically acceptable salts thereof, and novel compositions comprising at least one nitrosated and/or nitrosylated compound of the invention, and, optionally, at least one nitric oxide donor compound and/or at least one therapeutic agent. The invention also provides novel compositions comprising at least one compound of the invention, that is optionally nitrosated and/or nitrosylated, and at least one nitric oxide donor compound and/or at least one therapeutic agent. The compounds and compositions of the invention can also be bound to a matrix. The invention also provides methods for treating cardiovascular diseases, for inhibiting platelet aggregation and platelet adhesion caused by the exposure of blood to a medical device, for treating pathological conditions resulting from abnormal cell proliferation; transplantation rejections, autoimmune, inflammatory, proliferative, hyperproliferative or vascular diseases; for reducing scar tissue or for inhibiting wound contraction, particularly the prophylactic and/or therapeutic treatment of restenosis by administering at least one compound of the invention that is optionally nitrosated and/or nitrosylated, in combination with nitric oxide donors that are capable of releasing nitric oxide or indirectly delivering or transferring nitric oxide to targeted sites under physiological conditions. The compounds of the invention are preferably estradiol compounds, troglitazone compounds, tranilast compounds, retinoic acid compounds, resveratol compounds, myophenolic acid compounds, acid compounds, anthracenone compounds and trapidil compounds.

### RELATED APPLICATIONS

This application claims priority under 35 USC § 120 to PCT/US04/007943 filed Mar. 15, 2004, which claims priority under 35 USC § 119 to U.S. Application No. 60/453,963 filed Mar. 13, 2003 and U.S. Application No. 60/482,134 filed Jun. 25, 2003.

### FIELD OF THE INVENTION

The invention describes novel nitrosated and/or nitrosylated compounds of the invention, and pharmaceutically acceptable salts thereof, and novel compositions comprising at least one nitrosated and/or nitrosylated compound of the invention, and, optionally, at least one nitric oxide donor compound and/or at least one therapeutic agent. The invention also provides novel compositions comprising at least one compound of the invention, that is optionally nitrosated and/or nitrosylated, and at least one nitric oxide donor compound and/or at least one therapeutic agent. The compounds and compositions of the invention can also be bound to a matrix. The invention also provides methods for treating cardiovascular diseases, for inhibiting platelet aggregation and platelet adhesion caused by the exposure of blood to a medical device, for treating pathological conditions resulting from abnormal cell proliferation; transplantation rejections, autoimmune, inflammatory, proliferative, hyperproliferative or vascular diseases; for reducing scar tissue or for inhibiting wound contraction, particularly the prophylactic and/or therapeutic treatment of restenosis by administering at least one compound of the invention that is optionally nitrosated and/or nitrosylated, in combination with nitric oxide donors that are capable of releasing nitric oxide or indirectly delivering or transferring nitric oxide to targeted sites under physiological conditions. The compounds of the invention are preferably estradiol compounds, troglitazone compounds, tranilast compounds, retinoic acid compounds, resveratol compounds, myophenolic acid compounds, acid compounds, anthracenone compounds and trapidil compounds.

### BACKGROUND OF THE INVENTION

Endothelium-derived relaxing factor (EDRF) is a vascular relaxing factor secreted by the endothelium and is important in the control of vascular tone, blood pressure, inhibition of platelet aggregation, inhibition of platelet adhesion, inhibition of mitogenesis, inhibition of proliferation of cultured vascular smooth muscle, inhibition of leukocyte adherence and prevention of thrombosis. EDRF has been identified as nitric oxide (NO) or a closely related derivative thereof (Palmer et al, Nature, 327:524-526 (1987); Ignarro et al, Proc. Natl. Acad. Sci. USA, 84:9265-9269 (1987)).

Removal of the endothelium is a potent stimulus for neointimal proliferation, a common mechanism underlying the restenosis of atherosclerotic vessels after balloon angioplasty (Liu et al., Circulation, 79:1374-1387 (1989); Fems et al., Science, 253:1129-1132 (1991)). Balloon arterial injury results in endothelial denudation and subsequent regrowth of dysfunctional endothelium (Saville, Analyst, 83:670-672 (1958)) that may contribute to the local smooth muscle cell proliferation and extracellular matrix production that result in reocclusion of the arterial lumen. Nitric oxide dilates blood vessels (Vallance et al., Lancet, 2:997-1000 (1989)), inhibits platelet activation and adhesion (Radomski et al., Br. J Pharmacol, 92:181-187 (1987)), and nitric oxide limits the proliferation of vascular smooth muscle cells in vitro (Garg et al., J. Clin. Invest., 83:1774-1777 (1986)). Similarly, in animal models, suppression of platelet-derived mitogens decreases intimal proliferation (Fems et al., Science, 253:1129-1132 (1991)). The potential importance of endothelium-derived nitric oxide in the control of arterial remodeling after injury is further supported by recent preliminary reports in humans suggesting that systemic nitric oxide donors reduce angiographic restenosis six months after balloon angioplasty (The ACCORD Study Investigators, J. Am. Coil. Cardiol. 23:59A. (Abstr.) (1994)).

Another aspect of restenosis may simply be mechanical, e.g., caused by the elastic rebound of the arterial wall and/or by dissections in the vessel wall caused by the angioplasty procedure. These mechanical problems have been successfully addressed by the use of stents to tack-up dissections and prevent elastic rebound of the vessel thereby reducing the level of re-occlusion for many patients. The stent is typically inserted by catheter into a vascular lumen and expanded into contact with the diseased portion of the arterial wall, thereby providing internal support for the lumen. No material has, however, been developed that matches the blood-compatible surface of the endothelium. In fact, in the presence of blood and plasma proteins, artificial surfaces are an ideal setting for platelet deposition (Salzman et al, Phil. Trans. R. Soc. Lond., B294:389-398 (1981)). Exposure of blood to an artificial surface initiates reactions that lead to clotting or platelet adhesion and aggregation. Within seconds of blood contact, the artificial surface becomes coated with a layer of plasma proteins which serves as a new surface to which platelets readily adhere, become activated, and greatly accelerate thrombus formation (Forbes et al, Brit. Med. Bull., 34(2):201-207 (1978)).

Despite considerable efforts to develop nonthrombogenic materials, no synthetic material has been created that is free from this effect. In addition, the use of anticoagulant and platelet inhibition agents has been less than satisfactory in preventing adverse consequences resulting from the interaction between blood and artificial surfaces. Consequently, a significant need exists for the development of additional methods for inhibiting platelet deposition and thrombus formation on artificial surfaces.

There is a need in the art for effective methods of treating cardiovascular diseases and disorders, particularly, restenosis and atherosclerosis. The invention is directed to these, as well as other, important ends.

### SUMMARY OF THE INVENTION

The invention describes novel nitrosated and/or nitrosylated compounds of the invention and methods for treating cardiovascular diseases and disorders by administering one or more nitrosated and/or nitrosylated compounds of the invention, that are capable of releasing a therapeutically effective amount of nitric oxide to a targeted site effected by a cardiovascular disease or disorder. Preferably, the methods of the invention are treating restenosis and atherosclerosis.

One embodiment of the invention provides novel nitrosated and/or nitrosylated compounds. The compounds can be nitrosated and/or nitrosylated through one or more sites such as, oxygen (hydroxyl condensation), sulfur (sulfhydryl condensation) and/or nitrogen. The invention also provides compositions comprising a therapeutically effective amount of such compounds in a pharmaceutically acceptable carrier.

Another embodiment of the invention provides compositions comprising a therapeutically effective amount of at least one compound of the invention, that is optionally substituted with at least one NO and/or NO₂ group (i.e., nitrosylated and/or nitrosated), and at least one nitric oxide donor compound. The invention also provides for such compositions in a pharmaceutically acceptable carrier.

Yet another embodiment of the invention provides compositions comprising a therapeutically effective amount of at least one compound of the invention, that is optionally substituted with at least one NO and/or NO₂ group (i.e., nitrosylated and/or nitrosated), at least one therapeutic agent, and, optionally, at least one nitric oxide donor compound. The invention also provides for such compositions in a pharmaceutically acceptable carrier.

Another embodiment of the invention describes compositions and methods for making compositions comprising at least one compound of the invention, that is optionally substituted with at least one NO and/or NO₂ group (i.e., nitrosylated and/or nitrosated), and, optionally, at least one nitric oxide donor compound and/or at least one therapeutic agent, that are bound to a natural or synthetic matrix, which can be applied with specificity to a biological site of interest. For example, the matrix containing the compounds or compositions of the invention (e.g. nitrosated and/or nitrosylated compounds of the invention) can be used to coat the surface of a medical device that comes into contact with blood (including blood components, blood products and the like), vascular or non-vascular tissue.

Yet another embodiment of the invention provides methods for treating cardiovascular diseases and disorders by administering to a patient in need thereof a therapeutically effective amount of at least one nitrosated and/or nitrosylated compound of the invention, and, optionally, at least one nitric oxide donor compound. The methods can further comprise administering a therapeutically effective amount of at least one therapeutic agent. Alternatively, the methods for treating cardiovascular diseases and disorders can comprise administering a therapeutically effective amount of at least one nitrosated and/or nitrosylated compound of the invention, at least one therapeutic agent, and, optionally, at least one nitric oxide donor compound. Alternatively the methods can comprise administering at least one compound of the invention that is not nitrosated and/or nitrosylated and at least one NO donor, and, optionally, at least one therapeutic agent. The compound of the invention, that is optionally nitrosated and/or nitrosylated, nitric oxide donors, and/or therapeutic agents can be administered separately or as components of the same composition in one or more pharmaceutically acceptable carriers.

Yet another embodiment of the invention describes methods for inhibiting platelet aggregation and platelet adhesion caused by the exposure of blood to a medical device by incorporating at least one nitrosated and/or nitrosylated compound of the invention, that is capable of releasing a therapeutically effective amount of nitric oxide, into and/or on the portion(s) of the medical device that come into contact with blood (including blood components and blood products), vascular or non-vascular tissue. The methods can further comprise incorporating at least one nitric oxide donor compound, and, optionally, at least one therapeutic agent into and/or on the portion(s) of the medical device that come into contact with blood, vascular or non-vascular tissue. Alternatively the methods can comprise incorporating at least one compound of the invention that is not nitrosated and/or nitrosylated, and at least one NO donor, and, optionally, at least one therapeutic agent, into and/or on the portion(s) of the medical device that come into contact with blood (including blood components and blood products), vascular or non-vascular tissue.

Another embodiment of the invention relates to the systemic and/or local administration of at least one compound of the invention, that is optionally substituted with at least one NO and/or NO₂ group, and, optionally, at least one therapeutic agent and/or at least one nitric oxide donor, to treat injured tissue, such as damaged blood vessels.

The invention also provides methods using the compounds and compositions described herein to prevent or treat pathological conditions resulting from abnormal cell proliferation; transplantation rejections; autoimmune, inflammatory, proliferative, hyperproliferative or vascular diseases; for reducing scar tissue or for inhibiting wound contraction by administering to a patient in need thereof a therapeutically effective amount of at least one of the compounds and/or compositions described herein. In these methods, the compounds of the invention, that are optionally nitrosated and/or nitrosylated, nitric oxide donors and therapeutic agents can be administered separately or as components of the same composition in one or more pharmaceutically acceptable carriers.

These and other aspects of the invention are described in detail herein.

### DETAILED DESCRIPTION OF THE INVENTION

As used throughout the disclosure, the following terms, unless otherwise indicated, shall be understood to have the following meanings.

“Cardiovascular disease or disorder” refers to any cardiovascular disease or disorder known in the art, including, but not limited to, restenosis, coronary artery disease, atherosclerosis, atherogenesis, cerebrovascular disease, angina, (particularly chronic, stable angina pectoris), ischemic disease, congestive heart failure, pulmonary edema associated with acute myocardial infarction, aneurysm, thrombosis, hypertension (e.g. pulmonary hypertension, low-renin hypertension, salt-sensitive hypertension, low-renin, salt-sensitive hypertension, thromboembolic pulmonary hypertension; pregnancy-induced hypertension; renovascular hypertension; hypertension-dependent end-stage renal disease, hypertension associated with cardiovascular surgical procedures and the like), platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neointimal hyperplasia following percutaneous transluminal coronary angiograph, and the like. Complications associated with the use of medical devices may occur as a result of increased platelet deposition, activation, thrombus formation or consumption of platelets and coagulation proteins. Such complications, which are within the definition of “cardiovascular disease or disorder,” include, for example, myocardial infarction, pulmonary thromboembolism, cerebral thromboembolism, thrombophlebitis, thrombocytopenia, bleeding disorders and/or any other complications which occur either directly or indirectly as a result of the foregoing disorders.

“Restenosis” is a cardiovascular disease or disorder that refers to the closure of a peripheral or coronary artery following trauma to the artery caused by an injury such as, for example, angioplasty, balloon dilation, atherectomy, laser ablation treatment or stent insertion. For these angioplasty procedures, restenosis occurs at a rate of about 30-60% depending upon the vessel location, lesion length and a number of other variables. Restenosis can also occur following a number of invasive surgical techniques, such as, for example, transplant surgery, vein grafting, coronary artery bypass surgery, endarterectomy, heart transplantation, ballon angioplasty, atherectomy, laser ablation, endovascular stenting, and the like.

“Atherosclerosis” is a form of chronic vascular injury in which some of the normal vascular smooth muscle cells in the artery wall, which ordinarily control vascular tone regulating blood flow, change their nature and develop “cancer-like” behavior. These vascular smooth muscle cells become abnormally proliferative, secreting substances, such as growth factors, tissue-degradation enzymes and other proteins, which enable them to invade and spread into the inner vessel lining, blocking blood flow and making that vessel abnormally susceptible to being completely blocked by local blood clotting, resulting in the death of the tissue served by that artery.

“Autoimmune, inflammatory, proliferative, hyperproliferative or vascular diseases” refers to any autoimmune, inflammatory, proliferative or hyperproliferative disease or disorder known in the art whether of a chronic or acute nature, including, but not limited to, rheumatoid arthritis, restenosis, lupus erythematosus, systemic lupus erythematosus, Hashimotos thyroiditis, myasthenia gravis, diabetes mellitus, uveitis, nephritic syndrome, multiple sclerosis; inflammatory skin diseases, such as, for example, psoriasis, dermatitis, contact dermatitis, eczema and seborrhea; surgical adhesion; tuberculosis; inflammatory lung diseases, such as asthma, pneumoconiosis, chronic obstructive pulmonary disease, emphysema, bronchitis, nasal polyps and pulmonary fibrosis; inflammatory bowel disease, such as Crohn's disease and ulcerative colitis; graft rejections; inflammatory diseases that affect or cause obstruction of a body passageway, such as vasculitis, Wegener's granulomatosis and Kawasaki disease; inflammation of the eye, nose or throat, such as neovascular diseases of the eye including neovascular glaucoma, proliferative diabetic retinopathy, retrolental fibroblasia, macular degeneration, reduction of intraocular pressure, corneal neovascularization, such as corneal infections; immunological processes, such as graft rejection and Steven-Johnson's syndrome, alkali burns, trauma and inflammation (of any cause); fungal infections, such as, for example, infections caused by Candida, Trichophyton, Microsporum, Eepidermophyton, Cryptococcus, Aspergillus, Coccidiodes, Paracocciciodes, Histoplasma or Blastomyces spp; food related allergies, such as, for example, migraine, rhinitis and eczema; vascular diseases, such as arotic aneurysm. A description of inflammatory diseases can also be found in WO 92/05179, WO 98/09972, WO 98/24427, WO 99/62510 and U.S. Pat. No. 5,886,026, the disclosures of each of which are incorporated herein in their entirety.

“Pathological conditions resulting from abnormal cell proliferation” refers to any abnormal cellular proliferation of malignant or non-malignant cells in various tissues and/or organs, including but not limited to, muscle, bone, conjunctive tissues, skin, brain, lungs, sexual organs, lymphatic system, renal system, mammary cells, blood cells, liver, the digestive system, pancreas, thyroid, adrenal glands and the like. These pathological conditions can also include psoriasis; solid tumors; ovarian, breast, brain, prostate, colon, esophageal, lung, stomach, kidney and/or testicular cancer; Karposi's sarcoma, cholangiocarcinoma; choriocarcinoma; neoblastoma; Wilm's tumor; Hodgkin's disease; melanomas; multiple myelomas; chronic lymphocytic leukemias, and acute or chronic granulocytic lymphomas. The treatment of “pathological conditions resulting from abnormal cell proliferation” includes, but is not limited to, reduction of tumor size, inhibition of tumor growth and/or prolongation of the survival time of tumor-bearing patients

“Transplantation” refers to the transplant of any organ or body part, including but not limited to, heart, kidney, liver, lung, bone marrow, cornea and skin transplants.

“Artificial surface” refers to any natural or synthetic material contained in a device or apparatus that is in contact with blood, vasculature or other tissues.

“Blood” includes blood products, blood components and the like.

“Platelet adhesion” refers to the contact of a platelet with a foreign surface, including any artificial surface, such as a medical device, as well as injured vascular or non-vascular surfaces, such as collagen. Platelet adhesion does not require platelet activation. Unactivated, circulating platelets will adhere to injured vascular or non-vascular surfaces or artificial surfaces via binding interactions between circulating von Willdebrand factor and platelet surface glycoprotein Ib/IX.

“Platelet aggregation” refers to the binding of one or more platelets to each other. Platelet aggregation is commonly referred to in the context of generalized atherosclerosis, not with respect to platelet adhesion on vasculature damaged as a result of physical injury during a medical procedure. Platelet aggregation requires platelet activation which depends on the interaction between the ligand and its specific platelet surface receptor.

“Platelet activation” refers either to the change in conformation (shape) of a cell, expression of cell surface proteins (e.g., the IIb/IIIa receptor complex, loss of GPIb surface protein), and secretion of platelet derived factors (e.g., serotonin, growth factors).

“Passivation” refers to the coating of a surface which renders the surface non-reactive.

“Patient” refers to animals, preferably mammals, most preferably humans, and includes males and females, and children and adults.

“Therapeutically effective amount” refers to the amount of the compound and/or composition that is effective to achieve its intended purpose.

“Medical device” refers to any intravascular or extravascular medical devices, medical instruments, medical product, foreign bodies including implants and the like, having a surface that comes in contact with tissue, blood or bodily fluids in the course of its use or operation. Examples of intravascular medical devices and instruments include balloons or catheter tips adapted for insertion, prosthetic heart valves, sutures, surgical staples, synthetic vessel grafts, stents (e.g. Palmaz-Schatz, Wiktor, Crown, Mutlilink, GFX stents), stent grafts, vascular or non-vascular grafts, shunts, aneurysm fillers (including GDC, Guglilmi detachable coils), intraluminal paving systems, guide wires, embolic agents (for example, polymeric particles, spheres and liquid embolics), filters (for example, vena cava filters), arteriovenous shunts, artificial heart valves, artificial implants including, but not limited to, prostheses, foreign bodies introduced surgically into the blood vessels, at vascular or non-vascular sites, leads, pacemakers, implantable pulse generators, implantable cardiac defibrillators, cardioverter defibrillators, defibrillators, spinal stimulators, brain stimulators, sacral nerve stimulators, chemical sensors, breast implants, interventional cardiology devices, catheters, amniocentesis and biopsy needles, and the like. Examples of extravascular medical devices and instruments include plastic tubing, dialysis bags or membranes whose surfaces come in contact with the blood stream of a patient, blood oxygenators, blood pumps, blood storage bags, blood collection tubes, blood filters and/or filtration devices, drug pumps, contact lenses, and the like. The term “medical device” also includes bandages or any external device that can be applied directed to the skin.

“Antioxidant” refers to and includes any compound that can react and quench a free radical.

“Angiotensin converting enzyme (ACE) inhibitor” refers to compounds that inhibit an enzyme which catalyzes the conversion of angiotensin I to angiotensin II. ACE inhibitors include, but are not limited to, amino acids and derivatives thereof, peptides, including di- and tri-peptides, and antibodies to ACE which intervene in the renin-angiotensin system by inhibiting the activity of ACE thereby reducing or eliminating the formation of the pressor substance angiotensin II.

“Angiotensin II antagonists” refers to compounds which interfere with the function, synthesis or catabolism of angiotensin II. Angiotensin II antagonists include peptide compounds and non-peptide compounds, including, but not limited to, angiotensin II antagonists, angiotensin II receptor antagonists, agents that activate the catabolism of angiotensin II, and agents that prevent the synthesis of angiotensin I from angiotensin II. The renin-angiotensin system is involved in the regulation of hemodynamics and water and electrolyte balance. Factors that lower blood volume, renal perfusion pressure, or the concentration of sodium in plasma tend to activate the system, while factors that increase these parameters tend to suppress its function.

“Anti-hyperlipidemic drugs” refers to any compound or agent that has the effect of beneficially modifying serum cholesterol levels such as, for example, lowering serum low density lipoprotein (LDL) cholesterol levels, or inhibiting oxidation of LDL cholesterol, whereas high density lipoprotein (HDL) serum cholesterol levels may be lowered, remain the same, or be increased. Preferably, the anti-hyperlipidemic drug brings the serum levels of LDL cholesterol and HDL cholesterol (and, more preferably, triglyceride levels) to normal or nearly normal levels.

“Neutral endopeptidase inhibitors” refers to and includes compounds that are antagonists of the renin angiotensin aldosterone system including compounds that are dual inhibitors of neutral endopeptidases and angiotensin converting (ACE) enzymes.

“Renin inhibitors” refers to compounds which interfere with the activity of renin.

“Platelet reducing agents” refers to compounds that prevent the formation of a blood thrombus via any number of potential mechanisms. Platelet reducing agents include, but are not limited to, fibrinolytic agents, anti-coagulant agents and any inhibitors of platelet function. Inhibitors of platelet function include agents that impair the ability of mature platelets to perform their normal physiological roles (i.e., their normal function, such as, for example, adhesion to cellular and non-cellular entities, aggregation, release of factors such as growth factors) and the like.

“NSAID” refers to a nonsteroidal anti-inflammatory compound or a nonsteroidal anti-inflammatory drug. NSAIDs inhibit cyclooxygenase, the enzyme responsible for the biosyntheses of the prostaglandins and certain autocoid inhibitors, including inhibitors of the various isozymes of cyclooxygenase (including but not limited to cyclooxygenase-1 and -2), and as inhibitors of both cyclooxygenase and lipoxygenase.

“Cyclooxygenase-2 (COX-2) selective inhibitor” refers to a compound that selectively inhibits the cyclooxygenase-2 enzyme over the cyclooxygenase-1 enzyme. In one embodiment, the compound has a cyclooxygenase-2 IC₅₀ of less than about 2 μM and a cyclooxygenase-1 IC₅₀ of greater than about 5 μM, in the human whole blood COX-2 assay (as described in Brideau et al., Inflamm Res., 45: 68-74 (1996)) and also has a selectivity ratio of cyclooxygenase-2 inhibition over cyclooxygenase-1 inhibition of at least 10, and preferably of at least 40. In another embodiment, the compound has a cyclooxygenase-1 IC₅₀ of greater than about 1 μM, and preferably of greater than 20 μM. The compound can also inhibit the enzyme, lipoxygenase. Such selectivity may indicate an ability to reduce the incidence of common NSAID-induced side effects.

“Therapeutic agent” includes any therapeutic agent that can biologically stent a vessel and/or reduce or inhibit vascular remodeling and/or inhibit or reduce vascular or non-vascular smooth muscle proliferation following a procedural vascular trauma and includes the pro-drugs and pharmaceutical derivatives thereof including, but not limited to, the corresponding nitrosated and/or nitrosylated derivatives. Although nitric oxide donors have therapeutic activity, the term “therapeutic agent” does not include the nitric oxide donors described herein, since nitric oxide donors are separately defined.

“Prodrug” refers to a compound that is made more active in vivo.

“Carriers” or “vehicles” refers to carrier materials suitable for compound administration and include any such material known in the art such as, for example, any liquid, gel, solvent, liquid diluent, solubilizer, or the like, which is non-toxic and which does not interact with any components of the composition in a deleterious manner.

“Sustained release” refers to the release of a therapeutically active compound and/or composition such that the blood levels of the therapeutically active compound are maintained within a desirable therapeutic range over an extended period of time. The sustained release formulation can be prepared using any conventional method known to one skilled in the art to obtain the desired release characteristics.

“Nitric oxide adduct” or “NO adduct” refers to compounds and functional groups which, under physiological conditions, can donate, release and/or directly or indirectly transfer any of the three redox forms of nitrogen monoxide (NO⁺, NO⁻, NO·), such that the biological activity of the nitrogen monoxide species is expressed at the intended site of action.

“Nitric oxide releasing” or “nitric oxide donating” refers to methods of donating, releasing and/or directly or indirectly transferring any of the three redox forms of nitrogen monoxide (NO⁺, NO⁻, NO·), such that the biological activity of the nitrogen monoxide species is expressed at the intended site of action.

“Nitric oxide donor” or “NO donor” refers to compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo and/or are oxidized to produce nitric oxide and/or are substrates for nitric oxide synthase and/or cytochrome P450. “NO donor” also includes compounds that are precursors of L-arginine, inhibitors of the enzyme arginase and nitric oxide mediators.

“Alkyl” refers to a lower alkyl group, a substituted lower alkyl group, a haloalkyl group, a hydroxyalkyl group, an alkenyl group, a substituted alkenyl group, an alkynyl group, a bridged cycloalkyl group, a cycloalkyl group or a heterocyclic ring, as defined herein. An alkyl group may also comprise one or more radical species, such as, for example a cycloalkylalkyl group or a heterocyclicalkyl group.

“Lower alkyl” refers to branched or straight chain acyclic alkyl group comprising one to about ten carbon atoms (preferably one to about eight carbon atoms, more preferably one to about six carbon atoms). Exemplary lower alkyl groups include methyl, ethyl, n-propyl, isopropyl, n-butyl, isobutyl, sec-butyl, t-butyl, pentyl, neopentyl, iso-amyl, hexyl, octyl, and the like.

“Substituted lower alkyl” refers to a lower alkyl group, as defined herein, wherein one or more of the hydrogen atoms have been replaced with one or more R¹⁰⁰ groups, wherein each R¹⁰⁰ is independently a hydroxy, an ester, an amidyl, an oxo, a carboxyl, a carboxamido, a halo, a cyano, a nitrate or an amino group, as defined herein.

“Haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein. Exemplary haloalkyl groups include trifluoromethyl, chloromethyl, 2-bromobutyl, 1-bromo-2-chloro-pentyl, and the like.

“Alkenyl” refers to a branched or straight chain C₂-C₁₀ hydrocarbon (preferably a C₂-C₈ hydrocarbon, more preferably a C₂-C₆ hydrocarbon) that can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like.

“Lower alkenyl” refers to a branched or straight chain C₂-C₄ hydrocarbon that can comprise one or two carbon-carbon double bonds.

“Substituted alkenyl” refers to a branched or straight chain C₂-C₁₀ hydrocarbon (preferably a C₂-C₈ hydrocarbon, more preferably a C₂-C₆ hydrocarbon) which can comprise one or more carbon-carbon double bonds, wherein one or more of the hydrogen atoms have been replaced with one or more R¹⁰⁰ groups, wherein each R¹⁰⁰ is independently a hydroxy, an oxo, a carboxyl, a carboxamido, a halo, a cyano or an amino group, as defined herein.

“Alkynyl” refers to an unsaturated acyclic C₂-C₁₀ hydrocarbon (preferably a C₂-C₈ hydrocarbon, more preferably a C₂-C₆ hydrocarbon) that can comprise one or more carbon-carbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimethyl-butyn-1-yl, and the like.

“Bridged cycloalkyl” refers to two or more cycloalkyl groups, heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo(3.3.0)octane, 7-oxabicyclo(2.2.1)heptyl, 8-azabicyclo(3,2,1)oct-2-enyl and the like.

“Cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo, alkylsulfinyl, and nitro. Exemplary cycloalkyl groups include cyclopropyl, cyclobutyl, cyclopentyl, cyclohexyl, cyclohexenyl, cyclohepta-1,3-dienyl, and the like.

“Heterocyclic ring or group” refers to a saturated or unsaturated cyclic hydrocarbon group having about 2 to about 10 carbon atoms (preferably about 4 to about 6 carbon atoms) where 1 to about 4 carbon atoms are replaced by one or more nitrogen, oxygen and/or sulfur atoms. Sulfur maybe in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, alkylthio, aryloxy, arylthio, arylalkyl, hydroxy, oxo, thial, halo, carboxyl, carboxylic ester, alkylcarboxylic acid, alkylcarboxylic ester, aryl, arylcarboxylic acid, arylcarboxylic ester, amidyl, ester, alkylcarbonyl, arylcarbonyl, alkylsulfinyl, carboxamido, alkylcarboxamido, arylcarboxamido, sulfonic acid, sulfonic ester, sulfonamide nitrate and nitro. Exemplary heterocyclic groups include pyrrolyl, furyl, thienyl, 3-pyrrolinyl, 4,5,6-trihydro-2H-pyranyl, pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrahydrofuranyl, tetrazolyl, pyrrolinyl, pyrrolindinyl, oxazolindinyl 1,3-dioxolanyl, imidazolinyl, imidazolindinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, benzothiazolinyl, quinolinyl, 2,6-dioxabicyclo(3.3.0)octane, and the like.

“Heterocyclic compounds” refer to mono- and polycyclic compounds comprising at least one aryl or heterocyclic ring.

“Aryl” refers to a monocyclic, bicyclic, carbocyclic or heterocyclic ring system comprising one or two aromatic rings. Exemplary aryl groups include phenyl, pyridyl, napthyl, quinoyl, tetrahydronaphthyl, furanyl, indanyl, indenyl, indoyl, and the like. Aryl groups (including bicyclic aryl groups) can be unsubstituted or substituted with one, two or three substituents independently selected from alkyl, alkoxy, alkylthio, amino, alkylamino, dialkylamino, arylamino, diarylamino, alkylarylamino, halo, cyano, alkylsulfinyl, hydroxy, carboxyl, carboxylic ester, alkylcarboxylic acid, alkylcarboxylic ester, aryl, arylcarboxylic acid, arylcarboxylic ester, alkylcarbonyl, arylcarbonyl, amidyl, ester, carboxamido, alkylcarboxamido, carbomyl, sulfonic acid, sulfonic ester, sulfonamido and nitro. Exemplary substituted aryl groups include tetrafluorophenyl, pentafluorophenyl, sulfonamide, alkylsulfonyl, arylsulfonyl, and the like.

“Cycloalkenyl” refers to an unsaturated cyclic C₂-C₁₀ hydrocarbon (preferably a C₂-C₈ hydrocarbon, more preferably a C₂-C₆ hydrocarbon) which can comprise one or more carbon-carbon triple bonds.

“Alkylaryl” refers to an alkyl group, as defined herein, to which is appended an aryl group, as defined herein. Exemplary alkylaryl groups include benzyl, phenylethyl, hydroxybenzyl, fluorobenzyl, fluorophenylethyl, and the like.

“Arylalkyl” refers to an aryl radical, as defined herein, attached to an alkyl radical, as defined herein. Exemplary arylalkyl groups include benzyl, phenylethyl, 4-hydroxybenzyl, 3-fluorobenzyl, 2-fluorophenylethyl, and the like.

“Arylalkenyl” refers to an aryl radical, as defined herein, attached to an alkenyl radical, as defined herein. Exemplary arylalkenyl groups include styryl, propenylphenyl, and the like.

“Cycloalkylalkyl” refers to a cycloalkyl radical, as defined herein, attached to an alkyl radical, as defined herein.

“Cycloalkylalkoxy” refers to a cycloalkyl radical, as defined herein, attached to an alkoxy radical, as defined herein.

“Cycloalkylalkylthio” refers to a cycloalkyl radical, as defined herein, attached to an alkylthio radical, as defined herein.

“Heterocyclicalkyl” refers to a heterocyclic ring radical, as defined herein, attached to an alkyl radical, as defined herein.

“Arylheterocyclic ring” refers to a bi- or tricyclic ring comprised of an aryl ring, as defined herein, appended via two adjacent carbon atoms of the aryl ring to a heterocyclic ring, as defined herein. Exemplary arylheterocyclic rings include dihydroindole, 1,2,3,4-tetra-hydroquinoline, and the like.

“Alkylheterocyclic ring” refers to a heterocyclic ring radical, as defined herein, attached to an alkyl radical, as defined herein. Exemplary alkylheterocyclic rings include 2-pyridylmethyl, 1-methylpiperidin-2-one-3-methyl, and the like.

“Alkoxy” refers to R₅₀O—, wherein R₅₀ is an alkyl group, as defined herein (preferably a lower alkyl group or a haloalkyl group, as defined herein). Exemplary alkoxy groups include methoxy, ethoxy, t-butoxy, cyclopentyloxy, trifluoromethoxy, and the like.

“Aryloxy” refers to R₅₅O—, wherein R₅₅ is an aryl group, as defined herein. Exemplary arylkoxy groups include napthyloxy, quinolyloxy, isoquinolizinyloxy, and the like.

“Alkylthio” refers to R₅₀S—, wherein R₅₀ is an alkyl group, as defined herein.

“Lower alkylthio” refers to a lower alkyl group, as defined herein, appended to a thio group, as defined herein.

“Arylalkoxy” or “alkoxyaryl” refers to an alkoxy group, as defined herein, to which is appended an aryl group, as defined herein. Exemplary arylalkoxy groups include benzyloxy, phenylethoxy, chlorophenylethoxy, and the like.

“Alkoxyalkyl” refers to an alkoxy group, as defined herein, appended to an alkyl group, as defined herein. Exemplary alkoxyalkyl groups include methoxymethyl, methoxyethyl, isopropoxymethyl, and the like.

“Alkoxyhaloalkyl” refers to an alkoxy group, as defined herein, appended to a haloalkyl group, as defined herein. Exemplary alkoxyhaloalkyl groups include 4-methoxy-2-chlorobutyl and the like.

“Cycloalkoxy” refers to R₅₄O—, wherein R₅₄ is a cycloalkyl group or a bridged cycloalkyl group, as defined herein. Exemplary cycloalkoxy groups include cyclopropyloxy, cyclopentyloxy, cyclohexyloxy, and the like.

“Cycloalkylthio” refers to R₅₄S—, wherein R₅₄ is a cycloalkyl group or a bridged cycloalkyl group, as defined herein. Exemplary cycloalkylthio groups include cyclopropylthio, cyclopentylthio, cyclohexylthio, and the like.

“Haloalkoxy” refers to an alkoxy group, as defined herein, in which one or more of the hydrogen atoms on the alkoxy group are substituted with halogens, as defined herein. Exemplary haloalkoxy groups include 1,1,1-trichloroethoxy, 2-bromobutoxy, and the like.

“Hydroxy” refers to —OH.

“Oxo” refers to ═O.

“Oxy” refers to —O⁻R₇₇⁺ wherein R₇₇ is an organic or inorganic cation.

“Oxime” refers to ═N—OR₈₁ wherein R₈₁ is a hydrogen, an alkyl group, an aryl group, an alkylsulfonyl group, an arylsulfonyl group, a carboxylic ester, an alkylcarbonyl group, an arylcarbonyl group, a carboxamido group, an alkoxyalkyl group or an alkoxyaryl group.

“Hydrazone refers to ═N—N(R₈₁)(R′₈₁) wherein R′₈₁ is independently selected from R₈₁, and R₈₁ is as defined herein.

“Hydrazino” refers to H₂N—N(H)—.

“Organic cation” refers to a positively charged organic ion. Exemplary organic cations include alkyl substituted ammonium cations, and the like.

“Inorganic cation” refers to a positively charged metal ion. Exemplary inorganic cations include Group I metal cations such as for example, sodium, potassium, magnesium, calcium, and the like.

“Hydroxyalkyl” refers to a hydroxy group, as defined herein, appended to an alkyl group, as defined herein.

“Nitrate” refers to —O—NO₂.

“Nitrite” refers to —O—NO.

“Thionitrate” refers to —S—NO₂.

“Thionitrite” and “nitrosothiol” refer to —S—NO.

“Nitro” refers to the group —NO₂ and “nitrosated” refers to compounds that have been substituted therewith.

“Nitroso” refers to the group —NO and “nitrosylated” refers to compounds that have been substituted therewith.

“Nitrile” and “cyano” refer to —CN.

“Halogen” or “halo” refers to iodine (I), bromine (Br), chlorine (Cl), and/or fluorine (F).

“Amino” refers to —NH₂, an alkylamino group, a dialkylamino group, an arylamino group, a diarylamino group, an alkylarylamino group or a heterocyclic ring, as defined herein.

“Alkylamino” refers to R₅₀NH—, wherein R₅₀ is an alkyl group, as defined herein. Exemplary alkylamino groups include methylamino, ethylamino, butylamino, cyclohexylamino, and the like.

“Arylamino” refers to R₅₅NH—, wherein R₅₅ is an aryl group, as defined herein.

“Dialkylamino” refers to R₅₂R₅₃N—, wherein R₅₂ and R₅₃ are each independently an alkyl group, as defined herein. Exemplary dialkylamino groups include dimethylamino, diethylamino, methyl propargylamino, and the like.

“Diarylamino” refers to R₅₅R₆₀N—, wherein R₅₅ and R₆₀ are each independently an aryl group, as defined herein.

“Alkylarylamino or arylalkylamino” refers to R₅₂R₅₅N—, wherein R₅₂ is an alkyl group, as defined herein, and R₅₅ is an aryl group, as defined herein.

“Alkylarylalkylamino” refers to R₅₂R₇₉N—, wherein R₅₂ is an alkyl group, as defined herein, and R₇₉ is an arylalkyl group, as defined herein.

“Alkylcycloalkylamino” refers to R₅₂R₈₀N—, wherein R₅₂ is an alkyl group, as defined herein, and R₈₀ is an cycloalkyl group, as defined herein.

“Aminoalkyl” refers to an amino group, an alkylamino group, a dialkylamino group, an arylamino group, a diarylamino group, an alkylarylamino group or a heterocyclic ring, as defined herein, to which is appended an alkyl group, as defined herein. Exemplary aminoalkyl groups include dimethylaminopropyl, diphenylaminocyclopentyl, methylaminomethyl, and the like.

“Aminoaryl” refers to an aryl group to which is appended an alkylamino group, a arylamino group or an arylalkylamino group. Exemplary aminoaryl groups include anilino, N-methylanilino, N-benzylanilino, and the like.

“Thio” refers to —S—.

“Sulfinyl” refers to —S(O)—.

“Methanthial” refers to —C(S)—.

“Thial” refers to ═S.

“Sulfonyl” refers to —S(O)₂⁻.

“Sulfonic acid” refers to —S(O)₂OR₇₆, wherein R₇₆ is a hydrogen, an organic cation or an inorganic cation, as defined herein.

“Alkylsulfonic acid” refers to a sulfonic acid group, as defined herein, appended to an alkyl group, as defined herein.

“Arylsulfonic acid” refers to a sulfonic acid group, as defined herein, appended to an aryl group, as defined herein

“Sulfonic ester” refers to —S(O)₂OR₅₈, wherein R₅₈ is an alkyl group, an aryl group, or an aryl heterocyclic ring, as defined herein.

“Sulfonamido” refers to —S(O)₂—N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylsulfonamido” refers to a sulfonamido group, as defined herein, appended to an alkyl group, as defined herein.

“Arylsulfonamido” refers to a sulfonamido group, as defined herein, appended to an aryl group, as defined herein.

“Alkylthio” refers to R₅₀S—, wherein R₅₀ is an alkyl group, as defined herein (preferably a lower alkyl group, as defined herein).

“Arylthio” refers to R₅₅S—, wherein R₅₅ is an aryl group, as defined herein.

“Arylalkylthio” refers to an aryl group, as defined herein, appended to an alkylthio group, as defined herein.

“Alkylsulfinyl” refers to R₅₀—S(O)—, wherein R₅₀ is an alkyl group, as defined herein.

“Alkylsulfonyl” refers to R₅₀—S(O)₂—, wherein R₅₀ is an alkyl group, as defined herein.

“Alkylsulfonyloxy” refers to R₅₀—S(O)₂—O—, wherein R₅₀ is an alkyl group, as defined herein.

“Arylsulfinyl” refers to R₅₅—S(O)—, wherein R₅₅ is an aryl group, as defined herein.

“Arylsulfonyl” refers to R₅₅—S(O)₂—, wherein R₅₅ is an aryl group, as defined herein.

“Arylsulfonyloxy” refers to R₅₅—S(O)₂—O—, wherein R₅₅ is an aryl group, as defined herein.

“Amidyl” refers to R₅₁C(O)N(R₅₇)— wherein R₅₁ and R₅₇ are each independently a hydrogen atom, an alkyl group, an aryl group or an arylheterocyclic ring, as defined herein.

“Ester” refers to R₅₁C(O)R₇₆— wherein R₅₁ is a hydrogen atom, an alkyl group, an aryl group or an arylheterocyclic ring, as defined herein and R₇₆ is oxygen or sulfur.

“Carbamoyl” refers to —O—C(O)N(R₅₁)(R₅₇), wherein R₅₁ and R₅₇ are each independently a hydrogen atom, an alkyl group, an aryl group or an arylheterocyclic ring, as defined herein, or R₅₁ and R₅₇ taken together are a heterocyclic ring, a cycloalkyl group or a bridged cycloalkyl group, as defined herein.

“Carboxyl” refers to —C(O)OR₇₆, wherein R₇₆ is a hydrogen, an organic cation or an inorganic cation, as defined herein.

“Carbonyl” refers to —C(O)—.

“Alkylcarbonyl” refers to R₅₂—C(O)—, wherein R₅₂ is an alkyl group, as defined herein.

“Arylcarbonyl” refers to R₅₅—C(O)—, wherein R₅₅ is an aryl group, as defined herein.

“Arylalkylcarbonyl” refers to R₅₅—R₅₂—C(O)—, wherein R₅₅ is an aryl group, as defined herein, and R₅₂ is an alkyl group, as defined herein.

“Alkylarylcarbonyl” refers to R₅₂—R₅₅—C(O)—, wherein R₅₅ is an aryl group, as defined herein, and R₅₂ is an alkyl group, as defined herein.

“Heterocyclicalkylcarbonyl” refer to R₇₈C(O)— wherein R₇₈ is a heterocyclicalkyl group, as defined herein.

“Carboxylic ester” refers to —C(O)OR₅₈, wherein R₅₈ is an alkyl group, an aryl group or an aryl heterocyclic ring, as defined herein.

“Alkylcarboxylic acid” and “alkylcarboxyl” refer to an alkyl group, as defined herein, appended to a carboxyl group, as defined herein.

“Alkylcarboxylic ester” refers to an alkyl group, as defined herein, appended to a carboxylic ester group, as defined herein.

“Alkyl ester” refers to an alkyl group, as defined herein, appended to an ester group, as defined herein.

“Arylcarboxylic acid” refers to an aryl group, as defined herein, appended to a carboxyl group, as defined herein.

“Arylcarboxylic ester” and “arylcarboxyl” refer to an aryl group, as defined herein, appended to a carboxylic ester group, as defined herein.

“Aryl ester” refers to an aryl group, as defined herein, appended to an ester group, as defined herein.

“Carboxamido” refers to —C(O)N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylcarboxamido” refers to an alkyl group, as defined herein, appended to a carboxamido group, as defined herein.

“Arylcarboxamido” refers to an aryl group, as defined herein, appended to a carboxamido group, as defined herein.

“Urea” refers to —N(R₅₉)—C(O)N(R₅₁)(R₅₇) wherein R₅₁, R₅₇, and R₅₉ are each independently a hydrogen atom, an alkyl group, an aryl group or an arylheterocyclic ring, as defined herein, or R₅₁ and R₅₇ taken together are a heterocyclic ring, a cycloalkyl group or a bridged cycloalkyl group, as defined herein.

“Phosphoryl” refers to —P(R₇₀)(R₇₁)(R₇₂), wherein R₇₀ is a lone pair of electrons, thial or oxo, and R₇₁ and R₇₂ are each independently a covalent bond, a hydrogen, a lower alkyl, an alkoxy, an alkylamino, a hydroxy, an oxy or an aryl, as defined herein.

“Silyl” refers to —Si(R₇₃)(R₇₄)(R₇₅), wherein R₇₃, R₇₄ and R₇₅ are each independently a covalent bond, a lower alkyl, an alkoxy, an aryl or an arylalkoxy, as defined herein.

The invention is directed to the treatment of cardiovascular diseases and disorders in patients by administering one or more compounds of the invention, that are linked (directly or indirectly) to one or more nitric oxide adducts. Preferably, the compounds of the invention, that are linked to one or more nitric oxide adducts are administered in the form of a pharmaceutical composition that further comprises a pharmaceutically acceptable carrier or diluent. The novel compounds and novel compositions of the invention are described in more detail herein.

Another embodiment of the invention described nitrosated and/or nitrosylated estradiol compounds and pharmaceutically acceptable salts thereof, and/or stereoisomers thereof, of Formula (I): 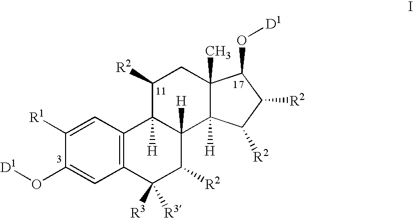
wherein:

R¹ is hydrogen, alkoxy, —O—(C(Rₑ)(R_{f}))ₕ—U—V or —(C(Rₑ)(R_{f}))ₕ—U—V;

R² at each occurrence is independently a hydrogen or —W′ₐ—U—V;

R³and R^{3′} are independently a hydrogen or —O-D¹;

R³and R^{3′} taken together are oxygen or ═N—O-D¹;

D¹ is a hydrogen, V or K;

V is —NO or —NO₂;

K is —W′ₐ-E_{b}-(C(Rₑ)(R_{f}))ₚ′-E_{c}-(C(Rₑ)(R_{f}))ₓ—W′_{d}(C(Rₑ)(R_{f}))_{y}—W′ᵢ-Eⱼ-W′_{g}—(C(Rₑ)(R_{f}))_{z}—U—V;

a, b, c, d, g, i and j are each independently an integer from 0 to 3;

p′, x, y and z are each independently an integer from 0 to 10;

W′ at each occurrence is independently —C(O)—, —C(S)—, -T″-, —(C(Rₑ)(R_{f}))ₕ—, an alkyl group, an aryl group, a heterocyclic ring, an arylheterocyclic ring, or —(CH₂CH₂O)_{q′}—;

E at each occurrence is independently -T″-, an alkyl group, an aryl group, —(C(Rₑ)(R_{f}))ₕ—, a heterocyclic ring, an arylheterocyclic ring, or —(CH₂CH₂O)_{q′}—;

T″ at each occurrence is independently a covalent bond, a carbonyl, an oxygen, —S(O)ₒ— or —N(Rₐ)Rᵢ;

h is an integer form 1 to 10;

q′ is an integer from 1 to 5;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, W′ₕ, —(CH₂)ₒ—U—V, or —(C(R_{g})(Rₕ))ₖ—U—V, or Rₑ and R_{f} taken together with the carbons to which they are attached form a carbonyl, a methanthial, a heterocyclic ring, a cycloalkyl group, an aryl group, an oxime, a hydrazone or a bridged cycloalkyl group;

R_{g} and Rₕ at each occurrence are independently Rₑ;

k is an integer from 1 to 3;

U at each occurrence is independently a covalent bond, a carbonyl, an oxygen, —S(O)ₒ— or —N(Rₐ)Rᵢ;

o is an integer from 0 to 2;

Rₐ is a lone pair of electrons, a hydrogen or an alkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U—V)(Rₑ)(R_{f}), a bond to an adjacent atom creating a double bond to that atom, —(N₂O₂—)⁻·M⁺, wherein M⁺ is an organic or inorganic cation; and

with the proviso that the compounds of Formula (I) must contain at least one NO group, or at least one NO₂ group wherein the at least one NO group or the at least one NO₂ group is linked to the compound of Formula (I) through an oxygen atom, a nitrogen atom or a sulfur atom;

and with the further proviso that one of the substituents selected from —OD¹, R¹, R², R³ and R^{3′} is not each independently —O—NO₂; —OD¹ at C-17 is not —O—(CH₂)ₙ₁—CH(ONO₂)—CH₂—ONO₂ or —O—(CH₂)ₙ₁—CH(ONO₂)—CH(C₁₋₄ lower alkyl)(—ONO₂), wherein n1 is an integer from 1 to 3.

In cases where Rₑ and R_{f} are a heterocyclic ring or taken together Rₑ and R_{f} are a heterocyclic ring, then Rᵢ can be a substituent on any disubstituted nitrogen contained within the radical where Rᵢ is as defined herein.

In cases where multiple designations of variables which reside in sequence are chosen as a “covalent bond” or the integer chosen is 0, the intent is to denote a single covalent bond connecting one radical to another. For example, E₀ would denote a covalent bond, while E₂ denotes (E-E) and (C(Rₑ)(R_{f}))₂ denotes —C(Rₑ)(R_{f})—C(Rₑ)(R_{f})—, where Rₑ and R_{f} at each occurrence are each independently selected from those moieties defined herein.

Another embodiment of the invention describes nitrosated and/or nitrosylated troglitazone compounds of Formula (II) and pharmaceutically acceptable salts thereof: 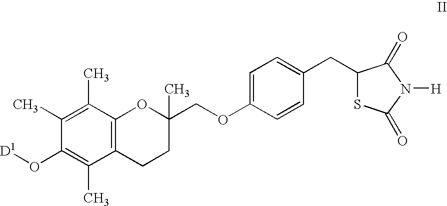
wherein:

D¹ is as defined herein; and

with the proviso that the compounds of Formula (II) must contain at least one NO group, or at least one NO₂ group wherein the at least one NO group or the at least one NO₂ group is linked to the compound of Formula (II) through an oxygen atom, a nitrogen atom or a sulfur atom.

In one embodiment, the invention describes nitrosated and/or nitrosylated tranilast compounds and pharmaceutically acceptable salts thereof, of Formula (III) and pharmaceutically acceptable salts thereof: 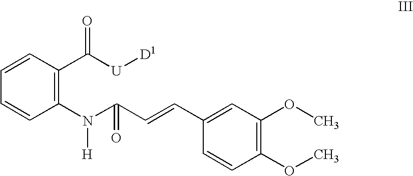
wherein:

D¹ and U are as defined herein; and

with the proviso that the compounds of Formula (III) must contain at least one NO group, or at least one NO₂ group wherein the at least one NO group or the at least one NO₂ group is linked to the compound of Formula (III) through an oxygen atom, a nitrogen atom or a sulfur atom.

Another embodiment of the invention described nitrosated and/or nitrosylated retinoic acid compounds of the Formula (IV) and pharmaceutically acceptable salts thereof: 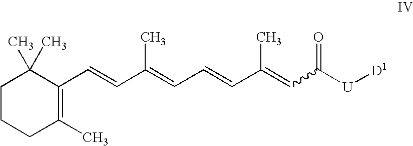
wherein:

U and D¹ are as defined herein; and

with the proviso that the compounds of Formula (IV) must contain at least one NO group, or at least one NO₂ group wherein the at least one NO group or the at least one NO₂ group is linked to the compound of Formula (IV) through an oxygen atom, a nitrogen atom or a sulfur atom.

Another embodiment of the invention described nitrosated and/or nitrosylated resveratrol compounds of Formula (V) and pharmaceutically acceptable salts thereof: 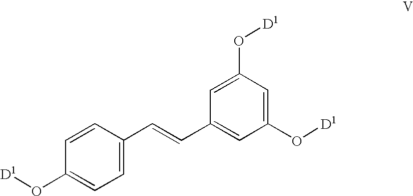
wherein:

D¹ is as defined herein; and

with the proviso that the compounds of Formula (V) must contain at least one NO group, or at least one NO₂ group wherein the at least one NO group or the at least one NO₂ group is linked to the compound of Formula (V) through an oxygen atom, a nitrogen atom or a sulfur atom.

Another embodiment of the invention described nitrosated and/or nitrosylated myophenolic acid compounds of the Formula (VI) and pharmaceutically acceptable salts thereof: 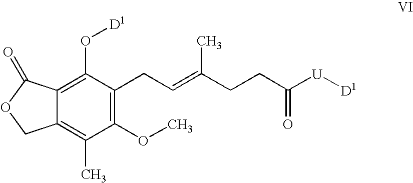
wherein:

U and D¹ are as defined herein; and

with the proviso that the compounds of Formula (VI) must contain at least one NO group, or at least one NO₂ group wherein the at least one NO group or the at least one NO₂ group is linked to the compound of Formula (VI) through an oxygen atom, a nitrogen atom or a sulfur atom.

Another embodiment of the invention described nitrosated and/or nitrosylated acids of Formula (VII) and pharmaceutically acceptable salts thereof: 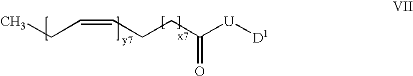
wherein:

x⁷is the integer 2 when y⁷ is the integer 6; or

x⁷is the integer 3 when y⁷ is the integer 5:

U and D¹ are as defined herein; and

with the proviso that the compounds of Formula (VII) must contain at least one NO group, or at least one NO₂ group wherein the at least one NO group or the at least one NO₂ group is linked to the compound of Formula (VII) through an oxygen atom, a nitrogen atom or a sulfur atom.

Another embodiment of the invention described nitrosated and/or nitrosylated anthracenone compounds of Formula (VD) and pharmaceutically acceptable salts thereof: 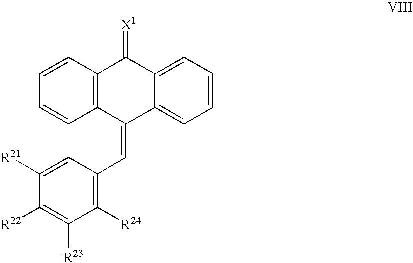
wherein:

X¹ is a oxygen, ═N—OD¹ or ═N—N(X² )D¹;

X² is a hydrogen or a lower alkyl group;

R²¹, R²², R²³ and R²⁴ are each independently a hydrogen, alkoxy, hydroxyl or —OD¹; D¹ is as defined herein; and

with the proviso that the compounds of Formula (VIII) must contain at least one NO group, or at least one NO₂ group wherein the at least one NO group or the at least one NO₂ group is linked to the compound of Formula (VIII) through an oxygen atom, a nitrogen atom or a sulfur atom.

Another embodiment of the invention described nitrosated and/or nitrosylated trapidil derivatives of the Formula (IX) and pharmaceutically acceptable salts thereof: 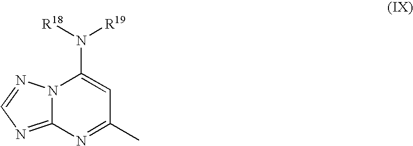
wherein:

R¹⁸ and R¹⁹ are each independently a hydrogen, an alkyl group or K;

K is as defined herein; and

with the proviso that the compounds of Formula (IX) must contain at least one NO group, or at least one NO₂ group wherein the at least one NO group or the at least one NO₂ group is linked to the compound of Formula (IX) through an oxygen atom, a nitrogen atom or a sulfur atom.

Compounds of the invention, which have one or more asymmetric carbon atoms can exist as the optically pure enantiomers, pure diastereomers, mixtures of enantiomers, mixtures of diastereomers, racemic mixtures of enantiomers, diastereomeric racemates or mixtures of diastereomeric racemates. It is to be understood that the invention anticipates and includes within its scope all such isomers and mixtures thereof.

In one embodiment of the invention describes nitrosated compounds of Formula (I), Formula (II), Formula (IV) and Formula (VI) wherein U is —S(O)ₒ— or —N(Rₐ)Rᵢ and V is —NO₂.

In another embodiment of the invention the acid compounds of Fomula (VII) (4Z,7Z,10Z,13Z,16Z,19Z)docosa-4,7,10,13,16,19-hexaenoic acid and nitrosylated (5Z,8Z,11Z,14Z,17Z)icosa-5,8,11,14,17-pentaenoic acid.

In one embodiment, the invention describes nitrosated compounds of the invention that are nitrosated estradiol compounds, nitrosated troglitazone compounds, nitrosated tranilast compounds, nitrosated retinoic acid compounds, nitrosated resveratol compounds, nitrosated mycophenolic acid compounds, nitrosated acid compounds, nitrosated anthracenone compounds and nitrosated trapidil compounds wherein the compounds of the invention are nitrosated by containing or modified to contain at least one nitrosated carboxylic acid group (—C(O)X), nitrosated hydroxyl group (—OX), nitrosated thiol group (—SX) and/or primary or secondary nitrosated amine group (—NX);

wherein X is:

(1) —Y—(CR₄R₄′)ₚ-T-(CR₄R₄′)ₚ—ONO₂;

(2) —Y—(CR₄R₄′)ₚ—ONO₂;

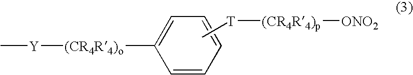

wherein T is ortho, meta or para;

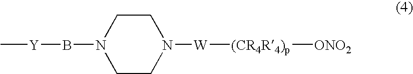

(5) —Y—(CR₄R₄′)ₚ—V—B-T-(CR₄R₄′)ₚ—ONO₂;

(6) —Y—(CR₄R₄′)ₚ-T-C(O)—(CR₄R₄′)ₒ—(CH₂)—ONO₂;

(7) —Y—(CR₄R₄′)ₚ—C(Z)-(CH₂)_{q}-T-(CR₄R₄′)_{q}—(CH₂)—ONO₂;

(8) —Y—(CR₄R₄′)ₚ-T-(CH₂)_{q}—V—(CR₄R₄′)_{q}—(CH₂)—ONO₂;

(9) —Y—(CR₄R₄′)ₚ—V—(CH₂)_{q}—V—(CR₄R₄′)_{q}—(CH₂)—ONO₂;

(10) —Y—(CR₄R₄′)ₒ—(W)_{q}—(CR₄R₄′)ₒ—(CH₂)—ONO₂;

(11) —NRⱼ—O—(CH₂)ₒ—V—(CR₄R₄′)_{q}—(CH₂)—ONO₂;

(12) —NRⱼ—O—(CH₂)ₒ—(W)_{q}—(CR₄R₄′)_{q}—(CH₂)—ONO₂;

(13) —O—NRⱼ—(CH₂)ₒ—(W)_{q}—(CR₄R₄′)_{q}—(CH₂)—ONO₂;

(14) —Y—(CH₂)ₒ—(W)_{q}—(CH₂)ₒ—V—(CR₄R₄′)ₒ-Q′-(CR₄R₄′)ₒ—(CH₂)—ONO₂;

(15) —Y—(CR₄R₄′)ₚ—V—(CH₂)ₒ—(W)_{q}—(CR₄R₄′)_{q}—(CH₂)—ONO₂;

(16) —O—NRⱼ—(CH₂)ₒ—V—(CR₄R₄′)_{q}—(CH₂)—ONO₂;

(17) —Y—(CR₄R₄′)ₒ-Q′-(CR₄R₄′)ₒ—V—(CR₄R₄′)ₒ—(CH₂)—ONO₂;

(18) —Y—(CR₄R₄′)ₒ-Q′-(CR₄R₄′)ₒ—(W)_{q}—(CR₄R₄′)ₒ—(CH₂)—ONO₂;

(19) —Y—(CR₄R₄′)ₚ-T-(CR₄R₄′)ₚ-Q′-(CR₄R₄′)ₒ—(CH₂)—ONO₂;

(20) —Y—(CR₄R₄′)_{q}—C(Z)-(CR₄R₄′)ₒ—(CH₂)—ONO₂;

(21) —Y—(CR₄R₄′)ₚ-Q′-(CR₄R₄′)ₒ—(CH₂)—ONO₂;

(22) —Y—(CR₄R₄′)_{q}—P(O)MM′;

(23) —Y—(CR₄R₄′)ₒ-Q′-(CR₄R₄′)ₒ—(CH₂)—ONO₂;

(24) —Y—(CR₄R₄′)ₒ-Q′-(CR₄R₄′)ₒ-T-(CR₄R₄′)ₒ—(CH₂)—ONO₂;

(25) —Y—(CR₄R₄′)_{q}—(W)_{q}—(CR₄R₄′)ₒ-Q′-(CR₄R₄′)ₒ—(CH₂)—ONO₂;

(26) —Y—(CR₄R₄′)_{q}—V—(CR₄R₄′)ₒ-Q′-(CR₄R₄′)ₒ—(CH₂)—ONO₂;

(27) —Y—(CR₄R₄′)ₚ-(T)ₒ-(W)_{q}—(CR₄R₄′)ₒ—(CH₂)—ONO₂;

(28) —Y—(CR₄R₄′)ₚ—(W)_{q}-(T)ₒ-(CR₄R₄′)ₒ—(CH₂)—ONO₂;

(29) —Y—(CR₄R₄′)_{q}—C(Z)-V—(CR₄R₄′)_{q}—(CH₂)—ONO₂;

(30) —Y—(CR₄R₄′)ₒ—C(R₄)(ONO₂)—(CR₄R₄′)_{q}-(T)ₒ-(W)_{q}-(T)ₒ-(CR₄R₄′)ₒ—R₅;

(31) —Y—(CR₄R₄′)ₒ—V—(CR₄R₄′)ₒ-Q′-(CR₄R₄′)ₒ—(CH₂)—ONO₂;

(32) —Y—(CR₄R₄′)_{q}—C(Z)-Q′-(CR₄R₄′)ₒ—(CH₂)—ONO₂;

(33) —Y—(CR₄R₄′)ₚ—V—(CR₄R₄′)ₚ—(CH₂)—ONO₂;

(34) —Y—(CR₄R₄′)ₚ—V—(CH₂)_{q}-(T)ₒ-(CR₄R₄′)_{q}—(CH₂)—ONO₂;

(35) —Y—(CR₄R₄′)ₚ-(T)ₒ-Q′-(T)ₒ-(CR₄R₄′)_{q}—(CH₂)—ONO₂;

(36) —Y—(CR₄R₄′)_{q}—C(Z)-(CR₄R₄′)_{q}—V—(CR₄R₄′)ₒ-Q′-(CR₄R₄′)ₒ—(CH₂)—ONO₂;

(37) —Y—(CR₄R₄′)_{q}—C(Z)-(CR₄R₄′)_{q}—(W)_{q}—(CR₄R₄′)ₒ-Q′-(CR₄R₄′)ₒ—(CH₂)—ONO₂;

(38) —NRⱼ—O—(CH₂)ₒ—V—(CR₄R₄′)ₒ-Q′-(CH₂)—ONO₂;

(39) —NRⱼ—O—(CH₂)ₒ—(W)_{q}—(CR₄R₄′)ₒ-Q′-(CH₂)—ONO₂;

(40) —O—NRⱼ—(CH₂)ₒ—(W)_{q}—(CR₄R₄′)ₒ-Q′-(CH₂)—ONO₂;

(41) —O—NRⱼ—(CH₂)ₒ—V—(CR₄R₄′)ₒ-Q′-(CH₂)—ONO₂;

(42) —NRⱼ—NRⱼ—(CR₄R₄′)ₚ—(W)_{q}-(T)ₒ-(CR₄R₄′)ₒ—(CH₂)—ONO₂;

(43) —Y—(CR₄R₄′)ₒ-Q′-(CR₄R₄′)ₒ—ONO₂; or

(44) —Y—(CR₄R₄′)ₒ—V—(CR₄R₄′)ₒ-Q-(CR₄R₄′)ₒ—ONO₂;

R₄ and R₄′ at each occurrence are independently a hydrogen, lower alkyl group, —OH, —CH₂OH, —ONO₂, —NO₂ or —CH₂ONO₂; or R₄ and R₄′ taken together with the carbon atom to which they are attached are a cycloalkyl group or a heterocyclic ring;

V is —C(O)-T-, -T-C(O)—, -T-C(O)-T or T-C(O)—C(O)-T;

W is a covalent bond or a carbonyl group;

T at each occurrence is independently an oxygen, (S(O)ₒ)ₒ or NRⱼ;

Rⱼ is a hydrogen, an alkyl group, an aryl group, a heterocyclic ring, an alkylcarbonyl group, an alkylaryl group, an alkylsulfinyl group, an alkylsulfonyl group, an arylsulfinyl group, an arylsulfonyl group, a sulfonamido group, a N-alkylsulfonamido group, a N,N-diarylsulfonamido group, a N-arylsulfonamido group, a N-alkyl-N-arylsulfonamido group, a carboxamido group or a hydroxyl group;

p at each occurrence is independently an integer from 1 to 6;

q at each occurrence is independently an integer from 1 to 3;

Y is oxygen, sulfur (—S—), NRⱼ or a covalent bond;

B is either phenyl or (CH₂)ₒ;

Q′ is a cycloalkyl group, a heterocyclic ring or an aryl group;

Z is (═O), (═N—OR₅), (═N—NR₅R′₅) or (═CR₅R′₅);

M and M′ are each independently —O⁻H₃N⁺—(CR₄R′₄)_{q}—CH₂ONO₂ or -T-(CR′4)ₒ—CH₂ONO₂;

R₅ and R₅′ at each occurrence are independently a hydrogen, a hydroxyl group, an alkyl group, an aryl group, an alkylsulfonyl group, an arylsulfonyl group, a carboxylic ester, an alkylcarbonyl group, an arylcarbonyl group, a carboxamido group, an alkoxyalkyl group, an alkoxyaryl group, a cycloalkyl group or a heterocyclic ring;

o is an integer from 0 to 2; and

with the proviso that the nitrosated compounds of the invention must contain at least one NO₂ group; wherein the at least one NO₂ group is linked to the compound through an oxygen atom, a nitrogen atom or a sulfur atom.

It is also to be understood that the invention is intended to include within its scope compounds which may exist in more than one resonance form and the effects that the resonance form may have on the positions at the X substituent designated in the compounds described herein.

In preferred embodiments of the invention for the nitrosated estradiol compounds, nitrosated troglitazone compounds, nitrosated tranilast compounds, nitrosated retinoic acid compounds, nitrosated resveratol compounds, nitrosated mycophenolic acid compounds, nitrosated acid compounds, nitrosated anthracenone compounds and nitrosated trapidil compounds and pharmaceutically acceptable salts thereof, X is: 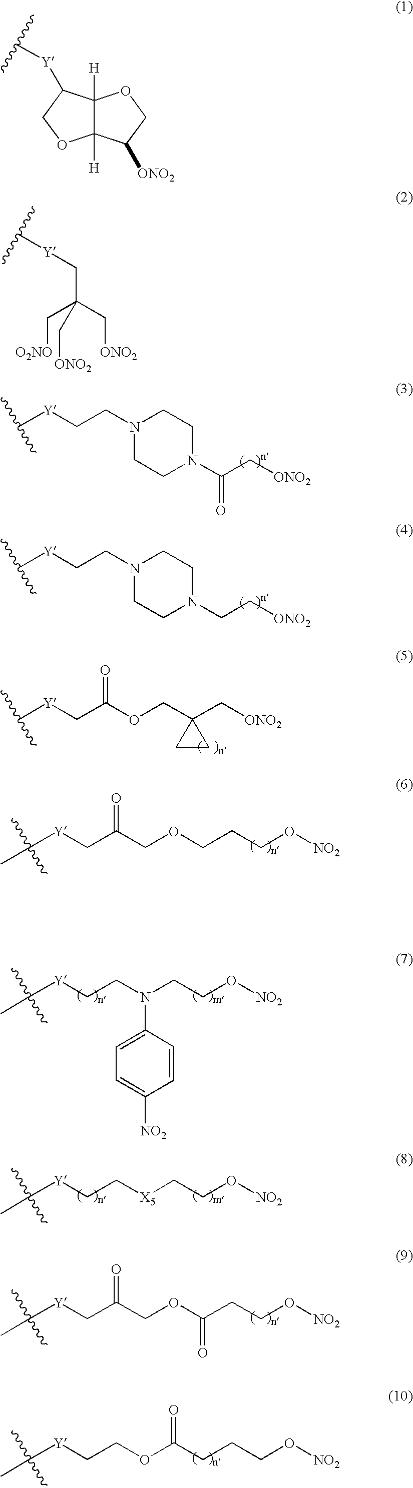 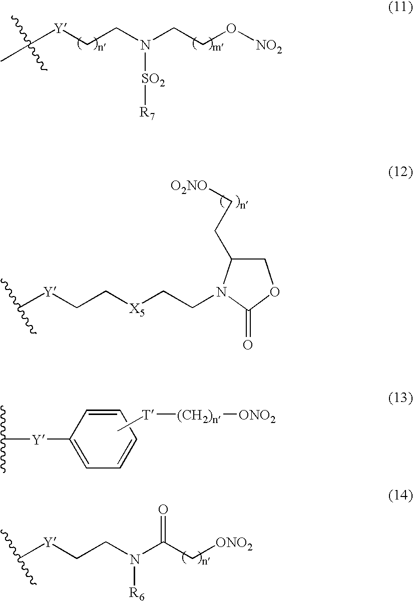
wherein T′ maybe ortho, meta or para 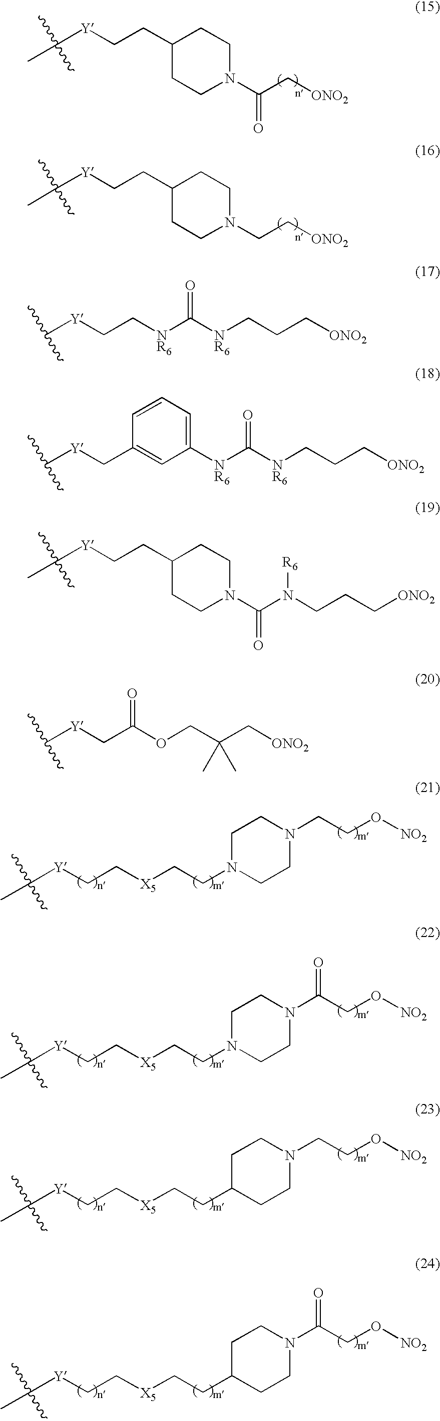 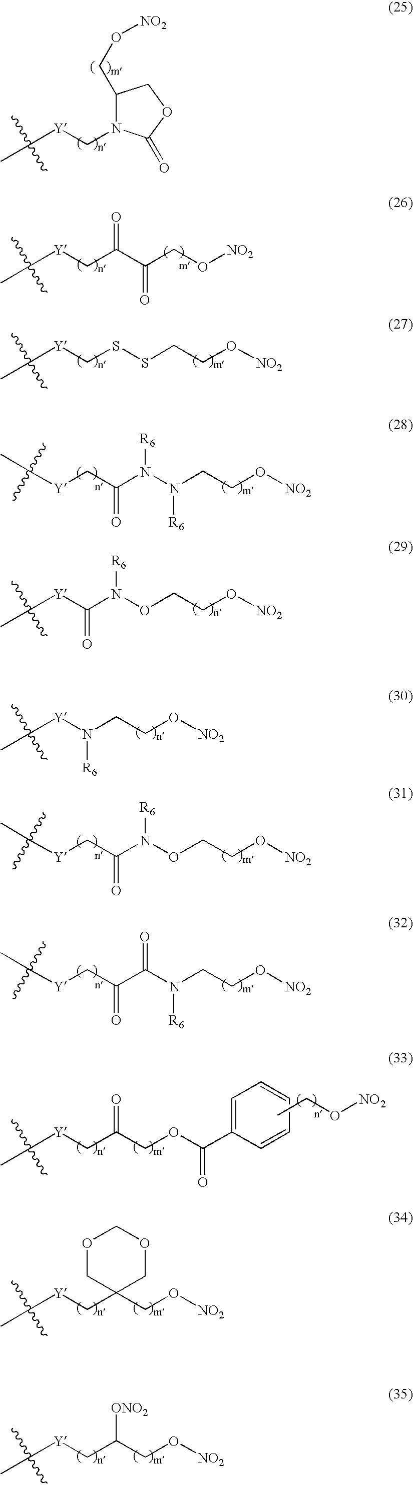 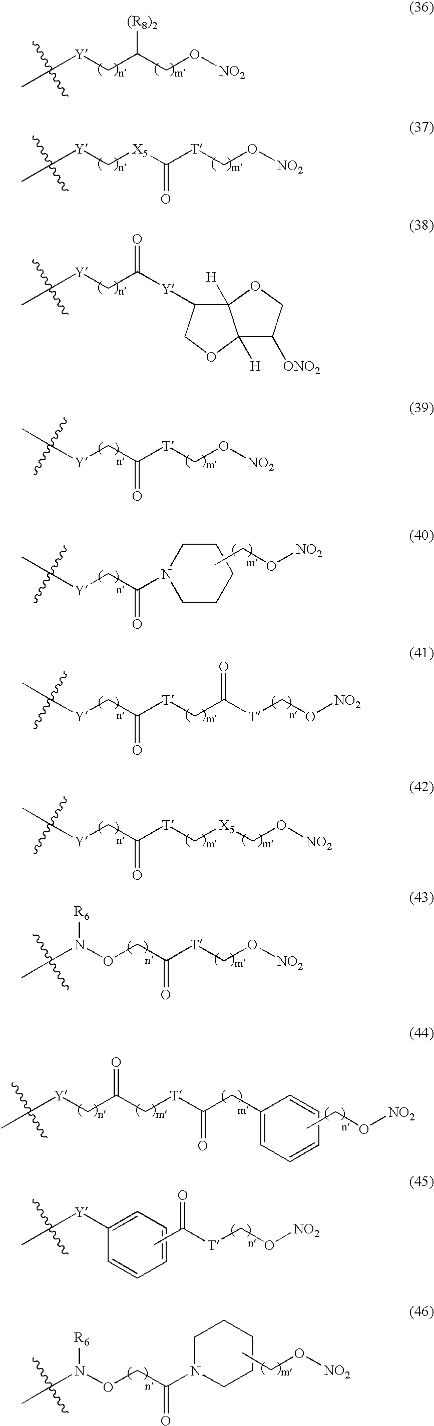 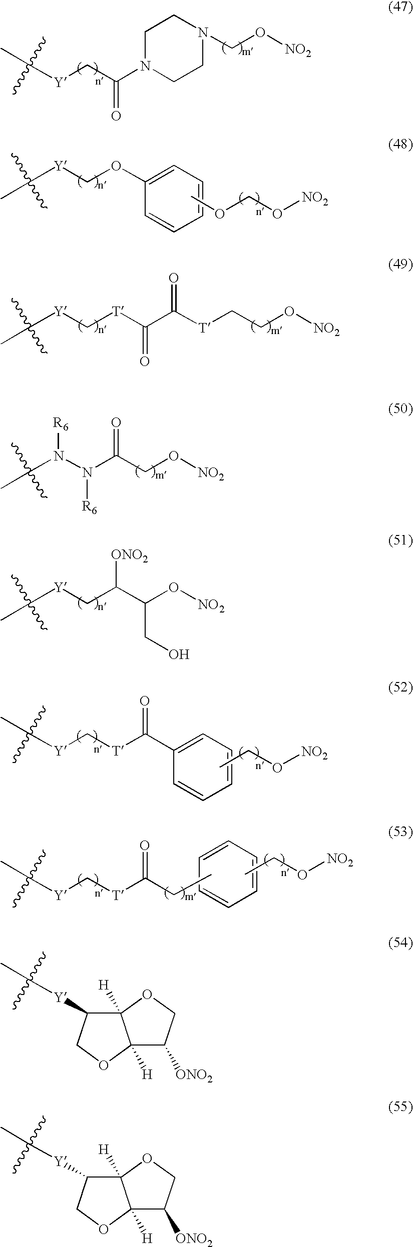
wherein:

Y′ is oxygen or sulfur;

T′ is oxygen, sulfur or NR₆;

X₅ is oxygen, (S(O)ₒ)ₒ or NR₆;

R₆ is a hydrogen, a lower alkyl group, an aryl group;

R₇ is a lower alkyl group or an aryl group;

R₈ at each occurrence is independently is a hydrogen, a hydroxyl group, a lower alkyl group, an aryl group, —NO₂, —CH₂—ONO₂ or —CH₂—OH;

n′ and m′ are each independently an integer from 0 to 10; and

o is as an integer from 0 to 2.

In another embodiment of the invention, the nitrosated compounds of the invention do not include the compounds disclosed in WO 02/51385, WO 01/54691, WO 00/61549, WO 00/61541, WO 00/61537, the disclosures of each of which are incorporated by reference herein in their entirety.

In yet another embodiment the nitrosylated estradiol compounds of Formula (I) are:

(1S,11S,14S,15S,10R)-14-Hydroxy-4-methoxy-15-methyltetracyclo(8.7.0.0<2,7>.0<11,15>)heptadeca-2(7),3,5-trien-5-yl 3-methyl-3-(nitrosothio)butanoate;

(1S,11S,14S,15S,10R)-4-methoxy-15-methyl-14-(nitrosooxy)tetracyclo(8.7.0.0<2,7>.0<11,15>)heptadeca-2(7),3,5-trien-5-yl 3-methyl-3-(nitrosothio)butanoate;

(1S,11S,14S,15S,10R)-4-Methoxy-15-methyl-14-(3-methyl-3-(nitrosothio)butanoyloxy)tetracyclo-(8.7.0.0<2,7>.0<11,15>)heptadeca-2(7),3,5-trien-5-yl 3-methyl-3-(nitrosothio)butanoate;

(1S,11S,14S,15S,10R)-14-Hydroxy-15-methyltetracyclo (8.7.0.0<2,7>.0<11,15>)heptadeca-2(7),3,5-trien-5-yl 3-methyl-3-(nitrosothio)butanoate;

(1S,11S,14S,15S,10R)-15-methyl-14-(nitrosooxy)tetracyclo(8.7.0.0<2,7>.0<11,15>)heptadeca-2(7),3,5-trien-5-yl 3-methyl-3-(nitrosothio)butanoate;

(1S,11S,14S,15S,10R)-15-methyl-5-(3-methyl-3-(nitrosothio)butanoyloxy)tetracyclo(8.7.0.0<2,7>.0<11,15>)heptadeca-2,4,6-trien-14-yl 3-methyl-3-(nitrosothio)butanoate;

(1S,11S,14S,15S,10R)-14-Hydroxy-15-methyltetracyclo(8.7.0.0<2,7>.0<11,15>)heptadeca-2(7),3,5-trien-5-yl 3-(N-(2-methyl-2-(nitrosothio)propyl)carbamoyl)propanoate;

(1S,11S,14S,15S,10R)-15-Methyl-5-(2-(2-(nitrosothio)adamantan-2-yl)acetyloxy)tetracyclo(8.7.0.0<2,7>.0<11,15>)heptadeca-2,4,6-trien-14-yl 2,2,2-trifluoroacetate;

(1S,11S,14S,15S,10R)-14-hydroxy-15-methyltetracyclo(8.7.0.0<2,7>.0<11,15>)heptadeca-2,4,6-trien-5-yl 2-(2-(nitrosothio)adamantan-2-yl)acetate;

(1S,11S,14S,15S,10R)-14-hydroxy-15-methyltetracyclo(8.7.0.0<2,7>.0<11,15>)heptadeca-2,4,6-trien-5-yl 3,3-dimethyl-4-(N-(2-methyl-2(nitrosothio)propyl)carbamoyl)butanoate;

(1S,11S,14S,15S,10R)-14-hydroxy-15-methyltetracyclo(8.7.0.0<2,7>.0<11,15>)heptadeca-2,4,6-trien-5-yl 3-(N-(2-methyl-2-(nitrosothio)propyl)-N-benzylcarbamoyl)propanoate;

(1S,11S,14S,15S,10R)-15-Methyl-5-(3-(N-(2-methyl-2-(nitrosothio)propyl)-N-benzylcarbamoyl)propanoyloxy)tetracyclo(8.7.0.0<2,7>.0<11,15>)heptadeca-2,4,6-trien-14-yl 3-(N-(2-methyl-2-(nitrosothio)propyl)-N-benzylcarbamoyl)propanoate;

(1S,11S,14S,15S,10R)-14-Hydroxy-15-methyltetracyclo(8.7.0.0<2,7>.0<11,15>)heptadeca-2,4,6-trien-5-yl 3-(N-(2,2-dimethylpropyl)-N-(2-methyl-2-(nitrosothio)propyl)carbamoyl)propanoate;

(1S,11S,14S,15S,10R)-14-Hydroxy-15-methyltetracyclo(8.7.0.0<2,7>.0<11,15>)heptadeca-2,4,6-trien-5-yl 2-(2-(nitrosothio)adamantan-2-yl)ethyl butane-1,4-dioate;

(1S,11S,14S,15S,10R)-15-Methyl-5-phenylcarbonyloxytetracyclo(8.7.0.0<2,7>.0<11,15>)heptadeca-2,4,6-trien-14-yl 2-(2-(nitrosothio)adamantan-2-yl)ethylbutane-1,4-dioate;

(2R)-2,3-Bis(nitrooxy)propyl(1S,11S,14S,15S,10R)-14-hydroxy-15-methyltetracyclo(8.7.0.0<2,7>.0<11,15>)heptadeca-2,4,6-trien-5-yl butane-1,4-dioate;

(1S,11S,14S,15S,10R)-14-Hydroxy-15-methyltetracyclo(8.7.0.0<2,7>.0<11,15>)heptadeca-2,4,6-trien-5-yl 2-(4,4-dimethyl-1-(nitrosothio)cyclohexyl)ethylbutane-1,4-dioate;

(1S,11S,14S,15S,10R)-15-methyl-14-(nitrosooxy)tetracyclo(8.7.0.0<2,7>.0<11,15>)heptadeca-2,4,6-trien-5-yl 2-(4,4-dimethyl-1-(nitrosothio)cyclohexyl)ethylbutane-1,4-dioate;

(1S,11S,14S,15S,10R)-14-hydroxy-15-methyltetracyclo(8.7.0.0<2,7>.0<11,-15>)heptadeca-2,4,6-trien-5-yl 4-(N-(((nitrosothio)cyclohexyl)methyl)-carbamoyl)butanoate;

2-(2-(Nitrosothio)adamantan-2-yl)ethyl 2-((1S,11S,14S,15S,10R)-14-hydroxy-15-methyltetracyclo(8.7.0.0<2,7>.0<11,15>)heptadeca-2,4,6-trien-5-yloxy)acetate;

2-(2-(Nitrosothio)adamantan-2-yl)ethyl 2-(((1S,11S,14S,15S,10R)-5,14-dihydroxy-15-methyltetracyclo(8.7.0.0<2,7>.0<11,15>)heptadeca-2,4,6-trien-8-ylidene)azamethoxy)acetate;

2-(((1S,11S,14S,15S,10R)-5,14-dihydroxy-15-methyltetracyclo(8.7.0.0<2,7>.0<11,15>)heptadeca-2,4,6-trien-8-ylidene)azamethoxy)-N-(2-methyl-2-(nitrosothio)propyl)acetamide;

2-((1S,11S,14S,15S,10R)-14-Hydroxy-15-methyltetracyclo(8.7.0.0<2,7>.0<11,15>)heptadeca-2,4,6-trien-5-yloxy)-N-(2-methyl-2-(nitrosothio)propyl)acetamide;

2-(4-(1-methyl-1-(nitrosothio)ethyl)-2-oxo-1,3-oxazolidin-3-yl)ethyl 2-(((1S,11S,14S,15S,10R)-5,14-dihydroxy-15-methyltetracyclo(8.7.0.0<2,7>.0<11,15>)heptadeca-2,4,6-trien-8-ylidene)azamethoxy)acetate;

2-(4-(1-methyl-1-(nitrosothio)ethyl)-2-oxo-1,3-oxazolidin-3-yl)ethyl 2-((1S,11S,14S,15S,10R)-14-hydroxy-15-methyltetracyclo(8.7.0.0<2,7>.0<11,15>)heptadeca-2,4,6-trien-5-yloxy)acetate;

the nitrosylated troglitazone compounds of Formula (II) are:

2-((4-((2,4-dioxo(1,3-thiazolidin-5-yl))methyl)phenoxy)methyl)-2,5,7,8-tetramethylchroman-6-yl 4-(N-(2-methyl-2-(nitrosothio)propyl)carbamoyl)butanoate;

2-((4-((2,4-dioxo(1,3-thiazolidin-5-yl))methyl)phenoxy)methyl)-2,5,7,8-tetramethylchroman-6-yl 2-(((N-(2-methyl-2-(nitrosothio)propyl)-N-benzylcarbamoyl)methyl)cyclopentyl)acetate;

the nitrosylated tranilast compounds of Formula (III) are:

(N-(2-Methyl-2-(nitrosothio)propyl)carbamoyl)methyl 2-((2E)-3-(3,4-dimethoxyphenyl)prop-2-enoylamino)benzoate;

3-Methyl-3-(nitrosothio)butyl 2-(2-((2E)-3-(3,4-dimethoxyphenyl)prop-2-enoylamino)phenylcarbonyloxy)acetate;

2-(4-(2-Methyl-2-(nitrosothio)propyl)piperazinyl)-2-oxoethyl 2-((2E)-3-(3,4-dimethoxyphenyl)prop-2-enoylamino)benzoate;

2-(4-(2-Methyl-2-(nitrosothio)propyl)piperazinyl)ethyl 2-(2-(92E)-3-(3,4-dimethoxyphenyl)prop-2-enoylamino)phenyloxycarbonyloxy)acetate;

the nitrosylated retinoic acid compounds of Formula (IV) are:

2-(2-(Nitroso)adamantan-2-yl)ethyl (2E,4E,6E,8E)-3,7-dimethyl-9-(2,6,6-trimethylcyclohex-1-enyl)nona-2,4,6,8-tetraenoate;

the nitrosylated anthracenone compounds of Formula (VIII) are:

2-((10-((3-Hydroxy-4-methoxyphenyl)methylene)(9-anthrylidene))-azamethoxy)-N-(2-methyl-2-(nitrosothio)propyl)acetamide;

the nitrosylated trapidil compounds of Formula (IX) are:

(7-Methyl(4-hydro-1,2,4-triazolo(1,5-a)pyrimidin-5-yl))(2-methyl-2(nitrosothio)propyl)amine;

2-(2-(Nitrosothio)adamantan-2-yl)ethyl 1-(7-methyl-4-hydro-1,2,4-triazolo(1,5-a)pyrimidin-5-yl)piperidine-4-carboxylate;

the nitrosated estradiol compounds of Formula (I) are:

(2R)-2,3-Bis(nitrooxy)propyl(1S,11S,14S,15S,10R)-15-methyl-5-phenylcarbonyloxytetracyclo(8.7.0.0<2,7>.0<11,1 5>)heptadeca-2,4,6-trien-14-yl butane-1,4-dioate;

(1S,11S,14S,15S,10R)-15-Methyl-5-phenylcarbonyloxytetracyclo(8.7.0.0<2,7>.0<11,15>)heptadeca-2,4,6-trien-14-yl (1S,2S,5S,6R)-6-(nitrooxy)-4,8-dioxabicyclo(3.3.0)oct-2-yl butane-1,4-dioate;

(1S,11S,14S,15S,10R)-15-Methyl-5-phenylcarbonyloxytetracyclo(8.7.0.0<2,7>.0<11,15>)heptadeca-2,4,6-trien-14-yl 3-(nitrooxy)propylbutane-1,4-dioate;

(1S,11S,14S,15S,10R)-14-hydroxy-15-methyltetracyclo(8.7.0.0<2,7>.0<11,15>)heptadeca-2,4,6-trien-5-yl 2-(2,2-dimethyl-3-(nitrooxy)propanoylamino)-3-((2,4,6-trimethoxyphenyl)methylthio)propanoate;

(1S,11S,14S,15S,10R)-14-hydroxy-15-methyltetracyclo(8.7.0.0<2,7>.0<11,15>)heptadeca-2,4,6-trien-5-yl 3-acetylthio-2-(2,2-dimethyl-3-(nitrooxy)propanoylamino)propanoate;

(1S,11S,14S,15S,10R)-14-Hydroxy-15-methyltetracyclo(8.7.0.0<2,7>.0<11,15>)heptadeca-2,4,6-trien-5-yl (1S,2S,5S,6R)-6-(nitrooxy)-4,8-dioxabicyclo(3.3.0)oct-2-yl butane-1,4-dioate;

(1S,2S,5S,6R)-6-(Nitrooxy)-4,8-dioxabicyclo(3.3.0)oct-2-yl 2-((((1S,11S,14S,15S,10R)-14-hydroxy-15-methyltetracyclo(8.7.0.0<2,7>.0<11,15>)heptadeca-2,4,6-trien-5-yl)oxycarbonyl)methoxy)acetate;

2-(((1S,11S,14S,15S,10R)-5,14-Dihydroxy-15-methyltetracyclo(8.7.0.0<2,7>.0<11,1 5>)heptadeca-2,4,6-trien-8-ylidene)azamethoxy)-N-methyl-N-(2-(nitrooxy)ethyl)acetamide;

2-(((1S,11S,14S,15S,10R)-5,14-dihydroxy-15-methyltetracyclo(8.7.0.0<2,7>.0<11,15>)heptadeca-2,4,6-trien-8-ylidene)azamethoxy)-1-(4-((nitrooxy)methyl)piperidyl)ethan-1-one;

2-(((1S,11S,14S,15S,10R)-5,14-dihydroxy-15-methyltetracyclo(8.7.0.0<2,7>.0<11,15>)heptadeca-2,4,6-trien-8-ylidene)azamethoxy)-N-(2-(nitrooxy)ethyl)acetamide;

2-(((1S,11S,14S,15S,10R)-5,14-dihydroxy-15-methyltetracyclo(8.7.0.0<2,7>.0<11,15>)heptadeca-2,4,6-trien-8-ylidene)azamethoxy)-1-(4-(2-(nitrooxy)ethyl)piperidyl)ethan-1-one;

(1S,11S,14S,15S,10R)-5-hydroxy-15-methyltetracyclo(8.7.0.0<2,7>.0<11,15>)heptadeca-2(7),3,5-trien-14-yl 5-(nitrooxy)pentanoate;

(1S,11S,14S,15S,10R)-5-hydroxy-15-methyltetracyclo(8.7.0.0<2,7>.0<11,15>)heptadeca-2(7),3,5-trien-14-yl 3-((nitrooxy)methyl)benzoate;

(1S,11S,14S,15S,10R)-5-hydroxy-15-methyltetracyclo(8.7.0.0<2,7>.0<11,15>)heptadeca-2(7),3,5-trien-14-yl 2-(6-((nitrooxy)methyl)-2-pyridyl)acetate;

(1S,11S,14S,15S,10R)-5-hydroxy-15-methyltetracyclo(8.7.0.0<2,7>.0<11,15>)heptadeca-2(7),3,5-trien-14-yl 3,4-bis(nitrooxy)butanoate;

(1S,11S,14S,15S,10R)-5-hydroxy-15-methyltetracyclo(8.7.0.0<2,7>.0<11,15>)heptadeca-2(7),3,5-trien-14-yl 2,4-bis(nitrooxy)butanoate;

(1S,11S,14S,15S,10R)-5-hydroxy-15-methyltetracyclo(8.7.0.0<2,7>.0<11,15>)heptadeca-2(7),3,5-trien-14-yl 3-(2-(nitrooxy)ethoxy)propanoate;

(1S,11S,14S,15S,10R)-5-hydroxy-15-methyltetracyclo(8.7.0.0<2,7>.0<11,15>)heptadeca-2(7),3,5-trien-14-yl 3-(methyl(2-(nitrooxy)ethyl)amino)propanoate;

(1S,11S,14S,15S,10R)-5-hydroxy-15-methyltetracyclo(8.7.0.0<2,7>.0<11,15>)heptadeca-2(7),3,5-trien-14-yl 3-(2-(nitrooxy)ethylthio)propanoate;

the nitrosated retinoic acid compounds of Formula (IV) are:

2,2-Bis((nitrooxy)methyl)-3-(nitrooxy)propyl(2E,4E,6E,8E)-3,7-dimethyl-9-(2,6,6-trimethylcyclohex-1-enyl)nona-2,4,6,8-tetraenoate;

(2R)-2,3-Bis(nitrooxy)propyl(2E,4E,6E,8E)-3,7-dimethyl-9-(2,6,6-trimethylcyclohex-1-enyl)nona-2,4,6,8-tetraenoate;

the nitrosated anthracenone compounds of Formula (VIII) are:

2-((10-((3-Hydroxy-4-methoxyphenyl)methylene)(9-anthrylidene))azamethoxy)-1-(4-((nitrooxy)methyl)piperidyl)ethan-1-one;

2-(2-Methoxy-5-((10-oxo(9-anthrylidene))methyl)phenoxy)-1-(4-((nitrooxy)methyl)piperidyl)ethan-1-one.

The compounds of Formula (I) to (IX) can be synthesized following the methods described herein. The reactions are performed in solvents appropriate to the reagents, and materials used are suitable for the transformations being effected. It is understood by one skilled in the art of organic synthesis that the functionality present in the molecule must be consistent with the chemical transformation proposed. This will, on occasion, necessitate judgment by the routineer as to the order of synthetic steps, protecting groups required, and deprotection conditions. Substituents on the starting materials may be incompatible with some of the reaction conditions required in some of the methods described, but alternative methods and substituents compatible with the reaction conditions will be readily apparent to one skilled in the art. The use of sulfur and oxygen protecting groups is known in the art for protecting thiol and alcohol groups against undesirable reactions during a synthetic procedure and many such protecting groups are known, e.g., T. H. Greene and P. G. M. Wuts, Protective Groups in Organic Synthesis, John Wiley & Sons, New York (1999), which is incorporated herein in its entirety.

The synthesis of the parent compound (i.e. non-nitrosated and/or non-nitrosylated compounds of the invention including the pro-drugs and pharmaceutical derivatives thereof) are disclosed in, for example, U.S. Pat. Nos. 4,623,724. 5,385,935 and 6,091,104 and in WO 97/28793 for the compounds of Formula II; U.S. Pat. No. 4,572,912 and in WO 00/43007 for the compounds of Formula III; U.S. Pat. Nos. 3,705,894, 3,705,946, 3,777,020, 3,868,454, 3,880,995, 3,903,071, 4,115,197, 4,234,684, 4,686,234, 4,727,069, 4,753,935, 4,786,637, 5,380,879, 5,441,953, 5,444,072, 5,493,030, 5,516,781, 5,536,747, 5,538,969, 5,554,612, 5,563,136, 5,646,160, 5,633,279, 5,807,876, 5,916,585, 6,107,052 and in WO 94/12184, WO 94/28892, WO 95/22534, WO 95/22535, WO 95/22536, WO 95/22537, WO 95/22538 for the compounds of Formula VI; the disclosure of each of these patents and applications is incorporated by reference herein in its entirety. The parent compound of Formula I, IV, V, VII and VIII are readily available from commercially sources or can be synthesis using known methods.

Some of the compounds of the invention, are synthesized as shown in Schemes 1 through 21 given below, in which D¹, E, K, U, V, W′, T″, Rₑ, R_{f}, Rₐ, Rᵢ, a, b, c, d, g, h, i, j, k, o, p′, q′, x, y and z are as defined herein or as depicted in the reaction schemes for compounds of Formula I-IX; P¹ is an oxygen protecting group; P² is a sulfur protecting group and P³ is a nitrogen protecting group. Nitroso compounds of Formula (I), wherein Rₑ, R_{f}, and p′ are as defined herein and a nitrite containing carboxylic ester is representative of the O-D¹ group as defined herein can be prepared as shown in Scheme 1. The acid of the compound of Formula 1 is converted into the ester of Formula 2 wherein p′, Rₑ, R_{f} and P¹ are defined as herein, by reaction with an appropriate monoprotected diol. Preferred methods for the preparation of esters are forming the mixed anhydride via reaction of the acid with a chloroformate, such as isobutylchloroformate, in the presence of a non-nucleophilic base, such as triethylamine, in an anhydrous inert solvent, such as dichloromethane, diethylether or THF. The mixed anhydride is then reacted with the monoprotected alcohol, preferably in the presence of a condensation catalyst, such as 4-dimethylamino pyridine (DMAP). Alternatively, the acid may first be converted to the acid chloride by treatment with oxalyl chloride in the presence of a catalytic amount of DMF. The acid chloride is then reacted with the monoprotected alcohol, preferably in the presence of a condensation catalyst, such as DMAP, and a tertiary amine base, such as triethyl amine, to produce the ester. Alternatively, the acid and monoprotected diol may be coupled to produce the ester by treatment with a dehydration agent, such as dicyclohexylcarbodiimide (DCC) or 1-(3-dimethylaminopropyl)-3-ethylcarbodiimide hydrochloride (EDAC.HCl) with or without a condensation catalyst, such as DMAP or 1-hydroxybenzotriazole (HOBt). Alternatively, the acid may first be converted into an alkali metal salt, such as the sodium, potassium or lithium salt, and reacted with an alkyl halide that also contains a protected hydroxyl group in a polar solvent, such as DMF, to produce the ester. Preferred protecting groups for the alcohol moiety are silyl ethers, such as a trimethylsilyl or a tert-butyldimethylsilyl ether. Deprotection of the hydroxyl moiety in the compound of Formula 2 (fluoride ion is the preferred method for removing silyl ether protecting groups) followed by reaction with a suitable nitrosylating agent, such as thionyl chloride nitrite, thionyl dinitrite or nitrosonium tetrafluoroborate, in a suitable anhydrous solvent, such as CH₂Cl₂, THF, DMF or acetonitrile, with or without an amine base, such as pyridine or triethylamine, produces the compound of Formula IA.

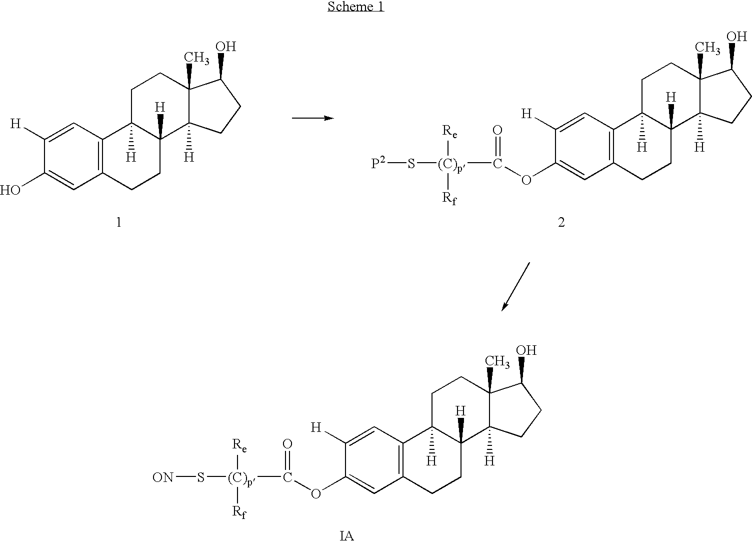

Nitroso compounds of Formula (I), wherein Rₑ, R_{f}, and p′ are as defined herein and a thionitrite containing carboxylic ester is representative of the O-D¹ group as defined herein can be prepared as shown in Scheme 2. The appropriate acid of the compound of Formula 1 is converted into the ester of Formula 3 wherein p′, Rₑ, R_{f} and P² are defined as herein, by reaction with an appropriate protected thiol containing alcohol. Preferred methods for the preparation of esters are initially forming the mixed anhydride via reaction of the acid with a chloroformate, such as isobutylchloroformate, in the presence of a non-nucelophilic base, such as triethylamine, in an anhydrous inert solvent, such as diethylether or THF. The mixed anhydride is then reacted with the protected thiol-containing alcohol, preferably in the presence of a condensation catalyst, such as DMAP. Alternatively, the acid may first be converted to the acid chloride by treatment with oxalyl chloride in the presence of a catalytic amount of DMF. The acid chloride is then reacted with the protected thiol containing alcohol, preferably in the presence of a condensation catalyst, such as DMAP, and a tertiary amine base, such as triethyl amine, to produce an ester. Alternatively, the appropriate acid and protected thiol-containing alcohol may be coupled to produce the ester by treatment with a dehydration agent, such as DCC or EDAC.HCl, with or without a condensation catalyst, such as DMAP or HOBt. Alternatively, the acid may first be converted into an alkali metal salt, such as the sodium, potassium or lithium salt, which is then reacted with an alkyl halide which also contains a protected thiol group in a polar solvent, such as DMF, to produce the ester. Preferred protecting groups for the thiol moiety are as a thioester, such as thioacetate or thiobenzoate, as a disulfide, as a thiocarbamate, such as N-methoxymethyl thiocarbamate, or as a thioether, such as paramethoxybenzyl thioether, a 2,4,6-trimethoxybenzyl thioether, a tetrahydropyranyl thioether, or a S-triphenylmethyl thioether. Deprotection of the thiol moiety in the compound of Formula 3 (zinc in dilute aqueous acid, triphenylphosphine in water and sodium borohydride are preferred methods for reducing disulfide groups, aqueous base or sodium methoxide in methanol is typically used to hydrolyze thioesters, aqueous base removes N-methoxymethyl thiocarbamates and mercuric trifluoroacetate, silver nitrate or strong acids such as trifluoroacetic or hydrochloric acid and heat are used to remove a paramethoxybenzyl thioether, 2,4,6-trimethoxybenzyl thioether, a tetrahydropyranyl thioether or a S-triphenylmethyl thioether group) followed by reaction with a suitable nitrosylating agent, such as thionyl chloride nitrite, thionyl dinitrite, a lower alkyl nitrite, such as tert-butyl nitrite, or nitrosium tetrafluoroborate, in a suitable anhydrous solvent, such as methylene chloride, THF, DMF or acetonitrile, with or without an amine base, such as pyridine or triethylamine, produces the compound of Formula IB. Alternatively, treatment of the deprotected thiol with a stoichiometric quantity of sodium nitrite in aqueous acid produces the compound of Formula IB.

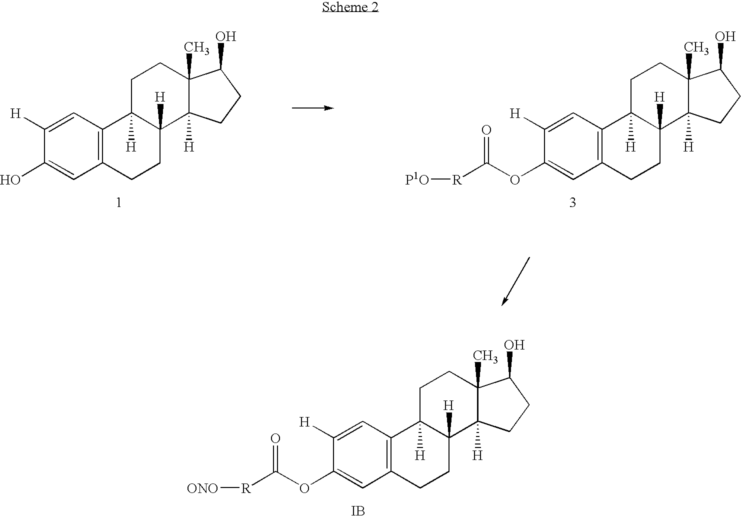

Nitro compounds of Formula (I), wherein Rₑ, R_{f}, and p are as defined herein and a nitrate containing carboxylic ester is representative of the O-D₁ group as defined herein can be prepared as shown in Scheme 3. The appropriate acid of the compound of Formula 1 is converted into the ester of Formula IC wherein p′, Rₑ and R_{f} defined as herein, by reaction with an appropriate nitrate containing alcohol. Preferred methods for the preparation of esters are initially forming the mixed anhydride via reaction of the acid with a chloroformate, such as isobutylchloroformate, in the presence of a non-nucelophilic base, such as triethylamine, in an anhydrous inert solvent, such as diethylether or THF. The mixed anhydride is then reacted with the nitrate containing alcohol, preferably in the presence of a condensation catalyst, such as DMAP. Alternatively, the acid may first be converted to the acid chloride by treatment with oxalyl chloride in the presence of a catalytic amount of DMF. The acid chloride is then reacted with the alcohol, preferably in the presence of a condensation catalyst, such as DMAP, and a tertiary amine base, such as triethyl amine, to produce an ester. Alternatively, the nitrite containing acid and alcohol may be coupled to produce the ester by treatment with a dehydration agent, such as DCC or EDAC.HCl with or without a condensation catalyst, such as DMAP or HOBt.

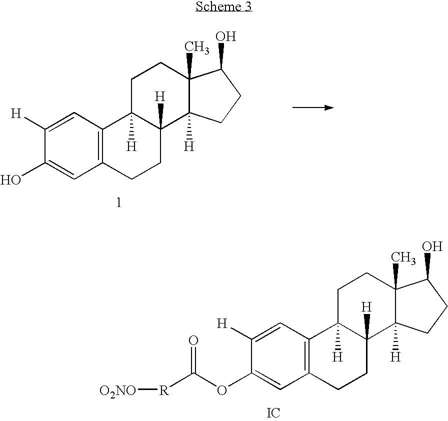

Nitroso compounds of Formula (II) wherein Rₑ, R_{f} and p′, are as defined herein, and an O-nitrosylated ester is representative of the D¹ group as defined herein may be prepared as outlined in Scheme 4. The phenolic group of Formula 4 is converted to the ester(s) of Formula 5 wherein p′, Rₑ and R_{f} are defined as herein by reaction with an appropriate protected alcohol containing activated acylating agent wherein P¹ is as defined above. Preferred methods for the formation of esters are reacting the alcohol with the preformed acid chloride or symmetrical anhydride of the protected alcohol containing acid or condensing the alcohol and protected alcohol containing acid in the presence of a dehydrating agent such as DCC or EDAC.HCl with or without a catalyst such as DMAP or HOBt. Preferred protecting groups for the alcohol moiety are silyl ethers such as a trimethylsilyl or tert-butyldimethylsilyl ether. Deprotection of the hydroxyl moieties (fluoride ion is the preferred method for removing silyl ether protecting groups) followed by reaction a suitable nitrosylating agent such as thionyl chloride nitrite, thionyl dinitrite, or nitrosonium tetrafluoroborate in a suitable anhydrous solvent such as, dichloromethane, THF, DMF, or acetonitrile, with or without an amine base such as pyridine or triethylamine gives the compound of Formula IIA.

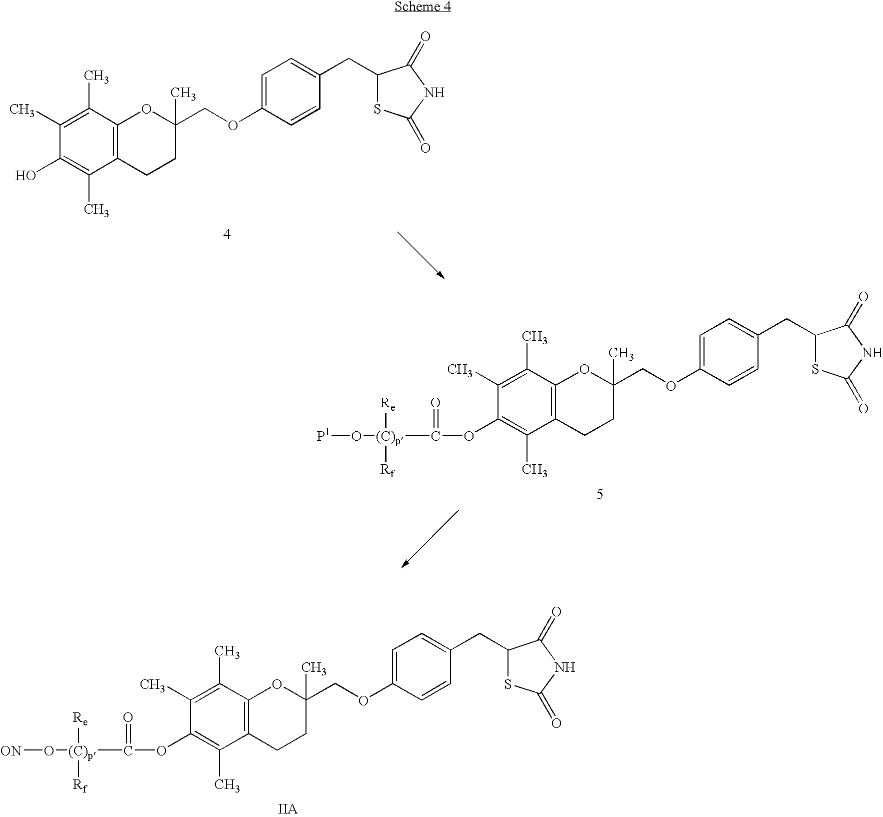

Nitroso compounds of Formula (II) wherein Rₑ, R_{f}, and p′ are defined as defined herein and a S-nitrosylated ester is representative of the D¹ group as defined herein may be prepared as outlined in Scheme 5. The phenolic group of Formula 4 is converted to the ester(s) of Formula 6 wherein p′, Rₑ and R_{f} are defined as herein by reaction with an appropriate protected thiol containing activated acylating agent wherein P2 is as defined herein. Preferred methods for the formation of esters are reacting the alcohol with the preformed acid chloride or symmetrical anhydride of the protected thiol containing acid or condensing the alcohol and protected thiol containing acid in the presence of a dehydrating agent such as DCC or EDAC.HCl with or without a catalyst such as DMAP or HOBt. Preferred protecting groups for the thiol moiety are as a thioester such as a thioacetate or thiobenzoate, as a disulfide, as a thiocarbamate such as N-methoxymethyl thiocarbamate, or as a thioether such as a paramethoxybenzyl thioether, a tetrahydropyranyl thioether or a 2,4,6-trimethoxybenzyl thioether. Deprotection of the thiol moiety (zinc in dilute aqueous acid, triphenylphosphine in water and sodium borohydride are preferred methods for reducing disulfide groups while aqueous base is typically utilized to hydrolyze thioesters and N-methoxymethyl thiocarbamates and mercuric trifluoroacetate, silver nitrate, or strong acids such as trifluoroacetic or hydrochloric acid and heat are used to remove a paramethoxybenzyl thioether, a tetrahydropyranyl thioether or a 2,4,6-trimethoxybenzyl thioether group) followed by reaction with a an eqimolar equivalent based upon thiol of a suitable nitrosylating agent such as thionyl chloride nitrite, thionyl dinitrite, a lower alkyl nitrite such as tert-butyl nitrite, or nitrosonium tetrafluoroborate in a suitable anhydrous solvent such as methylene chloride, THF, DMF, or acetonitrile with or without an amine base such as pyridine or triethylamine gives the compound of Formula IIB. Alternatively, treatment of the deptrotected thiol compound with a stoichiometric quantity of sodium nitrite in an acidic aqueous or alcoholic solution gives the compound of Formula IIB.

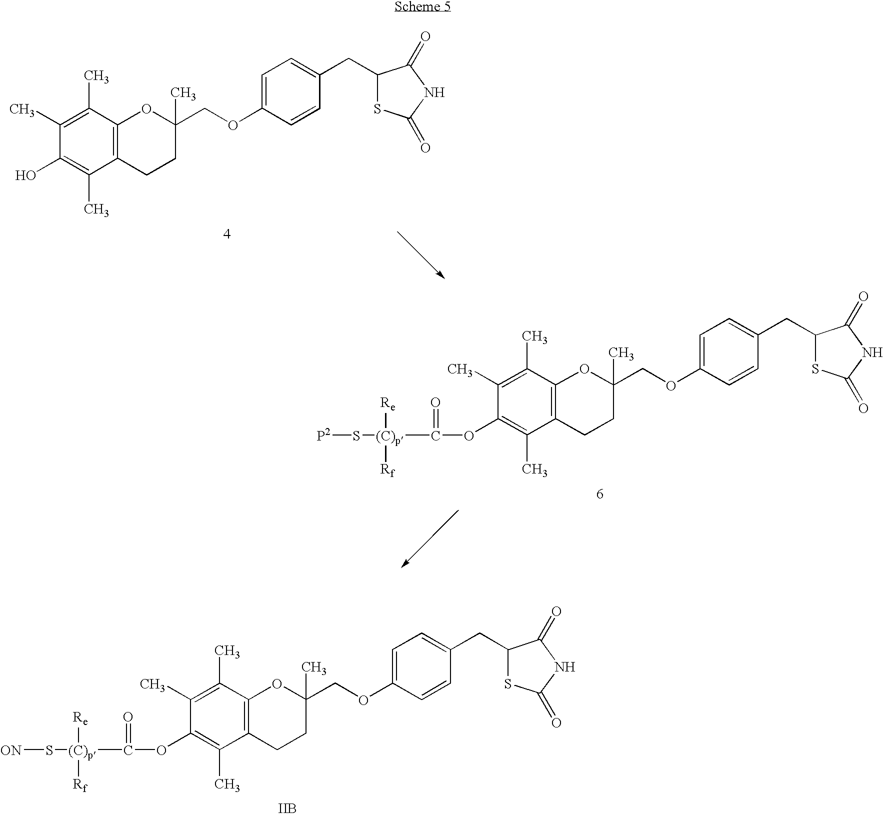

Nitro compounds of Formula (II), wherein Rₑ, R_{f}, and p are as defined herein and a nitrate containing carboxylic ester is representative of the U-D¹ group as defined herein can be prepared as shown in Scheme 6. The appropriate acid of the compound of Formula 4 is converted into the ester of Formula IIC wherein p′, Rₑ and R_{f} defined as herein, by reaction with an appropriate nitrate containing alcohol. Preferred methods for the preparation of esters are initially forming the mixed anhydride via reaction of the acid with a chloroformate, such as isobutylchloroformate, in the presence of a non-nucelophilic base, such as triethylamine, in an anhydrous inert solvent, such as diethylether or THF. The mixed anhydride is then reacted with the nitrate containing alcohol, preferably in the presence of a condensation catalyst, such as DMAP. Alternatively, the acid may first be converted to the acid chloride by treatment with oxalyl chloride in the presence of a catalytic amount of DMF. The acid chloride is then reacted with the alcohol, preferably in the presence of a condensation catalyst, such as DMAP, and a tertiary amine base, such as triethyl amine, to produce an ester. Alternatively, the nitrate containing acid and alcohol may be coupled to produce the ester by treatment with a dehydration agent, such as DCC or EDAC.HCl with or without a condensation catalyst, such as DMAP or HOBt.

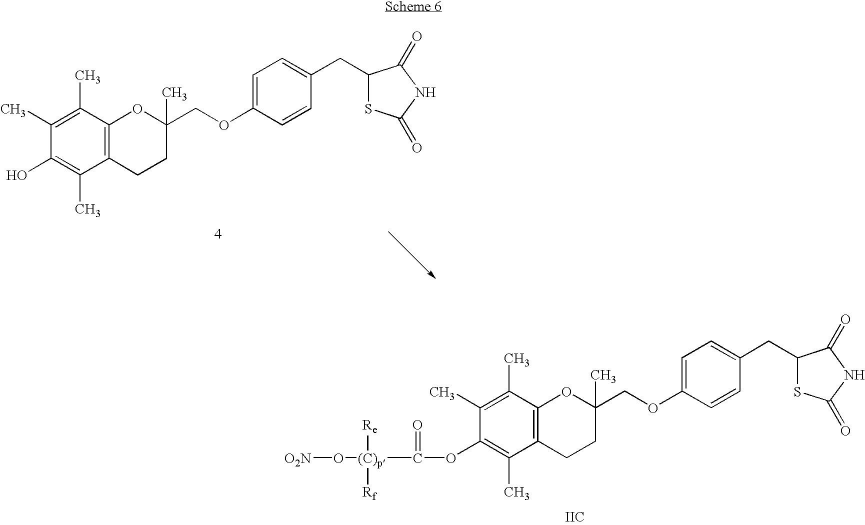

Nitroso compounds of Formula (III) wherein Rₑ, R_{f}, and p′ are defined as defined herein and a S-nitrosylated ester is representative of the D¹ group as defined herein may be prepared as outlined in Scheme 7. The phenolic group of Formula 7 is converted to the ester(s) of Formula 8 wherein p′, Rₑ and R_{f} are defined as herein by reaction with an appropriate protected thiol containing activated acylating agent wherein P² is as defined herein. Preferred methods for the formation of esters are reacting the alcohol with the preformed acid chloride or symmetrical anhydride of the protected thiol containing acid or condensing the alcohol and protected thiol containing acid in the presence of a dehydrating agent such as DCC or EDAC.HCl with or without a catalyst such as DMAP or HOBt. Preferred protecting groups for the thiol moiety are as a thioester such as a thioacetate or thiobenzoate, as a disulfide, as a thiocarbamate such as N-methoxymethyl thiocarbamate, or as a thioether such as a paramethoxybenzyl thioether, a tetrahydropyranyl thioether or a 2,4,6-trimethoxybenzyl thioether. Deprotection of the thiol moiety (zinc in dilute aqueous acid, triphenylphosphine in water and sodium borohydride are preferred methods for reducing disulfide groups while aqueous base is typically utilized to hydrolyze thioesters and N-methoxymethyl thiocarbamates and mercuric trifluoroacetate, silver nitrate, or strong acids such as trifluoroacetic or hydrochloric acid and heat are used to remove a paramethoxybenzyl thioether, a tetrahydropyranyl thioether or a 2,4,6-trimethoxybenzyl thioether group) followed by reaction with a an eqimolar equivalent based upon thiol of a suitable nitrosylating agent such as thionyl chloride nitrite, thionyl dinitrite, a lower alkyl nitrite such as tert-butyl nitrite, or nitrosonium tetrafluoroborate in a suitable anhydrous solvent such as methylene chloride, THF, DMF, or acetonitrile with or without an amine base such as pyridine or triethylamine gives the compound of Formula IIIA. Alternatively, treatment of the deptrotected thiol compound with a stoichiometric quantity of sodium nitrite in an acidic aqueous or alcoholic solution gives the compound of Formula IIIA.

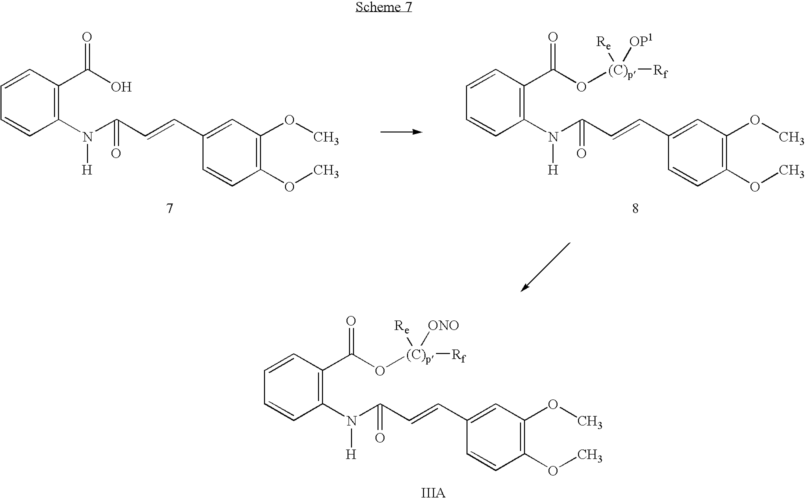

Nitroso compounds of Formula (III) wherein R¹ is a hydrogen, D¹ is a hydrogen or K and a nitrite containing ester is representative of the D¹ group as defined herein, may be prepared as outlined in Scheme 8. The compound of Formula 7 is converted to the ester of Formula 9, wherein R is —W′ₐ₋₁-E_{b}-(C(Rₑ)(R_{f}))_{p′}-E_{c}-(C(Rₑ)(R_{f}))ₓ—W′_{d}—(C(Rₑ)(R_{f}))_{y}—W′ᵢ-Eⱼ-W′_{g}—(C(Rₑ)(R_{f}))_{z}, by reaction with an appropriate protected alcohol containing active acylating agent, wherein P¹ is as defined herein. Preferred methods for the preparation of esters are initially forming the mixed anhydride via reaction of the acid with a chloroformate, such as isobutylchloroformate, in the presence of a non-nucleophilic base, such as triethylamine, in an anhydrous inert solvent, such as dichloromethane, diethylether or THF. The mixed anhydride is then reacted with the mono-phenolic group, preferably in the presence of a condensation catalyst, such as DMAP. Alternatively, the acid may first be converted to the acid chloride by treatment with oxalyl chloride in the presence of a catalytic amount of DMF. The acid chloride is then reacted with the mono-phenolic group, preferably in the presence of a condensation catalyst, such as DMAP, and a tertiary amine base, such as triethylamine, to produce the ester. Alternatively, the phenolic group may be coupled to produce the ester by treatment with a dehydration agent, such as dicyclohexylcarbodiimide (DCC) or 1-ethyl-3(3-dimethylaminopropyl)carbodiimide hydrochloride (EDAC.HCl) with a catalyst, such as DMAP or 1-hydroxybenzotriazole (HOBt). Preferred protecting groups for the alcohol moiety are as a benzyl ether or a benzyl carbonate. Deprotection of the hydroxyl moiety (hydrogenolysis using a palladium catalyst or electrolytic reduction are the preferred methods for removing benzyl ether and benzyl carbonate protecting groups) followed by reaction with a suitable nitrosylating agent, such as thionyl chloride nitrite, thionyl dinitrite, or nitrosonium tetrafluoroborate, in a suitable anhydrous solvent, such as dichloromethane, THF, DMF, or acetonitrile with or without an amine base such as, pyridine or triethylamine, gives the compounds of Formula IIIB.

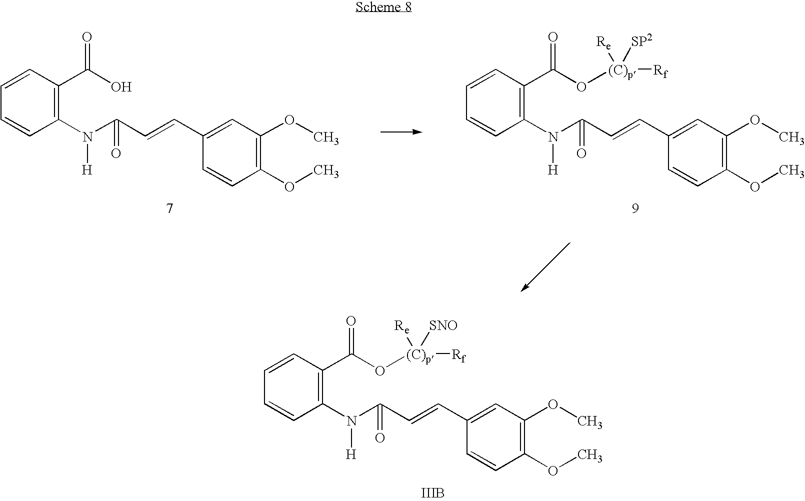

Nitro compounds of Formula (III) wherein R¹ is a hydrogen, D¹ is a hydrogen or K, and a nitrate containing ester is representative of the D¹ group, may be prepared as outlined in Scheme 9. The compound of Formula 7 is converted to the nitrate ester of Formula IIIC, wherein R is as defined herein by reaction with an appropriate protected nitrate containing active acylating agent. Preferred methods for the preparation of esters are initially forming the mixed anhydride via reaction of the acid with a chloroformate, such as isobutylchloroformate, in the presence of a non-nucleophilic base, such as triethylamine, in an anhydrous inert solvent, such as dichloromethane, diethylether or THF. The mixed anhydride is then reacted with the mono-phenolic group, preferably in the presence of a condensation catalyst, such as DMAP. Alternatively, the acid may first be converted to the acid chloride by treatment with oxalyl chloride in the presence of a catalytic amount of DMF. The acid chloride is then reacted with the mono-phenolic group, preferably in the presence of a condensation catalyst, such as DMAP, and a tertiary amine base, such as triethylamine, to produce the ester. Alternatively, the nitrate containing acid and mono-phenolic group may be coupled to produce the ester by treatment with a dehydration agent, such as DCC or EDAC.HCl, with a catalyst such as, DMAP or HOBt.

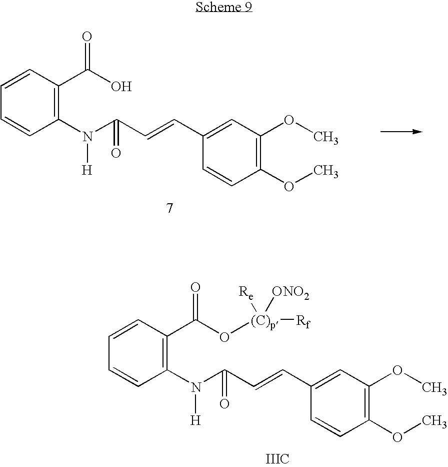

Nitroso compounds of Formula (IV), wherein Rₑ, R_{f}, and p′ are as defined herein and a nitrite containing carboxylic ester is representative of the U-D¹ group as defined herein can be prepared as shown in Scheme 10. The acid of the compound of Formula 10 is converted into the ester of Formula 11 wherein p′, Rₑ, R_{f} and P¹ are defined as herein, by reaction with an appropriate monoprotected diol. Preferred methods for the preparation of esters are forming the mixed anhydride via reaction of the acid with a chloroformate, such as isobutylchloroformate, in the presence of a non-nucleophilic base, such as triethylamine, in an anhydrous inert solvent, such as dichloromethane, diethylether or THF. The mixed anhydride is then reacted with the monoprotected alcohol, preferably in the presence of a condensation catalyst, such as 4-dimethylamino pyridine (DMAP). Alternatively, the acid may first be converted to the acid chloride by treatment with oxalyl chloride in the presence of a catalytic amount of DMF. The acid chloride is then reacted with the monoprotected alcohol, preferably in the presence of a condensation catalyst, such as DMAP, and a tertiary amine base, such as triethyl amine, to produce the ester. Alternatively, the acid and monoprotected diol may be coupled to produce the ester by treatment with a dehydration agent, such as dicyclohexylcarbodiimide (DCC) or 1-(3-dimethylaminopropyl)-3-ethylcarbodiimide hydrochloride (EDAC.HCl) with or without a condensation catalyst, such as DMAP or 1-hydroxybenzotriazole (HOBt). Alternatively, the acid may first be converted into an alkali metal salt, such as the sodium, potassium or lithium salt, and reacted with an alkyl halide that also contains a protected hydroxyl group in a polar solvent, such as DMF, to produce the ester. Preferred protecting groups for the alcohol moiety are silyl ethers, such as a trimethylsilyl or a tert-butyldimethylsilyl ether. Deprotection of the hydroxyl moiety in the compound of Formula 11 (fluoride ion is the preferred method for removing silyl ether protecting groups) followed by reaction with a suitable nitrosylating agent, such as thionyl chloride nitrite, thionyl dinitrite or nitrosonium tetrafluoroborate, in a suitable anhydrous solvent, such as methylene chloride, THF, DMF or acetonitrile, with or without an amine base, such as pyridine or triethylamine, produces the compound of Formula IVA.

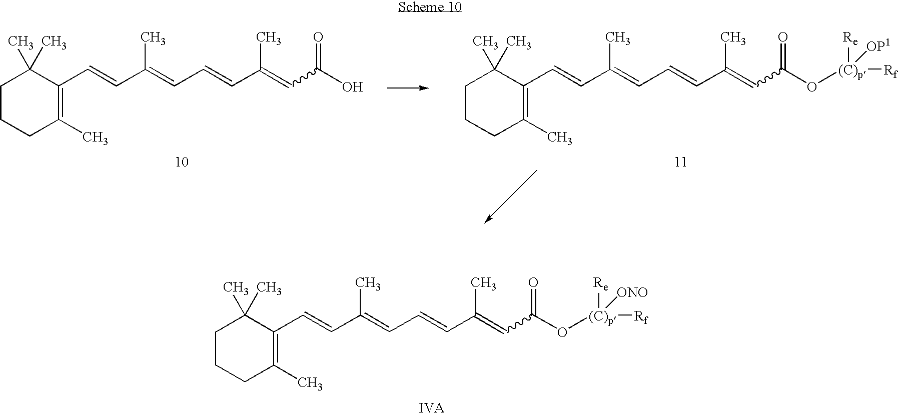

Nitroso compounds of Formula (IV), wherein Rₑ, R_{f}, and p are as defined herein a thionitrite containing carboxylic ester is representative of the U-D¹ group as defined herein can be prepared as shown in Scheme 11. The appropriate acid of the compound of Formula 10 is converted into the ester of Formula 12 wherein p′, Rₑ, R_{f} and P² are defined as herein, by reaction with an appropriate protected thiol containing alcohol. Preferred methods for the preparation of esters are initially forming the mixed anhydride via reaction of the acid with a chloroformate, such as isobutylchloroformate, in the presence of a non-nucelophilic base, such as triethylamine, in an anhydrous inert solvent, such as diethylether or THF. The mixed anhydride is then reacted with the protected thiol-containing alcohol, preferably in the presence of a condensation catalyst, such as DMAP. Alternatively, the acid may first be converted to the acid chloride by treatment with oxalyl chloride in the presence of a catalytic amount of DMF. The acid chloride is then reacted with the protected thiol containing alcohol, preferably in the presence of a condensation catalyst, such as DMAP, and a tertiary amine base, such as triethyl amine, to produce an ester. Alternatively, the appropriate acid and protected thiol-containing alcohol may be coupled to produce the ester by treatment with a dehydration agent, such as DCC or EDAC.HCl, with or without a condensation catalyst, such as DMAP or HOBt. Alternatively, the acid may first be converted into an alkali metal salt, such as the sodium, potassium or lithium salt, which is then reacted with an alkyl halide which also contains a protected thiol group in a polar solvent, such as DMF, to produce the ester. Preferred protecting groups for the thiol moiety are as a thioester, such as thioacetate or thiobenzoate, as a disulfide, as a thiocarbamate, such as N-methoxymethyl thiocarbamate, or as a thioether, such as paramethoxybenzyl thioether, a 2,4,6-trimethoxybenzyl thioether, a tetrahydropyranyl thioether, or a S-triphenylmethyl thioether. Deprotection of the thiol moiety in the compound of Formula 12 (zinc in dilute aqueous acid, triphenylphosphine in water and sodium borohydride are preferred methods for reducing disulfide groups, aqueous base or sodium methoxide in methanol is typically used to hydrolyze thioesters, aqueous base removes N-methoxymethyl thiocarbamates and mercuric trifluoroacetate, silver nitrate or strong acids such as trifluoroacetic or hydrochloric acid and heat are used to remove a paramethoxybenzyl thioether, 2,4,6-trimethoxybenzyl thioether, a tetrahydropyranyl thioether or a S-triphenylmethyl thioether group) followed by reaction with a suitable nitrosylating agent, such as thionyl chloride nitrite, thionyl dinitrite, a lower alkyl nitrite, such as tert-butyl nitrite, or nitrosium tetrafluoroborate, in a suitable anhydrous solvent, such as methylene chloride, THF, DMF or acetonitrile, with or without an amine base, such as pyridine or triethylamine, produces the compound of Formula IVB. Alternatively, treatment of the deprotected thiol with a stoichiometric quantity of sodium nitrite in aqueous acid produces the compound of Formula IVB.

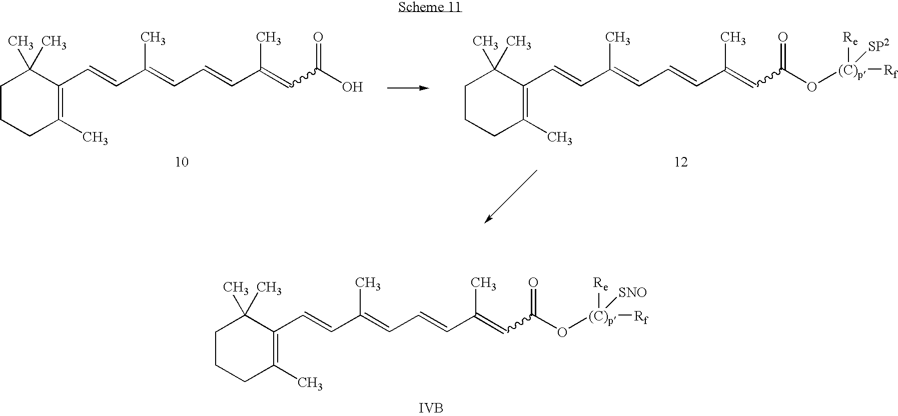

Nitro compounds of Formula (IV), wherein Rₑ, R_{f}, and p are as defined herein and a nitrate containing carboxylic ester is representative of the U-D¹ group as defined herein can be prepared as shown in Scheme 12. The appropriate acid of the compound of Formula 10 is converted into the ester of Formula IVC wherein p′, Rₑ and R_{f} defined as herein, by reaction with an appropriate nitrate containing alcohol. Preferred methods for the preparation of esters are initially forming the mixed anhydride via reaction of the acid with a chloroformate, such as isobutylchloroformate, in the presence of a non-nucelophilic base, such as triethylamine, in an anhydrous inert solvent, such as diethylether or THF. The mixed anhydride is then reacted with the nitrate containing alcohol, preferably in the presence of a condensation catalyst, such as DMAP. Alternatively, the acid may first be converted to the acid chloride by treatment with oxalyl chloride in the presence of a catalytic amount of DMF. The acid chloride is then reacted with the protected thiol containing alcohol, preferably in the presence of a condensation catalyst, such as DMAP, and a tertiary amine base, such as triethyl amine, to produce an ester. Alternatively, the appropriate acid and protected thiol-containing alcohol may be coupled to produce the ester by treatment with a dehydration agent, such as DCC or EDAC.HCl with or without a condensation catalyst, such as DMAP or HOBt.

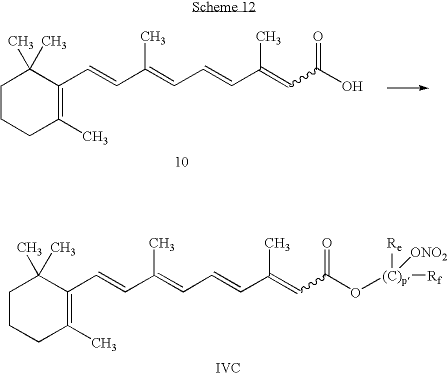

Nitroso compounds of Formula (V) wherein Rₑ, R_{f}, and p′ are defined as defined herein and a S-nitrosylated ester is representative of the D¹ group as defined herein may be prepared as outlined in Scheme 13. The phenolic group of Formula 13 is converted to the ester(s) of Formula 14 wherein p′, Rₑ and R_{f} are defined as herein by reaction with an appropriate protected thiol containing activated acylating agent wherein P² is as defined herein. Preferred methods for the formation of esters are reacting the alcohol with the preformed acid chloride or symmetrical anhydride of the protected thiol containing acid or condensing the alcohol and protected thiol containing acid in the presence of a dehydrating agent such as DCC or EDAC.HCl with or without a catalyst such as DMAP or HOBt. Preferred protecting groups for the thiol moiety are as a thioester such as a thioacetate or thiobenzoate, as a disulfide, as a thiocarbamate such as N-methoxymethyl thiocarbamate, or as a thioether such as a paramethoxybenzyl thioether, a tetrahydropyranyl thioether or a 2,4,6-trimethoxybenzyl thioether. Deprotection of the thiol moiety (zinc in dilute aqueous acid, triphenylphosphine in water and sodium borohydride are preferred methods for reducing disulfide groups while aqueous base is typically utilized to hydrolyze thioesters and N-methoxymethyl thiocarbamates and mercuric trifluoroacetate, silver nitrate, or strong acids such as trifluoroacetic or hydrochloric acid and heat are used to remove a paramethoxybenzyl thioether, a tetrahydropyranyl thioether or a 2,4,6-trimethoxybenzyl thioether group) followed by reaction with a an eqimolar equivalent based upon thiol of a suitable nitrosylating agent such as thionyl chloride nitrite, thionyl dinitrite, a lower alkyl nitrite such as tert-butyl nitrite, or nitrosonium tetrafluoroborate in a suitable anhydrous solvent such as methylene chloride, THF, DMF, or acetonitrile with or without an amine base such as pyridine or triethylamine gives the compound of Formula VA. Alternatively, treatment of the deptrotected thiol compound with a stoichiometric quantity of sodium nitrite in an acidic aqueous or alcoholic solution gives the compound of Formula VA.

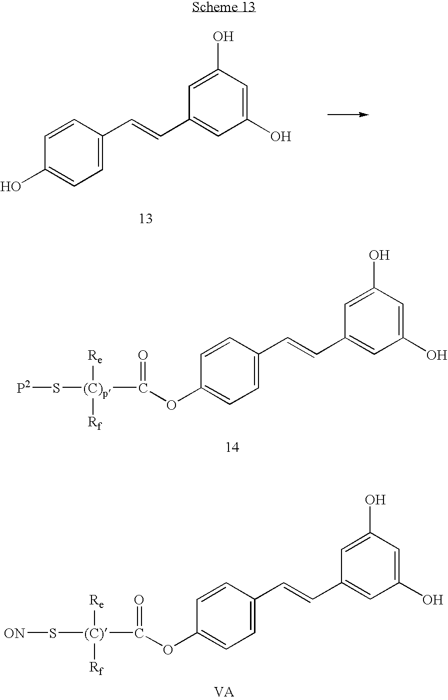

Nitroso compounds of Formula (V) wherein D¹ is a hydrogen or K and a nitrite containing ester is representative of the D¹ group as defined herein, may be prepared as outlined in Scheme 14. The compound of Formula 13 is converted to the ester of Formula 15, wherein R is —W′ₐ₋₁-E_{b}-(C(Rₑ)(R_{f}))_{p′}-E_{c}-(C(Rₑ)(R_{f}))ₓ—W′_{d}—(C(Rₑ)(R_{f}))_{y}—W′ᵢ-Eⱼ-W_{g}—(C(Rₑ)(R_{f}))_{z}, by reaction with an appropriate protected alcohol containing active acylating agent, wherein P¹ is as defined herein. Preferred methods for the preparation of esters are initially forming the mixed anhydride via reaction of the acid with a chloroformate, such as isobutylchloroformate, in the presence of a non-nucleophilic base, such as triethylamine, in an anhydrous inert solvent, such as dichloromethane, diethylether or THF. The mixed anhydride is then reacted with the mono-phenolic group, preferably in the presence of a condensation catalyst, such as DMAP. Alternatively, the acid may first be converted to the acid chloride by treatment with oxalyl chloride in the presence of a catalytic amount of DMF. The acid chloride is then reacted with the mono-phenolic group, preferably in the presence of a condensation catalyst, such as DMAP, and a tertiary amine base, such as triethylamine, to produce the ester. Alternatively, the mono-phenolic group may be coupled to produce the ester by treatment with a dehydration agent, such as dicyclohexylcarbodiimide (DCC) or 1-ethyl-3(3-dimethylaminopropyl)carbodiimide hydrochloride (EDAC.HCl) with a catalyst, such as DMAP or 1-hydroxybenzotriazole (HOBt). Preferred protecting groups for the alcohol moiety are as a benzyl ether or a benzyl carbonate. Deprotection of the hydroxyl moiety (hydrogenolysis using a palladium catalyst or electrolytic reduction are the preferred methods for removing benzyl ether and benzyl carbonate protecting groups) followed by reaction with a suitable nitrosylating agent, such as thionyl chloride nitrite, thionyl dinitrite, or nitrosonium tetrafluoroborate, in a suitable anhydrous solvent, such as dichloromethane, THF, DMF, or acetonitrile with or without an amine base such as, pyridine or triethylamine, gives the compounds of Formula VB.

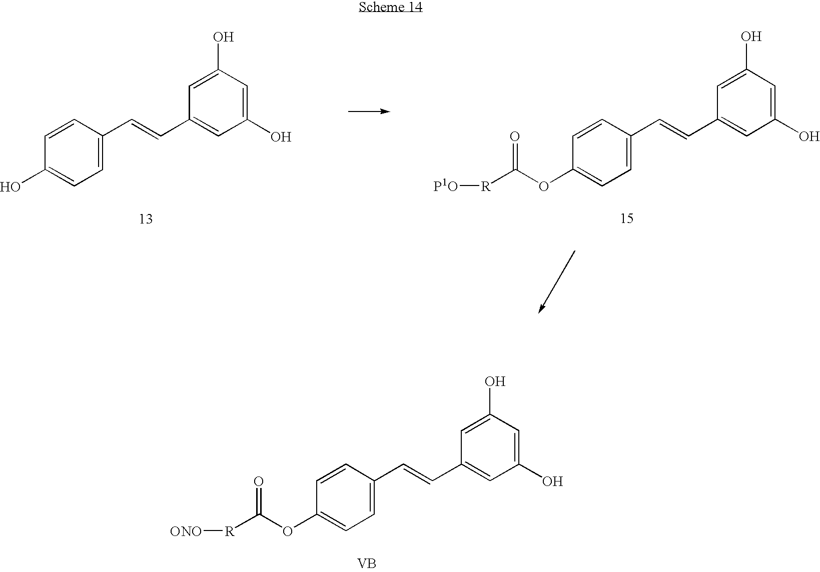

Nitro compounds of Formula (V) wherein D¹ is a hydrogen or K, and a nitrate containing ester is representative of the D¹ group, may be prepared as outlined in Scheme 15. The compound of Formula 13 is converted to the nitrate ester of Formula VC, wherein R is as defined herein by reaction with an appropriate protected nitrate containing active acylating agent. Preferred methods for the preparation of esters are initially forming the mixed anhydride via reaction of the acid with a chloroformate, such as isobutylchloroformate, in the presence of a non-nucleophilic base, such as triethylamine, in an anhydrous inert solvent, such as dichloromethane, diethylether or THF. The mixed anhydride is then reacted with the mono-phenolic group, preferably in the presence of a condensation catalyst, such as DMAP. Alternatively, the acid may first be converted to the acid chloride by treatment with oxalyl chloride in the presence of a catalytic amount of DMF. The acid chloride is then reacted with the mono-phenolic group, preferably in the presence of a condensation catalyst, such as DMAP, and a tertiary amine base, such as triethylamine, to produce the ester. Alternatively, the nitrate containing acid and mono-phenolic group may be coupled to produce the ester by treatment with a dehydration agent, such as DCC or EDAC.HCl, with a catalyst such as, DMAP or HOBt.

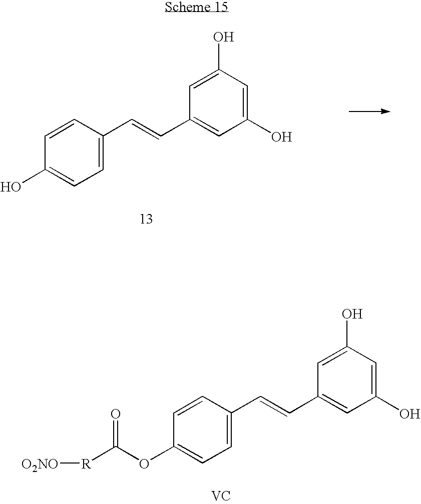

Nitroso compounds of Formula (VI), wherein Rₑ, R_{f}, and p are as defined herein and a nitrite containing carboxylic ester is representative of the U-D¹ group as defined herein can be prepared as shown in Scheme 16. The acid of the compound of Formula 16 is converted into the ester of Formula 17 wherein p′, Rₑ, R_{f} and P¹ are defined as herein, by reaction with an appropriate monoprotected diol. Preferred methods for the preparation of esters are forming the mixed anhydride via reaction of the acid with a chloroformate, such as isobutylchloroformate, in the presence of a non-nucleophilic base, such as triethylamine, in an anhydrous inert solvent, such as dichloromethane, diethylether or THF. The mixed anhydride is then reacted with the monoprotected alcohol, preferably in the presence of a condensation catalyst, such as 4-dimethylamino pyridine (DMAP). Alternatively, the acid may first be converted to the acid chloride by treatment with oxalyl chloride in the presence of a catalytic amount of DMF. The acid chloride is then reacted with the monoprotected alcohol, preferably in the presence of a condensation catalyst, such as DMAP, and a tertiary amine base, such as triethyl amine, to produce the ester. Alternatively, the acid and monoprotected diol may be coupled to produce the ester by treatment with a dehydration agent, such as dicyclohexylcarbodiimide (DCC) or 1-(3-dimethylaminopropyl)-3-ethylcarbodiimide hydrochloride (EDAC.HCl) with or without a condensation catalyst, such as DMAP or 1-hydroxybenzotriazole (HOBt). Alternatively, the acid may first be converted into an alkali metal salt, such as the sodium, potassium or lithium salt, and reacted with an alkyl halide that also contains a protected hydroxyl group in a polar solvent, such as DMF, to produce the ester. Preferred protecting groups for the alcohol moiety are silyl ethers, such as a trimethylsilyl or a tert-butyldimethylsilyl ether. Deprotection of the hydroxyl moiety in the compound of Formula 17 (fluoride ion is the preferred method for removing silyl ether protecting groups) followed by reaction with a suitable nitrosylating agent, such as thionyl chloride nitrite, thionyl dinitrite or nitrosonium tetrafluoroborate, in a suitable anhydrous solvent, such as methylene chloride, THF, DMF or acetonitrile, with or without an amine base, such as pyridine or triethylamine, produces the compound of Formula VIA.

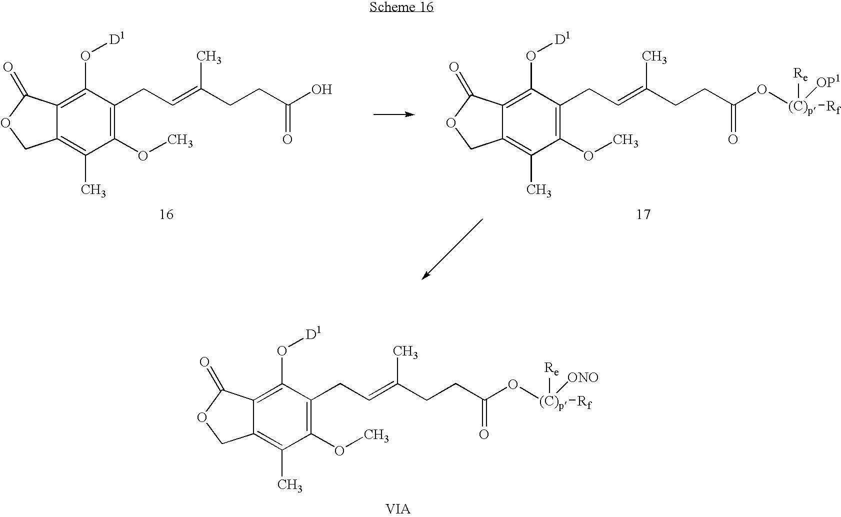

Nitroso compounds of Formula (VI), wherein Rₑ, R_{f}, and p are as defined herein a thionitrite containing carboxylic ester is representative of the U-D¹ group as defined herein can be prepared as shown in Scheme 17. The appropriate acid of the compound of Formula 16 is converted into the ester of Formula 18 wherein p′, Rₑ, R_{f} and P² are defined as herein, by reaction with an appropriate protected thiol containing alcohol. Preferred methods for the preparation of esters are initially forming the mixed anhydride via reaction of the acid with a chloroformate, such as isobutylchloroformate, in the presence of a non-nucelophilic base, such as triethylamine, in an anhydrous inert solvent, such as diethylether or THF. The mixed anhydride is then reacted with the protected thiol-containing alcohol, preferably in the presence of a condensation catalyst, such as DMAP. Alternatively, the acid may first be converted to the acid chloride by treatment with oxalyl chloride in the presence of a catalytic amount of DMF. The acid chloride is then reacted with the protected thiol containing alcohol, preferably in the presence of a condensation catalyst, such as DMAP, and a tertiary amine base, such as triethyl amine, to produce an ester. Alternatively, the appropriate acid and protected thiol-containing alcohol may be coupled to produce the ester by treatment with a dehydration agent, such as DCC or EDAC.HCl, with or without a condensation catalyst, such as DMAP or HOBt. Alternatively, the acid may first be converted into an alkali metal salt, such as the sodium, potassium or lithium salt, which is then reacted with an alkyl halide which also contains a protected thiol group in a polar solvent, such as DMF, to produce the ester. Preferred protecting groups for the thiol moiety are as a thioester, such as thioacetate or thiobenzoate, as a disulfide, as a thiocarbamate, such as N-methoxymethyl thiocarbamate, or as a thioether, such as paramethoxybenzyl thioether, a 2,4,6-trimethoxybenzyl thioether, a tetrahydropyranyl thioether, or a S-triphenylmethyl thioether. Deprotection of the thiol moiety in the compound of Formula 18 (zinc in dilute aqueous acid, triphenylphosphine in water and sodium borohydride are preferred methods for reducing disulfide groups, aqueous base or sodium methoxide in methanol is typically used to hydrolyze thioesters, aqueous base removes N-methoxymethyl thiocarbamates and mercuric trifluoroacetate, silver nitrate or strong acids such as trifluoroacetic or hydrochloric acid and heat are used to remove a paramethoxybenzyl thioether, 2,4,6-trimethoxybenzyl thioether, a tetrahydropyranyl thioether or a S-triphenylmethyl thioether group) followed by reaction with a suitable nitrosylating agent, such as thionyl chloride nitrite, thionyl dinitrite, a lower alkyl nitrite, such as tert-butyl nitrite, or nitrosium tetrafluoroborate, in a suitable anhydrous solvent, such as methylene chloride, THF, DMF or acetonitrile, with or without an amine base, such as pyridine or triethylamine, produces the compound of Formula VIB. Alternatively, treatment of the deprotected thiol with a stoichiometric quantity of sodium nitrite in aqueous acid produces the compound of Formula VIB.

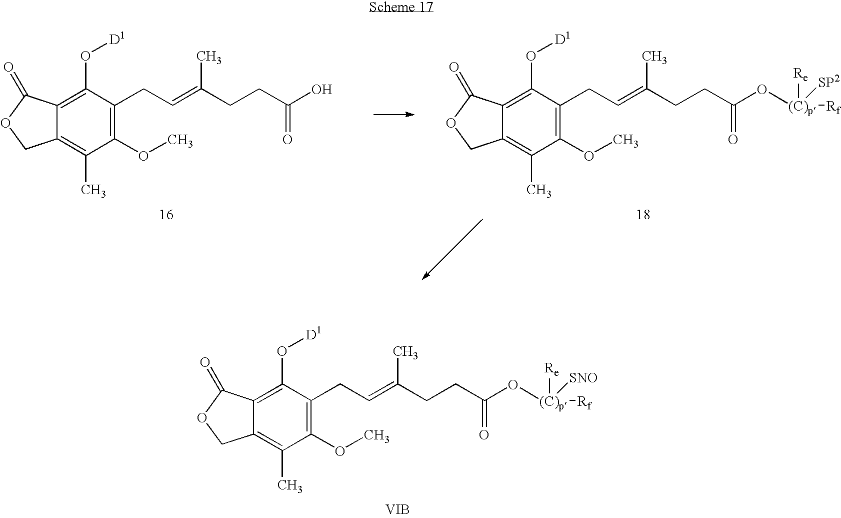

Nitro compounds of Formula (VI), wherein Rₑ, R_{f}, and p are as defined herein and a nitrate containing carboxylic ester is representative of the U-D¹ group as defined herein can be prepared as shown in Scheme 18. The appropriate acid of the compound of Formula 16 is converted into the ester of Formula VIC wherein p′, Rₑ and R_{f} defined as herein, by reaction with an appropriate nitrate containing alcohol. Preferred methods for the preparation of esters are initially forming the mixed anhydride via reaction of the acid with a chloroformate, such as isobutylchloroformate, in the presence of a non-nucelophilic base, such as triethylamine, in an anhydrous inert solvent, such as diethylether or THF. The mixed anhydride is then reacted with the nitrate containing alcohol, preferably in the presence of a condensation catalyst, such as DMAP. Alternatively, the acid may first be converted to the acid chloride by treatment with oxalyl chloride in the presence of a catalytic amount of DMF. The acid chloride is then reacted with the protected thiol containing alcohol, preferably in the presence of a condensation catalyst, such as DMAP, and a tertiary amine base, such as triethyl amine, to produce an ester. Alternatively, the appropriate acid and protected thiol-containing alcohol may be coupled to produce the ester by treatment with a dehydration agent, such as DCC or EDAC.HCl with or without a condensation catalyst, such as DMAP or HOBt.

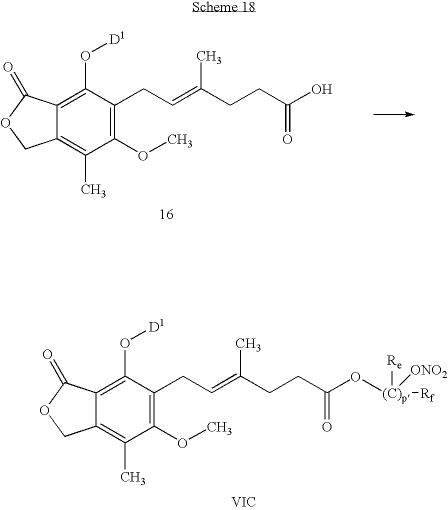

Nitroso compounds of Formula (VII), wherein Rₑ, R_{f}, and p are as defined herein, y⁷ is the integer 6, x⁷ is the integer 2, and a nitrite containing carboxylic ester is representative of the U-D¹ group as defined herein can be prepared as shown in Scheme 19. The acid of the compound of Formula 19 is converted into the ester of Formula 20 wherein p′, Rₑ, R_{f} and P¹ are defined as herein, by reaction with an appropriate monoprotected diol. Preferred methods for the preparation of esters are forming the mixed anhydride via reaction of the acid with a chloroformate, such as isobutylchloroformate, in the presence of a non-nucleophilic base, such as triethylamine, in an anhydrous inert solvent, such as dichloromethane, diethylether or THF. The mixed anhydride is then reacted with the monoprotected alcohol, preferably in the presence of a condensation catalyst, such as 4-dimethylamino pyridine (DMAP). Alternatively, the acid may first be converted to the acid chloride by treatment with oxalyl chloride in the presence of a catalytic amount of DMF. The acid chloride is then reacted with the monoprotected alcohol, preferably in the presence of a condensation catalyst, such as DMAP, and a tertiary amine base, such as triethyl amine, to produce the ester. Alternatively, the acid and monoprotected diol may be coupled to produce the ester by treatment with a dehydration agent, such as dicyclohexylcarbodiimide (DCC) or 1-(3-dimethylaminopropyl)-3-ethylcarbodiimide hydrochloride (EDAC.HCl) with or without a condensation catalyst, such as DMAP or 1-hydroxybenzotriazole (HOBt). Alternatively, the acid may first be converted into an alkali metal salt, such as the sodium, potassium or lithium salt, and reacted with an alkyl halide that also contains a protected hydroxyl group in a polar solvent, such as DMF, to produce the ester. Preferred protecting groups for the alcohol moiety are silyl ethers, such as a trimethylsilyl or a tert-butyldimethylsilyl ether. Deprotection of the hydroxyl moiety in the compound of Formula 20 (fluoride ion is the preferred method for removing silyl ether protecting groups) followed by reaction with a suitable nitrosylating agent, such as thionyl chloride nitrite, thionyl dinitrite or nitrosonium tetrafluoroborate, in a suitable anhydrous solvent, such as methylene chloride, THF, DMF or acetonitrile, with or without an amine base, such as pyridine or triethylamine, produces the compound of Formula VIIA.

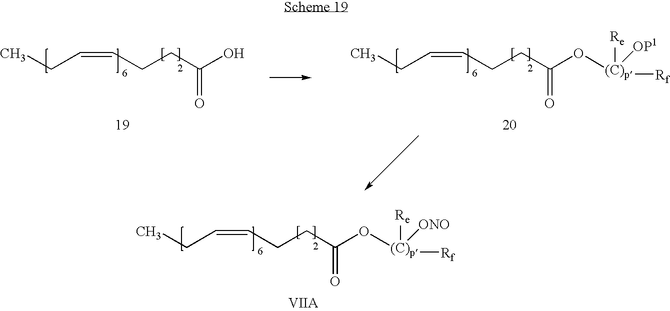

Nitroso compounds of Formula (VII), wherein Rₑ, R_{f}, and p are as defined herein, y⁷ is the integer 6, x⁷ is the integer 2, and a thionitrite containing carboxylic ester is representative of the U-D¹ group as defined herein can be prepared as shown in Scheme 20. The appropriate acid of the compound of Formula 19 is converted into the ester of Formula 21 wherein p′, Rₑ, R_{f} and P² are defined as herein, by reaction with an appropriate protected thiol containing alcohol. Preferred methods for the preparation of esters are initially forming the mixed anhydride via reaction of the acid with a chloroformate, such as isobutylchloroformate, in the presence of a non-nucelophilic base, such as triethylamine, in an anhydrous inert solvent, such as diethylether or THF. The mixed anhydride is then reacted with the protected thiol-containing alcohol, preferably in the presence of a condensation catalyst, such as DMAP. Alternatively, the acid may first be converted to the acid chloride by treatment with oxalyl chloride in the presence of a catalytic amount of DMF. The acid chloride is then reacted with the protected thiol containing alcohol, preferably in the presence of a condensation catalyst, such as DMAP, and a tertiary amine base, such as triethyl amine, to produce an ester. Alternatively, the appropriate acid and protected thiol-containing alcohol may be coupled to produce the ester by treatment with a dehydration agent, such as DCC or EDAC.HCl, with or without a condensation catalyst, such as DMAP or HOBt. Alternatively, the acid may first be converted into an alkali metal salt, such as the sodium, potassium or lithium salt, which is then reacted with an alkyl halide which also contains a protected thiol group in a polar solvent, such as DMF, to produce the ester. Preferred protecting groups for the thiol moiety are as a thioester, such as thioacetate or thiobenzoate, as a disulfide, as a thiocarbamate, such as N-methoxymethyl thiocarbamate, or as a thioether, such as paramethoxybenzyl thioether, a 2,4,6-trimethoxybenzyl thioether, a tetrahydropyranyl thioether, or a S-triphenylmethyl thioether. Deprotection of the thiol moiety in the compound of Formula 21 (zinc in dilute aqueous acid, triphenylphosphine in water and sodium borohydride are preferred methods for reducing disulfide groups, aqueous base or sodium methoxide in methanol is typically used to hydrolyze thioesters, aqueous base removes N-methoxymethyl thiocarbamates and mercuric trifluoroacetate, silver nitrate or strong acids such as trifluoroacetic or hydrochloric acid and heat are used to remove a paramethoxybenzyl thioether, 2,4,6-trimethoxybenzyl thioether, a tetrahydropyranyl thioether or a S-triphenylmethyl thioether group) followed by reaction with a suitable nitrosylating agent, such as thionyl chloride nitrite, thionyl dinitrite, a lower alkyl nitrite, such as tert-butyl nitrite, or nitrosium tetrafluoroborate, in a suitable anhydrous solvent, such as methylene chloride, THF, DMF or acetonitrile, with or without an amine base, such as pyridine or triethylamine, produces the compound of Formula VIIB. Alternatively, treatment of the deprotected thiol with a stoichiometric quantity of sodium nitrite in aqueous acid produces the compound of Formula VIIB.

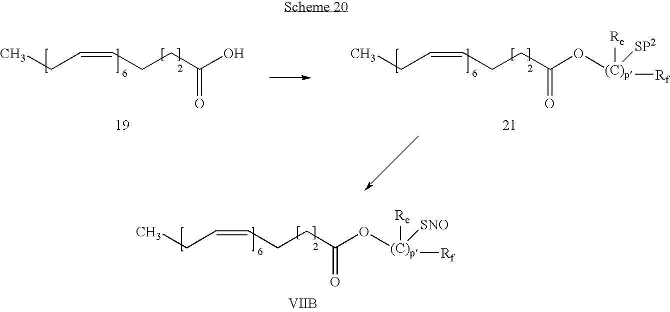

Nitro compounds of Formula (VII), wherein Rₑ, R_{f}, and p′ are as defined herein, y⁷is the integer 6, x⁷ is the integer 2, and a nitrate containing carboxylic ester is representative of the U-D¹ group as defined herein can be prepared as shown in Scheme 21. The appropriate acid of the compound of Formula 19 is converted into the ester of Formula VIIC wherein p′, Rₑ and R_{f} defined as herein, by reaction with an appropriate nitrate containing alcohol. Preferred methods for the preparation of esters are initially forming the mixed anhydride via reaction of the acid with a chloroformate, such as isobutylchloroformate, in the presence of a non-nucelophilic base, such as triethylamine, in an anhydrous inert solvent, such as diethylether or THF. The mixed anhydride is then reacted with the nitrate containing alcohol, preferably in the presence of a condensation catalyst, such as DMAP. Alternatively, the acid may first be converted to the acid chloride by treatment with oxalyl chloride in the presence of a catalytic amount of DMF. The acid chloride is then reacted with the protected thiol containing alcohol, preferably in the presence of a condensation catalyst, such as DMAP, and a tertiary amine base, such as triethyl amine, to produce an ester. Alternatively, the appropriate acid and protected thiol-containing alcohol may be coupled to produce the ester by treatment with a dehydration agent, such as DCC or EDAC.HCl with or without a condensation catalyst, such as DMAP or HOBt.

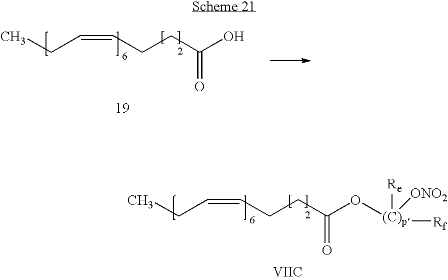

The compounds of the invention, including those described herein, which have been nitrosated and/or nitrosylated through one or more sites such as, oxygen (hydroxyl condensation), sulfur (sulfhydryl condensation) and/or nitrogen. The nitrosated and/or nitrosylated compounds of the invention donate, transfer or release a biologically active form of nitrogen monoxide (nitric oxide).

Nitrogen monoxide can exist in three forms: NO− (nitroxyl), NO· (nitric oxide) and NO⁺ (nitrosonium). NO· is a highly reactive short-lived species that is potentially toxic to cells. This is critical because the pharmacological efficacy of NO depends upon the form in which it is delivered. In contrast to the nitric oxide radical (NO·), nitrosonium (NO⁺) does not react with O₂ or O₂— species, and functionalities capable of transferring and/or releasing NO⁺ and NO− are also resistant to decomposition in the presence of many redox metals. Consequently, administration of charged NO equivalents (positive and/or negative) does not result in the generation of toxic by-products or the elimination of the active NO moiety.

Compounds contemplated for use in the invention (e.g., nitrosated and/or nitrosylated compounds of the invention and /or the compounds of the invention that are not nitrosated and/or nitrosylated ) are, optionally, used in combination with nitric oxide and compounds that release nitric oxide or otherwise directly or indirectly deliver or transfer nitric oxide to a site of its activity, such as on a cell membrane in vivo. In one embodiment the preferred compounds of the invention that are not nitrosated and/or nitrosylated are estradiol for the compound of Formula I, troglitazone for the compound of Formula II, tranilast for the compound of Formula III, retinoic acid for the compound of Formula IV, resveratrol for the compound of Formula V, mycophenolic acid for the compound of Formula VI, acids for the compounds of Formula VII, anthracenone for the compounds of Formula VIII and trapidil compounds of Formula IX.

The term “nitric oxide” encompasses uncharged nitric oxide (NO·) and charged nitrogen monoxide species, preferably charged nitrogen monoxide species, such as nitrosonium ion (NO⁺) and nitroxyl ion (NO−). The reactive form of nitric oxide can be provided by gaseous nitric oxide. The nitrogen monoxide releasing, delivering or transferring compounds have the structure F—NO, wherein F is a nitrogen monoxide releasing, delivering or transferring moiety, and include any and all such compounds which provide nitrogen monoxide to its intended site of action in a form active for its intended purpose. The term “NO adducts” encompasses any nitrogen monoxide releasing, delivering or transferring compounds, including, for example, S-nitrosothiols, nitrites, nitrates, S-nitrothiols, sydnonimines, 2-hydroxy-2-nitrosohydrazines, (NONOates), (E)-alkyl-2-((E)-hydroxyimino)-5-nitro-3-hexeneamide (FK-409), (E)-alkyl-2-((E)-hydroxyimino)-5-nitro-3-hexeneamines, N-((2Z, 3E)-4-ethyl-2-(hydroxyimino)-6-methyl-5-nitro-3-heptenyl)-3-pyridinecarboxamide (FR 146801), N-nitrosoamines, N-hydroxyl nitrosamines, nitrosimines, diazetine dioxides, oxatriazole 5-imines, oximes, hydroxylamines, N-hydroxyguanidines, hydroxyureas, benzofuroxanes, furoxans as well as substrates for the endogenous enzymes which synthesize nitric oxide.

Suitable NONOates include, but are not limited to, (Z)-1-(N-methyl-N-(6-(N-methyl-ammoniohexyl)amino))diazen-1-ium-1,2-diolate (“MAHMA/NO”), (Z)-1-(N-(3-ammoniopropyl)-N-(n-propyl)amino)diazen-1-ium-1,2-diolate (“PAPA/NO”), (Z)-1-(N-(3-aminopropyl)-N-(4-(3-aminopropylammonio)butyl)-amino) diazen-1-ium-1,2-diolate (spermine NONOate or “SPER/NO”) and sodium(Z)-1-(N,N-diethylamino)diazenium-1,2-diolate (diethylamine NONOate or “DEA/NO”) and derivatives thereof. NONOates are also described in U.S. Pat. Nos. 6,232,336, 5,910,316 and 5,650,447, the disclosures of which are incorporated herein by reference in their entirety. The “NO adducts” can be mono-nitrosylated, poly-nitrosylated, mono-nitrosated and/or poly-nitrosated at a variety of naturally susceptible or artificially provided binding sites for biologically active forms of nitrogen monoxide.

Suitable furoxanes include, but are not limited to, CAS 1609, C93-4759, C92-4678, S35b, CHF 2206, CHF 2363, and the like.

Suitable sydnonimines include, but are not limited to, molsidomine (N-ethoxycarbonyl-3-morpholinosydnonimine), SIN-1 (3-morpholinosydnonimine) CAS 936 (3-(cis-2,6-dimethylpiperidino)-N-(4-methoxybenzoyl)-sydnonimine, pirsidomine), C87-3754 (3-(cis-2,6-dimethylpiperidino)-sydnonimine, linsidomine), C4144 (3-(3,3-dimethyl-1,4-thiazane-4-yl)sydnonimine hydrochloride), C89-4095 (3-(3,3-dimethyl-1,1-dioxo-1,4-thiazane-4-yl)sydnonimine hydrochloride, and the like.

Suitable oximes, include but are not limited to, NOR-1, NOR-3, NOR-4, and the like.

One group of NO adducts is the S-nitrosothiols, which are compounds that include at least one —S—NO group. These compounds include S-nitroso-polypeptides (the term “polypeptide” includes proteins and polyamino acids that do not possess an ascertained biological function, and derivatives thereof); S-nitrosylated amino acids (including natural and synthetic amino acids and their stereoisomers and racemic mixtures and derivatives thereof); S-nitrosylated sugars; S-nitrosylated, modified and unmodified, oligonucleotides (preferably of at least 5, and more preferably 5-200 nucleotides); straight or branched, saturated or unsaturated, aliphatic or aromatic, substituted or unsubstituted S-nitrosylated hydrocarbons; and S-nitroso heterocyclic compounds. S-nitrosothiols and methods for preparing them are described in U.S. Pat. Nos. 5,380,758 and 5,703,073; WO 97/27749; WO 98/19672; and Oae et al, Org. Prep. Proc. Int., 15(3):165-198 (1983), the disclosures of each of which are incorporated by reference herein in their entirety.

Another embodiment of the invention is S-nitroso amino acids where the nitroso group is linked to a sulfur group of a sulfur-containing amino acid or derivative thereof. Such compounds include, for example, S-nitroso-N-acetylcysteine, S-nitroso-captopril, S-nitroso-N-acetylpenicillamine, S-nitroso-homocysteine, S-nitroso-cysteine, S-nitroso-glutathione, S-nitroso-cysteinyl-glycine, and the like.

Suitable S-nitrosylated proteins include thiol-containing proteins (where the NO group is attached to one or more sulfur groups on an amino acid or amino acid derivative thereof) from various functional classes including enzymes, such as tissue-type plasminogen activator (TPA) and cathepsin B; transport proteins, such as lipoproteins; heme proteins, such as hemoglobin and serum albumin; and biologically protective proteins, such as immunoglobulins, antibodies and cytokines. Such nitrosylated proteins are described in WO 93/09806, the disclosure of which is incorporated by reference herein in its entirety. Examples include polynitrosylated albumin where one or more thiol or other nucleophilic centers in the protein are modified.

Other examples of suitable S-nitrosothiols include:

(i) HS(C(Rₑ)(R_{f}))ₘSNO;

(ii) ONS(C(Rₑ)(R_{f}))ₘRₑ; or

(iii) H₂N—CH(CO₂H)—(CH₂)ₘ—C(O)NH—CH(CH₂SNO)—C(O)NH—CH₂—CO₂H;

wherein m is an integer from 2 to 20; Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, W′ₕ, —(CH₂)ₒ—U—V, or —(C(R_{g})(Rₕ))ₖ—U—V, or Rₑ and R_{f} taken together with the carbons to which they are attached form a carbonyl, a methanthial, a heterocyclic ring, a cycloalkyl group, an aryl group, an oxime, a hydrazone or a bridged cycloalkyl group;

R_{g} and Rₕ at each occurrence are independently Rₑ;

k is an integer from 1 to 3;

W′ is independently —C(O)—, —C(S)—, -T″-, —(C(Rₑ)(R_{f}))ₕ—, an alkyl group, an aryl group, a heterocyclic ring, an arylheterocyclic ring, or —(CH₂CH₂O)_{q′}—;

h is an integer form 1 to 10;

U at each occurrence is independently a covalent bond, a carbonyl, an oxygen, —S(O)ₒ— or —N(Rₐ)Rᵢ;

o is an integer from 0 to 2;

V is —NO or —NO₂;

Rₐ is a lone pair of electrons, a hydrogen or an alkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U—V)(Rₑ)(R_{f}), a bond to an adjacent atom creating a double bond to that atom, —(N₂O₂—)⁻·M⁺, wherein M⁺ is an organic or inorganic cation.

In cases where Rₑ and R_{f} are a heterocyclic ring or taken together Rₑ and R_{f} are a heterocyclic ring, then Rᵢ can be a substituent on any disubstituted nitrogen contained within the radical wherein Rᵢ is as defined herein.

Nitrosothiols can be prepared by various methods of synthesis. In general, the thiol precursor is prepared first, then converted to the S-nitrosothiol derivative by nitrosation of the thiol group with NaNO₂ under acidic conditions (pH is about 2.5) which yields the S-nitroso derivative. Acids which can be used for this purpose include aqueous sulfuric, acetic and hydrochloric acids. The thiol precursor can also be nitrosylated by reaction with an organic nitrite such as tert-butyl nitrite, or a nitrosonium salt such as nitrosonium tetrafluoroborate in an inert solvent.

Another group of NO adducts for use in the invention, where the NO adduct is a compound that donates, transfers or releases nitric oxide, include compounds comprising at least one ON—O— or ON—N-group. The compounds that include at least one ON—O— or ON—N-group are preferably ON—O— or ON—N-polypeptides (the term “polypeptide” includes proteins and polyamino acids that do not possess an ascertained biological function, and derivatives thereof); ON—O—or ON—N-amino acids (including natural and synthetic amino acids and their stereoisomers and racemic mixtures); ON—O— or ON—N-sugars; ON—O— or —ON—N-modified or unmodified oligonucleotides (comprising at least 5 nucleotides, preferably 5-200 nucleotides); ON—O— or ON—N-straight or branched, saturated or unsaturated, aliphatic or aromatic, substituted or unsubstituted hydrocarbons; and ON—O—, ON—N— or ON—C-heterocyclic compounds. Preferred examples of compounds comprising at least one ON—O— or ON—N-group include butyl nitrite, isobutyl nitrite, tert-butyl nitrite, amyl nitrite, isoamyl nitrite, N-nitrosamines, N-nitrosamides, N-nitrosourea, N-nitrosoguanidines, N-nitrosocarbamates, N-acyl-N-nitroso compounds (such as, N-methyl-N-nitrosourea); N-hydroxy-N-nitrosamines, cupferron, alanosine, dopastin, 1,3-disubstitued nitrosiminobenzimidazoles, 1,3,4-thiadiazole-2-nitrosimines, benzothiazole-2(3H)-nitrosimines, thiazole-2-nitrosimines, oligonitroso sydnonimines, 3-alkyl-N-nitroso-sydnonimines, 2H-1,3,4-thiadiazine nitrosimines.

Another group of NO adducts for use in the invention include nitrates that donate, transfer or release nitric oxide, such as compounds comprising at least one O₂N—O—, O₂N—N— or O₂N—S-group. Preferred among these compounds are O₂N—O—, O₂N—N— or O₂N—S-polypeptides (the term “polypeptide” includes proteins and also polyamino acids that do not possess an ascertained biological function, and derivatives thereof); O₂N—O—, O₂N—N— or O₂N—S-amino acids (including natural and synthetic amino acids and their stereoisomers and racemic mixtures); O₂N—O—, O₂N—N— or O₂N—S-sugars; O₂N—O—, O₂N—N— or O₂N—S-modified and unmodified oligonucleotides (comprising at least 5 nucleotides, preferably 5-200 nucleotides); O₂N—O—, O₂N—N— or O₂N—S-straight or branched, saturated or unsaturated, aliphatic or aromatic, substituted or unsubstituted hydrocarbons; and O₂N—O—, O₂N—N— or O₂N—S-heterocyclic compounds. Preferred examples of compounds comprising at least one O₂N—O—, O₂N—N— or O₂N—S-group include isosorbide dinitrate, isosorbide mononitrate, clonitrate, erythrityl tetranitrate, mannitol hexanitrate, nitroglycerin, pentaerythritoltetranitrate, pentrinitrol, propatylnitrate and organic nitrates with a sulfhydryl-containing amino acid such as, for example SPM 3672, SPM 5185, SPM 5186 and those disclosed in U.S. Pat. Nos. 5,284,872, 5,428,061, 5,661,129, 5,807,847 and 5,883,122 and in WO 97/46521, WO 00/54756 and in WO 03/013432, the disclosures of each of which are incorporated by reference herein in their entirety.

Another group of NO adducts are N-oxo-N-nitrosoamines that donate, transfer or release nitric oxide and are represented by the formula: R^{1″}R^{2″}N—N(O-M⁺)-NO, where R^{1″} and R^{2″} are each independently a polypeptide, an amino acid, a sugar, a modified or unmodified oligonucleotide, a straight or branched, saturated or unsaturated, aliphatic or aromatic, substituted or unsubstituted hydrocarbon, or a heterocyclic group, and where M⁺ is an organic or inorganic cation, such, as for example, an alkyl substituted ammonium cation or a Group I metal cation.

The invention is also directed to compounds that stimulate endogenous NO or elevate levels of endogenous endothelium-derived relaxing factor (EDRF) in vivo or are oxidized to produce nitric oxide and/or are substrates for nitric oxide synthase and/or cytochrome P450. Such compounds include, for example, L-arginine, L-homoarginine, and N-hydroxy-L-arginine, N-hydroxy-L-homoarginine, N-hydroxydebrisoquine, N-hydroxypentamidine including their nitrosated and/or nitrosylated analogs (e.g., nitrosated L-arginine, nitrosylated L-arginine, nitrosated N-hydroxy-L-arginine, nitrosylated N-hydroxy-L-arginine, nitrosated and nitrosylated L-homoarginine), N-hydroxyguanidine compounds, amidoxime, ketoximes, aldoxime compounds, that can be oxidized in vivo to produce nitric oxide or maybe substrates for a cytochrome P450, such as, for example, imino(benzylamino)methylhydroxylamine, imino(((4-methylphenyl)methyl)amino)methylhydroxylamine, imino(((4-methoxyphenyl)methyl)amino)methylhydroxylamine, imino(((4-(trifluoromethyl)phenyl)methyl)amino)methylhydroxylamine, imino(((4-nitrophenyl)methyl)amino)methylhydroxylamine, (butylamino)iminomethylhydroxylamine, imino(propylamino)methylhydroxylamine, imino(pentylamino)methylhydroxylamine, imino(propylamino)methylhydroxylamine, imino((methylethyl)amino)methylhydroxylamine, (cyclopropylamino)iminomethylhydroxylamine, imino-2-1,2,3,4-tetrahydroisoquinolylmethylhydroxylamine, imino(1-methyl(2-1,2,3,4-tetrahydroisoquinolyl))methylhydroxylamine, (1,3-dimethyl(2-1,2,3,4-tetrahydroisoquinolyl))iminomethylhydroxylamine, (((4-chlorophenyl)methyl)amino)iminomethylhydroxylamine, ((4-chlorophenyl)amino)iminomethylhydroxylamine, (4-chlorophenyl)(hydroxyimino)methylamine, and 1-(4-chlorophenyl)-1-(hydroxyimino)ethane, and the like, precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid), nitric oxide mediators and/or physiologically acceptable salts thereof, including, for example, pyruvate, pyruvate precursors, a-keto acids having four or more carbon atoms, precursors of α-keto acids having four or more carbon atoms (as disclosed in WO 03/017996, the disclosure of which is incorporated herein in its entirety), and the substrates for nitric oxide synthase, cytokines, adenosin, bradykinin, calreticulin, bisacodyl, and phenolphthalein. EDRF is a vascular relaxing factor secreted by the endothelium, and has been identified as nitric oxide (NO) or a closely related derivative thereof (Palmer et al, Nature, 327:524-526 (1987); Ignarro et al, Proc. Natl. Acad. Sci. USA, 84:9265-9269 (1987)).

The invention is also based on the discovery that the administration of a therapeutically effective amount of the compounds and compositions described herein is effective for treating or preventing cardiovascular diseases and disorders. For example, the patient can be administered a therapeutically effective amount of at least one nitrosated and/or nitrosylated compound of the invention. In another embodiment, the patient can be administered a therapeutically effective amount of at least one compound of the invention, optionally substituted with at least one NO and/or NO₂ group, and at least one nitric oxide donor compound. In yet another embodiment, the patient can be administered a therapeutically effective amount of at least one compound of the invention, optionally substituted with at least one NO and/or NO₂ group, and at least one therapeutic agent, and, optionally, at least one nitric oxide donor compound. The compounds can be administered separately or in the form of a composition.

A “therapeutic agent” useful in the invention includes, but is not limited to, agents which biologically stent a vessel and/or reduce or inhibit vascular or non-vascular remodeling and/or inhibit or reduce vascular or non-vascular smooth muscle proliferation following a procedural vascular or non-vascular trauma. The “therapeutic agents” of the invention include agents that inhibit the cellular activity of a vascular or non-vascular smooth muscle cell, for example, proliferation, migration, increase in cell volume, increase in extracellular matrix synthesis (e.g., collagens, proteoglycans, and the like), or secretion of extracellular matrix materials by the cell. Suitable “therapeutic agents” useful in the invention, include, but are not limited to, antithrombogenic agents (such as, for example, heparin, covalent heparin, hirudin, hirulog, coumadin, protamine, argatroban, D-phenylalanyl-L-poly-L-arginyl chloromethyl ketone, and the like); thrombolytic agents (such as, for example, urokinase, streptokinase, tissueplasminogen activators, and the like); fibrinolytic agents; vasospasm inhibitors; potassium channel blockers; calcium channel blockers; antihypertensive agents (such as, for example, HYTRIN®, and the like); antimicrobial agents or antibiotics (such as, for example, adriamycin, and the like); platelet reducing agents; antimitotic, antiproliferative agents or microtubule inhibitors (such as, for example, colchicine, methotrexate, azathioprine, vincristine, vinblastine, cytochalasin, fluorouracil, adriamycin, mutamycin, tubercidin, epothilone A or B, discodermolide, taxol, and the like); antisecretory agents (such as, for example, retinoid, and the like); remodeling inhibitors; antisense nucleotides (such as, for example, deoxyribonucleic acid, and the like); anti-cancer agents (such as, for example, tamoxifen citrate, acivicin, bizelesin, daunorubicin, epirubicin, mitoxantrone, and the like); steroids (such as, for example, dexamethasone, dexamethasone sodium phosphate, dexamethasone acetate, and the like); non-steroidal antiinflammatory agents (NSAID); COX-2 inhibitors; anti-hyperlipidemic drugs; immunosuppressive agents (such as, for example cyclosporin, and the like); growth factor antagonists or antibodies (such as, for example, trapidal (a PDGF antagonist)), angiopeptin (a growth hormone antagonist), angiogenin, and the like); dopamine agonists (such as, for example, apomorphine, bromocriptine, testosterone, cocaine, strychnine, and the like); radiotherapeutic agents (such as, for example, ⁶⁰Co (5.3 year half life), ¹⁹²Ir (73.8 days), ³²P (14.3 days), ¹¹¹In (68 hours), ⁹⁰Y (64 hours), ^{99m}Tc (6 hours), and the like); heavy metals functioning as radiopaque agents (such as, for example, iodine-containing compounds, barium-containing compounds, gold, tantalum, platinum, tungsten, and the like); biologic agents (such as, for example, peptides, proteins, enzymes, extracellular matrix components, cellular components, and the like); aldosterone antagonists, alpha-adrenergic receptor antagonists, angiotensin II antagonists, β-adrenergic agonists, anti-hyperlipidemic drugs, angiotensin converting enzyme (ACE) inhibitors, antioxidants, β-adrenergic antagonists, endothelin antagonists; neutral endopeptidase inhibitors; renin inhibitiors; free radical scavengers, iron chelators or antioxidants (such as, for example, ascorbic acid, alpha tocopherol, superoxide dismutase, deferoxamine, 21-aminosteroid, and the like); sex hormone (such as, for example, estrogen, and the like); antipolymerases (such as, for example, AZT, and the like); antiviral agents (such as, for example, acyclovir, famciclovir, rimantadine hydrochloride, ganciclovir sodium, Norvir®, Crixivan®, and the like); photodynamic therapy agents (such as, for example, 5-aminolevulinic acid, meta-tetrahydroxyphenylchlorin, hexadecafluoro zinc phthalocyanine, tetramethyl hematoporphyrin, rhodamine 123, and the like); antibody targeted therapy agents (such as, for example, IgG2 Kappa antibodies against Pseudomonas aeruginosa exotoxin A and reactive with A431 epidermoid carcinoma cells, monoclonal antibody against the noradrenergic enzyme dopamine beta-hydroxylase conjugated to saporin, and the like); gene therapy agents; hormone replacement therapy (such as, for example, estrogens, conjugated estrogens, ethinyl estradiol, 17-beta-estradiol, estradiol, estropipate, and the like); and mixtures of two or more thereof. The compounds of the invention, nitric oxide donors and/or therapeutic agents can be administered separately or in the form of a composition. The compounds and compositions of the invention can also be administered in combination with other medications used for the treatment of these diseases or disorders.

In one embodiment of the invention, the therapeutic agents are anticoagulants, aldosterones, alpha-adrenergic receptor antagonists, angiotensin II antagonists, β-adrenergic agonists, anti-hyperlipidemic drugs, angiotensin-converting enzyme inhibitors, antioxidants, β-adrenergic antagonists, endothelin antagonists, neutral endopeptidase inhibitors, nonsteroidal anti-inflammatory compounds (NSAIDs), potassium channel blockers, platelet reducing agents, renin inhibitors, selective cyclooxygenase-2 (COX-2) inhibitors, steroids, and mixtures of two or more thereof.

Suitable anticoagulants include, but are not limited to, heparin, coumarin, aspirin, protamine, warfarin, dicumarol, phenprocoumon, indan-1,3-dione, acenocoumarol, ansindione, and the like. Suitable anticoagulants are described more fully in the literature, such as in Goodman and Gilman, The Pharmacological Basis of Therapeutics (9th Edition), McGraw-Hill, 1995, Pgs. 1341-1359; the Merck Index on CD-ROM, Twelfth Edition, Version 12:1, 1996; STN express file reg and file phar.

Suitable aldosterone antagonists include, but are not limited to, canrenone, potassium canrenoate, spironolactone, eplerenone, pregn-4-ene-7,21-dicarboxylic acid, 9,11-epoxy-17-hydroxy-3-oxo, γ-lactone, methyl ester, (7α,11α,17α.)-; pregn-4-ene-7,21-dicarboxylic acid, 9,11-epoxy-17-hydroxy-3-oxo-dimethyl ester, (7α,11α,17α.)-; 3′H-cyclopropa(6,7)pregna-4,6-diene-21-carboxylic acid, 9,11-epoxy-6,7-dihydro-17-hydroxy-3-oxo-, γ-lactone, (6β,7β,11β,17β)-; pregn-4-ene-7,21-dicarboxylic acid, 9,11-epoxy-17-hydroxy-3-oxo-, 7-(1-methylethyl)ester, monopotassium salt, (7α,11α,17α.)-; pregn-4-ene-7,21-dicarboxylic acid, 9,11,-epoxy-17-hydroxy-3-oxo-, 7-methyl ester, monopotassium salt, (7α,11α,17α.)-; 3′H-cyclopropa(6,7)pregna-1,4,6-triene-21-carboxylic acid, 9,11-epoxy-6,7-dihydro-17-hydroxy-3-oxo-, γ-lactone, (6α,7α,11α)-; 3′H-cyclopropa(6,7)pregna-4,6-diene-21-carboxylic acid, 9,11-epoxy-6,7-dihydro-17-hydroxy-3-oxo-, methyl ester, ((6α,7α,11α,17α)-; 3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α.)-; 3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-; pregn-4-ene-7,21-dicarboxylic acid, 9,11-epoxy-17-hydroxy-3-oxo-, γ-lactone, ethyl ester, (7α,11α,17α.)-; pregn-4-ene-7,21-dicarboxylic acid, 9,11-epoxy-17-hydroxy-3-oxo-, γ-lactone, 1-methylethyl ester, (7α,11α,17α.)-; and the like. Suitable aldosterone antagonists are described more fully in the literature, such as in Goodman and Gilman, The Pharmacological Basis of Therapeutics (9th Edition), McGraw-Hill, 1995; and the Merck Index on CD-ROM, 13th Edition; and on STN Express, file phar and file registry.

Suitable alpha-adrenergic receptor antagonists include but are not limited to, phentolamine, tolazoline, idazoxan, deriglidole, RX 821002, BRL 44408, BRL 44409, BAM 1303, labetelol, ifenprodil, rauwolscine, corynathine, raubascine, tetrahydroalstonine, apoyohimbine, akuammigine, β-yohimbine, yohimbol, yohimbine, pseudoyohimbine, epi-3α-yohimbine, 10-hydroxy-yohimbine, 11-hydroxy-yohimbine, tamsulosin, benoxathian, atipamezole, BE 2254, WB 4101, HU-723, tedisamil, mirtazipine, setiptiline, reboxitine, delequamine, naftopil, saterinone, SL 89.0591, ARC 239, urapidil, 5-methylurapidil, monatepi, haloperidol, indoramin, SB 216469, moxisylyte, trazodone, dapiprozole, efaroxan, Recordati 15/2739, SNAP 1069, SNAP 5089, SNAP 5272, RS 17053, SL 89.0591, KMD 3213, spiperone, AH 11110A, chloroethylclonidine, BMY 7378, niguldipine, and the like. Suitable alpha-adrenergic receptor antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable angiotensin II antagonists include, but are not limited to, angiotensin, candesartan, candesartan cilexetil, eprosartan, irbesartan, isoteoline, losartan, olmesartan, medoxomil, remikirin, riposartan, saprisartan, saralasin, sarmesin, tasosartan, telmisartan, valsartan, zolasartin, 3-(2′(tetrazole-5-yl)-1,1′-biphen-4-yl)methyl-5,7-dimethyl-2-ethyl-3H-imidazo(4,5-b)pyridine, antibodies to angiotensin II, A-81282, A-81988, BAY-106734, BIBR-363, BIBS-39, BIBS-222, BMS-180560, BMS-184698, CGP-38560A, CGP-42112A, CGP-48369, CGP-49870, CGP-63170, CI-996, CP-148130, CL-329167, CV-11194, DA-2079, DE-3489, DMP-811, DuP-167, DuP-532, DuP-753, E-4177, E-4188, EMD-66397, EMD-73495, EMD-66684, EXP-063, EXP-929, EXP-3174, EXP-6155, EXP-6803, EXP-771 1, EXP-9270, EXP-9954, FK-739, FR-1153332, GA-0050, GA-0056, HN-65021, HOE-720, HR-720, ICI-D6888, ICI-D7155, ICI-D8731, KRI-1177, KT3-671, KT-3579, KW-3433, L-158809, L-158978,, L-159282, L-159689, L-159874, L-161177, L-162154, L-162234, L-162441, L-163007, L-163017, LF-70156, LR B087, LRB-057, LRB-081, LY-235656, LY-266099, LY-285434, LY-301875, LY-302289, LY-315995, ME-3221, MK-954, PD-123177, PD-123319, PD-126055, PD-150304, RG-13647, RWJ-38970, RWJ-46458, S-8307, S-8308, SC-51757, SC-54629, SC-52458, SL-910102, TAK-536, UP-2696, U-96849, U-97018, UK-77778, UP-275-22, WAY-126227, WK-1260, WK-1360, WK-1492, YH-1498, YM-358, YM-31472, X-6803, XH-148, XR-510, ZD-6888, ZD-7155, ZD-8731, and the like. Suitable angiotensin II antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable β-adrenergic agonists include, but are not limited to, albuterol, bambuterol, bitolterol, carbuterol, clenbuterol, dobutamnine, fenoterol, formoterol, hexoprenaline, isoprotenerol, mabuterol, metaproterenol, pirbuterol, prenalterol, procaterol, protokylol, ritodrine, rimiterol, reproterol, salmeterol, soterenol, terbutaline, tretoquinol, tulobuterol, and the like. Suitable β-adrenergic agonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable anti-hyperlipidemic drugs include, but are not limited to, statins or HMG-CoA reductase inhibitors, such as, for example, atorvastatin (LIPITOR®), bervastatin, cerivastatin (BAYCOL®), dalvastatin, fluindostatin (Sandoz XU-62-320), fluvastatin, glenvastatin, lovastatin (MEVACOR®), mevastatin, privastatin (PRAVACHOL®), rosuvastatin (CRESTOR®), simvastatin (ZOCOR®), velostatin (also known as synvinolin), GR-95030, SQ 33,600, BMY 22089, BMY 22,566, CI 980, and the like; gemfibrozil, cholystyramine, colestipol, nicotinic acid, bile acid sequestrants, such as, for example, cholestyramine, colesevelam, colestipol, poly(methyl-(3-trimethylaminopropyl) imino-trimethylene dihalide) and the like; probucol; fibric acid agents or fibrates, such as, for example, bezafibrate (Bezalip™), beclobrate, binifibrate, ciprofibrate, clinofibrate, clofibrate, etofibrate, fenofibrate (Lipidil™, Lipidil Micro™), gemfibrozil (Lopid™), nicofibrate, pirifibrate, ronifibrate, simfibrate, theofibrate and the like. Suitable anti-hyperlipidemic drugs are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable angiotensin-converting enzyme inhibitors (ACE inhibitors) include, but are not limited to, alacepril, benazepril, benazeprilat, captopril, ceronapril, cilazapril, delapril, duinapril, enalapril, enalaprilat, fosinopril, imidapril, lisinopril, moveltipril, moexipril, naphthopidil, pentopril, perindopril, quinapril, ramipril, rentipril, spirapril, temocapril, trandolapril, urapidil, zofenopril, acylmercapto and mercaptoalkanoyl pralines, carboxyalkyl dipeptides, carboxyalkyl dipeptide, phosphinylalkanoyl pralines, and the like.

Sutiable antioxidants include, but are not limited to, small-molecule antioxidants and antioxidant enzymes. Suitable small-molecule antioxidants include, but are not limited to, hydralazine compounds, glutathione, vitamin C, vitamin E, cysteine, N-acetyl-cysteine, β-carotene, ubiquinone, ubiquinol-10, tocopherols, coenzyme Q, superoxide dismutase mimetics and the like. Suitable antioxidant enzymes include, but are not limited to, superoxide dismutase, catalase, glutathione peroxidase, and the like. The antioxidant enzymes can be delivered by gene therapy as a viral vertor and/or a non-viral vector. Suitable antioxidants are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable β-adrenergic antagonists include, but are not limited to, acebutolol, alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butafilolol, carazolol, carteolol, carvedilol, celiprolol, cetamolol, cindolol, cloranolol, dilevalol, epanolol, esmolol, indenolol, labetalol, landiolol, mepindolol, metipranolol, metoprolol, moprolol, nadolol, nadoxolol, nebivolol, nifenalol, nipradilol, oxprenolol, penbutolol, pindolol, practolol, pronethalol, propranolol, sotalol, sulfinalol, talinolol, tertatolol, tilisolol, timolol, toliprolol, xibenolol, and the like. Suitable beta-adrenergic blockers are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable endothelin antagonists include, but are not limited to, bosentan, endothelin, sulfonamide endothelin antagonists, BQ-123, SQ 28608, and the like. Suitable endothelin antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable neutral endopeptidase inhibitors include, but are not limited to, atrial natriuretic peptides, diazapins, azepinones, ecadotril, omapatrilat, sampatrilat, BMS 189,921, and the like. Neutral endopeptidase inhibitors are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable NSAIDs include, but are not limited to, acetaminophen, acemetacin, aceclofenac, alminoprofen, amfenac, bendazac, benoxaprofen, bromfenac, bucloxic acid, butibufen, carprofen, cinmetacin, clopirac, diclofenac, etodolac, felbinac, fenclozic acid, fenbufen, fenoprofen, fentiazac, flunoxaprofen, flurbiprofen, ibufenac, ibuprofen, indomethacin, isofezolac, isoxepac, indoprofen, ketoprofen, lonazolac, loxoprofen, metiazinic acid, mofezolac, miroprofen, naproxen, oxaprozin, pirozolac, pirprofen, pranoprofen, protizinic acid, salicylamide, sulindac, suprofen, suxibuzone, tiaprofenic acid, tolmetin, xenbucin, ximoprofen, zaltoprofen, zomepirac, aspirin, acemetcin, bumadizon, carprofenac, clidanac, diflunisal, enfenarnic acid, fendosal, flufenamic acid, flunixin, gentisic acid, ketorolac, meclofenamic acid, mefenamic acid, mesalamine, prodrugs thereof, and the like. Suitable NSAIDs are described more fully in the literature, such as in Goodman and Gilman, The Pharmacological Basis of Therapeutics (9th Edition), McGraw-Hill, 1995, Pgs. 617-657; the Merck Index on CD-ROM, 13^{th} Edition; and in U.S. Pat. Nos. 6,057,347 and 6,297,260 assigned to NitroMed Inc., the disclosures of which are incorporated herein by reference in their entirety.

Suitable potassium channel blockers include but are not limited to, nicorandil, pinacidil, cromakalim (BRL 34915), aprikalim, bimakalim, emakalim, lemakalim, minoxidil, diazoxide, 9-chloro-7-(2-chlorophenyl)-5H-pyrimido(5,4,-d)(2)-benzazepine, Ribi, CPG-11952, CGS-9896, ZD 6169, diazixide, Bay X 9227, P1075, Bay X 9228, SDZ PCO 400, WAY-120,491, WAY-120,129, Ro 31-6930, SR 44869, BRL 38226, S 0121, SR 46142A, CGP 42500, SR 44994, artilide fumarate, lorazepam, temazepam, rilmazafone, nimetazepam, midazolam, lormetazepam, loprazolam, ibutilide fumarate, haloxazolam, flunitrazepam, estazolam, doxefazepam, clonazepam, cinolazepam, brotizolam, and the like. Suitable potassium channel blockers are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable platelet reducing agents include but are not limited to, fibrinolytic agents such as for example, ancrod, anistreplase, bisobrin lactate, brinolase, Hageman factor (i.e. factor XII) fragments, molsidomine, plasminogen activators such as, for example, streptokinase, tissue plasminogen activators (TPA), urokinase, pro-Urokinase, recombinant TPA, plasmin, plasminogen, and the like; anti-coagulant agents including but are not limited to, inhibitors of factor Xa, factor TFPI, factor VIIa, factor IXc, factor Va, factor VIIIa, inhibitors of other coagulation factors, and the like; vitamin K antagonists, such as, for example, coumarin, coumarin derivatives (e.g., warfarin sodium); glycosoaminoglycans such as, for example, heparins both in unfractionated form and in low molecular weight form; ardeparin sodium, bivalirudin, bromindione, coumarin, dalteparin sodium, danaparoid sodium; dazoxiben hydrochloride, desirudin, dicumarol, efegatran sulfate, enoxaparin sodium, ifetroban, ifetroban sodium, lyapolate sodium, nafamostat mesylate, phenprocoumon, sulfatide, tinzaparin sodium, retaplase; trifenagrel, warfarin, dextrans and the like; acadesine, anipamil, argatroban, aspirin, clopidogrel, diadenosine 5′,5′″-P1,P4-tetraphosphate (Ap4A) analogs, difibrotide, dilazep dihydrochloride, dipyridamole, dopamine, 3-methoxytyramine, glucagon, glycoprotein IIb/IIIa antagonists, such as, for example, Ro-43-8857, L-700,462, iloprost, isocarbacyclin methyl ester, itazigrel, ketanserin, BM-13.177, lamifiban, lifarizine, molsidomine, nifedipine, oxagrelate, prostaglandins, platelet activating factor antagonists such as, for example, lexipafant, prostacyclins, pyrazines, pyridinol carbamate, ReoPro (i.e., abciximab), sulfinpyrazone, synthetic compounds BN-50727, BN-52021, CV-4151, E-5510, FK-409, GU-7, KB-2796, KBT-3022, KC-404, KF-4939, OP-41483, TRK-100, TA-3090, TFC-612, ZK-36374, 2,4,5,7-tetrathiaoctane, 2,4,5,7-tetrathiaoctane 2,2-dioxide, 2,4,5-trithiahexane, theophyllin pentoxifyllin, thromboxane and thromboxane synthetase inhibitors such as, for example, picotamide, sulotroban, ticlopidine, tirofiban, trapidil, ticlopidine, trifenagrel, trilinolein, 3-substituted 5,6-bis(4-methoxyphenyl)-1,2,4-triazines; antibodies to glycoprotein IIb/IIIa; anti-serotonin drugs, such as, for example, clopridogrel; sulfinpyrazone and the like; aspirin; dipyridamole; clofibrate; pyridinol carbamate; glucagon, caffeine; theophyllin pentoxifyllin; ticlopidine, and the like.

Suitable renin inhibitors include, but are not limited to, aldosterone, aliskiren (SPP-100), enalkrein (A-64662), medullipin, tonin, RO 42-5892 (remikiren), A 62198, A 64662, A 65317, A 72517 (zankiren), A 74273, CP 80794, CGP 29287, CGP-38560A, CPG 29287, EMD 47942, ES 305, ES 1005, ES 8891, FK 906, H 113, H-142, KRI 1314, pepstatin A, RO 44-9375 (ciprokiren), SR-43845, SQ 34017, U 71038, YM-21095, YM-26365, urea derivatives of peptides, amino acids connected by nonpeptide bonds, di- and tri-peptide derivatives (e.g., Act-A, Act-B, Act-C, ACT-D, and the like), amino acids and derivatives thereof, diol sulfonamides and sulfinyls, modified peptides, peptidyl beta-aminoacyl aminodiol carbamates, monoclonal antibodies to renin, and the like. Suitable renin inhibitors are described more fully in U.S. Pat. Nos. 5,116,835, 5,114,937, 5,106,835, 5,104,869, 5,095,119, 5,098,924), 5,095,006, 5,089,471, 5,075,451, 5,066,643, 5,063,208, 4,845,079, 5,055,466, 4,980,283, 4,885,292), 4,780,401, 5,071,837, 5,064,965, 5,063,207, 5,036,054, 5,036,053, 5,034,512, and 4,894,437, the disclosures of each of which are incorporated herein by reference in their entirety; and in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable COX-2 inhibitors include, but are not limited to, NS-386, nimesulide, flosulide, celecoxib, rofecoxib, COX-189, etoracoxib, valdecoxib, Bextra, Dynastat, Arcoxia, SC-57666, DuP 697, GW-406381, SC-58125, SC-58635, and the like, and mixtures of two or more thereof. Suitable COX-2 inhibitors are in U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, and 5,639,780 and in WO 94/03387, WO 94/15723, WO 94/20480, WO 94/26731, WO 94/27980, WO 95/00501, WO 95/15316, WO 96/03387, WO 96/03388, WO 96/06840, WO 96/21667, WO 96/31509, WO 96/36623, WO 97/14691, WO 97/16435, WO 01/45703 and WO 01/87343, the disclosures of each of which are incorporated herein by reference in their entirety; and in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable steroids, include but are not limited to, 21-acetoxypregnenolone, alcolometasone, algestone, amcinonide, beclomethasone, betamethasone, budesonide, chloroprednisone, cidesamide, clobetasol, clobetasone, clocortolone, cloprednol, corticosterone, cortisone, cortivazol (cortivatol), dchenodeoxycholic acid, eflazacort, desonide, desoxycorticosterone, desoximethasone, dexamethasone, diflorasone, diflucortolone, difluprednate, enoxolone, estradiol, ethynylestradiol, fluzacort, fludrocortisone, flucloronide, flumethasone, flunisolide, flucinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, haloprednone acetate, hydrocortamate, hydrocortisone and its derivatives (such as phosphate, 21-sodium succinate and the like), hydrocortisone terbutate, isoflupredone, loteprednol etabonate, mestranol, mazipredone, medrysone, meprednisone, methylprednisolone, mitatrienediol, mometasone furoate, moxestrol, paramethasone, prednicarbate, prednisolone and its derivatives (such as 21-stearoylglycolate, sodium phosphate, 25-diethylaminoacetate, and the like), prednisone, prednival, prednylidene and its derivatives (such as 21-diethylaminoactetate and the like), rimexolone, tixocortol, triamcinolone and its derivatives (such as acetonide, benetonide, and the like), ursodeoxycholic acid, and the like. Suitable steroids are described more fully in the literature, such as in Goodman and Gilman, The Pharmacological Basis of Therapeutics (9th Edition), McGraw-Hill, 1995; the Merck Index on CD-ROM, 13^{th} Edition; the disclosures of which are incorporated herein by reference in their entirety.

Another embodiment of the invention provides compositions comprising at least one compound of the invention, that is optionally nitrosated and/or nitrosylated, and, optionally, at least one nitric oxide donor compound and/or at least one therapeutic agent, bound to a matrix. Preferably, the nitrosated and/or nitrosylated compounds of the invention are the compounds of Formulas (I), (II), (III), (IV), (V), (VI), (VII), (VIII) or (IX). Preferably, the nitric oxide donor compound and the therapeutic agents are those described herein.

The compound of the invention that is optionally nitrosated and/or nitrosylated, and, optionally, NO donors and/or therapeutic agents, can be incorporated into a natural or synthetic matrix which can then be applied with specificity to a biological site of interest. Accordingly the compound of the invention that is optionally nitrosated and/or nitrosylated, and optionally, NO donor and/or therapeutic agent is “bound to the matrix” which means that the compound of the invention that is optionally nitrosated and/or nitrosylated, and, optionally, NO donors and/or therapeutic agent, are physically and/or chemically associated with part of, incorporated with, attached to, or contained within the natural or synthetic matrix. In one embodiment, physical association or bonding can be achieved, for example, by coprecipitation of the compound of the invention, that is optionally nitrosated and/or nitrosylated, and, optionally, NO donor and/or therapeutic agent, with the matrix. In another embodiment, chemical association or bonding can be achieved by, for example, covalent bonding of a nucleophillic moiety of the compound of the invention that is optionally nitrosated and/or nitrosylated, and, optionally, NO donor, and/or therapeutic agent, to the matrix, such that the compound of the invention that is optionally nitrosated and/or nitrosylated, is part of the matrix itself. In yet another embodiment, the compound of the invention that is optionally nitrosated and/or nitrosylated, and, optionally, NO donor, and/or therapeutic agent can be incorporated into a porous layer of the matrix or into pores included in the natural or synthetic matrix. The manner in which the compound of the invention that is optionally nitrosated and/or nitrosylated, and, optionally, NO donor and/or therapeutic agent, is associated, part of, attached to, incorporated with or contained within (i.e. “bound to”) the matrix is inconsequential to the invention and all means of association, incorporation, attachment, and bonding are contemplated herein. Incorporation of the compound of the invention, that is optionally nitrosated and/or nitrosylated, and, optionally, NO donors, and/or therapeutic agents, into the matrix results in site-specific application, thereby enhancing selectivity of action for the released nitric oxide and the compound of the invention. Additionally, incorporation of the compound of the invention that is optionally nitrosated and/or nitrosylated, into the matrix reduces the rate of release of the nitric oxide and the compound of the invention. This prolongs the release of the nitric oxide and the compound of the invention thereby allowing for efficient dosing to achieve a desired biological effect so that the frequency of dosing can be reduced.

Any of a wide variety of natural or synthetic polymers can be used as the matrix in the context of the invention. It is only necessary for the matrix to be biologically acceptable. Exemplary matrixes suitable for use in the invention are polymers including, for example, polyolefins (such as, polystyrene, polyalkylenes, polypropylene, polyethylene, high molecular weight polyethylene, polyethylene oxides, high density polyethylene, polytetrafluorethylene, polyvinylidene diflouride and polyvinylchloride), polyethylenimine or derivatives thereof, polyethers (such as, polyethylene glycol), polyesters (such as, poly-L-lactic acid, poly-D, L-lactic, poly-D-lactic, polyglycolic acid, poly-(lactide/glycolide, polyethylene terephthalate), polyether sulfones, polyanhydrides, polyhydroxybutyrates, polyamides (such as, nylon), polyurethanes, polyurethane copolymers (such as, pellethane polymers), polyacrylates (such as, polymethacrylate, poly (2-(methacryloyloxyethyl)-2′-(trimethylammonium)ethyl phosphate inner salt-co-n-dodecyl methacrylate, methylmethacrylate), polyvinylpyrrolidones, cross-linked polyvinylpyrrolidones, polyvinyl alcohols, polyvinyl acetates, halogenated polyalkylenes, polyvinyl ethers, polyvinyl aromatics, polyurethanes, polyorthoesters, polycarbonates, polyalkylenes, polycarboxylic acids (such as, for example polyacrylic acids), polycaprolactone, polyhydroxybutyrate valerate, silicones, siloxane polymers, hyaluronic acid, mixtures of polymers (such as, polylactic acid/polylysine copolymers, polyalkylene/styrene copolymers, polyurethane/polyester copolymers, polyurethane/polyether copolymers, polyethylene oxide/polypropylene oxides, ethylene-vinyl acetate copolymers, nylon/polyether copolymers, such as vestamid), biopolymers (such as peptides, polypeptides, proteins, chitosan, chitosan derivatives, gelatin, oligonucleotides, antibodies, peptide hormones, glycoproteins, glycogen and nucleic acids, fibrin, collagen), glycosaminoglycans, polysaccharides (such as, for example, cellulose, starches, dextrans, alginates, derivatives such as, cellulose acetate, cellulose nitrate), starburst dendrimers, natural fibrous matrix (such as, filter paper), synthetic fibrous matrix materials (such as, three-dimensional lattice of synthetic polymers and copolymers) and the like. Exemplary polymers are described in U.S. Pat. Nos. 5,705,583, 5,770,645, 5,994,444, 6,087,479 and 6,153,252, the disclosures of each of which are incorporated by reference herein in their entirety. In preferred embodiments the matrix materials are polylactic acid, polyurethane and polyalkene polymers. In another embodiment the matrix material is nitrosated and/or nitrosylated.

The physical and structural characteristics of the matrixes suitable for use in the invention are not critical, but depend on the application. It will be appreciated by one skilled in the art that where the matrix-compound of the invention, that is optionally nitrosated and/or nitrosylated, composition of the invention is intended for local, relatively short term administration or similar administration they need not be biodegradable. For some uses, such as postangioplasty, coronary bypass surgery or intimal hyperplasia associated with vascular or non-vascular graft implants or the like, it may be desirable for the matrix to slowly dissolve in a physiological environment or to be biodegradable.

The nitrosated and/or nitrosylated compound of the invention or compound of the invention, and, optionally, the nitric oxide donor compound and/or therapeutic agent bound to the matrix may be administered in a wide variety of forms or delivery means. Any delivery means should adequately protect the integrity of the nitric oxide prior to its release and should control the release of the nitric oxide at such a rate, in such an amount, and in such a location as to serve as an effective means for prevention and/or treatment of cardiovascular diseases and disorders, including restenosis. Delivery means for local administration include, but are not limited to, those described herein. Delivery means for systemic administration include, for example, solutions, suspensions, emulsions, capsules, powders, sachets, tablets, effervescent tablets, topical patches, lozenges, aerosols, liposomes, microparticles, microspheres, beads and the like. The matrix itself may be structurally sufficient to serve as a delivery means.

The nitrosated and/or nitrosylated compound of the invention or compound of the invention and, optionally, the nitric oxide donor compound and/or therapeutic agent, bound to the matrix can also be used to coat all or a portion of the surface of a medical device that comes into contact with blood (including blood components and blood products), vascular or non-vascular tissue thereby rendering the surface passive. Alternatively the compound of the invention that is optionally nitrosated and/or nitrosylated, and the nitric oxide donor compound, and, optionally, the therapeutic agent, bound to the matrix can also be used to coat all or a portion of the surface of a medical device that comes into contact with blood (including blood components and blood products), vascular or non-vascular tissue thereby rendering the surface passive. U.S. Pat. Nos. 5,665,077, 5,797,887, 5,824,049 and 5,837,008, the disclosures of each of which are incorporated by reference herein in their entirety, describe methods for coating all or a portion of a surface of a medical device. Thus, for example, (i) all or a portion of the medical device may be coated with the compound of the invention that is optionally nitrosated and/or nitrosylated, and, optionally, NO donors and/or therapeutic agents, either as the coating per se or bound to a matrix, as described herein; or (ii) all or a portion of the medical device may be produced from a material which includes the compound of the invention that is optionally nitrosated and/or nitrosylated, and, optionally, NO donor and/or therapeutic agent, per se or bound to a matrix, as described herein.

It is also contemplated that artificial surfaces will vary depending on the nature of the surface, and such characteristics including contour, crystallinity, hydrophobicity, hydrophilicity, capacity for hydrogen bonding, and flexibility of the molecular backbone and polymers. Therefore, using routine methods, one of ordinary skill will be able to customize the coating technique by adjusting such parameters as the amount of adduct, length of treatment, temperature, diluents, and storage conditions, in order to provide optimal coating of each particular type of surface.

After the medical device or artificial material has been coated with the nitrosated and/or nitrosylated compound of the invention, and, optionally, NO donor and/or therapeutic agent, or with the compound of the invention, and NO donor, and, optionally, the therapeutic agent, it will be suitable for its intended use, including, for example, implantation as a heart valve, insertion as a catheter, insertion as a stent, or for cardiopulmonary oxygenation or hemodialysis.

In another embodiment, the compound of the invention, that is optionally nitrosated and/or nitrosylated, and, optionally, NO donor, and/or therapeutic agent can be directly incorporated into the pores or reservoirs of the medical device (i.e. without a matrix or polymer). A coating of a biocompatible polymer/material could be applied over the medical device which would control the diffusion of the compound of the invention, that is optionally nitrosated and/or nitrosylated, and, optionally, NO donor, and/or therapeutic agent from the pores or reservoirs of the medical device. The manner in which the compound of the invention that is optionally nitrosated and/or nitrosylated, and, optionally, NO donor and/or therapeutic agent, is associated, part of, attached to, incorporated with or contained within (i.e. “bound to”) the medical device is inconsequential to the invention and all means of association, incorporation, attachment, and bonding are contemplated herein. Incorporation of the compound of the invention that is optionally nitrosated and/or nitrosylated, and, optionally, NO donors, and/or therapeutic agents, into the pores or reservoirs of the medical device results in site-specific application, thereby enhancing selectivity of action for the released nitric oxide and compound of the invention. Additionally, incorporation of the compound of the invention, that is optionally nitrosated and/or nitrosylated, into the pores or reservoirs of the medical device reduces the rate of release of the nitric oxide and the compound of the invention. This prolongs the release of the nitric oxide and the compound of the invention thereby allowing for efficient dosing to achieve a desired biological effect so that the frequency of dosing can be reduced.

The invention also describes methods for the administration of a therapeutically effective amount of the compounds and compositions described herein for treating or preventing cardiovascular diseases and disorders including, for example, restenosis and atherosclerosis. For example, the patient can be administered a therapeutically effective amount of at least one nitrosated and/or nitrosylated compound of the invention. In another embodiment, the patient can be administered a therapeutically effective amount of at least one compound of the invention, optionally substituted with at least one NO and/or NO₂ group, and at least one nitric oxide donor compound. In yet another embodiment, the patient can be administered a therapeutically effective amount of at least one compound of the invention, optionally substituted with at least one NO and/or NO₂ group, and at least one therapeutic agent, and, optionally, at least one nitric oxide donor compound. The compounds can be administered separately or in the form of a composition.

Another embodiment of the invention provides methods for the prevention of platelet aggregation and platelet adhesion caused by the exposure of blood (including blood components or blood products) to a medical device by incorporating at least one nitrosated and/or nitrosylated compound of the invention or compound of the invention, and, optionally, at least one nitric oxide donor compound, and/or therapeutic agent, into and/or on the portion(s) of the medical device that come into contact with blood (including blood components or blood products), vascular or non-vascular tissue. The compound of the invention, that is optionally nitrosated and/or nitrosylated, and, optionally, NO donors, may be directly or indirectly linked to the natural or synthetic polymeric material from which all or a portion of the device is made, as disclosed in U.S. Pat. No. 6,087,479, assigned to NitroMed, the disclosure of which is incorporated by reference herein in its entirety. Alternatively, the compound of the invention that is optionally nitrosated and/or nitrosylated, and, optionally, NO donors, may be incorporated into the body of the device which is formed of a biodegradable or bioresorbable material, including the matrix described herein. Thus the nitric oxide is released over a sustained period of the resorption or degradation of the body of the device.

Another embodiment of the invention provides methods to prevent or treat pathological conditions resulting from abnormal cell proliferation, transplant rejections, autoimmune, inflammatory, proliferative, hyperproliferative or vascular diseases, to reduce scar tissue and to inhibit wound contraction by administering to a patient in need thereof a therapeutically effective amount of the compounds and/or compositions described herein. For example, the patient can be administered a therapeutically effective amount of at least one nitrosated and/or nitrosylated compound of the invention. In another embodiment, the patient can be administered a therapeutically effective amount of at least one compound of the invention, optionally substituted with at least one NO and/or NO₂ group, and at least one nitric oxide donor compound. In yet another embodiment, the patient can be administered a therapeutically effective amount of at least one compound of the invention, optionally substituted with at least one NO and/or NO₂ group, and at least one therapeutic agent, and, optionally, at least one nitric oxide donor compound. The compound of the invention optionally substituted with at least one NO and/or NO₂ group, nitric oxide donors and/or therapeutic agents can be administered separately or in the form of a composition. The compounds and compositions of the invention can also be administered in combination with other medications used for the treatment of these disorders.

Another embodiment of the invention relates to systemic and/or local administration of the nitrosated and/or nitrosylated compound of the invention and/or compound of the invention, and, optionally, at least one nitric oxide donor compound, to the site of injured or damaged tissue (e.g., damaged blood vessels) for the treatment of the injured or damaged tissue. Such damage may result from the use of a medical device in an invasive procedure. Thus, for example, in treating blocked vasculature by, for example, angioplasty, damage can result to the blood vessel. Such damage may be treated by use of the compounds and compositions described herein. In addition to repair of the damaged tissue, such treatment can also be used to prevent and/or alleviate and/or delay re-occlusions, for example, restenosis. The compounds and compositions can be locally delivered using any of the methods known to one skilled in the art, including but not limited to, a drug delivery catheter, an infusion catheter, a drug delivery guidewire, an implantable medical device, and the like. In one embodiment, all or most of the damaged area is coated with the nitrosated and/or nitrosylated compound of the invention described herein per se or in a pharmaceutically acceptable carrier or excipient which serves as a coating matrix, including the matrix described herein. This coating matrix can be of a liquid, gel or semisolid consistency. The nitrosated and/or nitrosylated compound of the invention can be applied in combination with one or more therapeutic agents, such as those listed above. The carrier or matrix can be made of or include agents which provide for metered or sustained release of the therapeutic agents.

In preventing and/or treating cardiovascular diseases and disorders, the nitrosated and/or nitrosylated compound of the invention and, optionally, at least one nitric oxide donor compound can be administered directly to the damaged vascular or non-vascular surface intravenously by using an intraarterial or intravenous catheter, suitable for delivery of the compounds to the desired location. The location of damaged arterial surfaces is determined by conventional diagnostic methods, such as X-ray angiography, performed using routine and well-known methods available to one skilled in the art. In addition, administration of the nitrosated and/or nitrosylated compound of the inventions, and, optionally, NO donors, using an intraarterial or intravenous catheter is performed using routine methods well known to one skilled in the art. Typically, the compound or composition is delivered to the site of angioplasty through the same catheter used for the primary procedure, usually introduced to the carotid or coronary artery at the time of angioplasty balloon inflation. The nitrosated and/or nitrosylated compounds of the invention, and, optionally, NO donors, slowly decompose at body temperature over a prolonged period of time releasing nitric oxide at a rate effective to prevent and/or treat cardiovascular diseases and disorders including, for example, restenosis.

When administered in vivo, the compounds and compositions of the invention, can be administered in combination with pharmaceutically acceptable carriers and in dosages described herein. When the compounds and compositions of the invention are administered as a mixture of at least one compound of the invention, that is optionally nitrosated and/or nitrosylated, and at least one nitric oxide donor, they can also be used in combination with one or more additional compounds which are known to be effective against the specific disease state targeted for treatment (e.g., therapeutic agents). The nitric oxide donors and/or therapeutic agents can be administered simultaneously with, subsequently to, or prior to administration of the compound of the invention, including those that are substituted with one or more NO and/or NO₂ groups, and/or other additional compounds.

The compounds and compositions of the invention can be administered by any available and effective delivery system including, but not limited to, orally, bucally, parenterally, by inhalation spray, by topical application, by injection or rectally (e.g., by the use of suppositories) in dosage unit formulations containing conventional nontoxic pharmaceutically acceptable carriers, adjuvants, and vehicles, as desired. Injection includes subcutaneous injections, intravenous, intramuscular, intrasternal injection, or infusion techniques.

Transdermal compound administration, which is known to one skilled in the art, involves the delivery of pharmaceutical compounds via percutaneous passage of the compound into the systemic circulation of the patient. Topical administration can also involve the use of transdermal administration such as, transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, pastes, sprays, lotions, gels, ointments, eye drops, nose drops, ear drops, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous crosslinking agent impregnated with the composition and laminated to an impermeable backing.

Solid dosage forms for oral administration can include capsules, tablets, effervescent tablets, chewable tablets, pills, powders, sachets, granules and gels. In such solid dosage forms, the active compounds can be admixed with at least one inert diluent such as, sucrose, lactose or starch. Such dosage forms can also comprise, as in normal practice, additional substances other than inert diluents, e.g., lubricating agents such as, magnesium stearate. In the case of capsules, tablets, effervescent tablets, and pills, the dosage forms can also comprise buffering agents. Soft gelatin capsules can be prepared to contain a mixture of the active compounds or compositions of the invention and vegetable oil. Hard gelatin capsules can contain granules of the active compound in combination with a solid, pulverulent carrier such as, lactose, saccharose, sorbitol, mannitol, potato starch, corn starch, amylopectin, cellulose derivatives of gelatin. Tablets and pills can be prepared with enteric coatings. Oral formulations containing compounds of the invention are disclosed in U.S. Pat. Nos. 5,559,121, 5,536,729, 5,989,591 and 5,985,325, the disclosures of each of which are incorporated by reference herein in their entirety.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Suppositories for vaginal or rectal administration of the compounds and compositions of the invention can be prepared by mixing the compounds or compositions with a suitable nonirritating excipient such as, cocoa butter and polyethylene glycols which are solid at room temperature but liquid at bodytemperature, such that they will melt and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium. Parenteral formulations containing compounds of the invention are disclosed in U.S. Pat. Nos. 5,530,006, 5,516,770 and 5,626,588, the disclosures of each of which are incorporated by reference herein in their entirety.

The compositions of this invention can further include conventional excipients, i.e., pharmaceutically acceptable organic or inorganic carrier substances suitable for parenteral application which do not deleteriously react with the active compounds. Suitable pharmaceutically acceptable carriers include, for example, water, salt solutions, alcohol, vegetable oils, polyethylene glycols, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances that increase the viscosity of the suspension and include, for example, sodium carboxymethyl cellulose, sorbitol and/or dextran. Optionally, the suspension may also contain stabilizers.

Solvents useful in the practice of this invention include pharmaceutically acceptable, water-miscible, non-aqueous solvents. In the context of this invention, these solvents should be taken to include solvents that are generally acceptable for pharmaceutical use, substantially water-miscible, and substantially non-aqueous. Preferably, these solvents are also non-phthalate plasticizer leaching solvents, so that, when used in medical equipment, they substantially do not leach phthalate plasticizers that may be present in the medical equipment. More preferably, the pharmaceutically-acceptable, water-miscible, non-aqueous solvents usable in the practice of this invention include, but are not limited to, N-methyl pyrrolidone (NMP); propylene glycol; ethyl acetate; dimethyl sulfoxide; dimethyl acetamide; benzyl alcohol; 2-pyrrolidone; benzyl benzoate; C₂₋₆ alkanols; 2-ethoxyethanol; alkyl esters such as, 2-ethoxyethyl acetate, methyl acetate, ethyl acetate, ethylene glycol diethyl ether, or ethylene glycol dimethyl ether; (S)-(−)-ethyl lactate; acetone; glycerol; alkyl ketones such as, methylethyl ketone or dimethyl sulfone; tetrahydrofuran; cyclic alkyl amides such as, caprolactam; decylmethylsulfoxide; oleic acid; aromatic amines such as, N,N-diethyl-m-toluamide; or 1-dodecylazacycloheptan-2-one.

The preferred pharmaceutically-acceptable, water-miscible, non-aqueous solvents are N-methyl pyrrolidone (NMP), propylene glycol, ethyl acetate, dimethyl sulfoxide, dimethyl acetamide, benzyl alcohol, 2-pyrrolidone, or benzyl benzoate. Ethanol may also be used as a pharmaceutically-acceptable, water-miscible, non-aqueous solvent according to the invention, despite its negative impact on stability. Additionally, triacetin may also be used as a pharmaceutically-acceptable, water-miscible, non-aqueous solvent, as well as functioning as a solubilizer in certain circumstances. NMP may be available as PHARMASOLVE® from International Specialty Products (Wayne, N.J.). Benzyl alcohol may be available from J. T. Baker, Inc. Ethanol may be available from Spectrum, Inc. Triacetin may be available from Mallinkrodt, Inc.

The compositions of this invention can further include solubilizers. Solubilization is a phenomenon that enables the formation of a solution. It is related to the presence of amphiphiles, that is, those molecules that have the dual properties of being both polar and non-polar in the solution that have the ability to increase the solubility of materials that are normally insoluble or only slightly soluble, in the dispersion medium. Solubilizers often have surfactant properties. Their function may be to enhance the solubility of a solute in a solution, rather than acting as a solvent, although in exceptional circumstances, a single compound may have both solubilizing and solvent characteristics. Solubilizers useful in the practice of this invention include, but are not limited to, triacetin, polyethylene glycols (such as, for example, PEG 300, PEG 400, or their blend with 3350, and the like), polysorbates (such as, for example, Polysorbate 20, Polysorbate 40, Polysorbate 60, Polysorbate 65, Polysorbate 80, and the like), poloxamers (such as, for example, Poloxamer 124, Poloxamer 188, Poloxamer 237, Poloxamer 338, Poloxamer 407, and the like), polyoxyethylene ethers (such as, for example, Polyoxyl 2 cetyl ether, Polyoxyl 10 cetyl ether, and Polyoxyl 20 cetyl ether, Polyoxyl 4 lauryl ether, Polyoxyl 23 lauryl ether, Polyoxyl 2 oleyl ether, Polyoxyl 10 oleyl ether, Polyoxyl 20 oleyl ether, Polyoxyl 2 stearyl ether, Polyoxyl 10 stearyl ether, Polyoxyl 20 stearyl ether, Polyoxyl 100 stearyl ether, and the like), polyoxylstearates (such as, for example, Polyoxyl 30 stearate, Polyoxyl 40 stearate, Polyoxyl 50 stearate, Polyoxyl 100 stearate, and the like), polyethoxylated stearates (such as, for example, polyethoxylated 12-hydroxy stearate, and the like), and Tributyrin.

Other materials that may be added to the compositions of the invention include cyclodextrins, and cyclodextrin analogs and derivatives, and other soluble excipients that could enhance the stability of the inventive composition, maintain the product in solution, or prevent side effects associated with the administration of the inventive composition. Cyclodextrins may be available as ENCAPSIN® from Janssen Pharmaceuticals.

The composition, if desired, can also contain minor amounts of wetting agents, emulsifying agents and/or pH buffering agents. The composition can be a liquid solution, suspension, emulsion, tablet, pill, capsule, sustained release formulation, or powder. The composition can be formulated as a suppository, with traditional binders and carriers such as, triglycerides. Oral formulations can include standard carriers such as, pharmaceutical grades of mannitol, lactose, starch, magnesium stearate, sodium saccharine, cellulose, magnesium carbonate, and the like.

Various delivery systems are known and can be used to administer the compounds or compositions of the invention, including, for example, encapsulation in liposomes, microbubbles, emulsions, microparticles, microcapsules, nanoparticles, and the like. The required dosage can be administered as a single unit or in a sustained release form.

The bioavailabilty of the compositions can be enhanced by micronization of the formulations using conventional techniques such as, grinding, milling, spray drying and the like in the presence of suitable excipients or agents such as, phospholipids or surfactants.

Sustained release dosage forms of the invention may comprise microparticles and/or nanoparticles having a therapeutic agent dispersed therein or may comprise the therapeutic agent in pure, preferably crystalline, solid form. For sustained release administration, microparticle dosage forms comprising pure, preferably crystalline, therapeutic agents are preferred. The therapeutic dosage forms of this aspect of the invention may be of any configuration suitable for sustained release. Preferred sustained release therapeutic dosage forms exhibit one or more of the following characteristics: microparticles (e.g., from about 0.5 micrometers to about 100 micrometers in diameter, preferably about 0.5 to about 2 micrometers; or from about 0.01 micrometers to about 200 micrometers in diameter, preferably from about 0.5 to about 50 micrometers, and more preferably from about 2 to about 15 micrometers) or nanoparticles (e.g., from about 1.0 nanometer to about 1000 nanometers in diameter, preferably about 50 to about 250 nanometers ; or from about 0.01 nanometer to about 1000 nanometers in diameter, preferably from about 50 to about 200 nanometers), free flowing powder structure; biodegradable structure designed to biodegrade over a period of time between from about 0.5 to about 180 days, preferably from about 1 to 3 to about 150 days, more preferably from about 3 to about 180 days, and most preferably from about 10 to about 21 days; or non-biodegradable structure to allow the therapeutic agent diffusion to occur over a time period of between from about 0.5 to about 180 days, more preferably from about 30 to about 120 days; or from about 3 to about 180 days, more preferably from about 10 to about 21 days; biocompatible with target tissue and the local physiological environment into which the dosage form to be administered, including yielding biocompatible biodegradation products; facilitate a stable and reproducible dispersion of therapeutic agent therein, preferably to form a therapeutic agent-polymer matrix, -with active therapeutic agent release occurring by one or both of the following routes: (1) diffusion of the therapeutic agent through the dosage form (when the therapeutic agent is soluble in the shaped polymer or polymer mixture defining the dimensions of the dosage form); or (2) release of the therapeutic agent as the dosage form biodegrades; and/or for targeted dosage forms, capability to have, preferably, from about 1 to about 10,000 binding protein/peptide to dosage form bonds and more preferably, a maximum of about 1 binding peptide to dosage form bond per 150 square angstroms of particle surface area. The total number of binding protein/peptide to dosage form bonds depends upon the particle size used. The binding proteins or peptides are capable of coupling to the particles of the therapeutic dosage form through covalent ligand sandwich or non-covalent modalities as set forth herein.

Nanoparticle sustained release therapeutic dosage forms are preferably biodegradable and, optionally, bind to the vascular or non-vascular smooth muscle cells and enter those cells, primarily by endocytosis. The biodegradation of the nanoparticles occurs over time (e.g., 30 to 120 days; or 10 to 21 days) in prelysosomic vesicles and lysosomes. Larger microparticle therapeutic dosage forms of the invention release the therapeutic agents for subsequent target cell uptake with only a few of the smaller microparticles entering the cell by phagocytosis. A practitioner in the art will appreciate that the precise mechanism by which a target cell assimilates and metabolizes a dosage form of the invention depends on the morphology, physiology and metabolic processes of those cells. The size of the particle sustained release therapeutic dosage forms is also important with respect to the mode of cellular assimilation. For example, the smaller nanoparticles can flow with the interstitial fluid between cells and penetrate the infused tissue. The larger microparticles tend to be more easily trapped interstitially in the infused primary tissue, and thus are useful to deliver anti-proliferative therapeutic agents.

Preferred sustained release dosage forms of the invention comprise biodegradable microparticles or nanoparticles. More preferably, biodegradable microparticles or nanoparticles are formed of a polymer containing matrix that biodegrades by random, nonenzymatic, hydrolytic scissioning to release therapeutic agent, thereby forming pores within the particulate structure.

The compounds and compositions of the invention can be formulated as pharmaceutically acceptable salts. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitrous (nitrite salt), nitric (nitrate salt), carbonic, sulfuric, phosphoric acid, and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesuifonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

While individual needs may vary, determination of optimal ranges for effective amounts of the compounds and/or compositons is within the skill of the art. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as, the activity, efficacy, pharmacokinetic and toxicology profiles of the particular compound used, whether a drug delivery system is used, and whether the compound is administered as part of a drug combination.

The usual doses of compound of the invention (including the nitrosated and/or nitrosylated compound of the invention) for intraveneous dosages, can be, but is not limited to about 0.001 mg/kg/day to about 25 mg/kg/day, preferably about 0.005 mg/kg/day to about 5 mg/kg/day and more preferably about 0.01 mg/kg/day to about 0.5 mg/kg/day. The usual doses of compound of the invention (including nitrosated and/or nitrosylated compound of the invention) for oral dosages, can be, but is not limited to about 0.005 mg/kg/day to about 150 mg/kg/day, preferably about 0.05 mg/kg/day to about 100 mg/kg/day and more preferably about 0.01 mg/kg/day to about 10 mg/kg/day.

The doses of nitric oxide donors in the pharmaceutical composition will be dependent on the specific nitric oxide donor compound and the mode of administration. For example, when L-arginine is the orally administered nitric oxide donor, it can be administered in an amount of about 3 grams to about 15 grams to provide a plasma level in the range of about 0.2 mM to about 30 mM. When L-arginine is delivered directly at the site of injury by local administration, the L-arginine is delivered in an amount of at least about 50 mg to about 500 mg, preferably about 100 mg to about 2 g. the time of the treatment will usually be at least about 2 minutes to about 30 minutes, more preferably about 5 minutes to about 15 minutes.

The doses of nitric oxide donors in the pharmaceutical composition will be dependent on the specific nitric oxide donor compound and the mode of administration. For example, when L-arginine is the orally administered nitric oxide donor, it can be administered in an amount of about 3 grams to about 15 grams to provide a plasma level in the range of about 0.2 mM to about 30 mM. When L-arginine is delivered directly at the site of injury by local administration, the L-arginine is delivered in an amount of at least about 50 mg to about 500 mg, preferably about 100 mg to about 2 g. The time of the treatment will usually be at least about 2 minutes to about 30 minutes, more preferably about 5 minutes to about 15 minutes.

The nitrosated and/or nitrosylated compounds of the invention of the invention are used at dose ranges and over a course of dose regimen and are administered in the same or substantially equivalent vehicles/carrier by the same or substantially equivalent as their non-nitrosated/nitrosylated counterparts. The nitrosated and/or nitrosylated compounds of the invention can also be used in lower doses and in less extensive regimens of treatment. The amount of active ingredient that can be combined with the carrier materials to produce a single dosage form will vary depending upon the host treated and the particular mode of administration, and is within the skill in the art.

The invention also provides pharmaceutical kits comprising one or more containers filled with one or more of the ingredients of the pharmaceutical compounds and/or compositions of the invention, including, one or more compounds of the invention, optionally substituted with one or more NO and/or NO₂ groups, and one or more of the NO donors, and one or more therapeutic agents described herein. Such kits can also include, for example, other compounds and/or compositions (e.g., therapeutic agents, permeation enhancers, lubricants, and the like), a device(s) for administering the compounds and/or compositions, and written instructions in a form prescribed by a governmental agency regulating the manufacture, use or sale of pharmaceuticals or biological products, which instructions can also reflects approval by the agency of manufacture, use or sale for human administration.

The disclosure of each patent, patent application and publication cited or described in the specification is hereby incorporated by reference herein in its entirety.

Although the invention has been set forth in detail, one skilled in the art will appreciate that numerous changes and modifications may be made without departing from the spirit and scope of the invention.

### EXAMPLES

The following non-limiting examples further describe and enable one of ordinary skill in the art to make and use the present invention.

### Example 1

### (N-(2-Methyl-2-(nitrosothio)propyl)carbamoyl)methyl 2-((2E)-3-(3,4-dimethoxyphenyl)prop-2-enoylamino)benzoate

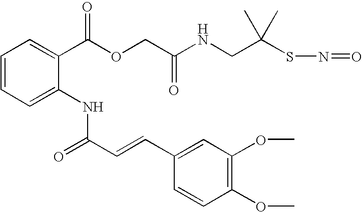

### 1a. 2-((2E)-3-(3,4-Dimethoxyphenyl)prop-2-enoylamino)benzoic acid

The title compound was prepared from 3,4-dimethoxycinnamyl chloride and anthranilic acid according to the procedure in U.S. Pat. No. 3,940,422. ¹H NMR (300 MHz, CDCl₃/d6-DMSO) δ 11.62 (s, 1H), 8.84 (d, J=8.5 Hz, 1H), 8.10 (d, J=8.0 Hz, 1H), 7.66 (d, J=15.5 Hz, 1H), 7.55 (t, J=7.7 Hz, 1H), 7.05-7.18 (m, 3H), 6.89 (d, J=8 Hz, 1H), 6.50 (d, J=15.5 Hz, 1H), 3.95 (s, 3H), 3.92 (s, 3H). Mass spectrum (API-TIS) m/z 328 (MH⁺).

### 1b. tert-Butyl 2-(2-((2E)-3-(3,4-dimethoxyphenyl)prop-2-enoylamino)phenyl carbonyloxy)acetate

The product of Example 1a (3.85 g, 11.8 mmol), potassium carbonate (1.62 g, 11.8 mmol) and tert-butyl bromoacetate (1.9 mL, 2.52 g, 13 mmol) in DMF (60 mL) was stirred at room temperature for 4 hours. The reaction mixture was diluted with a large volume of EtOAc, washed several times with water, satd. NaCl, dried with Na₂SO₄ and filtered. The solvent was evaporated to give the title compound (4.2 g, 81% yield). Mp 116-118° C. ¹H NMR (300 MHz, CDCl₃) δ 11.01 (s, 1H), 8.88 (d, J=8.5 Hz, 1H), 8.15 (dd, J=8.0 and 1.5 Hz, 1H), 7.70 (d, J=15.5 Hz, 1H), 7.55-7.64 (m, 1H), 7.08-7.19 (m, 3H), 6.88 (d, J=8.3 Hz, 1H), 6.51 (d, J=15.5 Hz, 1H), 4.78 (s, 2H), 3.95 (s, 3H), 3.92 (s, 3H), 1.52 (s, 9H). ¹³C NMR (75 MHz, CDCl₃) δ 172.2, 168.0, 166.9, 165.1, 151.3, 149.67, 142.7, 142.3, 135.4, 131.6, 128.1, 122.9, 121.0, 120.0, 114.8, 111.4, 110.2, 83.3, 62.1, 56.4, 56.3, 28.4. Anal. calcd for C₂₄H₂₇NO₇: C, 65.29; H, 6.17; N, 3.17, Found: C, 65.50; H, 6.47; N, 3.06. Mass spectrum (API-TIS) m/z 442 (MH⁺).

### 1c. 2-(2-((2E)-3-(3,4-Dimethoxyphenyl)prop-2-enoylamino)phenylcarbonyloxy)acetic acid

The product of Example 1b (4 g, 9.1 mmol) in a mixture of CH₂Cl₂ (30 mL) and trifluoroacetic acid (20 mL) was stirred at room temperature for 2.5 hours. The volatile material was evaporated to give the title compound (3.5 g, 100% yield). Mp 206-209° C. ¹H NMR (300 MHz, CDCl₃/d₆-DMSO) δ 11.05 (s, 1H), 8.87 (d, J=8.5 Hz, 1H), 8.19 (d, J=8 Hz, 1H), 7.69 (d, J=15.5 Hz, 1H), 7.62 (t, J=7.5 Hz, 1H), 7.46-7.53 (br, s, 1H), 7.10-7.20 (m, 2H), 6.92 (d, J=8.1 Hz, 1H), 6.57 (d, J=15.5 Hz, 1H), 4.90 (s, 2H), 3.98 (s, 3H), 3.94 (s, 3H). ¹³C NMR (75 MHz, CDCl₃) δ 168.8, 167.0. 164.1, 150.4, 148.7, 141.7, 141.3, 134.4, 130.8, 127.1, 122.2, 122.0, 120.0, 119.1, 114.1, 110.6, 109.2, 60.8, 55.5, 55.4. Mass spectrum (API-TIS) m/z 386 (MH⁺).

### 1d. (N-(2-Methyl-2-sulfanylpropyl)carbamoyl)methyl 2-((2E)-3-(3,4-dimethoxyphenyl)prop-2-enoylamino)benzoate

The product of Example 1c (1.2 g, 3.1 mmol), triethylamine (480 μL, 345 mg, 3.4 mmol), 4-dimethylaminopyridine (75 mg, 0.6 mmol) and 2-mercapto-2-methyl-1-propylamine hydrochloride (482 mg, 3.4 mmol) in DMF (15 mL) was treated with 1-(3-(dimethylamino) propyl)-3-ethylcarbodiimide hydrochloride (653 mg, 3.4 mmol). The reaction mixture was stirred at room temperature overnight, diluted with a large volume of EtOAc, washed several times with water, satd. NaCl, dried with Na₂SO₄ and filtered. The residue after evaporation was chromatographed on silica gel, eluting with EtOAc:Hexane 1:1 to give the title compound (0.3 g, 21% yield). Mp 148-150° C. ¹H NMR (300 MHz, CDCl₃) δ 11.02 (s, 1H), 8.92 (d, J=8.5 Hz, 1H), 8.14 (d, J=7.9 Hz, 1H), 7.71 (d, J=14.7 Hz, 1H), 7.64 (t, J=8.2 Hz, 1H), 7.08-7.20 (m, 3H), 6.89 (d, J=8.2 Hz, 1H), 6.68-6.78 (br s, 1H), 6.48 (d, J=15.5 Hz, 1H), 4.88 (s, 2H), 3.96 (s, 3H), 3.92 (s, 3H), 3.42 (d, J=6.1 Hz, 2H), 1.61 (s, 1H), 1.39 (s, 6H). ¹³C NMR (75 MHz, CDCl₃) δ 167.4, 167.1, 165.1, 151.4, 149.6, 143.0, 142.7, 135.9, 131.0, 127.9, 123.0, 121.3, 119.7, 114.1, 111.5, 110.1, 63.9, 56.4, 56.3, 52.0, 45.8, 30.3, 26.2. Anal. calcd for C₂₄H₂₈N₂O₆S: C, 61.0; H, 5.97; N, 5.93, Found: C, 60.92; H, 5.85; N, 5.81. Mass spectrum (API-TIS) m/z 473 (MH⁺).

### 1e. (N-(2-Methyl-2-(nitrosothio)propyl)carbamoyl)methyl 2-((2E)-3-(3,4-dimethoxyphenyl)prop-2-enoylamino)benzoate

The product of Example 1d (115 mg, 0.24 mmol) in CH₂Cl₂ (1 mL) was added to a solution of tert-butyl nitrate (90% solution, 63 μL, 54 mmol) in CH₂Cl₂ (1 mL). The reaction mixture was stirred at room temperature for 30 minutes in the dark, the solvent evaporated and the residue chromatographed (EtOAc:Hexane 3:1) to give the title compound (75 mg, 62% yield). Mp. 135-137° C. ¹H NMR (300 MHz, CDCl₃) δ 10.94 (s, 1H), 8.90 (d, J=8.5 Hz, 1H), 7.92 (dd, J=8.0 and 1.4 Hz, 1H), 7.70 (d, J=15.5 Hz, 1H), 7.65 (dt, J=7.5 and 1.4 Hz, 1H), 7.11-7.21 (m, 3H), 6.90 (d, J=8.3 Hz, 1H), 6.62 (br s, 1H), 6.47 (d, J=15.5 Hz, 1H), 4.87 (s, 2H), 4.17 (d, J=6.4 Hz, 2H), 3.98 (s, 3H), 3.95 (s, 3H), 1.92 (s, 6H). ¹³C NMR (75 MHz, CDCl₃) δ 167.5, 167.4, 165.1, 151.4, 149.6, 143.0, 142.7, 136.0, 130.9, 127.9, 123.0, 121.3, 119.7, 114.0, 111.5, 110.2, 63.9, 57.3, 56.4, 56.3, 49.8, 27.3. Anal. calcd for C₂₄H₂₇N₃O₇S: C, 57.47; H, 5.43; N, 8.28, Found: C, 57.53; H, 5.34; N, 8.28. Mass spectrum (API-TIS) m/z 502 (MH⁺), 472 (M−NO).

### Example 2

### 3-Methyl-3-(nitrosothio)butyl 2-(2-((2E)-3-(3,4-dimethoxyphenyl)prop-2-enoylamino)phenylcarbonyloxy)acetate

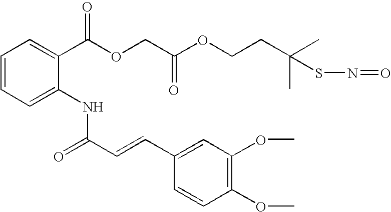

### 2a. 3-Methyl-3(2,4,6-trimethoxyphenylmethylthio)butan-1-ol

To a solution of 3-methyl-3(2,4,6-trimethoxyphenylmethylthio)butyric acid (prepared as described by Lin et al., Tet. Letts., 43: 4531-4533 (2002), (5 g, 16 mmol) in THF (50 mL) was added carefully, in portions, lithium aluminium hydride (0.9 g, 23 mmol). The reaction mixture was refluxed for 4 hours, cooled to room temperature, quenched with water and extracted with EtOAc. The aqueous phase was acidified with 2N HCl and extracted with EtOAc. The combined extracts were washed with satd sodium bicarbonate, satd. NaCl, dried with Na₂SO₄, filtered and evaporated to give the title compound (4.5 g, 90% yield). Mp 69-72° C. ¹H NMR (300 MHz, CDCl₃) δ 6.09 (s, 2H), 3.75-3.90 (m, 13H), 3.11 (t, J=5.1 Hz, 1H), 1.91 (t, J=5.8 Hz, 2H), 1.38 (s, 6H). ¹³C NMR (75 MHz, CDCl₃) δ 160.4, 158.8, 106.1, 90.6, 60.8, 55.8, 55.2, 44.4, 43.2, 29.3, 20.7. Anal. calcd for C₁₅H₂₄O₄S: C, 60.00; H, 8.05; Found: C, 60.13; H, 8.26.

### 2b. 3-Methyl-3-sulfanylbutyl 2-(2-((2E)-3-(3,4-dimethoxyphenyl)prop-2-enoylamino) phenylcarbonyloxy)acetate

A solution of the product of Example 1c (0.77 g, 2 mmol), the product of Example 2a (0.6 g, 2 mmol) and 4-dimethylaminopyridine (0.25 g, 2 mmol) in DMF (13 mL) was treated with 1-(3-(dimethylamino)propyl)-3-ethylcarbodiimide hydrochloride (0.67 g, 3.5 mmol). The reaction mixture was stirred at room temperature for 16 hours, diluted with a large volume of EtOAc and washed several times with water, satd. NaCl, dried with Na₂SO₄, filtered and evaporated. The crude product was mixed with phenol (250 mg), anisole (250 μL), and water (300 μL) and finally trifluoroacetic acid (10 mL) was added. The reaction mixture was stirred at room temperature for 45 minutes, the solvent evaporated and the residue neutralized with satd sodium bicarbonate solution and extracted with EtOAc. The combined organic layers were dried with Na₂SO₄, filtered and evaporated. The residue was chromatographed on silica gel, eluting with EtOAc:Hexane 1:2 to give the title compound (0.3 g, 31% yield). ¹H NMR (300 MHz, CDCl₃) δ 11.00 (s, 1H), 8.89 (d, J=8.6 Hz, 1H), 8.14 (d, J=8.1 Hz, 1H), 7.70 (d, J=15.5 Hz, 1H), 7.61 (t, J=7.9 Hz, 1H), 7.08-7.18 (m, 3H), 6.87 (d, J=8.2 H), 1H), 6.49 (d, J=15.5 Hz, 1H), 4.88 (s, 2H), 4.44 (t, J=7.2 Hz, 2H), 3.95 (s, 3H), 3.91 (s, 3H), 1.97 (t, J=7.0 Hz, 2H), 1.74 (s, 1H), 1.41 (s, 6H). ¹³C NMR (75 MHz, CDCl₃) δ 167.5, 167.3, 164.6, 150.8, 149.1, 142.4, 142.1, 135.2, 131.2, 127.5, 122.5, 122.4, 120.6, 119.4, 114.0. 111.0, 109.6, 63.2, 61.3, 55.9, 55.8, 44.1, 42.7, 33.0. Mass spectrum (API-TIS) m/z 488 (MH⁺).

### 2c. 3-Methyl-3-(nitrosothio)butyl 2-(2-((2E)-3-(3,4-dimethoxyphenyl)prop-2-enoylamino)phenylcarbonyloxy)acetate

A solution of the product of Example 2b (65 mg, 0.13 mmol) in CH₂Cl₂ (1 mL) was added dropwise to a solution of tert-butyl nitrite (90% solution, 39 μL, 34 mg, 0.33 mmol) in CH₂Cl₂ (1 mL). The reaction mixture was stirred at room temperature in the dark for 40 min, the solvent evaporated and the residue chromatographed (EtOAc:Hexane 2:3) to give the title compound (40 mg, 58% yield). ¹H NMR (300 MHz, CDCl₃) δ 11.00 (s, 1H), 8.89 (d, J=8.5 Hz, 1H), 8.14 (d, J=7.8 Hz, 1H), 7.69 (d, J=15.5 Hz, 1H), 7.61 (t, J=8.1 Hz, 1H), 7.08-7.18 (m, 3H), 6.87 (d, J=8.2 Hz, 1H), 6.49 (d, J=15.5 Hz, 1H), 4.86 (s, 2H), 4.45 (t, J=6.9 Hz, 2H), 3.95 (s, 3H), 3.92 (s, 3H), 2.62 (t, J=6.9 Hz, 2H), 1.90 (s, 6H); ¹³C NMR (75 MHz, CDCl₃) δ 167.6, 167.3, 164.7, 151.0, 149.2, 142.4, 142.2, 135.3, 131.2, 127.6, 122.6, 122.5, 120.6, 119.5, 113.9, 111.0, 109.8, 62.4, 61.2, 56.0, 55.9, 54.6, 41.3, 29.2.

### Example 3

### 2-(4-(2-Methyl-2-(nitrosothio)propyl)piperazinyl)-2-oxoethyl 2-((2E)-3-(3,4-dimethoxyphenyl)prop-2-enoylamino)benzoate

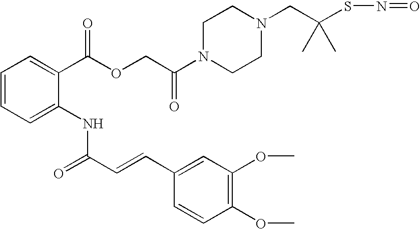

### 3a. 2,2-Dimethylthiirane

A mixture of 2,2-dimethyloxirane (25 g, 346 mmol), water (50 ml), and potassium thiocyanate (67 g, 692 mmol) was stirred at room temperature for 20 hours. The organic phase was removed, dried over Na₂SO₄, and filtered to give title compound (26.4 g, 87% yield). ¹H NMR (300 MHz, CDCl₃) δ 2.41(s, 2H), 1.62 (s, 6H).

### 3b. 2-Methyl-1-piperazinylpropane-2-thiol

A mixture of piperazine (44.7 g, 0.52 mol) and the product of Example 3a (15.2 g, 0.17 mmol) in toluene (70 mL) was heated at 80° C. for 6 hours. The reaction mixture was cooled, poured into water and extracted with CH₂Cl₂. The combined extracts were dried over Na₂SO₄, filtered and the solvent evaporated to give the title compound (30.5 g, 100% yield). ¹H NMR (300 MHz, CDCl₃) δ 2.80-2.90 (m, 4H), 2.50-2.60 (m, 4H), 2.35 (s, 2H), 1.52 (br s, 1H), 1.29 (s, 6H).

### 3c. 2-(4-(2-Methyl-2-sulfanylpropyl)piperazinyl)-2-oxoethyl 2-((2E)-3-(3,4 dimethoxyphenyl)prop-2-enoylamino)benzoate

A solution of the product of Example 3b (0.34 g, 1.94 mmol), the product of Example 1c (0.75 g, 1.94 mmol) and 4-dimethylaminopyridine (0.24 g, 1.94 mmol) in DMF (10 mL) was treated with 1-(3-(dimethylamino)propyl)-3-ethylcarbodiimide hydrochloride (0.56 g, 2.9 mmol). The reaction mixture was stirred at room temperature for 2 hours, diluted with a large volume of EtOAc, washed several times with water, satd. NaCl and dried over Na₂SO₄. The residue after filtration and evaporation was chromatographed on silica gel, eluting with EtOAc:Hexane 2:1 to give the title compound (0.4 g, 72% yield). ¹H NMR (300 MHz, CDCl₃) δ 10.98 (s, 1H), 8.88 (d, J=8.5 Hz, 1H), 8.12 (dd, J=8.5 and 1.4 Hz, 1H), 7.69 (d, J=15.5 Hz, 1H), 7.59 (dt, J=7.9 and 1.4 Hz, 1H), 7.08-7.19 (m, 3H), 6.87 (d, J=8.2 Hz, 1H), 6.61 (d, J=15.5 Hz, 1H), 5.02 (s, 2H), 3.95 (s, 3H), 3.92 (s, 3H), 3.68 (br s, 2H), 3.47 (br s, 2H), 2.62-2.76 (m, 4H), 2.45 (s, 2H), 2.06 (s, 1H), 1.34 (s, 6H). ¹³C NMR (75 MHz, CDCl₃) δ 167.6, 164.9, 164.6, 150.9, 149.2, 142.2, 141.7, 134.9, 131.3, 127.9, 122.6, 120.9, 119.9, 115.1, 111.1, 109.9, 71.1, 62.1, 56.0. 55.9, 55.1, 54.9, 46.1, 45.0, 42.5, 30.2. Mass spectrum (API-TIS) m/z 542 (MH⁺).

### 3d. 2-(4-(2-Methyl-2-(nitrosothio)propyl)piperazinyl)-2-oxoethyl 2-((2E)-3-(3,4-dimethoxyphenyl)prop-2-enoylamino)benzoate

A solution of the product of Example 3c (250 mg, 0.46 mmol) in CH₂Cl₂ (2 mL) at 0° C. was added to an ice cold solution of tert-butyl nitrite (90% solution, 110 μL, 95 mg, 0.92 mmol) in a mixture of CH₂Cl₂ (4 mL) and HCl in ether (2 mL). The reaction mixture was stirred over ice for 40 minutes, at room temperature for 10 minutes then diluted with more CH₂Cl₂ and washed with satd sodium bicarbonate. The organic phase was dried over Na₂SO₄, filtered and evaporated. The residue was chromatographed on silica gel, eluting with MeOH:CH₂Cl₂ 7:93 to give the title compound (950 mg, 19% yield). ¹H NMR (300 MHz, CDCl₃) δ 11.00 (s, 1H), 8.89 (d, J=8.5 Hz, 1H), 8.23 (d, J=7.7Hz, 1H), 7.70 (d, J=15.5 Hz, 1H), 7.60 (t, J=7.7Hz, 1H), 7.05-7.19 (m, 3H), 6.88 (d, J=8.2 Hz, 1H), 6.62 (d, J=15.5 Hz, 1H), 5.00 (s, 2h), 3.95 (s, 3H), 3.93 (s, 3H), 3.64 (br s, 2H), 3.41 (br s, 2H), 3.06 (s, 2H), 2.67 (br s, 4H), 1.91 (s, 6H). Mass spectrum (API-TIS) m/z 571 (MH⁺).

### Example 4

### 2-(4-(2-Methyl-2-(nitrosothio)propyl)piperazinyl)ethyl 2-(2-(92E)-3-(3,4-dimethoxyphenyl)prop-2-enoylamino)phenyloxycarbonyloxy)acetate

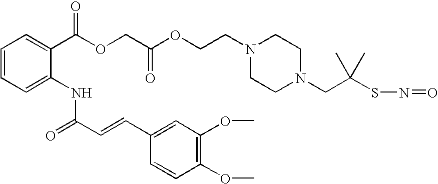

### 4a. 2-(4-(2-Methyl-2-sulfanylpropyl)piperazinyl)ethan-1-ol

The solution of the product of Example 3a (1.0 g, 11.3 mmol) and 1-(2-hydroxyethyl) piperazine (2.95 g, 22.7 mmol) in benzene (1.5 ml) was heated to 80° C. for 2 hours. The mixture was cooled to room temperature diluted with EtOAc and washed with water. The organic layer was dried over Na₂SO₄, filtered and evaporated to give the title compound (2.06 g, 83% yield) as a white solid. ¹H NMR (300 MHz, CDCl₃) δ 3.61 (t, J=5.4 Hz, 2H), 2.66-2.71 (m, 4H), 2.52-2.56 (m, 6H), 2.47 (s, 2H), 1.31 (s, 6H). ¹³C NMR (75 MHz, CDCl₃) δ 7.10, 59.2, 57.6, 55.5, 53.2, 46.4, 30.1.

### 4b. 2-(4-(2-Methyl-2-(nitrosothio)propyl)piperazinyl)ethyl 2-(2-(2E)-3-(3,4-dimethoxyphenyl)prop-2-enoylamino)phenylcarbonyloxy)acetate

The product of Example 4a (3 g, 13 mmol) was dissolved in CH₂Cl₂ (13 mL) and cooled to 0° C. After 10 minutes, trifluoroacetic acid (2.1 mL) was added dropwise and after a further 10 min tert-butyl nitrite (90% solution, 2 mL, 1.54 g, 15.7 mmol) was added. The resultant solution was stirred at 0° C. for 40 minutes in the dark then washed with 10% sodium carbonate solution and dried over Na₂SO₄. Half of this solution was added to the product of Example 1c (0.9 g, 2.3 mmol) in a mixture of CH₂Cl₂ (10 mL), THF (10 mL) with enough DMF to cause dissolution. The reaction mixture was cooled to 0° C. and after 10 minutes a solution of 1,3-dicyclohexylcarbodiimide (0.57 g, 2.75 mmol) in CH₂Cl₂ (5 mL) was added dropwise over 5 minutes. The reaction mixture was stirred over ice for 1 hour, cooled to −78° C. and filtered. The solvent was evaporated and the residue chromatographed (EtOAc:Hexane 3:2). The product was further purified by trituration with ether to remove residual N,N′-dicyclohexylurea and give the title compound (200 mg, 14% yield). ¹H NMR (300 MHz, CDCl₃) δ 11.0 (s, 1H), 8.90 (d, J=8.5 Hz, 1H), 8.16 (d, J=8.0 Hz, 1H), 7.70 (d, J=15.5 Hz, 1H), 7.62 (t, J=8.4 Hz, 1H), 7.09-7.21 (m, 3H), 6.89 (d, J=8.2 Hz, 1H), 6.50 (d, J=15.5 Hz, 1H), 4.91 (s, 2H), 4.35 (t, J=5.6 Hz, 2H), 3.97 (s, 3H), 3.93 (s, 3H), 2.95 (s, 2H), 2.41-2.72 (m, 10H), 1.85 (s, 6H). ¹³C NMR (75 MHz, CDCl₃) δ 167.6, 167.4, 164.7, 151.0, 149.3, 142.5, 142.2, 135.3, 131.2, 127.6, 122.6, 122.5, 120.7, 119.5, 114.0, 111.1, 109.8, 68.1, 62.9, 61.4, 58.8, 56.3, 56.0, 55.9, 55.0, 53.5, 27.0. Mass spectrum (API-TIS) m/z 615 (MH⁺).

### Example 5

### 2-((4-((2,4-dioxo(1,3-thiazolidin-5-yl))methyl)phenoxy)methyl)-2,5,7,8-tetramethylchroman-6-yl 4-(N-(2-methyl-2-(nitrosothio)propyl)carbamoyl)butanoate

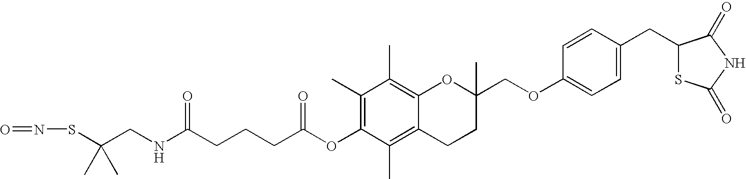

### 5a. 5-((4-((6-Hydroxy-2,5,7,8-tetramethylchroman-2-yl)methoxy)phenyl)methyl)-1,3-thiazolidine-2,4-dione (troglitazone)

The title compound was prepared according to the method described in Yoshioka et al J. Med. Chem. 32:421-428, (1989).

### 5b. 4-((2-((4-((2,4-Dioxo(1,3-thiazolidin-5-yl))methyl)phenoxy)methyl)2,5,7,8-tetramethylchroman-6-yl)oxycarbonyl)butanoic acid

The product of Example 5a (1.26 g, 2.8 mmol), glutaric anhydride (0.33 g, 2.8 mmol) and 4-dimethylaminopyridine (0.35 g, 2.8 mmol) in CH₂Cl₂ (15 mL) was stirred at room temparature overnight. The reaction mixture was diluted with more CH₂Cl₂, washed with 2N HCl, dried over Na₂SO₄, filtered and evaporated to give the title compound (1.4 g, 80% yield) which was used in the next step without purification.

### 5c. 2((4-((2,4-Dioxo(1,3-thiazolidin-5-yl))methyl)phenoxy)methyl)-2,5,7,8-tetramethylchroman-6-yl 4-(N-(2-methyl-2-sulfanylpropyl)carbamoyl)butanoate

A mixture of the product of Example 5b (1.3 g, 2.3 mmol), 4-dimethylaminopyridine (0.11 g, 0.94 mmol), triethylamine (0.59 mL, 425 mg, 4.2 mmol), and 2-mercapto-2-methyl-1-propylamine hydrochloride (0.6 g, 4.2 mmol) in DMF (15 mL) was treated with 1-(3-(dimethylamino)propyl)-3-ethylcarbodiimide hydrochloride (0.8 g, 4.2 mmol). The reaction mixture was stirred at room temperature for 6 hours, diluted with a large volume of EtOAc, washed several times with water, satd. NaCl and dried with Na₂SO₄. The residue after filtration and evaporation was chromatographed on silica gel, eluting with EtOAc:Hexane 1:1 to 2:1 to give the title compound (0.5 g, 33% yield). ¹H NMR (300 MHz, CDCl₃) δ 9.41 (br s, 1H), 7.11 (d, J=7.7 Hz, 2H), 6.86 (d, J=7.7 Hz, 2H), 6.17 (t, J=5.7 Hz, 1H), 4.43 (dd, J=9.7 and 3.1 Hz, 1H), 3.92 (dd, J=29.6 Hz and 9.1 Hz, 2H), 3.44 (dd, J=14.1 and 3.3 Hz, 1H), 3.35 (d, J=5.7 Hz, 2H), 3.03 (dd, J=13.8 and 10.0 Hz, 1H), 2.72 (t, J=6.9 Hz, 2H), 2.63 (t, J=6.2 Hz, 2H), 2.43 (t, J=7.2 Hz, 2H), 2.15 (m, 2H), 2.05 (s, 3H), 2.02 (s, 3H), 1.98 (s, 3H), 1.80-2.00 (m, 1H), 1.41 (s, 3H), 1.36 (s, 6H). ¹³C NMR (75 MHz, CDCl₃) δ174.4, 172.4, 172.0, 169.7, 158.4 148.8, 140.7, 130.1, 128.2, 126.9, 125.0, 123.1, 117.4, 115.0, 74.5, 72.6, 53.7, 52.1, 45.3, 37.7, 35.4, 32.9, 29.9, 28.2, 21.0, 20.1, 12.9, 12.1, 11.8. Mass spectrum (API-TIS) m/z 643 (MH⁺).

### 5d. 2((4-((2,4-Dioxo(1,3-thiazolidin-5-yl))methyl)phenoxy)methyl)-2,5,7,8-tetramethylchroman-6-yl 4-(N-(2-methyl-2-(nitrosothio)propyl)carbamoyl)butanoate

A solution of the product of Example 5c (230 mg, 0.36 mmol), in CH₂Cl₂ (3 mL) was added to a solution of tert-butyl nitrite (90% solution, 109 μL, 85 mg, 0.82 mmol) in CH₂Cl₂. The reaction mixture was stirred at room temperature for 40 minutes in the dark, evaporated and chromatographed on silica gel eluting with EtOAc:Hexane 3:1 to give the title compound (115 mg, 48% yield). ¹H NMR (300 MHz, CDCl₃) δ 8.57 (br s, 1H), 7.13 (d, J=8.5 Hz, 2H), 6.87 (d, J=8.5 Hz, 2H), 5.93 (t, J=6.4 Hz, 1H), 4.47 (dd, J=9.6 and 3.8 Hz, 1H), 4.06 (d, J=6.4 Hz, 2H), 3.93 (dd, J=30.5 and 9.0 Hz, 2H), 3.45 (dd, J=14.1 and 3.0 Hz, 1H), 3.08 (dd, J=14.0 and 9.7 Hz, 1H), 2.67 (t, J=7.1 Hz, 2H), 2.63 (t, J=6.8 Hz, 2H), 2.36 (t, J=7.2 Hz, 2H), 2.11 (m, 2H), 2.08 (s, 3H), 1.99 (s, 3H), 1.95 (s, 3H), 1.89 (s, 6H), 1.42 (s, 3H). ¹³C NMR (75 MHz, CDCl₃) δ 174.1, 172.5, 170.3, 158.5, 148.9, 140.6, 130.2, 128.2, 126.9, 124.9, 123.2, 117.5, 115.0, 74.6, 57.2, 53.6, 49.4, 37.8, 35.4, 32.8, 28.3, 26.9, 21.0, 20.1, 13.0, 12.2, 11.9. Mass spectrum (API-TIS) m/z 672 (MH⁺)

### Example 6

### (1S,11S,14S,15S,10R)-14-Hydroxy-4-methoxy-15-methyltetracyclo(8.7.0.0<2,7>0.0<11,15>)heptadeca-2(7),3,5-trien-5-yl 3-methyl-3-(nitrosothio)butanoate and (1S,11S,14S,15S,10R)-4-methoxy-15-methyl-14-(nitrosooxy)tetracyclo(8.7.0.0<2,7>0.0<11,15>)heptadeca-2(7),3,5-trien-5-yl 3-methyl-3-(nitrosothio)butanoate

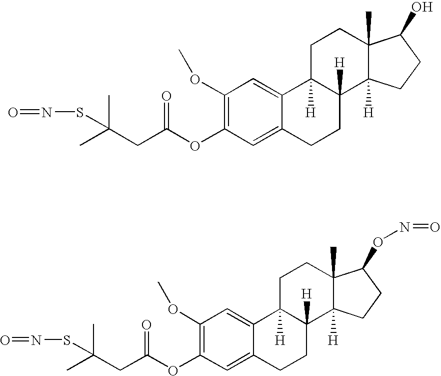

### 6a. (1S,11S,14S,15S,10R)-14-Hydroxy-4-methoxy-15-methyltetracyclo(8.7.0.0<2,7>0.0<11,15>)heptadeca-2(7),3,5-trien-5-yl 3-methyl-3-((2,4,6-trimethoxyphenyl)methylthio)butanoate and (1S,11S,14S,15S,10R)-4-methoxy-15-methyl-14-(3-methyl-3-((2,4,6-trimethoxyphenyl)methylthio)butanoyloxy)tetracyclo (8.7.0.0<2,7>0.0<11,15>)heptadeca-2(7),3,5-trien-5-yl 3-methyl-3-((2,4,6-trimethoxyphenyl)methylthio)butanoate

A mixture of 2-methoxyestradiol (401 mg, 1.33 mmol), 1-(3-(dimethylamino)propyl)-3-ethylcarbodiimide hydrochloride (351 mg, 1.83 mmol), 4-dimethylaminopyridine (204 mg, 1.67 mmol) and 3-methyl-3(2,4,6-trimethoxyphenylmethylthio)butyric acid (prepared as described by Lin et al., Tet. Letts., 43: 4531-4533 (2002),(451 mg, 1.43 mmol) in DMF (15 mL) was stirred at room temperature overnight and then concentrated to dryness. The residue was treated with EtOAc and water and the organic phase was washed with satd. NaCl, dried with Na₂SO₄, filtered, and the solvent evaporated. The residue was chromatographed on silica gel, eluting with EtOAc:Hexane (1:3 to 1:1) to give the monoester (0.54 g, 68% yield) and the diester (0.14 g, 12% yield). Monoester ¹H NMR (300 MHz, CDCl₃) δ 6.89 (s, 1H), 6.74 (s, 1H), 6.12 (s, 2H), 3.68-3.96 (m, 15H), 2.88-3.04 (m, 2H), 2.71-2.88 (m, 2H), 2.03-2.37 (m, 3H), 1.77-2.03 (m, 2H), 1.15-1.77 (m, 15H), 0.78 (s, 3H). ¹³C NMR (75 MHz, CDCl₃) δ 169.0, 160.0, 158.4, 148.4, 138.4, 137.2, 128.8, 122.5, 109.5, 107.1, 90.4, 81.3, 55.5, 55.0, 49.8, 46.2, 44.2, 43.8, 42.9, 38.2, 36.4, 30.2, 28.4, 27.6, 27.3, 26.9, 26.1, 22.8, 20.7, 10.8. Mass spectrum (API-TIS) m/z 599 (MH⁺), 616 (MNH₄⁺), 621 (MNa⁺). Diester ¹H NMR (300 MHz, CDCl₃) δ 6.88 (s, 1H), 6.74 (s, 1H), 6.11 (s, 4H), 4.71 (t, J=7.5 Hz, 1H), 3.95-3.71 (m, 26H), 3.00-2.88 (m, 2H), 2.88-2.68 (m, 4H), 2.33-2.18 (m, 3H), 1.97-1.82 (m, 2H), 1.82-1.20 (m, 20H), 0.85 (s, 3H). ¹³C NMR (300 MHz, CDCl₃) δ 171.0, 169.2, 160.2, 158.6, 148.6, 138.5, 137.4, 129.0, 122.7, 109.7, 107.34, 107.30, 90.5, 82.7, 55.7, 55.2, 49.6, 47.2, 46.3, 44.2, 44.0, 43.8, 42.7, 38.1, 36.8, 28.6, 28.2, 28.1, 27.8, 27.6, 27.0, 26.2, 23.2, 20.8, 20.7, 12.2. Mass spectrum (API-TIS) m/z 895 (MH⁺), 912 (MNH₄⁺), 917 (MNa⁺).

### 6b. (1S,11S,14S,15S,10R)-14-Hydroxy-4-methoxy-15-methyltetracyclo (8.7.0.0<2,7>0.0<11,15>)heptadeca-2(7),3,5-trien-5-yl 3-methyl-3-sulfanylbutanoate

To a mixture of the monoester from Example 6a (517 mg, 0.86 mmol) and phenol (134 mg, 1.43 mmol) in CH₂Cl₂ (3 mL) was added anisole (120 μL, 119 mg, 0.92 mmol), water (120 μL) and trifluoroacetic acid (4 mL). The reaction mixture was stirred at room temperature for 20 minutes and evaporated to dryness. The residue was treated with EtOAc, washed with satd. NaCl, satd sodium bicarbonate solution and satd. NaCl. The organic phase was dried with Na₂SO₄, filtered, evaporated and the residue chromatographed on silica gel elutinh with EtOAc:Hexane (1:9 to 1:4 to 1:1) to give the title compound (232 mg, 64% yield). Mp 115-118° C. ¹H NMR (300 MHz, CDCl₃) δ 6.90 (s, 1H), 6.75 (s, 1H), 3.79 (s, 3H), 3.72 (t, J=8.5 Hz, 1H), 2.91 (s, 2H), 2.77 (m, 2H), 2.53 (s, 1H), 1.64-2.35 (m, 7H), 1.59 (s, 6H), 1.12-1.54 (m, 7H), 0.77 (s, 3H). ¹³C NMR (75 MHz, CDCl₃) δ 169.0, 148.4, 138.8, 137.0, 129.1, 122.6, 109.6, 81.7, 55.7, 50.2, 49.9, 44.4, 43.1, 41.7, 38.3, 36.6, 32.3, 30.5, 28.6, 27.0, 26.3, 23.0, 11.0. Mass spectrum (API-TIS) m/z 419 (MH⁺), 436 (MNH₄⁺).

### 6c. (1S,11S,14S,15S,10R)-14-Hydroxy-4-methoxy-15-methyltetracyclo(8.7.0.0<2,7>0.0<11,15>)heptadeca-2(7),3,5-trien-5-yl 3-methyl-3-(nitrosothio)butanoate and (1S,11S,14S,15S,10R)-4-methoxy-15-methyl-14-(nitrosooxy)tetracyclo(8.7.0.0<2,7>0.0<11,1 5>)heptadeca-2(7),3,5-trien-5-yl 3-methyl-3-(nitrosothio)butanoate

To the product of Example 6b (117 mg, 0.28 mmol) in CH₂Cl₂ (3.5 mL) was added tert-butyl nitrite (90% solution, 40 μL, 35 mg, 0.34 mmol). The reaction mixture was stirred at room temperature for 20 minutes, evaporated and the residue chromatographed on silica gel elutinh with neat CH₂Cl₂ to give the nitrosothiol (71.5 mg, 57% yield) and the nitrite nitrosothiol (25 mg, 19% yield). Nitrosothiol Mp 102-105° C. ¹H NMR (300 MHz, CDCl₃) δ 6.90 (s, 1H), 6.70 (s, 1H), 3.78 (s, 3H), 3.71 (t, J=8.5 Hz, 1H), 3.52 (s, 2H), 2.77 (m, 2H), 2.15-2.36 (m, 2H), 2.10 (s, 6H), 1.02-2.02 (m, 12H), 0.77 (s, 3H). ¹³C NMR (75 MHz, CDCl₃) δ 168.2, 148.4, 138.9, 137.1, 129.1, 122.4, 109.7, 81.7, 55.7, 53.6, 49.9, 46.8, 44.4, 43.1, 38.3, 36.6, 30.5, 28.64, 28.58, 27.0, 26.3, 23.0, 11.0. Mass spectrum (API-TIS) m/z 448 (MH⁺), 465 (MNH₄⁺). Nitrite nitrosothiol ¹H NMR (300 MHz, CDCl₃) δ 6.89 (s, 1H), 6.71 (s, 1H), 5.37 (t, J=8.5 Hz, 1H), 3.78 (s, 3H), 3.52 (s, 2H), 2.77-2.83 (m, 2H), 2.18-2.34 (m, 3H), 2.09 (s, 6H), 1.66-1.97 (m, 4H), 1.31-1.66 (m, 6H), 0.77 (s, 3H). ¹³C NMR (75 MHz, CDCl₃) δ 168.3, 148.5, 138.6, 137.2, 129.1, 122.5, 109.7, 87.7, 55.8, 53.6, 50.3, 46.9, 44.3, 43.4, 38.1, 36.6, 28.7, 28.6, 27.3, 27.1, 26.0, 23.2, 11.9. Mass spectrum (API-TIS) m/z 477 (MH⁺), 494 (MNH₄⁺).

### Example 7

### (1S,11S,14S,15S,10R)-4-Methoxy-15-methyl-14-(3-methyl-3-(nitrosothio)butanoyloxy)tetracyclo-(8.7.0.0<2,7>0.0<11,15>)heptadeca-2(7),3,5-trien-5-yl 3-methyl-3-(nitrosothio)butanoate

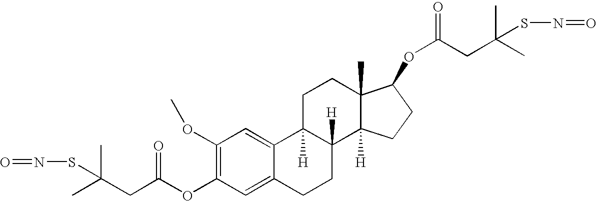

### 7a. (1S,11S,14S,15S,10R)-4-Methoxy-15-methyl-14-(3-methyl-3-sulfanylbutanoyloxy) tetracyclo-(8.7.0.0<2,7>0.0<11,15>)heptadeca-2(7),3,5-trien-5-yl 3-methyl-3-sulfanylbutanoate

To the diester from Example 6a (0.16 g, 0.18 mmol) and phenol (66 mg, 0.7 mmol) in CH₂Cl₂ (1.5 mL) was added water (60 μL) and trifluoroacetic acid (2 mL). The reaction mixture was stirred at room temperature for 20 minutes and evaporated to dryness. The residue was diluted with EtOAc, washed with potassium carbonate solution, dried with Na₂SO₄, filtered and evaporated. The residue was chromatographed on silica gel, eluting with EtOAc:Hexane (1:19) to give the title compound (55 mg, 57% yield). ¹H NMR (300 MHz, CDCl₃) δ 6.89 (s, 1H), 6.75 (s, 1H), 4.72 (t, J=8.5 Hz, 1H), 3.79 (s, 3H), 2.91 (s, 2H), 2.78 (m, 2H), 2.66 (s, 2H), 2.54 (s, 1H), 2.32 (s, 1H), 2.19-2.32 (m, 3H), 1.81-1.94 (m, 2H), 1.67-1.81 (m, 1H), 1.60 (s, 6H), 1.52 (s, 6H), 1.22-1.52 (m, 7H), 0.85 (s, 3H). ¹³C NMR (75 MHz, CDCl₃) δ 170.8, 169.0, 148.5, 138.6, 137.1, 129.1, 122.6, 109.6, 82.7, 55.8, 50.7, 50.2, 49.6, 44.2, 42.8, 41.7, 41.6, 38.0, 36.8, 32.7, 32.6, 32.3, 28.6, 27.5, 27.0, 26.2, 23.2, 12.2. Mass spectrum (API-TIS) m/z 535 (MH⁺), 552 (MNH₄⁺), 557 (MNa⁺).

### 7b. (1S,11S,14S,15S,10R)-4-Methoxy-15-methyl-14-(3-methyl-3-(nitrosothio)butanoyloxy)tetracyclo-(8.7.0.0<2,7>0.0<11,15>)heptadeca-2(7),3,5-trien-5-yl 3-methyl-3-(nitrosothio)butanoate

To the product of Example 7a (28.6 mg, 0.05 mmol) in CH₂Cl₂ (1 mL) was added tert-butyl nitrite (90% solution, 26 μL, 22 mg, 0.21 mmol). The reaction mixture was stirred at room temperature for 20 minutes and evaporated to dryness. The residue was chromatographed on silica gel, eluting with EtOAc:Hexane (1:19) to give the title compound (20.3 mg, 64% yield). ¹H NMR (300 MHz, CDCl₃) δ 6.88 (s, 1H), 6.70 (s, 1H), 4.68 (t, J=8.5 Hz, 1H), 3.78 (s, 3H), 3.53 (s, 2H), 3.26 (s, 2H), 2.73-2.81 (m, 2H), 2.15-2.32 (m, 2H), 2.10 (s, 6H), 2.01 (s, 6H), 1.59-1.90 (m, 4H), 1.21-1.59 (m, 7H), 0.79 (s, 3H). ¹³C NMR (75 MHz, CDCl₃) δ 170.1, 168.3, 148.5, 138.7, 137.1, 129.1, 122.5, 109.7, 83.3, 55.8, 53.63, 53.55, 49.6, 47.6, 46.9, 44.2, 42.8, 38.0, 36.8, 29.1, 29.0, 28.7, 28.6, 27.5, 27.0, 26.2, 23.2, 12.2 Mass spectrum (API-TIS) m/z 593 (MH⁺), 610 (MNH₄⁺).

### Example 8

### (1S,11S,14S,15S,10R)-14-Hydroxy-15-methyltetracyclo (8.7.0.0<2,7>0.0<11,15>)heptadeca-2(7),3,5-trien-5-yl 3-methyl-3-(nitrosothio)butanoate and (1S,11S,14S,15S,10R)-15-methyl-14-(nitrosooxy)tetracyclo(8.7.0.0<2,7>0.0<11,15>)heptadeca-2(7),3,5-trien-5-yl 3-methyl-3-(nitrosothio)butanoate

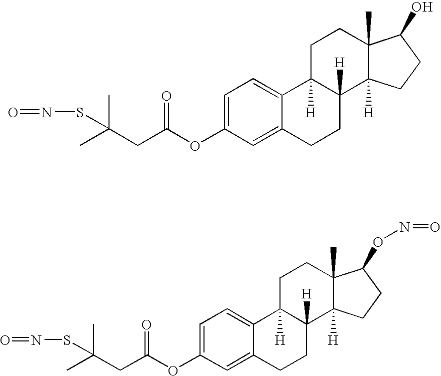

### 8a. (1S,11S,14S,15S,10R)-14-Hydroxy-15-methyltetracyclo(8.7.0.0<2,7>0.0<11,15>) heptadeca-2(7),3,5-trien-5-yl 3-methyl-3-((2,4,6-trimethoxyphenyl)methylthio)butanoate and (1S,11S,14S,15S,10R)-15-methyl-5-(3-methyl-3-((2,4,6-trimethoxyphenyl)methylthio)butanoyloxy)tetracyclo(8.7.0.0<2,7>0.0<11,15>) heptadeca-2(7),3,5-trien-14-yl 3-methyl-3-((2,4,6-trimethoxyphenyl)methylthio) butanoate

A mixture of β-estradiol (201 mg, 1.1 mmol), 1-(3-(dimethylamino)propyl)-3-ethylcarbodiimide hydrochloride (258 mg, 1.34 mmol), 4-dimethylaminopyridine (145 mg, 1.19 mmol) and 3-methyl-3(2,4,6-trimethoxyphenylmethylthio)butyric acid (prepared as described by Lin et al., Tet. Letts., 43: 4531-4533 (2002), (375 mg, 1.19 mmol) was stirred in DMF (10 mL) overnight at room temperature and then evaporated. The residue was treated with EtOAc and water, the organic phase washed with 0.2 M citric acid, satd. NaCl, satd sodium bicarbonate solution, satd. NaCl and dried over MgSO₄. The residue after filtration and evaporation was chromatographed on silica gel, eluting with EtOAc:Hexane (1:4, to 3:7 to 2:3) to give the monoester (440 mg, 70% yield) and the diester (121 mg). The latter was chromatographed again on silica gel, eluting with EtOAc:Hexane: (1:9 then 3:17) to give pure diester (89 mg, 9% yield). Monoester ¹H NMR (300 MHz, CDCl₃) δ 7.26 (d, J=8.3 Hz, 1H), 6.86 (d, J=8.3 Hz, 1H), 6.79 (s, 1H), 6.10 (s, 2H), 3.65-3.86 (m, 12H), 2.90 (s, 2H), 2.84 (m, 2H), 1.92-2.23 (m, 6H), 1.15-1.92 (m, 14H), 0.75 (s, 3H). ¹³C NMR (75 MHz, CDCl₃) δ 169.6, 160.2, 158.5, 148.3, 137.9, 137.7, 126.1, 121.4, 118.5, 107.2, 90.5, 81.5, 55.6, 55.1, 49.9, 46.6, 43.9, 43.0, 38.3, 36.5, 30.3, 29.4, 28.2, 26.9, 26.0, 23.0, 20.8, 10.9. Mass spectrum (API-TIS) m/z 569 (MH⁺), 586 (MNH₄⁺), 591 (MNa⁺). Diester ¹H NMR (300 MHz, CDCl₃) δ 7.27 (d, J=8.4 Hz, 1H), 6.86 (d, J=8.4 Hz, 1H), 6.80 (s, 1H), 6.11 (s, 4H), 4.70 (t, J=8.4 Hz, 1H), 3.89-3.76 9m, 22H), 2.91 (s, 2H), 2.85 (m, 2H), 2.72 (s, 2H), 2.26-2.20 (m, 3H), 1.90-1.20 (m, 22H), 0.85 (s, 3H). ¹³C NMR (75 MHz, CDCl₃) δ 171.1, 169.7, 160.34, 160.30, 158.68, 158.65, 148.4, 138.0, 137.7, 126.3, 121.6, 118.7, 107.6, 107.4, 90.7, 82.8, 55.8, 55.3, 49.8, 47.3, 46.7, 44.0, 43.8, 42.8, 38.2, 36.9, 29.5, 28.30, 28.23, 28.16, 27.6, 27.0, 26.0, 23.3, 20.9, 20.8, 12.2. Mass spectrum (API-TIS) m/z 865 (MH⁺), 882 (MNH₄⁺), 887 (MNa⁺).

### 8b. (1S,11S,14S,15S,10R)-14-Hydroxy-15-methyltetracyclo(8.7.0.0<2,7>0.0<11,15>)heptadeca-2(7),3,5-trien-5-yl 3-methyl-3-sulfanylbutanoate

L-Cysteine (846 mg, 7 mmol) was dissolved in trifluoroacetic acid (15 mL) and to it was added the monoester from Example 8a (396 mg, 0.70 mmol) in CH₂Cl₂ (15 mL). The reaction mixture was stirred at room temperature for 10 minutes, evaporated, treated three times with EtOAc and evaporated. The solid was suspended in EtOAc and washed with satd sodium bicarbonate solution. The organic phase was dried with Na₂SO₄, filtered and evaporated. The residue was chromatographed on silica gel, eluting with EtOAc:Hexane (1:9) to give the title compound (177 mg, 65% yield). ¹H NMR (300 MHz, CDCl₃) δ 7.28 (d, J=8.4 Hz, 1H), 6.86 (d, J=8.4 Hz, 1H), 6.81 (s, 1H), 3.73 (t, J=8.4 Hz, 1H), 2.87 (m, 4H), 2.39 (s, 1H), 2.05-2.39 (m, 3H), 1.82-2.05 (m, 2H), 1.62-1.82 (m, 1H), 1.58 (s, 6H), 1.15-1.58 (m, 7H), 0.77 (s, 3H). ¹³C NMR (75 MHz, CDCl₃) δ 169.4, 148.0, 138.1, 138.0, 126.3, 121.4, 118.5, 81.6, 50.2, 50.0, 44.0, 43.1, 41.7, 38.4, 36.6, 32.5, 30.4, 29.4, 26.9, 26.1, 23.0, 10.9. Mass spectrum (API-TIS) m/z 389 (MH⁺), 406 (MNH₄⁺), 411 (MNa⁺).

### 8c. (1S,11S,14S,15S,10R)-14-Hydroxy-15-methyltetracyclo(8.7.0.0<2,7>0.0<11,15>) heptadeca-2(7),3,5-trien-5-yl 3-methyl-3-(nitrosothio)butanoate and (1S,11S,14S,15S,10R)-15-methyl-14-(nitrosooxy)tetracyclo(8.7.0.0<2,7>0.0<11,15>)heptadeca-2(7),3,5-trien-5-yl 3-methyl-3-(nitrosothio)butanoate

To the product of Example 8b (113 mg, 0.29 mmol) in CH₂Cl₂ (2 mL) was added tert-butyl nitrite (90% solution, 144 μL, 125 mg, 1.21 mmol). The reaction mixture was stirred at room temperature for 20 minutes, evaporated and chromatographed on silica gel elutinh with neat CH₂Cl₂ to give the nitrosothiol (30 mg, 24% yield) and the nitrite nitrosothiol (88 mg, 67% yield). Nitrosothiol ¹H NMR (300 MHz, CDCl₃) δ 7.27 (d, J=8.4 Hz, 1H), 6.80 (d, J=8.4 Hz, 1H), 6.75 (s, 1H), 3.73 (t, J=8.4 Hz, 1H), 3.48 (s, 2H), 2.85 (m, 2H), 2.08-2.37 (m, 2H), 2.08 (s, 6H), 1.80-2.02 (m, 2H), 1.63-1.80 (m, 1H), 1.14-1.63 (m, 9H), 0.78 (s, 3H). ¹³C NMR (75 MHz, CDCl₃) δ 168.8, 148.0, 138.3, 138.2, 126.4, 121.3, 118.4, 81.8, 53.6,50.1, 47.2, 44.1, 43.2, 38.5, 36.6, 30.6, 29.5, 29.0, 27.0, 26.1, 23.1, 11.0. Mass spectrum (API-TIS) m/z 435 (MNH₄⁺). Nitrite nitrosothiol ¹H NMR (300 MHz, CDCl₃) δ 7.27 (d, J=8.4 Hz, 1H), 6.81 (d, J=8.4 Hz, 1H), 6.76 (s, 1H), 5.34 (t, J=8.7 Hz, 1H), 3.72 (s, 2H), 2.87 (m, 2H), 2.37-2.20 (m, 3H), 2.08 (s, 6H), 1.30-2.00 (m, 10H), 0.78 (s, 3H). ¹³C NMR (75 MHz, CDCl₃) δ 168.7, 148.1, 138.1, 137.8, 126.3, 121.3, 118.4, 87.6, 53.5, 50.2, 47.2, 43.9, 43.4, 38.1, 36.6, 29.4, 28.9, 27.2, 26.9, 25.8, 23.2, 11.8. Mass spectrum (API-TIS) m/z 464 (MNH₄⁺).

### Example 9

### (1S,11S,14S,15S,10R)-15-methyl-5-(3-methyl-3-(nitrosothio)butanoyloxy)tetracyclo(8.7.0.0<2,7>0.0<11,15>)heptadeca-2,4,6-trien-14-yl 3-methyl-3-(nitrosothio)butanoate

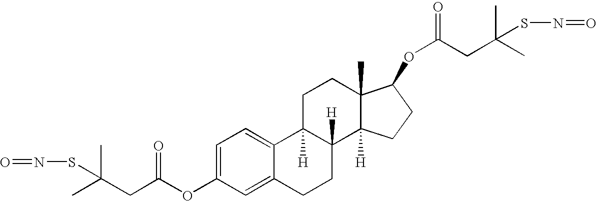

### 9a. (1S,11S,14S,15S,10R)-15-Methyl-5-(3-methyl-3-sulfanylbutanoyloxy)tetracyclo-(8.7.0.0<2,7>0.0<11,15>)heptadeca-2(7),3,5-trien-14-yl 3-methyl-3-sulfanylbutanoate

L-Cysteine (214 mg, 1.77 mmol) was dissolved in trifluoroacetic acid (4.2 mL) and to it was added a solution of the diester from Example 8a (76 mg, 0.09 mmol) in CH₂Cl₂ (2 mL). The reaction mixture was stirred at room temperature for 10 minutes and evaporated to dryness. The resulting residue was treated with EtOAc and concentrated to dryness three times. The residue was treated with EtOAc and satd sodium bicarbonate solution. The organic phase was washed with satd. NaCl, dried over MgSO₄, filtered, evaporated and chromatographed on silica gel elution with CH₂Cl₂:Hexane (1:4) then EtOAc/Hexane (1:19), to give the title compound (23 mg, 51% yield). ¹H NMR (300 MHz, CDCl₃) δ 7.28 (d, J=8.4Hz, 1H), 6.86 (d, J=8.4 Hz, 1H), 6.81 (s, 1H), 4.72 (t, J=8.4 Hz, 1H), 2.86 (m, 4H), 2.65 (s, 2H), 2.39 (s, 1H), 2.20-2.30 (m, 4H), 1.9 (m, 2H), 1.76 (m, 1H), 1.58 (s, 6H), 1.52 (s, 6H), 1.25-1.50 (m, 7H), 0.84 (s, 3H). ¹³C NMR (75 MHz, CDCl₃) δ 170.7, 169.5, 148.2, 138.2, 137.9, 126.4, 121.5, 118.6, 82.9, 50.8, 50.3, 49.7, 43.9, 42.9, 41.7, 38.2, 36.9, 32.75, 32.67, 32.58, 29.5, 27.6, 27.0, 26.0, 23.3, 12.2. Mass spectrum (API-TIS) m/z 505 (MH⁺), 522 (MNH₄⁺), 527 (MNa⁺).

### 9b. (1S,11S,14S,15S,10R)-15-methyl-5-(3-methyl-3-(nitrosothio)butanoyloxy)tetracyclo(8.7.0.0<2,7>0.0<11,15>)heptadeca-2,4,6-trien-1 4-yl 3-methyl-3-(nitrosothio)butanoate

To a solution of the product of Example 9a (22.5 mg, 0.045 mmol) in CH₂Cl₂ (1 mL) was added tert-butyl nitrite (90% solution, 22 μL, 19 mg, 0.19 mmol). The reaction mixture was stirred at room temperature for 5 minutes, evaporated, diluted with CH₂Cl₂, and washed with water and satd. NaCl. The organic phase was dried over MgSO₄, filtered, evaporated and the residue chromatographed on silica gel eluting wiht EtOAc:Hexane (1:3), to give the title compound (17.6 mg, 70% yield). ¹H NMR (300 MHz, CDCl₃) δ 7.26 (d, J=8.4 Hz, 1H), 6.80 (d, J=8.4 Hz, 1H), 6.75 (s, 1H), 4.69 (t, J=8.3 Hz, 1H), 3.48 (s, 2H), 3.25 (s, 2H), 2.85 (m, 2H), 2.23 (m, 3H), 2.08 (s, 6H), 2.01 (s, 6H), 1.35-1.86 (m, 10H), 0.78 (s, 3H). ¹³C NMR (75 MHz, CDCl₃) δ 170.0, 168.8, 148.1, 138.2, 138.0, 126.4, 121.3, 118.5, 83.3, 53.6, 49.7, 47.6, 47.3, 43.9, 42.8, 38.2, 36.8, 29.5, 29.09, 29.04, 28.97, 27.5, 27.0, 26.0, 23.3, 12.2. Mass spectrum (API-TIS) m/z 563 (MH⁺), 580 (MNH₄⁺).

### Example 10

### (1S,11S,14S,15S,10R)-14-Hydroxy-15-methyltetracyclo(8.7.0.0<2,7>0.0<11,15>)heptadeca-2(7), 3,5-trien-5-yl 3-(N-(2-methyl-2-(nitrosothio)propyl)carbamoyl)propanoate

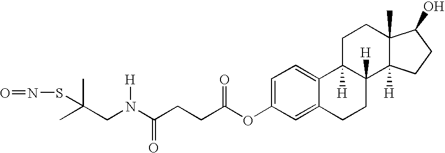

### 10a. 3-(N-(2-Methyl-2-sulfanylpropyl)carbamoyl)propanoic acid

To an ice-cooled suspension of 1-amino-2-methyl-2-propanethiol hydrochloride (5.06 g, 35.72 mmol) in CH₂Cl₂ (100 mL) was added triethylamine (5.0 mL, 35.87 mmol) followed by succinic anhydride (3.50 g, 34.96 mmol). The resulting clear solution was stirred at 0° C. for 10 minutes, then at room temperature for 2 hours. Evaporation of the volatiles under reduced pressure gave a residue which was partitioned between 2 N HCl (100 mL) and EtOAc (100 mL). The aqueous layer was extracted with EtOAc (3×100 mL). The combined organic layers were washed with satd. NaCl (50 mL), dried over Na₂SO₄ and evaporated to give a residue which was triturated with ether-hexane to give the title compound as a white solid (6.78 g, 94.4% yield). Mp 86-87° C. ¹H NMR (300 MHz, CDCl₃) δ 1.34 (s, 6H), 1.55 (s, 1H), 2.59 (t, J=6.6 Hz, 2H), 2.70 (t, J=6.6 Hz, 2H), 3.32 (d, J=8.0 Hz, 2H), 6.58 (br t, J=5.9 Hz, 1H), 10.73 (br s, 1H). ¹³C NMR (75 MHz, CDCl₃) δ 29.57, 29.79, 30.79, 172.50, 176.81. Mass spectrum (API-TIS) m/z 223 (MNH₄), 206 (MH+).

### 10b. (1S,11S,14S,15S,10R)-14-Hydroxy-15-methyltetracyclo(8.7.0.0<2,7>0.0<11,15>)heptadeca-2(7), 3,5-trien-5-yl 3-(N-(2-methyl-2-sulfanylpropyl)carbamoyl)propanoate.

To a mixture of β-estradiol (545 mg, 2.0 mmol), the product of Example 10a (657 mg, 3.2 mmol), and 4-dimethylaminopyridine (98 mg, 0.8 mmol) in CH₂Cl₂ at room temperature was added dicyclohexylcarbodiimide (1 M in CH₂Cl₂, 3.2 mL, 3.2 mmol). The reaction mixture was stirred overnight at room temperature, filtered, and then treated with water. The organic phase was washed with 0.1 M hydrochloric acid, water, satd sodium bicarbonate solution, satd. NaCl and dried over MgSO₄. The residue after filtration and evaporation was chromatographed on silica gel, eluting with EtOAc:CH₂Cl₂ 1:4 to give the monoester as an oil (762 mg, 83% yield). ¹H NMR (300 MHz, d₆-DMSO) δ 7.99 (t, J=6.0 Hz, 1H), 7.28 (d, J=8.5 Hz, 1H), 6.80 (d, J=8.4 Hz, 1H), 6.74 (d, J=1.7 Hz, 1H), 4.47 (d, J=4.8 Hz, 1H), 3.52 (m, 1H), 3.29 (s, 1H), 3.22 (d, J=6.2 Hz, 1H), 2.69-2.76 (m, 4H), 2.49-2.55 (m, 5H), 2.28 (m, 1H), 2.16 (m, 1H), 1.90-1.78 (m, 3H), 1.59 (m, 1H), 1.11-1.40 (m, 13H), 0.67 (s, 3H). Mass spectrum (API-TIS) m/z 460 (MH⁺).

### 10c. (1S,11S,14S,15S,10R)-14-Hydroxy-15-methyltetracyclo(8.7.0.0<2,7>0.0<11,15>)heptadeca-2(7), 3,5-trien-5-yl 3-(N-(2-methyl-2-(nitrosothio)propyl)carbamoyl)propanoate

To a solution of the product of Example 10b (887 mg, 1.93 mmol) in CH₂Cl₂ (5 mL) was added one drop of 6.5M HCl in isopropanol followed by tert-butyl nitrite (90% solution, 0.23 mL, 221 mg, 2.14 mmol). The reaction mixture was stirred at room temperature for 90 minutes, and washed with satd NaHCO₃ solution and satd. NaCl. The organic phase was dried over MgSO₄, filtered, evaporated and the residue chromatographed on silica gel eluting with EtOAc:CH₂Cl₂ 1:4, to give the title compound as a dark green oil (613 mg, 65% yield): ¹H NMR (300 MHz, CDCl₃) δ 6.81 (d, J=8.5 Hz, 1H), 6.76 (s, 1H), 6.02 (m, 1H), 4.05 (d, J=6.4 Hz, 2H), 3.72 (t, J=8.4 Hz, 1H), 2.91 (t, J=6.5 Hz, 2H), 2.84 (m, 2H), 2.56 (t, J=6.5 Hz, 2H), 2.04-2.28 (m, 4H), 1.86-1.97 (m, 8H), 1.56-1.70 (m, 1H), 1.17-1.54 (m, 9H), 0.77 (s, 3H). Mass spectrum (API-TIS) m/z 489 (MH⁺), 459 (M−NO).

### Example 11

### 2-(2-(Nitroso)adamantan-2-yl)ethyl(2E,4E,6E,8E)-3,7-dimethyl-9-(2,6,6-trimethylcyclohex-1-enyl)nona-2,4,6,8-tetraenoate

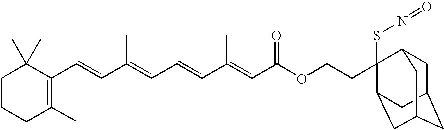

To a solution of (2E,4E,6E,8E)-3,7-dimethyl-9-(2,6,6-trimethylcyclohex-1-enyl)nona-2,4,6,8-tetraenoic acid (all trans retinoic acid) (100 mg, 0.33 mmol) and 2-(2-(nitrosothio)adamantan-2-yl)ethan-1-ol (prepared as described in U.S. Pat. No. 6,469,065, Example 12a), (560 mg, 2.33 mmol) in CH₂Cl₂ (5 mL), cooled to 0° C., was added a solution of 1,3-dicyclohexylcarbodiimide (86 mg, 0.42 mmol) and 4-dimethylaminopyridine (51 mg, 0.42 mmol) in CH₂Cl₂ (3 mL). The reaction mixture was stirred over ice for 1 hour, filtered and the residue after evaporation chromatographed on silica gel eluting with CH₂Cl₂:Hexane (1:1) to give the title compound (65 mg, 38% yield). ¹H NMR (300 MHz, CDCl₃) δ 7.00 (dd, J=15.0 and 11.3 Hz, 1H), 6.08-6.32 (m, 4H), 5.71 (s, 1H), 4.29 (t, J=7.3 Hz, 2H), 3.10 (t, J=7.4 Hz, 2H), 2.57 (br s, 2H), 2.42-2.51 (m, 2H), 2.34 (s, 3H), 2.00 (s, 3H), 1.71 (s, 3H), 1.67-2.15 (m, 14H), 1.58-1.67 (m, 1H), 1.45-1.49 (m, 1H), 1.03 (s, 6H). Mass spectrum (API-TIS) m/z 493 (M−NO⁺).

### Example 12

### 2,2-Bis((nitrooxy)methyl)-3-(nitrooxy)propyl(2E,4E,6E,8E)-3,7-dimethyl-9-(2,6,6-trimethylcyclohex-1-enyl)nona-2,4,6,8-tetraenoate

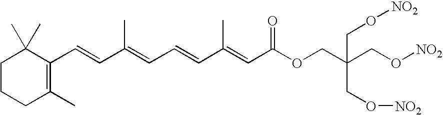

A solution of (2E,4E,6E,8E)-3,7-dimethyl-9-(2,6,6-trimethylcyclohex-1-enyl)nona-2,4,6,8-tetraenoic acid (all trans retinoic acid) (42 mg, 0.14 mmol), 4-dimethylaminopyridine (21 mg, 0.17 mmol) and 2,2-bis((nitrooxy)methyl)-3-(nitrooxy)propan-1-ol (prepared as described in WO 00/51978 as Example 11c, 27 μL, 39 mg, 0.14 mmol) in CH₂Cl₂ (1.0 mL) was cooled to 0° C. A solution of dicyclohexylcarbodiimide (35 mg, 0.17 mmol) in CH₂Cl₂ (0.5 mL) was slowly added in the dark. The reaction solution was stirred at 0° C. for 4 hours and at room temperature overnight in the dark, filtered, and evaporated. The residue was chromatographed on silica gel twice, eluting with neat CH₂Cl₂ followed by EtOAc:Hexane (1:19), to give the title compound (41 mg, 53% yield). ¹H NMR (300 MHz, CDCl₃) δ 7.07 (dd, J=15.0 and 11.4 Hz, 1H), 6.26-6.34 (m, 2H), 6.12-6.18 (m, 2H), 5.74 (s, 1H), 4.58 (s, 6H), 4.24 (s, 2H), 2.36 (s, 3H), 2.02 (m, 5H), 1.72 (s, 3H), 1.55-1.66 (m, 2H), 1.45-1.49 (m, 2H), 1.03 (s, 6H). ¹³C NMR (75 MHz, CDCl₃) δ 165.7, 155.9, 140.8, 137.6, 137.1, 134.3, 132.4, 130.3, 129.4, 129.2, 115.8, 69.5, 60.7, 42.2, 39.6, 34.3, 33.1, 28.9, 21.7, 19.2, 14.1, 12.9. Mass spectrum (API-TIS) m/z 554 (MH⁺), 571 (MNH₄⁺).

### Example 13

### (2R)-2,3-Bis(nitrooxy)propyl(2E,4E,6E,8E)-3,7-dimethyl-9-(2,6,6-trimethylcyclohex-1-enyl)nona-2,4,6,8-tetraenoate

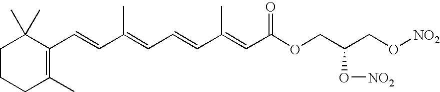

A solution of (2E,4E,6E,8E)-3,7-dimethyl-9-(2,6,6-trimethylcyclohex-1-enyl)nona-2,4,6,8-tetraenoic acid (all trans retinoic acid) (106 mg, 0.35 mmol), 4-dimethylaminopyridine (54 mg, 0.44 mmol) and (2R)-2,3-bis(nitroxy)propan-1-ol (prepared as described in U.S. application No. 2004/0024057 A, Example 5d, 300 μL, 459 mg, 2.52 mmol) in CH₂Cl₂ (5 mL) was cooled to 0° C. A solution of 1,3-dicyclohexylcarbodiimide (90 mg, 0.44 mmol) in CH₂Cl₂ (1 mL) was slowly added. The reaction mixture was stirred at 0° C. for 1 hour, filtered and evaporated. The residue was chromatographed on silica gel twice, eluting with EtOAc:Hexane 1:19 to 1:9 followed by neat CH₂Cl₂, to give the title compound (75 mg, 46% yield). ¹H NMR (300 MHz, CDCl₃) δ 7.06 (dd, J=15.0 and 11.4 Hz, 1H), 6.26-6.34 (m, 2H), 6.12-6.17 (m, 2H), 5.76 (s, 1H), 5.47-5.53 (m, 1H), 4.81 (dd, J=12.9 and 3.5 Hz, 1H), 4.65 (dd, J=12.9 and 6.7 Hz, 1H), 4.48 (dd, J=12.6 and 4.3 Hz, 1H), 4.32 (dd, J=12.6 and 5.3 Hz, 1H), 2.36 (s, 3H), 2.02 (m, 5H), 1.72 (s, 3H), 1.60-1.64 (m, 2H), 1.49-1.45 (m, 2H), 1.03 (s, 6H). ¹³C NMR (75 MHz, CDCl₃) δ 165.9, 155.5, 140.6, 137.6, 137.1, 134.4, 132.2, 130.2, 129.2, 116.1, 76.53, 68.8, 59.7, 39.6, 34.2, 33.1, 28.9, 21.7, 19.2, 14.0, 12.9. Mass spectrum (API-TIS) m/z 465 (MH⁺), 482 (MNH₄+).

### Example 14

### (2R)-2,3-Bis(nitrooxy)propyl(1S,11S,14S,15S,10R)-15-methyl-5-phenylcarbonyloxytetracyclo(8.7.0.0<2,7>0.0<11,15>)heptadeca-2,4,6-trien-14-yl butane-1,4-dioate

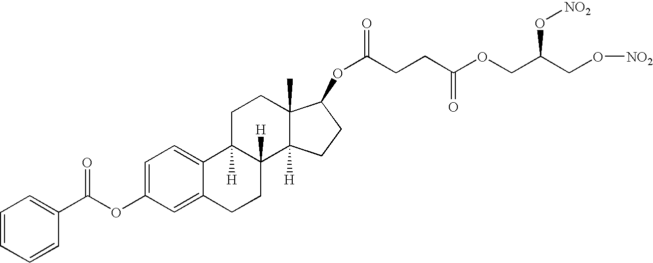

### 14a. 3-(((1S,11S,14S,15S,10R)-15-Methyl-5-phenylcarbonyloxytetracyclo(8.7.0.0<2,7>0.0<11,15>)heptadeca-2,4,6-trien-14-yl)oxycarbonyl)propanoic acid

β-Estradiol 3-benzoate (Aldrich, Wis., U.S., 5.0 g, 13.3 mmol) and succinic anhydride (Aldrich, Wis., US., 1.6 g, 15.9 mmol) were dissolved in THF (30 mL) and heated at reflux for 24 hours. The solvent was removed under reduced pressure and the residue dissolved in chloroform (20 mL). The sample was washed with water and satd. NaCl, and dried (MgSO₄). The residue after evaporation was chromatographed on silica gel eluting with Hexanes:EtOAc (3:1 to 2:1) to give the title compound (3.4 g, 53% yield) as a white solid. Mp 101-105° C. ¹H NMR (300 MHz, CDCl₃) δ 8.20 (d, J=7.3 Hz, 2H), 7.64 (t, J=7.3 Hz, 1H), 7.51 (t, J=7.6 Hz, 2H), 7.34 (d, J=8.5 Hz, 1H), 6.99 (d, J=8.5 Hz, 1H), 6.94 (br s, 1H), 4.74 (t, J=8.3 Hz, 1H), 2.91-2.88 (m, 2H), 2.69-2.67 (m, 4H), 2.34-2.20 (m, 4H), 1.93-1.89 (m, 2H), 1.77-1.75 (m, 1H), 1.60-1.31 (m, 6H), 0.85 (s, 3H).

### 14b. (2R)-2,3-Bis(nitrooxy)propyl(1S,11S,14S,15S,10R)-15-methyl-5-phenylcarbonyloxytetracyclo(8.7.0.0<2,7>0.0<11,15>)heptadeca-2,4,6-trien-14-yl butane-1,4-dioate

The product of Example 14a (519.0 mg, 1.1 mmol), (2R)-2,3-bis(nitrooxy)propan-1-ol (prepared as described in US patent WO2004/004648, Example 5d, 218.1 mg, 2.2 mmol), and DMAP (26.3 mg, 0.2 mmol) were dissolved in CH₂Cl₂ (30 mL) and EDAC (249.5 mg, 1.3 mmol) was added. The reaction mixture was stirred at room temperature for 2.5 hours and washed with water and satd. NaCl, and dried over MgSO₄. The residue after evaporation was filtered through a silica gel plug eluting with Hexanes:EtOAc (1:1) to give the title compound (440.0 mg, 63% yield) as a white solid. Mp 123-125° C. ¹H NMR (300 MHz, CDCl₃) δ 8.19 (d, J=7.1 Hz, 2H), 7.64 (br t, J=7.4 Hz, 1H), 7.51 (br t, J=8.1 Hz, 2H), 7.24 (d, J=8.4 Hz, 1H), 6.98 (dd, J=2.4, 8.4 Hz, 1H), 6.93 (br s, 1H), 5.51-5.46 (m, 1H), 4.84-4.62 (m, 3H), 4.50-4.43 (m, 1H), 4.37-4.23 (m, 1H), 2.91-2.88 (m, 2H), 2.66 (br s, 4H), 2.35-2.19 (m, 4H), 1.93-1.87 (m, 2H), 1.79-1.71 (m, 1H), 1.59-1.31 (m, 6H), 0.84 (s, 3H).

### Example 15

### (1S,11S,14S,15S,10R)-15-Methyl-5-phenylcarbonyloxytetracyclo(8.7.0.0<2,7>0.0<11,15>)heptadeca-2,4,6-trien-14-yl(1S,2S,5S,6R)-6-(nitrooxy)-4,8-dioxabicyclo(3.3.0)oct-2-yl butane-1,4-dioate

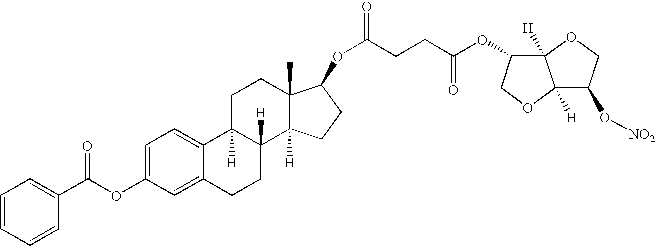 The product of Example 14a (480.0 mg, 1.0 mmol), isosorbide mononitrate (prepared as described in U.S. Pat. No. 4,431,830, Example 1, 211.6 mg, 1.1 mmol), and DMAP (24.3 mg, 0.20 mmol) were dissolved in CH₂Cl₂ (30 mL) and EDAC (230.8 mg, 1.2 mmol) was added. The reaction mixture was stirred at room temperature overnight. The sample was diluted with H₂O and extracted with additional CH₂Cl₂. The organics were combined, dried over MgSO₄, and the solvent removed under reduced pressure. The sample was purified via filtration through a silica gel plug eluting with Hexanes:EtOAc (1:1) to give the title compound (404.5 mg, 62% yield) as a white solid. Mp 146-148° C. ¹H NMR (300 MHz, CDCl₃) δ 8.21 (d, J=7.4 Hz, 2H), 7.65 (t, J=7.4 Hz, 1H), 7.52 (t, J=7.4 Hz, 2H), 7.34 (d, J=8.6 Hz, 2H), 6.98 (dd, J=2.5, 8.3 Hz, 1H), 6.93 (br s, 1H), 5.39-5.34 (m, 1H), 5.27 (br d, J=2.5 Hz, 1H), 5.00 (t, J=4.9 Hz, 1H), 4.75-4.69 (m, 1H), 4.51 (d, J=4.9 Hz, 1H), 4.07-3.89 (m, 3H), 2.91-2.89 (m, 2H), 2.66 (br s, 4H), 2.35-2.17 (m, 4H), 1.92-1.88 (m, 2H), 1.81-1.76 (m, 1H), 1.59-1.24 (m, 6H), 0.85 (s, 3H).

### Example 16

### (1S,11S,14S,15S,10R)-15-Methyl-5-phenylcarbonyloxytetracyclo(8.7.0.0<2,7>0.0<11,15>)heptadeca-2,4,6-trien-14-yl 3-(nitrooxy)propyl butane-1,4-dioate

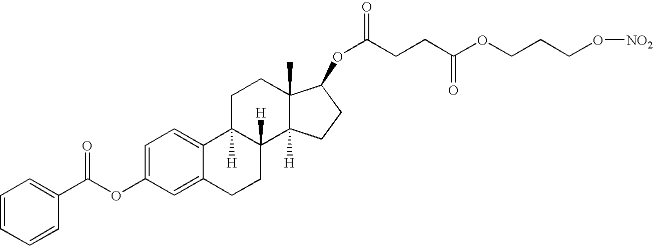

The product of Example 14a (490.0 mg, 1.1 mmol), 3-(nitrooxy)propan-1-ol (prepared as described in U.S. application No. 2004/0024057 A1, Example 40a,136.8 mg, 1.1 mmol), and DMAP (24.9 mg, 0.21 mmol) were dissolved in CH₂Cl₂ (30 mL) and EDAC (235.6 mg, 1.2 mmol) was added. The reaction mixture was stirred at room temperature for 3.5 hours, washed with H₂O and satd. NaCl, and dried over MgSO₄. The sample was purified via filtration through a silica gel plug eluting with Hexanes:EtOAc (1:1) to give the title compound (376.0 mg, 63% yield) as a white solid. Mp 86-88° C. ¹H NMR (300 MHz, CDCl₃) δ 8.15 (d, J=7.3 Hz, 2H), 7.59 (t, J=7.3 Hz, 1H), 7.46 (t, J=7.3 Hz, 2H), 7.29 (d, J=8.5 Hz, 1H), 6.94 (dd, J=2.5, 8.5 Hz, 1H), 6.88 (br s, 1H), 4.69 (m, 1H), 4.52 (t, J=6.2 Hz, 2H), 4.19 (t, J=6.2 Hz, 2H), 2.85 (m, 2H), 2.61 (br s, 4H), 2.31-2.05 (m, 4H), 2.04 (t, J=6.2 Hz, 2H), 1.92-1.80 (m, 2H), 1.72-1.68 (m, 1H), 1.55-1.26 (m, 6H), 0.82 (s, 3H).

### Example 17

### (1S,11S,14S,15S,10R)-15-Methyl-5-(2-(2-(nitrosothio)adamantan-2-yl)acetyloxy)tetracyclo(8.7.0.0<2,7>0.0<11,15>)heptadeca-2,4,6-trien-14-yl 2,2,2-trifluoroacetate

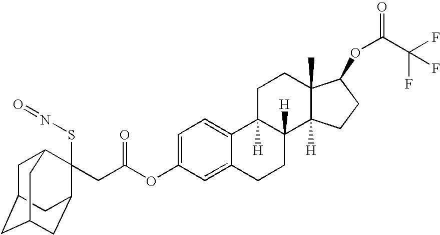

### 17a. (1S,11S,14S,15S,10R)-14-hydroxy-15-methyltetracyclo(8.7.0.0<2,7>0.0<11,15>)heptadeca-2,4,6-trien-5-yl 2-(2-((2,4,6-trimethoxyphenyl)methylthio)adamantan-2-yl)acetate

To β-estradiol (1.17 g, 4.29 mmol) and the product of Example 56b (1.93 g, 4.75 mmol) in DMF (60 mL) was added EDAC (1.08 g, 5.62 mmol) and DMAP (525.9 mg, 4.30 mmol). The reaction was stirred at room temperature overnight and concentrated to dryness under high vacuum at 40° C. The residue was treated with EtOAc and water. The organic phase was washed with 0.2 M citric acid, satd. NaCl, sodium bicarbonate, and satd. NaCl. The EtOAc solution was dried over MgSO₄, filtered, and concentrated. The crude product was purified by chromatography (silica gel, EtOAc:Hexane 1:10; 1:5; then 1:4) to give the title compound (1.86 g, 66% yield). ¹H NMR (300 MHz, CDCl₃) δ 7.28-7.25 (m, 1H), 6.99-6.95 (m, 1H), 6.88 (s, 1H), 6.09 (s, 2H), 3.83-3.74 (m, 12H), 3.22 (s, 2H), 2.86-2.83 (m, 2H), 2.74-2.61 (m, 2H), 2.36-1.11 (m, 26H), 0.78 (s, 3H). ¹³C NMR (75 MHz, CDCl₃) δ 169.9, 160.2, 158.9, 148.8, 137.8, 137.5, 126.0, 121.8, 119.1, 107.2, 90.5, 81.8, 55.6, 55.3, 55.0, 44.1, 43.2, 41.5, 39.3, 38.5, 36.6, 34.2, 33.1, 32.9, 30.5, 29.5, 27.53, 27.50, 27.1, 26.2, 23.1, 11.0. Mass spectrum (API-TIS) m/z 661 (MH⁺), 678 (MNH₄^{+),} 683 (MNa⁺).

### 17b. (1S,11S,14S,15S,10R)-15-methyl-5-(2-(2-sulfanyladamantan-2-yl)acetyloxy)tetracyclo(8.7.0.0<2,7>0.0<11,15>)heptadeca-2,4,6-trien-14-yl 2,2,2-trifluoroacetate

L-Cysteine (3.30 g, 27.2 mmol) was dissolved in TFA (10 mL). The product of Example 17a (1.80 g, 2.72 mmol) in CH₂Cl₂ (10 mL) was added. The reaction was stirred at room temperature overnight and concentrated to dryness. The residue was treated with CH₂Cl₂ and concentrated to dryness three times, dissolved in EtOAc and water, and washed with water, satd. NaCl, sodium bicarbonate, and satd. NaCl. The organic phase was dried over MgSO₄ and concentrated. The crude product was dissolved in acetone, and water was added to give crystals. The crystals were collected by filtration, washed with acetone-water, and dried in vacuum to give the title compound (1.15 g, 73% yield). ¹H NMR (300 MHz, CDCl₃) δ 7.29-7.27 (m, 2H), 6.90-6.83 (m, 2H), 4.91-4.86 (m, 1H), 3.20 (s, 2H), 2.89-2.86 (m, 2H), 2.54-2.50 (m, 2H), 2.30-1.38 (m, 25H), 0.88 (s, 3H). ¹³C NMR (75 MHz, CDCl₃) δ 170.0, 158.2, 157.6, 157.1, 156.5, 148.3, 137.9, 137.3, 126.2, 121.5, 120.2, 118.7, 116.4, 112.6, 108.8, 86.6, 53.9, 49.4, 46.3, 43.7, 43.2, 38.8, 38.0, 36.5, 33.8, 33.2, 30.7, 29.3, 27.4, 27.0, 26.8, 26.7, 25.8, 23.0, 11.7. Mass spectrum (API-TIS) m/z 594 (MNH₄⁺), 1170 (2MNH₄⁺).

### 17c. (1S,11S,14S,15S,10R)-15-methyl-5-(2-(2-(nitrosothio)adamantan-2-yl)acetyloxy)tetracyclo(8.7.0.0<2,7>0.0<11,15>)heptadeca-2,4,6-trien-14-yl 2,2,2-trifluoroacetate

To the product of Example 17b (1.08 g, 1.86 mmol) in CH₂Cl₂ (10 mL) was added tert-butyl nitrite (90% solution, 0.35 mL, 2.94 mmol). The reaction was stirred at room temperature for 10 minutes and concentrated to dryness. The residue was dissolved in EtOAc and washed with water, and satd. NaCl. The organic phase was dried over MgSO₄, filtered, and concentrated. The crude product was dissolved in acetone, and water was added to give crystals. Crystals were collected by filtration, washed with acetone-water, and dried in vacuum to give the title compound (1.02 g, 90% yield). ¹H NMR (300 MHz, CDCl₃) δ 7.23-7.20 (m, 1H), 6.70-6.62 (m, 2H), 4.89-4.84 (m, 1H), 3.95 (s, 2H), 2.86-2.81 (m, 2H), 2.49-2.45 (m, 2H), 2.28-1.42 (m, 25H), 0.87 (s, 3H). ¹³C NMR (75 MHz, CDCl₃) δ 169.1, 158.2, 157.7, 157.1, 156.6, 148.1, 137.8, 137.4, 126.2, 121.3, 120.2, 118.5, 116.4, 112.7, 108.9, 86.6, 65.9, 49.5, 43.7, 43.3, 42.4, 38.7, 38.0, 36.5, 36.6, 33.7, 33.1, 29.3, 27.0, 26.8, 25.8, 23.1, 11.7. Mass spectrum (API-TIS) m/z 623 (MNH₄⁺).

### Example 18

### (1S,11S,14S,15S,10R)-14-hydroxy-15-methyltetracyclo(8.7.0.0<2,7>0.0<11,15>)heptadeca-2,4,6-trien-5-yl 2-(2-(nitrosothio)adamantan-2-yl)acetate

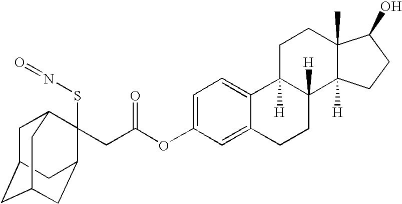

The product of Example 17b (650 mg, 1.07 mmol) in THF (30 mL), water (1 mL), and sodium bicarbonate solution (1 mL) was stirred at room temperature for 4 hours and concentrated. The resultant aqueous phase was extracted with CH₂Cl₂ twice. The combined organic phase was dried over MgSO₄, filtered, and concentrated. The crude product was purified by chromatography (silica gel, EtOAc:Hexane 1:3) to give the title compound (278 mg, 50% yield). ¹H NMR (300 MHz, CDCl₃) δ 7.23-7.20 (m, 1H), 6.70-6.62 (m, 2H), 4.89-4.84 (m, 1H), 3.95 (s, 2H), 2.86-2.81 (m, 2H), 2.49-2.45 (m, 2H), 2.28-1.42 (m, 26H), 0.87 (s, 3H). ¹³C NMR (75 MHz, CDCl₃) δ 169.3, 148.0, 138.1, 138.0, 126.3, 121.3, 118.4, 81.7, 65.9, 50.0, 44.1, 43.1, 42.5, 38.8, 38.4, 36.6, 35.6, 34.8, 33.8, 33.1, 30.4, 29.5, 27.1, 27.0, 26.1, 23.1, 11.0. Mass spectrum (API-TIS) m/z 527 (MNH₄⁺).

### Example 19

### (1S,11S,14S,15S,10R)-14-hydroxy-15-methyltetracyclo(8.7.0.0<2,7>0.0<11,15>)heptadeca-2,4,6-trien-5-yl 3,3-dimethyl-4-(N-(2-methyl-2-(nitrosothio)propyl)carbamoyl)butanoate

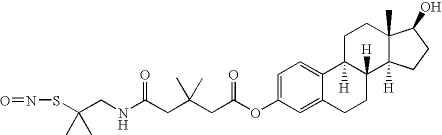

To a mixture of β-estradiol (981.9 mg, 3.61 mmol) and 3-(N-(2,2-dimethylpropyl)-N-(2-methyl-2-(nitrosothio)propyl)carbamoyl)propanoic acid (997.9 mg, 3.61 mmol) in DMF (15 mL) was added DCC (774.0 mg, 3.75 mmol) in CH₂Cl₂ (3 mL). The reaction was stirred at room temperature for half an hour, and DMAP (434.1 mg, 3.55 mmol) was added. The reaction was then stirred at room temperature for 4 hours and filtered to remove DCU. The filtrate was concentrated and precipitated with water. The aqueous phase was discarded, and the green oil was collected and dissolved in EtOAc. The EtOAc solution was washed with 0.2 M citric acid, and satd. NaCl. The organic phase was dried over MgSO₄, filtered, and concentrated. The resultant oil was treated with CH₂Cl₂ and filtered again to remove DCU. The filtrate was concentrated and purified by chromatography on silica gel eluting with EtOAc:Hexane (1:4; then 32:68) to give the title compound (993.1 mg, 52% yield). ¹H NMR (300 MHz, CDCl₃) δ 7.32-7.29 (m, 1H), 6.85-6.75 (m, 2H), 4.05-4.00 (m, 2H), 3.72-3.70 (m, 1H), 2.89-2.86 (m, 2H), 2.54 (s, 2H), 2.34 (s, 2H), 2.34-1.18 (m, 15H), 1.89 (s, 6H), 1.21 (s, 6H), 0.76 (s, 3H). ¹³C NMR (75 MHz, CDCl₃) δ 172.1, 171.4, 147.9, 138.41, 138.36, 126.5, 121.3, 118.4, 81.8, 57.1, 50.0, 49.3, 47.0, 44.6, 44.1, 43.1, 38.4, 36.6, 34.0, 30.5, 29.5, 28.8, 27.02, 26.95, 26.1, 23.1, 11.0. Mass spectrum (API-TIS) m/z 531 (MH⁺).

### Example 20

### (1S,11S,14S,15S,10R)-14-hydroxy-15-methyltetracyclo(8.7.0.0<2,7>0.0<11,15>)heptadeca-2,4,6-trien-5-yl 3-(N-(2-methyl-2-(nitrosothio)propyl)-N-benzylcarbamoyl)propanoate

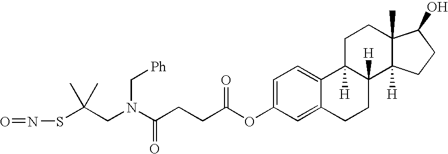

To β-estradiol (641.7 mg, 2.35 mmol) and 3-(N-(2-methyl-2-(nitrosothio)propyl)-N-benzylcarbamoyl)propanoic acid (987.5 mg, 3.04 mmol) in DMF (15 mL) was added EDAC (690.9 mg, 3.60 mmol) in CH₂Cl₂ (20 mL). After 10 minutes, DMAP (173.9 mg, 1.42 mmol) was added. The reaction was stirred at room temperature for additional 40 minutes and stored at −20° C. overnight. The reaction solution was concentrated, and the product was precipitated by adding water. The solid collected by filtration, washed with water, and dissolved in EtOAc. The EtOAc solution was washed with 0.5 M citric acid, satd. NaCl, sodium bicarbonate, and satd. NaCl. The organic phase was dried over MgSO₄, filtered, and concentrated to give a crude product. The crude product was purified by chromatography (silica gel, EtOAc:CH₂Cl₂ 1:19) to give the title compound (712.6 mg, 52% yield) and the product of Example 21 (64.4 mg, 3% yield). ¹H NMR (300 MHz, CDCl₃) δ 7.35-7.26 (m, 4H), 7.13-7.07 (m, 2H), 6.86-6.79 (m, 2H), 4.63 (s, 2H), 4.20 (s, 2H), 3.72 (t, J=8.4 Hz, 1H), 2.90-2.72 (m, 6H), 2.38-1.07 (m, 14H), 1.92 (s, 6H), 0.77 (s, 3H). ¹³C NMR (75 MHz, CDCl₃) δ 173.4, 171.8, 148.4, 138.1, 137.9, 136.2, 129.0, 127.7, 126.3, 125.9, 121.5, 118.5, 81.8, 58.5, 55.5, 52.9, 50.0, 44.1, 43.1, 38.4, 36.6, 30.5, 29.5, 28.4, 28.0, 27.6, 27.0, 26.1, 23.1, 11.0. Mass spectrum (API-TIS) m/z 579 (MH⁺), 596 (MNH₄⁺), 1174 (2MNH₄⁺).

### Example 21

### (1S,11S,14S,15S,10R)-15-Methyl-5-(3-(N-(2-methyl-2-(nitrosothio)propyl)-N-benzylcarbamoyl)propanoyloxy)tetracyclo(8.7.0.0<2,7>0.0<11,15>)heptadeca-2,4,6-trien-14-yl 3-(N-(2-methyl-2-(nitrosothio)propyl)-N-benzylcarbamoyl)propanoate

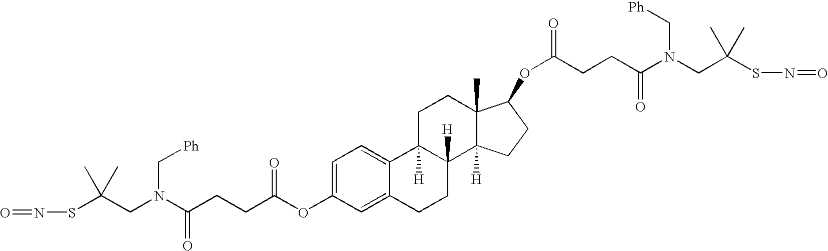

The crude product of Example 20 was purified by chromatography (silica gel, EtOAc:CH₂Cl₂ 1:19) to give the product of Example 20 (712.6 mg, 52% yield) and the title compound (64.4 mg, 3% yield). ¹H NMR (300 MHz, CDCl₃) δ 7.36-7.26 (m, 8H), 7.09-7.07 (m, 3H), 6.85-6.79 (m, 2H), 4.70 (m, 1H), 4.63 (s, 4H), 4.20 (m, 4H), 2.88-2.65 (m, 10H), 2.38-1.07 (m, 25H), 0.77 (s, 3H). ¹³C NMR (75 MHz, CDCl₃) δ 173.5, 173.3, 172.8, 171.8, 148.4, 137.9, 137.7, 136.3, 136.1, 28.97, 128.95, 127.57, 127.55, 126.3, 125.9, 121.4, 118.5, 82.7, 77.2, 60.3, 58.54, 58.49, 55.5, 52.92, 52.86, 49.7, 43.9, 42.9, 38.1, 36.8, 29.50, 29.46, 29.38, 29.4, 27.6, 27.5, 27.4, 26.9, 25.9, 23.2, 21.0, 14.1, 12.0. Mass spectrum (API-TIS) m/z 885 (MH⁺), 902 (MNH₄⁺).

### Example 22

### (1S,11S,14S,15S,10R)-14-Hydroxy-15-methyltetracyclo(8.7.0.0<2,7>0.0<11,15>)heptadeca-2,4,6-trien-5-yl 2-(2,2-dimethyl-3-(nitrooxy)propanoylamino)-3-((2,4,6-trimethoxyphenyl)methylthio)propanoate

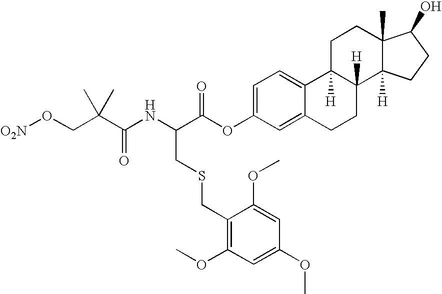

### 22a. 2-amino-3-((2,4,6-trimethoxyphenyl)methylthio)propanoic acid

To L-cysteine (8.17 g, 67.45 mmol) in TFA (80 mL) was added 2,4,6-trimethoxybenzyl alcohol (13.37 g, 67.46 mmol) in CH₂Cl₂ (60 mL). The reaction solution was stirred at room temperature for 5 minutes, concentrated to dryness. The resultant product was treated with EtOAc and concentrated to dryness three times to give white solid. The white solid was dissolved in hot water (750 mL, 90° C.), and the pH was adjusted to 6.3 with KOH solution to give precipitate. The precipitate was collected by filtration and dried in vacuum at 40° C. to give the title compound (15.73 g, 77% yield). ¹H NMR (300 MHz, CDCl₃) δ 6.20 (s, 2H), 3.84-3.71 (m, 11H), 3.31-3.19 (m, 2H), 2.76-2.72 (m, 1H). ¹³C NMR (75 MHz, CDCl₃) δ 173.0, 162.3, 160.2, 108.2, 91.7, 56.2, 55.8, 55.4, 34.3, 24.4. Mass spectrum (API-TIS) m/z 302 (MH⁺), 324 (MNa⁺), 603 (2MH⁺).

### 22b. 2-(2,2-dimethyl-3-(nitrooxy)propanoylamino)-3-((2,4,6-trimethoxyphenyl)methylthio)propanoic acid

The product of Example 22a (6.00 g, 19.91 mmol) was suspended in CH₂Cl₂ (18 mL) under argon was added N,O-bis(trimethylsilyl)acetamide (10 mL, 40.5 mmol), and the reaction was stirred at room temperature till obtaining a homogeneous solution. In a separate flask, 2,2-dimethyl-3-(nitrooxy)propanoic acid (3.25 g, 19.91 mmol) and EDAC (4.12 g, 21.49 mmol) in CH₂Cl₂ was stirred under argon at room temperature for 10 minutes and then transferred to the previous solution under argon. The resultant solution was stirred at room temperature for 2 hours. Water was added to the reaction solution to give precipitate, and CH₂Cl₂ was removed by evaporation. The resultant solid was dissolved in EtOAc. The EtOAc solution was washed with water, 0.2 M citric acid, and satd. NaCl. The organic phase was dried over MgSO₄, filtered, and concentrated to give a crude product (7 g). The crude product was purified by chromatography (silica gel, EtOAc:Hexane:HOAc 35:65:0.5; then 50:50:0.5) to give the title compound (2.38 g, 27% yield). ¹H NMR (300 MHz, CDCl₃) δ 6.67 (d, J=6.4 Hz, 1H), 6.12 (s, 2H), 4.74 (m, 1H), 4.50 (m, 2H), 3.80 (m, 11H), 3.10 (m, 1H), 2.92 (m, 1H), 1.30 (s, 6H). ¹³C NMR (75 MHz, CDCl₃) δ 174.7, 174.1, 160.5, 158.7, 107.1, 90.5, 77.9, 55.7, 55.3, 51.7, 41.9, 32.8, 23.9, 22.3, 22.2. Mass spectrum (API-TIS) m/z 445 (M−H⁻), 891 (2M−H⁻).

### 22c. (1S,11S,14S,15S,10R)-14-hydroxy-15-methyltetracyclo(8.7.0.0<2,7>0.0<11,15>)heptadeca-2,4,6-trien-5-yl 2-(2,2-dimethyl-3-(nitrooxy)propanoylamino)-3-((2,4,6-trimethoxyphenyl)methylthio)propanoate

The product of Example 22b (1.23 g, 2.76 mmol) and β-estradiol (750.4 mg, 2.76 mmol) in DMF (10 mL) under argon added EDAC (616.4 mg, 3.22 mmol) in CH₂Cl₂ (25 mL). The reaction was stirred at room temperature for 10 minutes, and DMAP (324.1 mg, 2.65 mmol) was added. The reaction was then stirred at room temperature for three days and then concentrated to dryness under vacuum. The resultant oil dissolved in EtOAc and washed with water, 0.5 M citric acid, sodium bicarbonate, and satd. NaCl. The organic phase was dried over MgSO₄, filtered, and concentrated. The resultant organic was stirred in CH₂Cl₂ to give precipitate. The precipitate (386.4 mg) was the un-reacted β-estradiol and was removed by filtration. The filtrate was concentrated and purified by chromatography (silica gel, EtOAc:Hexane 1:3; 8:17; 2:3) to give the title compound (631.0 mg, 33% yield). ¹H NMR (300 MHz, CDCl₃) δ 6.85-6.79 (m, 2H), 6.66-6.64 (m, 1H), 6.13 (s, 2H), 4.98 (m, 1H), 4.58-4.48 (m, 2H), 3.89-3.75 (m, 12H), 3.16 (m, 1H), 3.08 (m, 1H), 2.84 (m, 2H), 2.43-1.09 (m, 15H), 1.31 (s, 6H), 0.77 (s, 3H). ¹³C NMR (75 MHz, CDCl₃) δ 174.1, 170.1, 160.5, 158.7, 148.1, 138.3, 126.4, 121.2, 118.3, 107.3, 90.5, 81.8, 78.0, 55.7, 55.3, 52.2, 50.0, 44.1, 43.2, 41.9, 38.4, 36.6, 33.3, 30.5, 29.5, 27.0, 26.1, 24.3, 23.1, 22.6, 22.3, 11.0. Mass spectrum (API-TIS) m/z 701 (MH⁺), 718 (MNH₄⁺).

### Example 23

### (1S,11S,14S,15S,10R)-14-hydroxy-15-methyltetracyclo(8.7.0.0<2,7>0.0<11,15>)heptadeca-2,4,6-trien-5-yl 3-acetylthio-2-(2,2-dimethyl-3-(nitrooxy)propanoylamino)propanoate

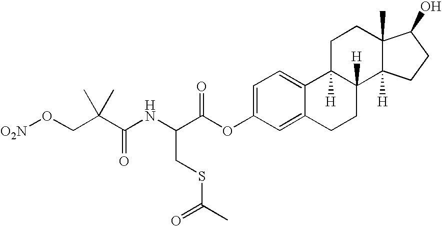

To L-cysteine (1.20 g, 9.90 mmol) in TFA (7 mL) was added the product of Example 22c (688 mg, 0.98 mmol) in CH₂Cl₂ (7 mL). The reaction was stirred at room temperature for 10 minutes, concentrated to dryness, treated with EtOAc and concentrated to dryness three times. The resultant product was dissolved in EtOAc and washed with water, sodium bicarbonate, and satd. NaCl. The organic phase was dried over MgSO₄, filtered, and concentrated. The resultant product was immediately treated a pre-mixed acetic anhydride (0.75 mL, 7.93 mmol) and pyridine (7 mL). The reaction was then stirred at room temperature for half an hour and then concentrated to dryness under vacuum. The resultant product was dissolved in EtOAc and washed with 0.5 M citric acid, satd. NaCl, sodium bicarbonate, and satd. NaCl. The organic phase was dried over MgSO₄, filtered, and concentrated to give a crude product (523.7 mg). The crude product was purified by chromatography (silica gel, EtOAc:Hexane 25:75; 32:68; 40:60) to give a product which was purified again by chromatography (silica gel, MeOH:CH₂Cl₂ 0.7:99.3) to give the title compound (293.7 mg, 53% yield). ¹H NMR (300 MHz, CDCl₃) δ 7.29 (d, J=8.8Hz, 1H), 6.89-6.77 (m, 3H), 4.93 (m, 1H), 4.55 (d, J=10Hz, 1H), 4.45(d, J=10 Hz, 1H), 3.72 (m, 1H), 3.53-3.52 (m, 2H), 2.87-2.83 (m, 2H),2.41 (s, 3H), 3.89-3.75 (m, 14H), 1.30 (s, 6H), 0.77 (s, 3H). ¹³C NMR (75 MHz, CDCl₃) δ 196.6, 174.3, 168.8, 147.9, 138.5, 138.4, 126.4, 121.0, 118.1, 81.7, 77.8, 53.2, 49.9, 44.0, 43.1, 41.8, 38.3, 36.6, 30.4, 30.1, 29.4, 26.9, 26.1, 23.0, 22.5, 22.1, 11.0. Mass spectrum (API-TIS) m/z 563 (MH⁺), 580 (MNH₄⁺), 585 (MNa⁺), 1142 (2MNH₄⁺).

### Example 24

### (1S,11S,14S,15S,10R)-14-Hydroxy-15-methyltetracyclo(8.7.0.0<2,7>0.0<11,15>)heptadeca-2,4,6-trien-5-yl 3-(N-(2,2-dimethylpropyl)-N-(2-methyl-2-(nitrosothio)propyl)carbamoyl)propanoate

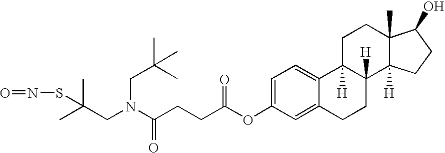

To β-estradiol (448.3 mg, 1.65 mmol) and 3-(N-(2,2-dimethylpropyl)-N-(2-methyl-2-(nitrosothio)propyl)carbamoyl)propanoic acid (502.1 mg, 1.65 mmol). 3-(N-(2,2-dimethylpropyl)-N-(2-methyl-2-(nitrosothio)propyl)carbamoyl)propanoic acid) in DMF (15 mL) was added EDAC (369.1 mg, 1.92 mmol) in CH₂Cl₂. After 10 minutes, DMAP was added, and the reaction was stirred at room temperature overnight. The reaction solution was concentrated, and water was added to give precipitate. The precipitate was collected, washed with water, and dissolved in EtOAc. The EtOAc solution was washed with 0.5 M citric acid, satd. NaCl, sodium bicarbonate, and satd. NaCl. The organic phase was dried over MgSO₄, filtered, and concentrated. The crude product was purified by chromatography (silica gel, EtOAc:CH₂Cl₂ 1:49; then 1:14) to give the title compound (83.2 mg, 9% yield). ¹H NMR (300 MHz, CDCl₃) δ 7.28-7.25 (m, 1H), 6.85-6.77 (m, 2H), 4.33 (br, 1H), 3.73 (t, J=8.4 Hz, 1H), 3.26 (s, 2H), 2.93-2.79 (m, 6H), 2.37-1.11 (m, 21H), 0.94 (s, 9H), 0.77 (s, 3H). ¹³C NMR (75 MHz, CDCl₃) δ 173.6, 172.0, 148.4, 138.1, 137.9, 126.3, 121.5, 118.5, 81.8, 59.3, 59.0, 55.6, 50.0, 44.1, 43.1, 38.4, 36.7, 34.7, 30.5, 30.1, 29.5, 28.8, 28.7, 27.7, 27.0, 26.1, 23. 1, 11.0. Mass spectrum (API-TIS) m/z 559 (MH⁺), 576 (MNH₄⁺).

### Example 25

### (1S,11S,14S,15S,10R)-14-Hydroxy-15-methyltetracyclo(8.7.0.0<2,7>0.0<11,15>)heptadeca-2,4,6-trien-5-yl 2-(2-(nitrosothio)adamantan-2-yl)ethyl butane-1,4-dioate

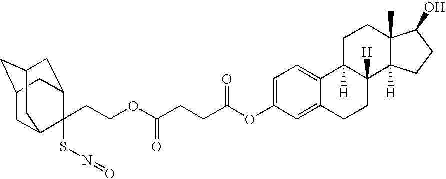

To a mixture of β-estradiol (454 mg, 1.667 mmol), 3-((2-(2-(nitrosothio)adamantan-2-yl)ethyl)oxycarbonyl)propanoic acid (prepared as described in U.S. Pat. No. 6,469,065, Example 10e) (683 mg, 2.0 mmol), and 4-dimethylaminopyridine(DMAP) (41 mg, 0.33 mmol) in CH₂Cl₂ at room temperature was added 1-(3-(dimethylamino)propyl)-3-ethylcarbodiimide hydrochloride (EDAC) (383 mg, 2.0 mmol). The reaction mixture was stirred for 90 minutes at room temperature, at which time the reaction was complete as monitored by TLC. The reaction mixture was washed with 0.1 M hydrochloric acid, water, satd. NaCl and dried over MgSO₄. The residue after filtration and evaporation was purified via chromatography on silica gel (EtOAc:CH₂Cl₂ 1:9) to give the title compound as a green oil (830 mg, 1.39 mmol, 84% yield). ¹H NMR (300 MHz, d₆-DMSO) δ 7.29 (d, J=8.5 Hz, 1H), 6.80 (dd, J=8.5, 2.3 Hz, 1H), 6.73 (d, J=2.3 Hz, 1H), 4.51 (d, J=4.7 Hz, 1H), 4.21 (t, J=7.2 Hz, 2H), 3.52 (m, 1H), 3.33 (s, 2H), 2.99 (t, J=7.2 Hz, 2H), 2.78 (m, 4H), 2.61 (m, 2H), 2.36 (m, 3H), 2.27 (m, 1H), 1.99-1.70 (m, 15H), 1.40-1.09 (m, 6H), 0.67 (s, 3H). Mass spectrum (API-TIS) m/z 613 (MNH₄⁺), 583 (MNH₄⁺—NO).

### Example 26

### (1S,11S,14S,15S,10R)-15-Methyl-5-phenylcarbonyloxytetracyclo(8.7.0.0<2,7>0.0<11,15>)heptadeca-2,4,6-trien-14-yl 2-(2-(nitrosothio)adamantan-2-yl)ethyl butane-1,4-dioate

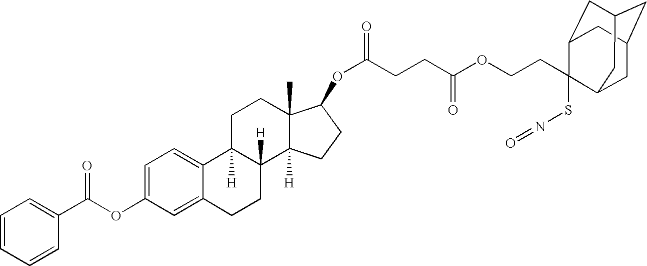

To a mixture of β-estradiol-3-benzoate (628 mg, 1.667 mmol), 3-((2-(2-(nitrosothio)adamantan-2-yl)ethyl)oxycarbonyl)propanoic acid (prepared as described in U.S. Pat. No. 6,469,065, Example 10e, 683 mg, 2.0 mmol), and 4-dimethylaminopyridine(DMAP) (41 mg, 0.33 mmol) in CH₂Cl₂ at room temperature was added 1-(3-(dimethylamino)propyl)-3-ethylcarbodiimide hydrochloride (EDAC) (383 mg, 2.0 mmol). The reaction mixture was stirred for 2 hours at room temperature, at which time the reaction was complete as monitored by TLC. The reaction mixture was washed with 0.1 M hydrochloric acid, water, satd. NaCl and dried over MgSO₄. The residue after filtration and evaporation was purified via chromatography on silica gel eluting with CH₂Cl₂ to give the title compound as a green oil (520 mg, 44% yield). ¹H NMR (300 MHz, d₆-DMSO) δ 8.11 (d, J=8.5 Hz, 2H), 7.75 (t, J=7.4 Hz, 1H), 7.60 (t, J=7.6 Hz, 2H), 7.34 (d, J=8.6 Hz, 1H), 7.02 (dd, J=2.3, 8.4 Hz, 1H), 6.96 (d, J=2.3 Hz, 1H), 4.62 (t, J=8.2 Hz, 2H), 4.18 (t, J=7.2 Hz, 2H), 3.33 (s, 2H), 3.00 (t, J=7.2 Hz, 2H), 2.84 (m, 2H), 2.53 (m, 3H), 2.40 (m, 2H), 2.29 (m, 2H), 2.15-1.55 (m, 14H), 1.50-1.20 (m, 7H), 0.77 (s, 3H). Mass spectrum (API-TIS) m/z 717 (MNH₄⁺), 687 (MNH₄⁺—NO).

### Example 27

### (2R)-2,3-Bis(nitrooxy)propyl(1S,11S,14S,15S,10R)-14-hydroxy-15-methyltetracyclo(8.7.0.0<2,7>0.0<11,15>)heptadeca-2,4,6-trien-5-yl butane-1,4-dioate

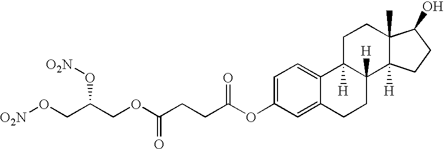

### 27a: 3-(((2R)-2,3-Bis(nitrooxy)propyl)oxycarbonyl)propanoic acid

To a mixture of succinic anhydride (1.71 g, 9.39 mmol) and DMAP (1.377 g, 11.27 mmol) in THF (50 mL) was added (2R)-2,3-bis(nitrooxy)propan-1-ol (prepared as described in U.S. application No. 2004/0024057, Example 5d; 1.13 g, 11.27 mmol). The solution was heated at 60° C. for 18 hours and cooled to room temperature. The residue was partitioned between EtOAc and water, acidifying the water layer to pH 1 with 3N HCl as needed. The layers were separated, and the organic layer was washed with water, satd. NaCl, and dried over MgSO₄. Removal of the solvent under reduced pressure gave the title compound (2.41 g, 8.5 mmol, 91% yield) as a colorless oil. ¹H NMR (300 MHz, CDCl₃) δ 5.49 (m, 1H), 4.80 (dd, J=3.6, 12.9 Hz, 1H), 4.64 (dd, J=6.5, 12.9 Hz, 1H), 4.50 (dd, J=4.1, 12.6 Hz, 1H), 4.35 (dd, J=5.4, 12.6 Hz, 1H), 2.71 (m, 4H). Mass spectrum (API-TIS) m/z 300 (MNH₄⁺), 283 (MH⁺).

### 27b. (2R)-2,3-Bis(nitrooxy)propyl(1S,11S,14S,15S,10R)-14-hydroxy-15-methyltetracyclo(8.7.0.0<2,7>0.0<11,15>)heptadeca-2,4,6-trien-5-yl butane-1,4-dioate

To a mixture of β-estradiol (1.10 g, 4.04 mmol), the product of Example 27a (1.14 g, 4.04 mmol), and 4-dimethylaminopyridine(DMAP) (99 mg, 0.81 mmol) in CH₂Cl₂ at room temperature was added 1-(3-(dimethylamino)propyl)-3-ethylcarbodiimide hydrochloride (EDAC) (383 mg, 2.0 mmol). The reaction mixture was stirred for 18 hours at room temperature, at which time the reaction was complete as monitored by TLC. The reaction mixture was washed with 0.1 M hydrochloric acid, water, satd. NaCl and dried over MgSO₄. The residue after filtration and evaporation was purified via chromatography on silica gel (5% EtOAc in CH₂Cl₂ to 10% EtOAc in CH₂Cl₂) to give the title compound as a thick colorless oil (1.26 g, 2.35 mmol, 58% yield). ¹H NMR (300 MHz, CDCl₃) δ 7.29 (d, J=8.4 Hz, 1H), 6.84 (dd, J=2.5, 8.4 Hz, 1H), 6.80 (d, J=2.5 Hz, 1H), 5.48 (m, 1H), 4.79 (dd, J=3.5, 12.9 Hz, 1H), 4.63 (dd, J=6.5, 12.9 Hz, 1H), 4.50 (dd, J=4.2, 12.5 Hz, 1H), 4.35 (dd, J=5.3, 12.5 Hz, 1H), 3.73 (t, J=8.3 Hz, 1H), 2.93-2.84 (m, 4H), 2.78 (m, 2H), 2.35-2.08 (m, 2H), 1.99-1.85 (m, 2H), 1.75-1.17 (m, 10H), 0.78 (s, 3H). Mass spectrum (API-TIS) m/z 554 (MNH₄⁺).

### Example 28

### (1S,11S,14S,15S,10R)-14-Hydroxy-15-methyltetracyclo(8.7.0.0<2,7>0.0<11,15>)heptadeca-2,4,6-trien-5-yl 2-(4,4-dimethyl-1-(nitrosothio)cyclohexyl)ethyl butane-1,4-dioate

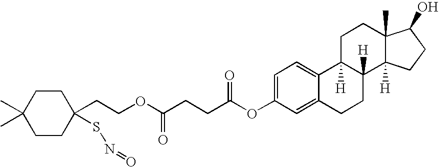

### 28a. 4,4-Dimethylcyclohexan-1-one

4,4-Dimethyl-2-cyclohexen-1-one (Aldrich, Wis., U.S., 25.0 g, 201.6 mmol) was placed in a Parr shaker hydrogenation apparatus and 100 mL of EtOAc was added. Palladium catalyst (Aldrich, Wis., U.S., 10 wt % on activated carbon, 1.4 g) was added. Hydrogen gas (25 psi) was added and the reaction flask shaken for 1 hour. The solid was removed via filtration through Celite. The solvent was removed from the filtrate via evaporation under reduced pressure to give the title compound (23.2 g, 91% yield). ¹H NMR (300 MHz, CDCl₃) δ 2.29 (t, J=6.9, 4H), 1.62 (t, J=6.9, 4H), 1.04 (s, 6H).

### 28b. Methyl 2-(4,4-dimethylcyclohexylidene)acetate

Trimethylphosphonoacetate (Aldrich, Wis., U.S., 38.5 mL, 238.4 mmol) was dissolved in DMF (150 mL) and NaH (Aldrich, Wis., U.S., 60 wt % in mineral oil, 8.80 g, 220.1 mmol) was added. The solution was stirred at room temperature for 20 minutes, cooled to 0° C., and the product of Example 28a (23.2 g, 183.4 mmol) was added. The reaction mixture was stirred at room temperature for 24 hours. Water was added (200 mL) and the sample extracted with hexanes (3×100 mL). The organic layers were combined, washed with satd. NaCl, dried over MgSO₄, and the solvent removed under reduced pressure to give the title compound (29.8 g, 89% yield). ¹H NMR (300 MHz, CDCl₃) δ 5.57 (s, 1H), 3.63 (s, 3H), 2.91-2.79 (m, 2H), 2.20-2.16 (m, 2H), 1.42-1.36 (m, 4H), 0.94 (s, 6H).

### 28c. 2-(4,4-Dimethylcyclohexylidene)acetic acid

The product of Example 28b (33.9 g, 185.99 mmol) was dissolved in MeOH (100 mL) and 2N NaOH (100 mL) was added. The reaction mixture was stirred at reflux for 2.5 hours and the MeOH was removed under reduced pressure. Cold HCl was added until a pH of 1 was achieved and the mixture was extracted with CH₂Cl₂. The extracts were combined, washed with satd. NaCl, and the solvent removed under reduced pressure to give the title compound (22.1 g, 70% yield) as a white solid. ¹H NMR (300 MHz, CDCl₃) δ 5.63 (s, 1H), 2.87-2.83 (m, 2H), 2.27-2.23 (m, 2H), 1.47-1.40 (m, 4H), 0.98 (s, 3H).

### 28d. 2-(4,4-Dimethyl-1-(phenylmethylthio)cyclohexyl)acetic acid

The product of Example 28c (22.1 g, 131.3 mmol) was dissolved in piperidine (80 mL) and benzyl mercaptan (Aldrich, Wis., U.S., 21.5 mL, 183.9 mmol) was added. The mixture was heated at reflux for 24 hours and the solvent was removed under reduced pressure. The mixture was diluted with ice water (200 mL) and concentrated HCl was added until a pH of 1 was achieved. The mixture was extracted with CH₂Cl₂, the organics were collected, washed with satd. NaCl, and dried over MgSO₄. The solvent was removed under reduced pressure and hexane (100 mL) was added. The resulting precipitate was collected via filtration, and washed with additional hexane to give the title compound (24.6 g, 64% yield) as a white solid. ¹H NMR (300 MHz, CDCl₃) δ 7.37-7.18 (m, 5H), 3.76 (s, 2H), 2.69 (s, 2H), 1.81-1.75 (m, 4H), 1.66-1.61 (m, 2H), 1.25-1.19 (m, 2H), 0.93 (s, 3H), 0.89 (s, 3H).

### 28e. 2-(4,4-Dimethyl-1-(phenylmethylthio)cyclohexyl)ethan-1-ol

The product of Example 28d (24.6 g, 84.11 mmol) was dissolved in THF (200 mL) and cooled to 0° C. Lithium aluminum hydride (1M in THF, 168.2 mL, 168.2 mmol) was added dropwise and the mixture stirred at 0° C. for 1 hour, then at room temperature for an additional 4 hours. The sample was cooled to 0° C. and neutralized with 3N HCl. The organics were separated and the resulting precipitate removed via filtration. The filtrate was collected, washed with water and satd. NaCl, and dried over MgSO₄. The solvent was removed under reduced pressure to give the title compound (22.0 g, 94% yield) as a white solid. ¹H NMR (300 MHz, CDCl₃) δ 7.35-7.19 (m, 5H), 3.87 (t, J=6.3, 2H), 3.67 (s, 2H), 2.63 (br s, 1H), 1.88 (t, J=6.3, 2H), 1.73-1.51 (m, 6H), 1.25-1.19 (m, 2H), 0.94 (s, 3H), 0.88 (s, 3H).

### 28f. 2-(4,4-Dimethyl-1-sulfanylcyclohexyl)ethan-1-ol

The product of Example 28e (22.8 g, 8.9 mmol) was cooled to −78° C. and dissolved in Et₂O (30 mL) and NH₃ (50 mL). Sodium (10.7 g, 467.8 mmol) was added portionwise and the mixture stirred for 30 minutes. A dry ice condenser was placed on the flask and the mixture stirred at room temperature for an additional 30 minutes. The reaction mixture was again cooled to −78° C. and NH₄Cl was added and the mixture stirred at room temperature overnight. The volatiles were evaporated under reduced pressure and the residue was diluted with water (50 mL) and cold concentrated HCl (50 mL). The sample was extracted with CH₂Cl₂ and the organics combined, washed with satd. NaCl, and dried over MgSO₄. The solvent was removed under reduced pressure and the residue washed with hexanes to give the title compound as a colorless oil which solidified upon cooling (13.6 g, 88% yield). ¹H NMR (300 MHz, CDCl₃) δ 3.90 (t, J=6.4, 2H), 1.97 (br s, 1H), 1.92 (t, J=6.4, 2H), 1.65-1.59 (m, 4H), 1.56-1.53 (m, 2H), 1.27-1.24 (m, 2H), 0.94 (s, 3H), 0.86 (s, 3H).

### 28g. 3-((2-(4,4-dimethyl-1-sulfanylcyclohexyl)ethyl)oxycarbonyl)propanoic acid

To a solution of the product of Example 28f (1.88 g, 10 mmol) in THF (50 mL) was added succinic anhydride (1.20 g, 12 mmol) and DMAP (1.466 g, 12 mmol). The solution was heated at 60° C. for 18 hours and cooled to room temperature. The residue was partitioned between EtOAc and water, acidifying the water layer to pH 1 with 3N HCl as needed. The layers were separated, and the organic layer was washed with water, satd. NaCl, and dried over MgSO₄. Removal of the solvent under reduced pressure gave the title compound (3.04 g, 100% yield) as a colorless oil which slowly solidified. Mp 55-60° C. ¹H NMR (300 MHz, CDCl₃) δ 4.39 (t, J=7.2 Hz, 2H), 2.67 (m, 4H), 1.96 (t, J=7.2 Hz, 2H), 1.62 (m, 6H), 1.46 (s, 1H), 1.27 (m, 2H), 0.95 (s, 3H), 0.87 (s, 3H). Mass spectrum (API-TIS) m/z 306 (MNH₄⁺).

### 28h. (1S,11S,14S,15S,10R)-14-hydroxy-15-methyltetracyclo(8.7.0.0<2,7>0.0<11,15>)heptadeca-2,4,6-trien-5-yl 2-(4,4-dimethyl-1-sulfanylcyclohexyl)ethyl butane-1,4-dioate

To a mixture of β-estradiol (1.362 g, 5.0 mmol), the product of Example 28 g (1.442 g, 5.0 mmol), and 4-dimethylaminopyridine(DMAP) (122 mg, 1.0 mmol) in THF at room temperature was added 1-(3-(dimethylamino)propyl)-3-ethylcarbodiimide hydrochloride (EDAC) (1.150 g, 6.0 mmol). The reaction mixture was stirred for 18 hours at room temperature, at which time the reaction was complete as monitored by TLC. The reaction mixture was washed with 0.1 M HCl, water, satd. NaCl and dried over MgSO₄. The residue after filtration and evaporation was purified via chromatography on silica gel (5% EtOAc in CH₂Cl₂ to 10% EtOAc in CH₂Cl₂) to give the title compound as a thick colorless oil (1.47 g, 54% yield). ¹H NMR (300 MHz, CDCl₃) δ 7.28 (d, J=8.4 Hz, 1H), 6.85 (dd, J=2.3, 8.4 Hz, 1H), 6.80 (d, J=2.3 Hz, 1H), 4.40 (t, J=7.2 Hz, 2H), 3.74 (t, J=8.1 Hz, 1H), 2.86 (m, 4H), 2.73 (t, J=6.8 Hz, 2H), 2.30-2.05 (m, 3H), 1.97 (t, J=7.2 Hz, 2H), 1.92 (m, 2H), 1.72-1.18 (m, 18 H), 0.95 (s, 3H), 0.86 (s, 3H), 0.78 (s, 3H). Mass spectrum (API-TIS) m/z 560 (M+NH₄⁺).

### 28i. (1S,11S,14S,15S,10R)-14-Hydroxy-15-methyltetracyclo(8.7.0.0<2,7>0.0<11,15>)heptadeca-2,4,6-trien-5-yl 2-(4,4-dimethyl-1-(nitrosothio)cyclohexyl)ethyl butane-1,4-dioate

To a solution of the product of Example 28h (895 mg, 1.65 mmol) in CH₂Cl₂ (5 mL) was added four drops of 6.5 M HCl in isopropanol followed by tert-butyl nitrite (90% solution, 0.24 mL, 1.814 mmol). The reaction mixture was stirred at room temperature for 30 min, and the solvent was evaporated under reduced pressure. The green residue was purified via chromatography on silica gel (CH₂Cl₂ to 10% EtOAc in CH₂Cl₂) to give two compounds. The upper R_{f} product was identified as Example 29. The lower R_{f} product was identified as the title compound as a dark green oil (638 mg, 67% yield) ¹H NMR (300 MHz, CDCl₃) δ 7.29 (d, J=8.4 Hz, 1H), 6.84 (dd, J=2.4, 8.4 Hz, 1H), 6.79 (d, J=2.4 Hz, 1H), 4.33 (t, J=7.1 Hz, 2H), 3.74 (t, J=8.2 Hz, 1H), 2.86 (m, 4H), 2.69 (m, 4H), 2.45-2.06 (m, 6H), 1.98-1.82 (m, 2H), 1.71 (m, 1H), 1.56-1.21 (m, 13H), 1.03 (s, 3H), 0.95 (s, 3H), 0.78 (s, 3H). Mass spectrum (API-TIS) m/z 589 (MNH₄⁺), 559 (MNH₄⁺—NO).

### Example 29

### (1S,11S,14S,15S,10R)-15-methyl-14-(nitrosooxy)tetracyclo(8.7.0.0<2,7>0.0<11,15>)heptadeca-2,4,6-trien-5-yl 2-(4,4-dimethyl-1-(nitrosothio)cyclohexyl)ethyl butane-1,4-dioate

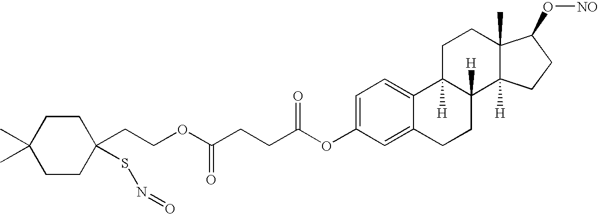

The title compound was isolated as the upper R_{f} product of Example 28i. The compound was a dark green oil (275 mg, 28% yield). ¹H NMR (300 MHz, CDCl₃) δ 7.27 (d, J=8.4 Hz, 1H), 6.84 (dd, J=2.4, 8.4 Hz, 1H), 6.80 (d, J=2.4 Hz, 1H), 5.35 (t, J=8.4 Hz, 1H), 4.32 (t, J=7.1 Hz, 2H), 2.85 (m, 4H), 2.68 (m, 4H), 2.45-2.09 (m, 7H), 1.94-1.74 (m, 4H), 1.54-1.36 (m, 10H), 1.02 (s, 3H), 0.94 (s, 3H), 0.79 (s, 3H). Mass spectrum (API-TIS) m/z 618 (MNH₄⁺), 589 (MNH₄⁺-11).

### Example 30

### (1S,11S,14S,15S,10R)-14-Hydroxy-15-methyltetracyclo(8.7.0.0<2,7>0.0<11,15>)heptadeca-2,4,6-trien-5-yl 2-(2-sulfanyladamantan-2-yl)ethyl butane-1,4-dioate

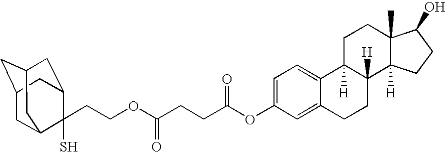

To a mixture of β-estradiol (454 mg, 1.667 mmol), 3-((2-(2-sulfanyladamantan-2-yl)ethyl)oxycarbonyl)propanoic acid (prepared as described in U.S. Pat. No. 6,469,065, Example 10d) (625 mg, 2.0 mmol), and 4-dimethylaminopyridine(DMAP) (40 mg, 0.33 mmol) in CH₂Cl₂/THF at room temperature was added 1-(3-(dimethylamino)propyl)-3-ethylcarbodiimide hydrochloride (EDAC) (383 mg, 2.0 mmol). The reaction mixture was stirred for 18 hours at room temperature, at which time the reaction was complete as monitored by TLC. The reaction mixture was washed with 0.1 M hydrochloric acid, water, satd. NaCl and dried over MgSO₄. The residue after filtration and evaporation was purified via chromatography on silica gel (10% EtOAc in CH₂Cl₂) to give the title compound as a white solid (740 mg, 78% yield). Mp 133-136° C. ¹H NMR (300 MHz, CDCl₃) δ 7.28 (d, J=8.4 Hz, 1H), 6.84 (dd, J=2.5, 8.4 Hz, 1H), 6.80 (d, J=2.5 Hz, 1H), 4.45 (t, J=7.5 Hz, 2H), 3.73 (t, J=8.1 Hz, 1H), 2.86 (m, 4H), 2.72 (t, J=6.5 Hz, 2H), 2.43 (m, 2H), 2.35-2.05 (m, 5H), 1.99-1.61 (m, 14H), 1.58-1.16 (m, 8H), 0.77 (s, 3H). Mass spectrum (API-TIS) m/z 584 (MNH₄⁺).

### Example 31

### (1S,11S,14S,15S,10R)-15-methyl-5-phenylcarbonyloxytetracyclo(8.7.0.0<2,7>0.0<11,15>)heptadeca-2,4,6-trien-14-yl 2-(2-sulfanyladamantan-2-yl)ethyl butane-1,4-dioate

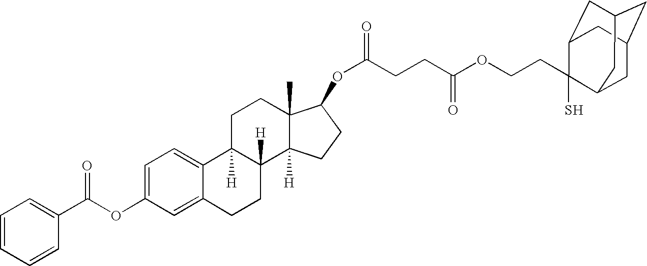

To a mixture of β-estradiol-3-benzoate (628 mg, 1.667 mmol), 3-((2-(2-sulfanyladamantan-2-yl)ethyl)oxycarbonyl)propanoic acid (prepared as described in U.S. Pat. No. 6,469,065, Example 10d, 625 mg, 2.0 mmol), and 4-dimethylaminopyridine(DMAP) (41 mg, 0.33 mmol) in CH₂Cl₂ at room temperature was added 1-(3-(dimethylamino)propyl)-3-ethylcarbodiimide hydrochloride (EDAC) (383 mg, 2.0 mmol). The reaction mixture was stirred for 18 hours at room temperature, at which time the reaction was complete as monitored by TLC. The reaction mixture was washed with 0.1 M HCl, water, satd. NaCl and dried over MgSO₄. The residue after filtration and evaporation was purified via chromatography on silica gel (5% EtOAc in CH₂Cl₂ to 10% EtOAc in CH₂Cl₂) to give the title compound as a white solid (590 mg, 53% yield). Mp 140-143° C. ¹H NMR (300 MHz, CDCl₃) δ 8.20 (d, J=7.4 Hz, 2H), 7.63 (t, J=7.5 Hz, 1H), 7.50 (t, J=7.5 Hz, 2H), 7.33 (d, J=8.4 Hz, 1H), 6.97 (dd, J=2.2, 8.4 Hz, 1H), 6.93 (d, J=2.2 Hz, 1H), 4.72 (t, J=8.1 Hz, 1H), 4.43. (t, J=7.5 Hz, 2H), 2.89 (m, 2H), 2.64 (m, 4H), 2.43 (m, 2H), 2.35-2.21 (m, 2H), 2.25 (t, J=7.5 Hz, 2H), 2.12 (m, 2H), 1.92-1.30 (m, 22H), 0.84 (s, 3H). Mass spectrum (API-TIS) m/z 688 (MNH₄⁺), 671 (MH⁺), 637, 477.

### Example 32

### (1S,11S,14S,15S,10R)-14-Hydroxy-15-methyltetracyclo(8.7.0.0<2,7>0.0<11,15>)heptadeca-2,4,6-trien-5-yl(1S,2S,5S,6R)-6-(nitrooxy)-4,8-dioxabicyclo(3.3.0)oct-2-yl butane-1,4-dioate

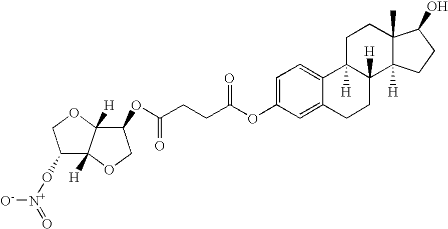

### 32a. 3-(((1S,2S,5S,6R)-6-(Nitrooxy)-4,8-dioxabicyclo(3.3.0)oct-2-yl)oxycarbonyl)propanoic acid

Isosorbide 5-mononitrate (prepared as described in U.S. Pat. No. 4,431,830; 2.01 g, 10.54 mmol), succinic anhydride (Aldrich, Wis., US; 1.27 g, 12.64 mmol, 1.2 eq), and DMAP (1.56 g, 12.64 mmol) were all mixed together at ambient temperature in a 100 mL round-bottomed flask. The mixture was then slurried in 30 mL of dry THF, the flask fitted with a reflux condenser and heated to the reflux temperature overnight. The resultant clear solution was cooled to ambient temperature at which point it became turbid. The reaction mixture was diluted with EtOAc, washed twice with 3N HCl, and then finally satd. NaCl. The organic layer was dried over Na₂SO₄, filtered, and the solvent was removed in vacuo to give the title compound (2.0 g, 65% yield) as a thick pale yellow oil. ¹H NMR (300 MHz, CDCl₃) δ 9.90 (br s, 1H), 5.36 (dt, J=2.8, 5.4 Hz, 1H), 5.25 (d, J=2.4 Hz, 1H), 4.98 (t, J=5.2 Hz, 1H), 4.48 (d, J=4.9 Hz, 1H), 4.01 (m, 3H), 3.91 (m, 1H), 2.67 (m, 4H). Mass spectrum (API-TIS) m/z 292 (MH⁺), 309 (MNH₄⁺).

### 32b. (1S,11S,14S,15S,10R)-14-Hydroxy-15-methyltetracyclo(8.7.0.0<2,7>0.0<11,15>)heptadeca-2,4,6-trien-5-yl(1S,2S,5S,6R)-6-(nitrooxy)-4,8-dioxabicyclo(3.3.0)oct-2-yl butane-1,4-dioate

β-Estradiol (Steraloids, R.I., US; 624 mg, 2.29 mmol) and the product of Example 32a (700 mg, 2.40 mmol, 1.05 eq) were taken up in 20 mL of dry CH₂Cl₂. A catalytic amount of DMAP (10 mmol) was added followed by the addition at room temperature of EDAC (475 mg, 2.40 mmol, 1.05 eq). The reaction mixture was stirred at ambient temperature overnight, diluted with CH₂Cl₂, washed twice with H₂O and satd. NaCl. The organic layer was dried over Na₂SO₄, filtered, and the solvent was removed in vacuo. The product was chromatography on silica gel eluting with 2:3 (250 mL) then 7:3 (250 mL) EtOAc/Hexane and finally EtOAc (250 mL) to give the title compound (900 mg, 72% yield) as a white solid. Mp 163-165° C. ¹H NMR (300 MHz, CDCl₃) δ 7.28 (m, 1H), 6.84 (m, 1H), 6.79 (m, 1H), 5.33 (dt, J=2.8, 5.5 Hz, 1H), 5.27 (d, J=2.6 Hz, 1H), 4.95 (t, J=5.2 Hz, 1H), 4.48 (d, J=4.9 Hz, 1H), 4.02 (m, 3H), 3.88 (m, 1H), 3.73 (m, 1H), 2.87 (m, 4H), 2.73 (m, 2H), 2.42-2.05 (m, 4H), 1.98-1.86 (m, 2H), 1.72 (m, 1H), 1.54-1.17 (m, 7H), 0.78 (s, 3H). Mass spectrum (API-TIS) m/z 546 (MH⁺), 563 (MNH₄⁺).

### Example 33

### (1S,2S,5S,6R)-6-(Nitrooxy)-4,8-dioxabicyclo(3.3.0)oct-2-yl 2-((((1S,11S,14S,15S,10R)-14-hydroxy-15-methyltetracyclo(8.7.0.0<2,7>0.0<11,15>)heptadeca-2,4,6-trien-5-yl)oxycarbonyl)methoxy)acetate

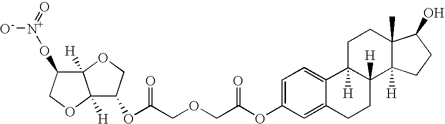

### 33a. 2-((((1S,2S,5S,6R)-6-(Nitrooxy)-4,8-dioxabicyclo(3.3.0)oct-2-yl)oxycarbonyl)methoxy)acetic acid

Isosorbide 5-mononitrate (prepared as described in U.S. Pat. No. 4,431,830; 2.11 g, 11.04 mmol), glutaric anhydride (Aldrich, Wis., US; 1.54 g, 13.25 mmol, 1.2 eq), and DMAP (1.62 g, 13.25 mmol, 1.2 eq) were all mixed together at room temperature and then slurried in dry THF (60 mL), and refluxed overnight. The reaction mixture was diluted with EtOAc, washed twice with 3N HCl, and then finally satd. NaCl. The organic layer was dried over Na₂SO₄, filtered, and the solvent was removed in vacuo giving the title compound (3.3 g, 97% yield) as a thick pale yellow oil. NMR (300 MHz, CDCl₃) δ 6.55 (br s, 1H), 5.37 (dt, J=2.7, 5.5 Hz, 1H), 5.30 (d, J=2.4 Hz, 1H), 4.99 (t, J=5.5 Hz, 1H), 4.51 (d, J=4.9 Hz, 1H), 4.27 (s, 2H), 4.25 (ABq, J_{AB}=7.4 Hz, Δν=7.1 Hz, 2H), 4.02 (m, 3H), 3.92 (m, 1H). Mass spectrum (API-TIS) m/z 308 (MH⁺), 325 (MNH₄⁺).

### 33b. (1S,2S,5S,6R)-6-(Nitrooxy)-4,8-dioxabicyclo(3.3.0)oct-2-yl 2-((((1S,11S,14S,15S,10R)-14-hydroxy-15-methyltetracyclo(8.7.0.0<2,7>0.0<11,15>)heptadeca-2,4,6-trien-5-yl)oxycarbonyl)methoxy)acetate

β-Estradiol (Steraloids, R.I., US; 510 mg, 1.87 mmol) and the product of Example 33a (690 mg, 2.25 mmol, 1.2 eq) were taken up in dry THF (20 mL). A catalytic amount of DMAP (10 mg) was added followed by EDAC (444 mg, 2.25 mmol, 1.2 eq). The reaction mixture was stirred at room temperature overnight, diluted with CH₂Cl₂, washed twice with H₂O and finally satd. NaCl. The organic layer was dried over Na₂SO₄, filtered, and the solvent was removed in vacuo. The product was chromatography on silica gel column eluting with EtOAc/Hexane (2:3 then 7:3) and finally EtOAc to give the title compound (422 mg, 40% yield) as a white solid. Mp 146-149° C. ¹H NMR (300 MHz, CDCl₃) δ 7.16 (m, 1H), 6.63 (m, 1H), 6.57 (m, 1H), 5.36 (dt, J=2.8, 5.5 Hz, 1H), 5.31 (m, 1H), 4.99 (t, J=5.3 Hz, 1H), 4.50 (d, J=4.9 Hz, 1H), 4.25 (ABq, J_{AB}=17.0 Hz, Δν=9.9 Hz, 2H), 4.04 (s, 2H), 4.03 (m, 3H), 3.91 (m, 1H), 3.74 (t, J=8.4 Hz, 1H), 2.80 (m, 2H), 2.39-1.13 (m, 14H), 0.78 (s, 3H). Mass spectrum (API-TIS) m/z 562 (MH⁺), 579 (MNH₄⁺).

### Example 34

### (1S,11S,14S,15S,10R)-14-hydroxy-15-methyltetracyclo(8.7.0.0<2,7>0.0<11,15>)heptadeca-2,4,6-trien-5-yl 4-(N-(((nitrosothio)cyclohexyl)methyl)-carbamoyl)butanoate

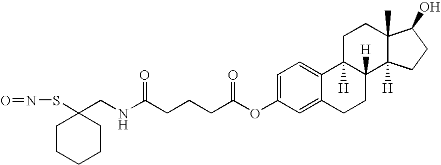

To a mixture of 17β-estradiol (Spectrum) (2.5 g, 9.2 mmol), 4-(N-(((nitrosothio)cyclohexyl)methyl)carbamoyl)butanoic acid, prepared as described in U.S. application No. 2003/0203915, Example 33e, 2.36 g, 8.19 mmol) and N,N-dimethylaminopyridine (DMAP, 1.12 g, 9.2 mmol) in CH₂Cl₂ (24 mL) at 0° C. was added dropwise dicyclohexylcarbodiimide (1.89 g, 9.2 mmol) in CH₂Cl₂ (24 mL). The resultant solution was stirred at 0° C. for 5 hours and at room temperature in the dark for 16 hours. The residue after filtration and evaporation was chromatographed on silica gel eluting with EtOAc:Hexane (1:2 to 1:1 to 2:1) to give the title compound (2.2 g, 44% yield) as a green foam. Mp 50-52° C. ¹H NMR (300 MHz, CDCl₃) δ 7.28-7.38 (m, 1H), 6.80-6.88 (m, 1H), 6.72-6.80 (m, 1H), 5.78-5.88 (bs, 1H), 4.17 (d, J=6.4 Hz, 2H), 3.74 (d, J=8.5 Hz, 1H), 2.80-2.90 (m, 2H), 2.59 (t, J=7.1 Hz, 2H), 2.38-2.52 (m, 2H), 1.83-2.38 (m, 12H), 1.60-1.83 (m, 4H), 1.10-1.60 (m, 10H), 0.78 (s, 3H). ¹³C NMR (75 MHz, CDCl₃) δ 172.6, 172.2, 148.4, 138.4, 138.2, 126.6, 121.6, 118.7, 82.0, 62.7, 50.2, 49.2, 44.3, 43.4, 38.6, 36.8, 35.5, 34.8, 33.4, 30.7, 29.7, 27.2, 26.3, 25.6, 23.3, 22.1, 21.0, 11.2. Mass spectrum (API-TIS) m/z 543 (MH⁺), 560 (MNH₄⁺). Anal. calcd. for C₃₀H₄₂N₂O₅S: C, 66.39; H, 7.80; N, 5.16. Found: C, 66.24; H, 7.89; N, 4.99.

### Example 35

### 2-(((1S,11S,14S,15S,10R)-5,14-Dihydroxy-15-methyltetracyclo(8.7.0.0<2,7>0.0<11,15>)heptadeca-2,4,6-trien-8-ylidene)azamethoxy)-N-methyl-N-(2-(nitrooxy)ethyl)acetamide

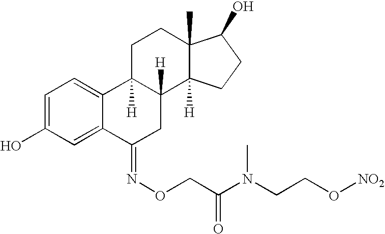

### 35a. 2-(((1S,11S,14S,15S,10R)-5,14-dihydroxy-15-methyltetracyclo(8.7.0.0<2,7>0.0<11-15>)heptadeca-2,4,6-trien-8-ylidene)azamethoxy)acetic acid

This compound was synthesized as described by Mons, S. et al, Syn. Comm., 28(2): 213-218, (1998).

### 35b. 2-(((1S,11S,14S,15S,10R)-5,14-dihydroxy-15-methyltetracyclo(8.7.0.0<2,7>0.0<11-15>)heptadeca-2,4,6-trien-8-ylidene)azamethoxy)-N-methyl-N-(2-(nitrooxy)ethyl)-acetamide

To a solution of methyl(2-(nitrooxy)ethyl)ammonium nitrate (prepared as described in U.S. application No. 2004/0024057, Example 17c, 0.31 g, 1.8 mmol) in CH₂Cl₂ (5 mL) and DMF (2.5 mL) was added N,N-dimethylaminopyridine (DMAP, 85 mg, 0.70 mmol) at 0° C. The mixture was stirred at 0° C. for 5 minutes. To this solution the product of Example 35a (0.25 g, 0.70 mmol) was added followed by 1-(3-(dimethylamino)propyl)-3-ethylcarbodiimide hydrochloride (0.13 g, 0.70 mmol). The reaction mixture was stirred at 0° C. for 4 hours. The solvent was evaporated. The residue was diluted with more CH₂Cl₂, washed with water, satd. NaCl and dried over Na₂SO₄. The residue after filtration and evaporation was chromatographed on silica gel eluting with EtOAc:CH₂Cl₂ (1:2 to 1:1) to give the title compound (77 mg, 24% yield) as off-white solid. Mp 125-127° C. with decomposition. ¹H NMR (300 MHz, CDCl₃) δ 7.30-7.38 (m, 1H), 7.07-7.18 (m, 1H), 6.78-6.85 (m, 1H), 4.84 (s, 2H), 4.67 (t, J=5.1 Hz, 2H), 3.63-3.78 (m, 3H), 3.13 (s, 3H), 3.00-3.08 (m, 1H), 1.85-2.32 (m, 4H), 1.03-1.80 (m, 10H), 0.73 (s, 3H). Mass spectrum (API-TIS) m/z 462 (MH⁺).

### Example 36

### 2-(2-(Nitrosothio)adamantan-2-yl)ethyl 2-((1S,11S,14S,15S,10R)-14-hydroxy-15-methyltetracyclo(8.7.0.0<2,7>0.0<11,15>)heptadeca-2,4,6-trien-5-yloxy)acetate

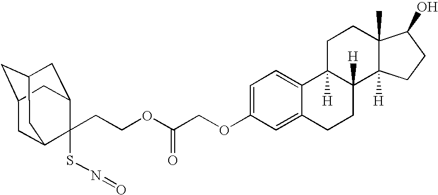

### 36a. 2-((1S,11S,14S,15S,10R)-14-hydroxy-15-methyltetracyclo(8.7.0.0<2,7>0.0<11,15>)heptadeca-2,4,6-trien-5-yloxy)acetic acid

This compound was synthesized as described by Dhar, T. K. et al, Steroids, 51(5-6): 519-526, (1998).

### 36b. 2-(2-(Nitrosothio)adamantan-2-yl)ethyl 2-((1S,11S,14S,15S,10R)-14-hydroxy-15-methyltetracyclo(8.7.0.0<2,7>0.0<11,15>)heptadeca-2,4,6-trien-5-yloxy)acetate

To a solution of the product of Example 36a (0.27 g, 0.82 mmol) and 2-(2-(nitrosothio)adamantan-2-yl)ethan-1-ol (prepared as described in U.S. Pat. No. 6,469,065, Example 12a), (0.2 g, 0.83 mmol) in CH₂Cl₂ (5 mL) was added N,N-dimethylaminopyridine (DMAP, 85 mg, 0.70 mmol) at 0° C. To this solution dicyclohexylcarbodiimide (0.17 g, 0.83 mmol) in CH₂Cl₂ (1.5 mL) was added dropwise. The reaction mixture was stirred at 4° C. for 5 hours. The solid was filtered. The filtrate was diluted with more CH₂Cl₂, washed with water, satd. NaCl and dried over Na₂SO₄. The residue after filtration and evaporation of the solvent was purified by preparative layer chromatography eluting with EtOAc:CH₂Cl₂ (1:3) to give the title compound (0.15 g, 33% yield) as a green foam. Mp 48-50° C. ¹H NMR (300 MHz, CDCl₃) δ 7.17-7.23 (m, 1H), 6.65-6.70 (m, 1H), 6.57-6.62 (m, 1H), 4.55 (s, 2H), 4.38 (t, J=7.3 Hz, 2H), 3.73 (t, J=8.5 Hz, 1H), 3.09 (t, J=7.3 Hz, 2H), 2.70-2.85 (m, 2H), 2.45-2.55 (m, 2H), 1.97-2.45 (m, 7H), 1.60-1.97 (m, 10H), 1.05-1.52 (m, 9H), 0.77 (s, 3H). ¹³C NMR (75 MHz, CDCl₃) δ 169.4, 155.7, 138.3, 134.0, 126.6, 114.7, 112.1, 82.0, 67.7, 65.5, 62.1, 50.1, 44.1, 43.4, 39.0, 38.9, 36.8, 35.7, 35.6, 34.0, 33.2, 30.7, 29.9, 27.4, 27.3, 26.4, 23.3, 11.2. Mass spectrum (API-TIS) m/z 524 (M−NO), 571 (MNH₄⁺).

### Example 37

### 2-(2-(Nitrosothio)adamantan-2-yl)ethyl 2-(((1S,11S,14S,15S,10R)-5,14-dihydroxy-15-methyltetracyclo(8.7.0.0<2,7>0.0<11,15>)heptadeca-2,4,6-trien-8-ylidene)azamethoxy)acetate

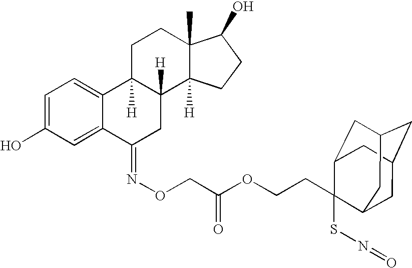

To a solution of the product of Example 35a (123 mg, 0.34 mmol) and 2-(2-(nitrosothio)adamantan-2-yl)ethan-1-ol (prepared as described in U.S. Pat. No. 6,469,065, Example 12a), (0.2 g, 0.83 mmol) in CH₂Cl₂ (5 mL) was added N,N-dimethylaminopyridine (DMAP, 41 mg, 0.34 mmol) at 0° C. To this solution dicyclohexylcarbodiimide (71 mg, 0.34 mmol) in CH₂Cl₂ (2 mL) was added dropwise. The reaction mixture was stirred at 4° C. for 5 hours and at room temperature for 16 hours. The residue after filtration and evaporation of the solvent was chromatographed on silica gel eluting with EtOAc:Hexane (1:10 to 3:10) to give the title compound (22 mg, 1% yield) as a green solid. Mp 75-80° C. ¹H NMR (300 MHz, CDCl₃) δ 7.27-7.32 (m, 1H), 7.08-7.15 (m, 1H), 6.82 (dd, J=2.7 and 8.5 Hz, 1H), 6.22-6.28 (bs, 1H), 4.70 (s, 2H), 4.32-4.45 (m, 2H), 3.75 (t, J=8.3 Hz, 1H), 3.13 (t, J=7.3 Hz, 2H), 2.98-3.05 (m, 1H), 2.50-2.58 (m, 2H), 2.32-2.50 (m, 2H), 1.62-2.30 (m, 18H), 1.08-1.60 (m, 5H), 0.76 (s, 3H). ¹³C NMR (75 MHz, CDCl₃) δ 171.1, 156.0, 154.3, 134.9, 130.8, 125.9, 117.3, 110.6, 81.8, 70.9, 67.8, 62.0, 50.5, 43.1, 41.5, 39.0, 36.9, 36.2, 35.7, 35.6, 33.9, 33.2, 30.5, 29.8, 27.4, 27.3, 25.6, 23.1, 11.1. Mass spectrum (API-TIS) m/z 553 (M−NO), 583 (MH⁺).

### Example 38

### 2-(((1S,11S,14S,15S,10R)-5,14-dihydroxy-15-methyltetracyclo(8.7.0.0<2,7>0.0<11,15>)heptadeca-2,4,6-trien-8-ylidene)azamethoxy)-1-(4-((nitrooxy)methyl)piperidyl)ethan-1-one

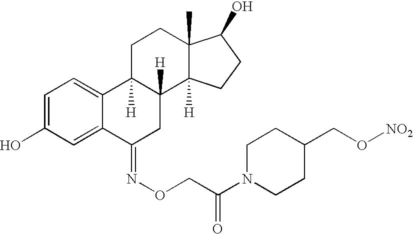

A mixture of nitrooxy(4-piperidylmethyl)hydrogen nitrate (prepared as described in U.S. application No. 2004/0024057, Example 19a, 0.25 g, 1.1 mmol) and N,N-dimethylaminopyridine (DMAP, 0.13 g, 1.1 mmol) in CH₂Cl₂ (5 mL) at 0° C., was treated with the product of Example 35a (0.2 g, 0.56 mmol) and 1-(3-(dimethylamino)propyl)-3-ethylcarbodiimide hydrochloride (0.11 g, 0.57 mmmol). The reaction mixture was warmed from 0° C. to room temperature over 5 hours and diluted with CH₂Cl₂, washed with water, satd. NaCl and dried over Na₂SO₄. The residue after filtration and evaporation was chromatographed on silica gel eluting with EtOAc:CH₂Cl₂ (1:3 to 1:1) to give the title compound (57 mg, 20% yield) as a white solid. Mp 143-145° C. ¹H NMR (300 MHz, CDCl₃/d₄-MeOH) δ 7.35 (s, 1H), 7.16 (d, J=8.5 Hz, 1H), 6.85 (dd, J=2.3 and 8.4 Hz, 1H), 4.70-4.90 (bs, 2H), 4.45-4.70 (m, 2H), 4.20-4.40 (m, 2H), 4.00-4.18 (m, 1H), 3.71 (t, J=8.3 Hz, 1H), 2.98-3.15 (m, 2H), 2.66 (t, J=12.8 Hz, 1H), 2.15-2.32 (m, 1H), 1.15-2.15 (m, 16H), 0.75 (s, 3H). ¹³C NMR (75 MHz, CDCl₃/d₄-MeOH) δ 168.2, 155.9, 155.0, 134.2, 130.5, 126.0, 117.5, 110.0, 81.2, 72.6, 50.3, 44.9, 43.0, 41.8, 41.5, 37.1, 36.1, 34.2, 29.9, 29.6, 29.1, 28.1, 25.5, 23.0, 11.0. Mass spectrum (API-TIS) m/z 502 (MH⁺). LCMS (98.8%).

### Example 39

### 2-(((1S,11S,14S,15S,10R)-5,14-dihydroxy-15-methyltetracyclo(8.7.0.0<2,7>0.0<11,15>)heptadeca-2,4,6-trien-8-ylidene)azamethoxy)-N-(2-(nitrooxy)ethyl)acetamide

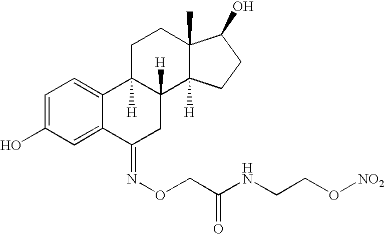

A mixture of 2-(nitrooxy)ethylammonium nitrate (prepared as described in U.S. application No. 2004/0024057, Example 22a, 0.19 g, 1.1 mmol) and N,N-dimethylaminopyridine (DMAP, 0.20 g, 1.7 mmol) in CH₂Cl₂ (3 mL) at 0° C., was treated with the product of Example 35a (0.2 g, 0.56 mmol) and 1-(3-(dimethylamino)propyl)-3-ethylcarbodiimide hydrochloride (0.13 g, 0.67 mmmol). The reaction mixture was stirred at 0° C. to 4° C. for 4 hours, diluted with CH₂Cl₂, washed with water, satd. NaCl and dried over Na₂SO₄. The residue after filtration and evaporation was chromatographed on silica gel eluting with EtOAc:CH₂Cl₂ (1:3 to 1:1) to give the title compound (68 mg, 27% yield) as a white solid. Mp 140° C. with decomposition. ¹H NMR (300 MHz, CDCl₃/d₄-MeOH) δ 7.31-7.35 (m, 1H), 7.18-7.21 (m, 1H), 6.87 (dd, J=2.4 and 8.5 Hz, 1H), 6.70-6.80 (bs, 1H), 4.63 (s, 2H), 4.58 (t, J=5.1 Hz, 1H), 3.60-3.78 (m, 3H), 3.05-3.20 (m, 1H), 2.20-2.52 (m, 4H), 1.92-2.23 (m, 4H), 1.18-1.88 (m, 7H), 0.78 (s, 3H). ¹³C NMR (75 MHz, CDCl₃/d₄-MeOH) δ 171.5, 157.0, 154.9, 134.5, 130.2, 126.1, 117.7, 110.0, 81.1, 72.7, 71.4, 50.3, 42.9, 41.5, 37.0, 36.4, 36.0, 29.8, 25.4, 22.9, 10.8. Mass spectrum (API-TIS) m/z 448 (MH⁺), 470 (MNa+). LCMS (98.8%).

### Example 40

### 2-(((1S,11S,14S,15S,10R)-5,14-dihydroxy-15-methyltetracyclo(8.7.0.0<2,7>0.0<11,15>)heptadeca-2,4,6-trien-8-ylidene)azamethoxy)-1-(4-(2-(nitrooxy)ethyl)piperidyl)ethan-1-one

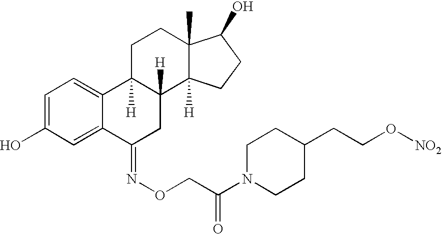

A mixture of nitrooxy(2-(4-piperidyl)ethyl)hydrogen nitrate (prepared as described in U.S. application No. 2004/0024057, Example 31a, 0.25 g, 1.1 mmol) and N,N-dimethylaminopyridine (DMAP, 0.2 g, 1.6 mmol) in CH₂Cl₂ (3 mL) at 0° C., was treated with the product of Example 35a (0.2 g, 0.56 mmol) and 1-(3-(dimethylamino)propyl)-3-ethylcarbodiimide hydrochloride (0.13 g, 0.67 mmol). The reaction mixture was stirred at 0° C. to 4° C. for 3 hours, diluted with CH₂Cl₂, washed with water, satd. NaCl and dried over Na₂SO₄. The residue after filtration and evaporation was chromatographed on silica gel eluting with EtOAc:CH₂Cl₂ (1:2 to 1:1) to give the title compound (68 mg, 24% yield) as a white solid. Mp 102-105° C. ¹H NMR (300 MHz, CDCl₃/d₄-MeOH) δ 7.36 (s, 1H), 7.16 (d, J=8.5 Hz, 1H), 6.84 (dd, J=2.7 and 8.5 Hz, 1H), 4.70-4.83 (bs, 2H), 4.45-4.62 (m, 3H), 3.92-4.10 (m, 1H), 3.80 (bs, 3H), 3.69 (t, J=8.5 Hz, 1H), 2.97-3.18 (m, 2H), 2.55-2.72 (m, 1H), 2.20-2.32 (m, 1H), 1.88-2.15 (m, 4H), 1.07-1.87 (m, 13H), 0.74 (s, 3H). ¹³C NMR (75 MHz, CDCl₃/d₄-MeOH) δ 168.1, 155.8, 154.9, 134.2, 130.5, 125.9, 117.4, 109.9, 81.4, 72.6, 70.7, 50.3, 46.4, 45.3, 42.9, 42.2, 41.5, 37.0, 36.0, 32.9, 32.7, 31.4, 29.7, 29.5, 25.4, 22.9, 10.8. Mass spectrum (API-TIS) m/z 516 (MH⁺).

### Example 41

### 2-(((1S,11S,14S,15S,10R)-5,14-dihydroxy-15-methyltetracyclo(8.7.0.0<2,7>0.0<11,15>)heptadeca-2,4,6-trien-8-ylidene)azamethoxy)-N-(2-methyl-2-(nitrosothio)propyl)acetamide

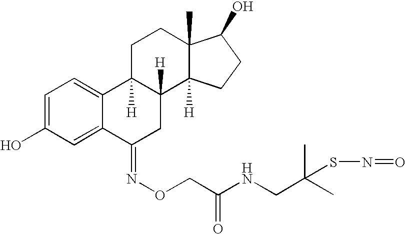

tert-Butyl nitrite (90% solution, 0.8 g, 7.7 mmol) was added dropwise to a suspension of 2-mercapto-2-methyl-1-propylamine hydrochloride (Aldrich) (1 g, 7.09 mmol) in CH₂Cl₂ (0.6 mL) and DMF (2 mL) at −10° C. The resultant solution was stirred at −10° C. for 5 minutes and diluted with CH₂Cl₂ and hexane. The green oil was separated, washed with hexane and dried under vacuo to give 2-methyl-2-nitrosomercapto-1-propylamine (˜0.5 g). Mass spectrum (API-TIS) m/z 135 (MH⁺). This was dissolved in CH₂Cl₂ (3 mL), cooled to 0° C. and treated portionwise with the product of Example 35a (0.2 g, 0.56 mmol) and 1-(3-(dimethylamino)propyl)-3-ethylcarbodiimide hydrochloride (0.13 g, 0.67 mmmol). To this reaction mixture, N,N-dimethylaminopyridine (DMAP, 70 mg, 0.57 mmol) in CH₂Cl₂ (1 mL) was added dropwise at 0° C. and stirred at 0° C. for 2.5 hours, diluted with CH₂Cl₂, washed with water, satd. NaCl and dried over Na₂SO₄. The residue after filtration and evaporation was chromatographed on silica gel eluting with EtOAc:CH₂Cl₂ (2:3) to give the title compound (70 mg, 26% yield) as a green solid. Mp 145-150° C. ¹H NMR (300 MHz, CDCl₃) δ 7.60-7.87 (bs, 1H), 7.34-7.38 (m, 1H), 7.12-7.23 (m, 1H), 6.86-6.95 (m, 1H), 6.75 (t, J=6.2 Hz, 1H), 4.68 (bs, 2H), 4.07-4.15 (m, 2H), 3.80 (t, J=8.4 Hz, 1H), 2.90-3.05 (m, 1H), 1.88 (s, 3H), 1.84 (s, 3H), 1.10-2.40 (m, 13H), 0.75 (s, 3H). ¹³C NMR (75 MHz, CDCl₃) δ 171.3, 156.9, 155.0, 134.7, 130.1, 126.4, 118.1, 110.2, 81.7, 73.0, 57.2, 50.4, 49.3, 43.2, 41.6, 37.2, 36.3, 30.5, 29.8, 26.9, 25.6, 23.1, 11.1. Mass spectrum (API-TIS) m/z 476 (MH⁺).

### Example 42

### 2-((1S,11S,14S,15S,10R)-14-Hydroxy-15-methyltetracyclo(8.7.0.0<2,7>0.0<11,15>)heptadeca-2,4,6-trien-5-yloxy)-N-(2-methyl-2-(nitrosothio)propyl)acetamide

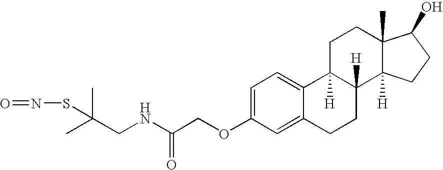

tert-Butyl nitrite (90% solution, 0.8 g, 7.7 mmol) was added dropwise to a suspension of 2-mercapto-2-methyl-1-propylamine hydrochloride (Aldrich) (1 g, 7.09 mmol) in CH₂Cl₂ (0.6 mL) and DMF (2 mL) at −10° C. The resultant solution was stirred at −10° C. for 5 minutes and diluted with CH₂Cl₂ and hexane. The green oil was separated and washed with hexane and dried under vacuo to give 2-methyl-2-nitrosomercapto-1-propylamine (˜0.5 g). Mass spectrum (API-TIS) m/z 135 (MH⁺). This was dissolved in CH₂Cl₂ (3 mL), cooled to 0° C. and treated portionwise with the product of Example 36a (0.6 g, 1.8 mmol) and 1-(3-(dimethylamino)propyl)-3-ethylcarbodiimide hydrochloride (0.35 g, 1.8 mmmol). To this reaction mixture, N,N-dimethylaminopyridine (DMAP, 0.22 g, 1.8 mmol) in CH₂Cl₂ (1 mL) was added dropwise at 0° C. and stirred at 0° C. for 2 hours, diluted with CH₂Cl₂, washed with water, satd. NaCl and dried over Na₂SO₄. The residue after filtration and evaporation was chromatographed on silica gel eluting with EtOAc:CH₂Cl₂ (1:1) to give the title compound (0.25 g, 31% yield) as a green foam. Mp 40° C. ¹H NMR (300 MHz, CDCl₃) δ 7.13-7.30 (m, 1H), 6.85-6.97((bs, 1H), 6.50-6.70 (m, 2H), 4.48 (s, 2H), 4.11(d, J=6.5 Hz, 2H), 3.53-3.70 (m, 1H), 2.75-2.92 (m, 2H), 2.00-2.39 (m, 3H), 1.87 (s, 6H), 1.80-2.00 (m, 1H), 1.00-1.80 (m, 10H), 0.78 (s, 3H). ¹³C NMR (75 MHz, CDCl₃) δ 169.1, 155.0, 138.6, 134.5, 126.8, 114.7, 112.2, 81.9, 67.4, 57.0, 50.1, 49.1, 44.0, 43.3, 38.8, 36.8, 30.7, 29.8, 27.2, 26.9, 26.4, 23.2, 11.2. Mass spectrum (API-TIS) m/z 464 (MNH₄⁺), 417 (M−NO).

### Example 43

### 2-(4-(1-methyl-1-(nitrosothio)ethyl)-2-oxo-1,3-oxazolidin-3-yl)ethyl 2-(((1S,11S,14S,15S,10R)-5,14-dihydroxy-15-methyltetracyclo(8.7.0.0<2,7>0.0<11,15>)heptadeca-2,4,6-trien-8-ylidene)azamethoxy)acetate

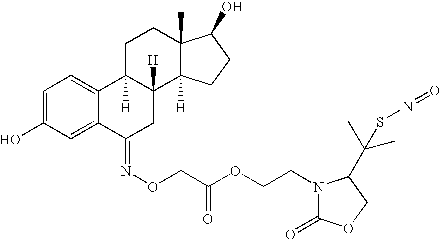

### 43a. 4-(1-Methyl-1-((2,4,6-trimethoxyphenyl)methylthio)ethyl)-3-(2-(1,1,2,2-tetramethyl-1-silapropoxy)ethyl)-1,3-oxazolidin-2-one

NaH (60% in oil) was washed once with hexane and the hexane removed under vacuo. The solid (1.6 g, 66.7 mmol) was then added in portions to a solution of 4-(1-methyl-1-((2,4,6-trimethoxyphenyl)methylthio)ethyl)-1,3-oxazolidin-2-one (prepared as described in WO 01/85013, Example 2c, 15.3 g, 44.9 mmol) in dry DMF (50 mL) under nitrogen at 0° C. The resulting suspension was stirred at 0° C. for 20 minutes to give a brown red solution. 2-Bromo-1-(1-,1-,2-,2-tetramethyl-1-silapropoxy)ethane (Aldrich) (12.9 g, 53.8 mmol) in DMF (10 mL) was added dropwise and stirred at room temperature for 16 hours. The solvent was evaporated. The residue was partitioned with EtOAc:water (1:1) and the organic layer was separated. The aqueous layer was extracted with EtOAc and the combined organic layers were washed with water, dried over Na₂SO₄, and filtered. The residue after evaporation of the solvent was chromatographed on silica gel eluting with EtOAc:Hexane (1:1) to give the title compound (18 g, 80% yield) as a white foam. ¹H NMR (300 MHz, CDCl₃) δ 6.12 (s, 2H), 4.38-4.47 (m, 1H), 4.09-4.21 (m, 3H), 3.83 (s, 9H), 3.79 (s, 2H), 3.71-3.79 (m, 2H), 3.42-3.53 (m, 1H), 1.50 (s, 3H), 1.29 (s, 3H), 0.95 (s, 9H), 0.08 (s, 6H). ¹³C NMR (75 MHz, CDCl₃) δ 160.8, 159.7, 158.9, 106.5, 90.9, 65.9, 62.3, 60.5, 56.0, 55.5, 48.6, 47.5, 26.4, 26.0, 22.6, 21.2, 20.3, 18.3, 14.4, −5.3. Mass spectrum (API-TIS) m/z 500 (MH⁺).

### 43b. 3-(2-Hydroxyethyl)-4-( 1-methyl-1-sulfanylethyl)-1,3-oxazolidin-2-one

The product of Example 43a (14.9 g, 29.8 mmol) was treated with water (11.8 mL), phenol (11.8 g), anisole (11.8 mL) and finally trifluoroacetic acid (147 mL). The resultant solution was stirred at room temperature for 1 hour and then the solvent was evaporated to give a yellow oil which was chromatographed on silica gel eluting with EtOAc:Hexane (1:1) to MeOH:CH₂Cl₂ (5:95) to give the title compound (4.2 g, 69% yield) as a pale yellow oil. ¹H NMR (300 MHz, CDCl₃) δ 4.33-4.43 (m, 2H), 3.72-3.92 (m, 4H), 3.50-3.59 (m, 1H), 2.55-2.80 (br s, 1H), 1.78 (s, 1H), 1.41 (s, 6H). ¹³C NMR (75 MHz, CDCl₃) δ 160.6, 66.2, 66.0, 60.4, 48.3, 47.6, 29.0, 27.8. Mass spectrum (API-TIS) m/z 206 (MH⁺), 223 (MNH₄+). Anal. calcd for C₈H₁₅NO₃S: C, 46.81; H, 7.37; N, 6.82. Found: C, 46.81; H, 7.11; N, 6.61.

### 43c. 3-(2-Hydroxyethyl)-4-( 1-methyl-1-(nitrosothio)ethyl)-1,3-oxazolidin-2-one

To a solution of tert-butyl nitrite (4.45 mL of 90% solution, 3.5 g, 34.1 mmol) in CH₂Cl₂ (28 mL) was added dropwise a solution of the product of Example 43b (3.88 g, 18.9 mmol) in CH₂Cl₂ (58 mL) at 0° C. The resulting green solution was stirred at 0° C. for 1 hour and then at room temperature for 20 minutes in the dark. The residue after evaporation of the solvent was chromatographed on silica gel eluting with EtOAc:CH₂Cl₂ (1:1) to MeOH:CH₂Cl₂ (5:95) to give the title compound (3.7 g, 84% yield) as a green oil. ¹H NMR (300 MHz, CDCl₃) δ 4.70-4.74 (m, 1H), 4.41-4.52 (m, 2H), 3.77-3.89 (m, 3H), 3.44-3.50 (m, 1H), 1.99 (s, 3H), 1.96 (s, 3H). ¹³C NMR (75 MHz, CDCl₃) δ 160.4, 65.8, 63.9, 60.0, 59.3, 48.1, 25.7, 24.8. Mass spectrum (API-TIS) m/z 205 (M−NO), 235 (MH³⁰ ), 252 (MNH₄⁺). Anal. calcd for C₈H₁₄N₂O₄S: C, 41.02; H, 6.02; N, 11.96. Found: C, 41.30; H, 5.87; N, 11.68.

### 43d. 2-(4-(1-methyl-1-(nitrosothio)ethyl)-2-oxo-1,3-oxazolidin-3-yl)ethyl 2-(((1S,11S,14S,15S,10R)-5,14-dihydroxy-15-methyltetracyclo(8.7.0.0<2,7>0.0<11,1515>)heptadeca-2,4,6-trien-8-ylidene)azamethoxy)acetate

A mixture of the product of Example 43c (0.14 g, 0.61 mmol), N,N-dimethylaminopyridine (DMAP, 68 mg, 0.55 mmol) and the product of Example 35a (0.2 g, 0.56 mmol) in CH₂Cl₂ (3 mL) at 0° C. was treated with 1-(3-(dimethylamino)propyl)-3-ethylcarbodiimide hydrochloride (0.11 g, 0.56 mmmol). The reaction mixture was stirred at 0° C. to 4° C. for 3 hours, diluted with CH₂Cl₂, washed with water, satd. NaCl and dried over Na₂SO₄. The residue after filtration and evaporation was chromatographed on silica gel eluting with EtOAc:CH₂Cl₂ (1:3 to 1:1) to give the title compound (72 mg, 22% yield) as a green solid. Mp 75-77° C. ¹H NMR (300 MHz, CDCl₃) δ 7.40 (s, 1H), 7.18 (d, J=8.5 Hz, 1H), 6.86 (dd, J=2.7 and 8.5 Hz, 1H), 4.72-4.88 (m, 1H), 4.68 (bs, 2H), 4.50-4.65 (m, 1H), 4.28-4.35 (m, 2H), 4.04-4.28 (m, 2H), 3.76 (t, J=8.4 Hz, 1H), 3.40-3.56 (m, 1H), 3.02-3.23 (m, 1H), 2.01-2.38 (m, 4H), 1.95 (s, 3H), 1.90 (s, 3H), 1.67-1.84 (m, 1H), 1.12-1.67 (m, 7H), 0.77 (s, 3H). ¹³C NMR (75 MHz, CDCl₃) δ 170.9, 159.6, 155.7, 154.6, 134.6, 130.6, 126.2, 117.5, 110.3, 81.7, 70.8, 65.6, 62.5, 60.6, 59.0, 50.4, 44.4, 43.1, 41.8, 41.6, 37.2, 36.2, 30.4, 29.5, 25.6, 24.8, 23.1, 11.1. Mass spectrum (API-TIS) m/z 576 (MH⁺).

### Example 44

### 2-(4-(1-Methyl-1-(nitrosothio)ethyl)-2-oxo-1,3-oxazolidin-3-yl)ethyl 2-((1S,11S,14S,15S,10R)-14-hydroxy-15-methyltetracyclo(8.7.0.0<2,7>0.0<11,15>)heptadeca-2,4,6-trien-5-yloxy)acetate

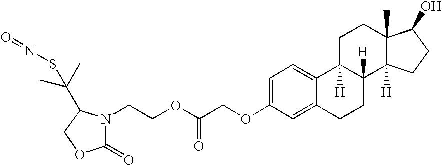

A mixture of the product of Example 43c (0.23 g, 0.98 mmol), N,N-dimethylaminopyridine (DMAP, 0.11 g, 0.91 mmol) and the product of Example 36a (0.3 g, 0.91 mmol) in CH₂Cl₂ (3 mL) at 0° C. was treated with 1-(3-(dimethylamino)propyl)-3-ethylcarbodiimide hydrochloride (174 mg, 0.91 mmmol). The reaction mixture was stirred at 0° C. to 4° C. for 3 hours, diluted with CH₂Cl₂, washed with water, satd. NaCl and dried over Na₂SO₄. The residue after filtration and evaporation was chromatographed on silica gel eluting with EtOAc:CH₂Cl₂ (1:3 to 1:1) to give the title compound (80 mg, 16% yield) as a green foam. Mp 40° C. ¹H NMR (300 MHz, CDCl₃) δ 7.15-7.21 (m, 1H), 6.64 (d, J=2.8 and 8.6 Hz, 1H), 6.50-6.55 (m, 1H), 4.59 (s, 2H), 4.52-4.70 (m, 1H), 4.40-4.52 (m, 1H), 4.21-4.40 (m, 2H), 4.10-4.21 (m, 1H), 3.92-4.18 (m, 1H), 4.16 (t, J=8.9 Hz, 1H), 3.40-3.58 (m, 1H), 2.71-2.82 (m, 2H), 2.19-2.35 (m, 1H), 1.93-2.18 (m, 2H), 1.93 (s, 3H), 1.91 (s, 3H), 1.60-1.75 (m, 1H), 1.02-1.60 (m, 10H), 0.77 (s, 3H). ¹³C NMR (75 MHz, CDCl₃) δ 169.4, 159.0, 155.5, 138.5, 134.0, 126.6, 114.5, 111.6, 81.8, 65.3, 63.0, 61.9, 59.0, 50.1, 44.1, 43.3, 38.8, 36.7, 30.6, 29.8, 27.2, 26.4, 25.2, 25.1, 23.2, 11.2. Mass spectrum (API-TIS) m/z 564 (MNH₄⁺). LCMS (100%).

### Example 45

### 2-(2-Sulfanyladamantan-2-yl)ethyl 2-((1S,11S,14S,15S,10R)-14-hydroxy-15-methyltetracyclo(8.7.0.0<2,7>0.0<11,15>)heptadeca-2,4,6-trien-5-yloxy)acetate

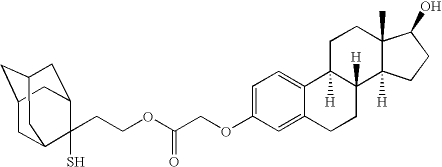

To a solution of the product of Example 36a (0.27 g, 0.82 mmol) and 2-(2-(sulfanyladamantan-2-yl)ethan-1-ol (prepared as described in U.S. Pat. No. 6,469,065, Example 10c), (0.17 g, 0.82 mmol) in CH₂Cl₂ (5 mL) was added N,N-dimethylaminopyridine (DMAP, 0.1 g, 0.82 mmol) at 0° C. To this solution dicyclohexylcarbodiimide (0.17 g, 0.82 mmol) in CH₂Cl₂ (1.5 mL) was added dropwise. The reaction mixture was stirred at 0° C. for 3 hours and at room temperature for 16 hours. The solid was filtered. The filtrate was diluted with more CH₂Cl₂, washed with water, satd. NaCl and dried over Na₂SO₄. The residue after filtration and evaporation of the solvent was purified by preparative layer chromatography eluting with EtOAc:CH₂Cl₂ (1:9 to 3:7) to give the title compound (90 mg, 23% yield) as a white solid. Mp 78-80° C. ¹H NMR (300 MHz, CDCl₃) δ 7.16-7.23 (m, 1H), 6.70 (d, J=2.6 and 8.6 Hz, 1H), 6.60-6.65 (m, 1H), 4.58 (s, 2H), 4.55 (t, J=3.3 Hz, 2H), 3.72 (t, J=8.2 Hz, 1H), 2.78-2.90 (m, 2H), 2.35-2.48 (m, 2H), 2.27 (t, J=7.3 Hz, 2H), 2.00-2.35 (m, 5H), 1.05-2.00 (m, 22H), 0.77 (s, 3H). ¹³C NMR (75 MHz, CDCl₃) δ 169.4, 155.6, 138.2, 133.8, 126.5, 114.6, 112.0, 81.8, 65.5, 62.8, 55.4, 55.0, 43.9, 43.2, 39.5, 39.0, 38.7, 38.2, 36.7, 34.0, 33.2, 30.5, 29.8, 27.7, 27.2, 26.8, 26.3, 23.1, 11.1. Mass spectrum (API-TIS) m/z 542 (MNH₄⁺). LCMS (100%).

### Example 46

### 2-(2-sulfanyladamantan-2-yl)ethyl 2-(((1S,11S,14S,15S,10R)-5,14-dihydroxy-15-methyltetracyclo(8.7.0.0<2,7>0.0<11,15>)heptadeca-2,4,6-trien-8-ylidene)azamethoxy)acetate

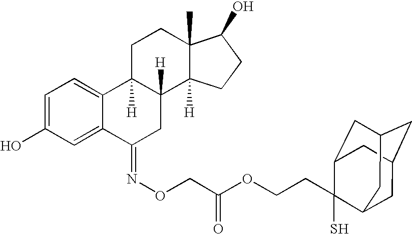

To a solution of the product of Example 36a (0.12 g, 0.34 mmol) and and 2-(2-(sulfanyladamantan-2-yl)ethan-1-ol (prepared as described in U.S. Pat. No. 6,469,065, Example 10c), (0.15 g, 0.68 mmol) in CH₂Cl₂ (5 mL) was added N,N-dimethylaminopyridine (DMAP, 41 mg, 0.34 mmol) at 0° C. To this solution dicyclohexylcarbodiimide (71 mg, 0.34 mmol) in CH₂Cl₂ (2 mL) was added dropwise. The reaction mixture was stirred at 0° C. for 3 hours and at room temperature for 16 hours. The solid was filtered. The filtrate was diluted with more CH₂Cl₂, washed with water, satd. NaCl and dried over Na₂SO₄. The residue after filtration and evaporation of the solvent was purified by preparative layer chromatography eluting with EtOAc:Hexane (1:3) to give the title compound (25 mg, 14% yield) as a white solid. Mp 85-90° C. ¹H NMR (300 MHz, CDCl₃) δ 7.30-7.44 (m, 1H), 7.05-7.12 (m, 1H), 6.76-6.83 (m, 1H), 4.72 (s, 2H), 4.42-4.62 (m, 2H), 3.73 (t, J=8.2 Hz, 1H), 3.03-3.17 (m, 1H), 2.35-2.49 (m, 2H), 2.30 (t, J=7.2 Hz, 2H), 1.41-2.22 (m, 22H), 1.00-1.41 (m, 5H), 0.73 (s, 3H). ¹³C NMR (75 MHz, CDCl₃) δ 171.3, 156.0, 154.3, 134.9, 130.9, 125.9, 117.3, 110.7, 81.8, 71.0, 62.8, 55.6, 50.5, 43.1, 41.5, 39.7, 39.1, 38.4, 38.3, 36.9, 36.2, 34.2, 33.3, 30.6, 29.8, 27.8, 26.9, 25.6, 23.2, 11.2. Mass spectrum (API-TIS) m/z 554 (MH⁺). LCMS (99.1%).

### Example 47

### 2-(((1S,11S,14S,15S,10R)-5,14-dihydroxy-15-methyltetracyclo(8.7.0.0<2,7>0.0<11,15>)heptadeca-2,4,6-trien-8-ylidene)azamethoxy)-N-(2-methyl-2-sulfanylpropyl)acetamide

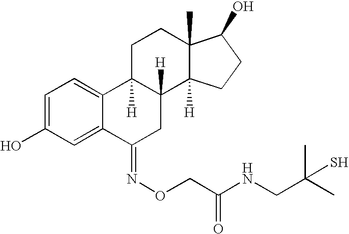

A mixture of 2-mercapto-2-methyl-1-propylamine hydrochloride (Aldrich) (0.16 g, 1.1 mmol) and N,N-dimethylaminopyridine (DMAP, 0.2 g, 1.6 mmol) in CH₂Cl₂ (3 mL) at 0° C. was treated with the product of Example 36a (0.2 g, 0.56 mmol). To this reaction mixture, a solution of 1-(3-(dimethylamino)propyl)-3-ethylcarbodiimide hydrochloride (0.13 g, 0.67 mmmol) in CH₂Cl₂ (2 mL) was added dropwise. The reaction mixture was stirred at 0° C. for 4 hours, diluted with more CH₂Cl₂, washed with water, satd. NaCl and dried over Na₂SO₄. The residue after filtration and evaporation of the solvent was purified by preparative layer chromatography eluting with EtOAc:CH₂Cl₂ (1:1) to give the title compound (48 mg, 19% yield) as a white solid. Mp 87-90° C. ¹H NMR (300 MHz, d₄-MeOH) δ 7.39 (d, J=2.7 Hz, 1H), 7.20 (d, J=8.5 Hz, 1H), 6.86 (dd, J=2.7 and 8.5 Hz, 1H), 4.67 (s, 2H), 3.70 (t, J=8.5 Hz, 1H), 3.36 (bs, 2H), 3.15-3.25 (m, 1H), 2.19-2.31 (m, 1H), 1.98-2.18 (m, 4H), 1.59-1.75 (m, 1H), 1.32-1.58 (m, 3H), 1.37 (s, 3H), 1.34 (s, 3H), 1.15-1.32 (m, 3H), 0.76 (s, 3H). ¹³C NMR (75 MHz, CDCl₃/d₄-MeOH) δ 170.9, 156.8, 154.8, 134.2, 130.0, 125.8, 117.4, 109.9, 80.7, 72.4, 51.2, 50.1, 44.8, 42.7, 41.3, 36.9, 35.8, 29.3, 29.2, 25.2, 22.7, 10.5. Mass spectrum (API-TIS) m/z 447 (MH⁺). LCMS (98%).

### Example 48

### 2-((1S,11S,14S,15S,10R)-14-hydroxy-15-methyltetracyclo(8.7.0.0<2,7>0.0<11,15>)heptadeca-2,4,6-trien-5-yloxy)-N-(2-methyl-2-sulfanylpropyl)acetamide

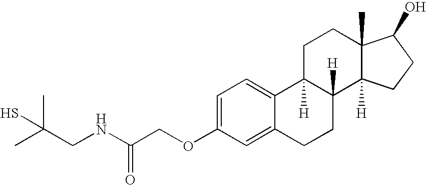

A mixture of 2-mercapto-2-methyl-1-propylamine hydrochloride (Aldrich) (0.26 g, 1.8 mmol) and N,N-dimethylaminopyridine (DMAP, 0.66 g, 5.4 mmol) in CH₂Cl₂ (6 mL) at 0° C. was treated with the product of Example 36a (0.6 g, 1.8 mmol). To this reaction mixture, a solution of 1-(3-(dimethylamino)propyl)-3-ethylcarbodiimide hydrochloride (0.35 g, 1.8 mmmol) in CH₂Cl₂ (3 mL) was added dropwise. The reaction mixture was stirred at 0° C. for 3 hours, diluted with more CH₂Cl₂, washed with water, satd. NaCl and dried over Na₂SO₄. The residue after filtration and evaporation of the solvent was purified by preparative layer chromatography eluting with EtOAc:CH₂Cl₂ (5:6) to give the title compound (0.15 g, 20% yield) as a white solid. Mp 50-52° C. ¹H NMR (300 MHz, CDCl₃) δ 7.23 (bs, 1H), 6.95-7.10 (bs, 1H), 6.75 (dd, J=2.6 and 8.6 Hz, 1H), 6.68 (d, J=2.6 Hz, 1H), 4.58 (s, 2H), 3.70-3.79 (m, 1H), 3.41 (d, J=6.4 Hz, 2H), 3.77-3.90 (m, 2H), 2.02-2.18 (m, 3H), 1.78-2.00 (m, 2H), 1.60-1.78 (m, 1H), 1.37 (s, 6H), 1.05-1.60 (m, 9H), 0.79 (s, 3H). ¹³C NMR (75 MHz, CDCl₃) δ 168.8, 155.2, 138.7, 134.5, 126.9, 114.8, 112.3, 82.0, 67.5, 51.7, 50.1, 45.4, 44.0, 43.4, 38.9, 36.8, 30.7, 30.0, 29.9, 27.2, 26.4, 23.2, 11.2. Mass spectrum (API-TIS) m/z 418 (MH⁺), 435 (MNH₄⁺).

### Example 49

### (1S,11S,14S,15S,10R)-14-hydroxy-15-methyltetracyclo(8.7.0.0<2,7>0.0<11,15>)heptadeca-2,4,6-trien-5-yl 2-(2-sulfanyladamantan-2-yl)acetate

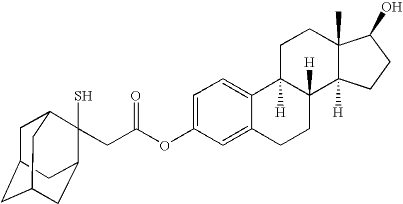

To L-cysteine (795.4 mg, 6.56 mmol) in TFA (5 mL) was added the product of Example 17a (402.0 mg, 0.61 mmol) in CH₂Cl₂ (5 mL). The reaction was stirred at room temperature for 10 minutes, concentrated to dryness, treated with EtOAc and concentrated to dryness three times. The resultant product was dissolved in EtOAc and washed with sodium bicarbonate twice, and satd. NaCl. The organic phase was dried over MgSO₄, filtered, and concentrated. The crude product was purified by chromatography (silica gel, EtOAc:Hexane 1:9; 3:14; 1:4; and then 1:3) to give the product of Example 17b (137.2 mg, 39%) and the title compound (106.1 mg, 36%). ¹H NMR (300 MHz, CDCl₃) δ 7.30-7.26 (m, 1H), 6.90-6.82 (m, 2H), 3.72 (t, J=8.4 Hz, 1H), 3.19 (s, 2H), 2.87-2.84 (m, 2H), 2.51 (m, 2H), 2.28-1.42 (m, 27H), 0.87 (s, 3H). ¹³C NMR (75 MHz, CDCl₃) δ 170.1, 148.2, 138.2, 138.0, 126.3, 121.5, 118.6, 81.8, 54.0, 50.0, 46.4, 44.1, 43.1, 38.9, 38.4, 38.1, 36.6, 33.8, 33.3, 30.5, 29.5, 27.4, 27.0, 26.7, 26.1, 23.1, 11.0. Mass spectrum (API-TIS) m/z 481 (MH⁺), 498 (MNH₄⁺).

### Example 50

### 2-((10-((3-Hydroxy-4-methoxyphenyl)methylene)(9-anthrylidene))azamethoxy)-1-(4-((nitrooxy)methyl)piperidyl)ethan-1-one

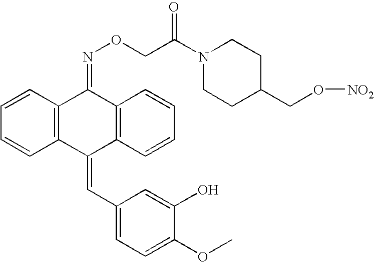

### 50a. 10-((3-hydroxy-4-methoxyphenyl)methylene)anthracen-9-one

This compound was synthesized as described by Prinz, H. et al, J. Med. Chem., 46(15): 3382-3394, (2003).

### 50b. 2-((10-((3-hydroxy-4-methoxyphenyl)methylene)-9-anthrylidene)azamethoxy)acetic acid

A mixture of the product of Example 50a (1 g, 3 mmol) and O-carboxymethyl hydroxylamine hemihydrochloride (TCI) (1.73 g, 15.8 mmol) in anhydrous MeOH (5 mL) was stirred at room temperature for four days. The solid was filtered and washed with CH₂Cl₂. The residue after evaporation of the solvent was chromatographed on silica gel eluting with EtOAc:CH₂Cl₂:Hexane (1:1:1) to EtOAc:CH₂Cl₂:MeOH (3:3:1) to give the title compound (0.5 g, 41% yield) as a pale yellow solid. Mp 232-234° C. with decompostion. ¹H NMR (300 MHz, d₆-DMSO) δ 6.60-8.52 (m, 12H), 4.45 (s, 2H), 3.35 (s, 3H). ¹³C NMR (75 MHz, d₆-DMSO) δ 172.5, 147.5, 146.4, 146.3, 146.2, 138.9, 136.8, 132.9, 132.6, 131.3, 131.2, 130.5, 129.2, 129.1, 128.9, 128.2, 127.7, 127.0, 126.0, 120.7, 116.0, 112.0, 74.7, 55.5. Mass spectrum (API-TIS) m/z 402 (MH⁺).

### 50c. 2-((10-((3-hydroxy-4-methoxyphenyl)methylene)(9-anthrylidene))azamethoxy)-1-(4-((nitrooxy)methyl)piperidyl)ethan-1-one

A mixture of nitrooxy(4-piperidylmethyl)hydrogen nitrate (prepared as described in U.S. application No. 2004/0024057, Example 19a, 0.14 g, 0.62 mmol) and N,N-dimethylaminopyridine (DMAP, 76 mg, 0.62 mmol) in CH₂Cl₂ (3 mL) at 0° C., was treated with the product of Example 50b (0.13 g, 0.32 mmol) and 1-(3-(dimethylamino)propyl)-3-ethylcarbodiimide hydrochloride (0.12 g, 0.62 mmmol). The reaction mixture was warmed from 0° C. to room temperature over 2 hours, diluted with CH₂Cl₂, washed with water, 1% hydrochloric acid, satd. NaCl and dried over Na₂SO₄. The residue after filtration and evaporation was chromatographed on silica gel eluting with EtOAc:MeOH:CH₂Cl₂ (1:0.1:1) to give the title compound (0.1 g, 57% yield) as a white solid. Mp 143-145° C. ¹H NMR (300 MHz, CDCl₃) δ 6.80-8.60 (m, 13H), 5.45-5.75 (brs, 1H), 4.98 (s, 2H), 4.58-4.80 (m, 1H), 4.20 (d, J=6.6 Hz, 2H), 3.94-4.08 (m, 1H), 3.87 (s, 3H), 2.92-3.13 (m, 1H), 2.55-2.70 (m, 1H), 1.60-2.10 (m, 4H). ¹³C NMR (75 MHz, CDCl₃) δ 167.2, 150.1, 146.3, 145.5, 133.0, 130.7, 130.3, 130.1, 129.2, 129.0, 128.7, 128.2, 127.9, 127.4, 127.3, 122.9, 122.0, 121.8, 115.4, 110.6, 73.9, 56.1, 44.9, 41.8, 34.4, 29.2, 28.4. Mass spectrum (API-TIS) m/z 544 (MH⁺).

### Example 51

### 2-((10-((3-Hydroxy-4-methoxyphenyl)methylene)(9-anthrylidene))-azamethoxy)-N-(2-methyl-2-(nitrosothio)propyl)acetamide

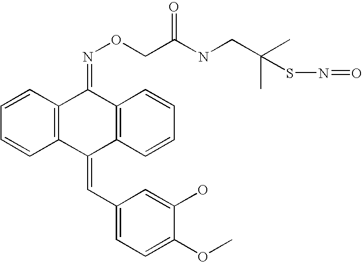

tert-Butyl nitrite (90% solution, 0.4 g, 3.9 mmol) was added dropwise to a suspension of 2-mercapto-2-methyl-1-propylamine hydrochloride (Aldrich) (0.5 g, 3.5 mmol) in CH₂Cl₂ (0.3 mL) and DMF (1 mL) at −10° C. The resultant solution was stirred at −10° C. for 5 minutes and diluted with CH₂Cl₂ and hexane. The green oil was separated, washed with hexane and dried under vacuo to give 2-methyl-2-nitrosomercapto-1-propylamine (˜0.25 g). Mass spectrum (API-TIS) m/z 135 (MH⁺). This was dissolved in CH₂Cl₂ (3 mL) and cooled to 0° C., treated portionwise with the product of Example 50b (0.13 g, 0.32 mmol), N,N-dimethylaminopyridine (DMAP, 44 mg, 0.36 mmol) and 1-(3-(dimethylamino)propyl)-3-ethylcarbodiimide hydrochloride (0.12 g, 0.62 mmmol). The reaction mixture was stirred at 0° C. for 1 hour, diluted with CH₂Cl₂, washed with water, satd. NaCl and dried over Na₂SO₄. The residue after filtration and evaporation was chromatographed on silica gel eluting with EtOAc:CH₂Cl₂ (1:1) to give the title compound (30 mg, 18% yield) as a mixture of isomers. Mp 143-145° C. ¹H NMR (300 MHz, CDCl₃) δ 6.50-8.28 (m, 13H), 5.70 (brs, 1H), 4.83 (s, 2H), 4.09 (d, J=6.4 Hz, 1H), 4.05 (d, J=6.4 Hz, 1H), 3.88 (s, 1.5H), 3.89 (s, 1.5H), 1.80 (s, 3H), 1.76 (s, 3H). ¹³C NMR (75 MHz, CDCl₃) δ 170.2, 151.2, 146.5, 145.6, 135.5, 130.2, 129.8, 129.5, 129.3, 129.1, 128.1, 128.0, 127.4, 127.3, 124.8, 123.0, 122.0, 121.8, 115.5, 110.7, 110.6, 73.9, 57.1, 56.8, 56.1, 49.0, 26.9. Mass spectrum (API-TIS) m/z 518 (MH⁺), 488 (M−NO).

### Example 52

### 5-((10-(Hydroxyimino)(9-anthrylidene))methyl)-2-methoxyphenol

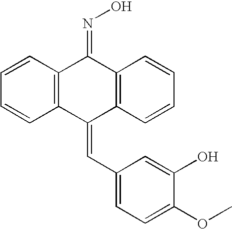

A mixture of the product of Example 50a (0.13 g, 0.39 mmol) and hydroxylamine hydrochloride (TCI) (0.15 g, 2.2 mmol) in anhydrous MeOH (4 mL) was stirred at room temperature for seven days. The solid was filtered and washed with CH₂Cl₂/MeOH. The filtrate was evaporated in vacuo. The residue after evaporation was chromatographed on preparative layer chromatography eluting with EtOAc:CH₂Cl₂ (1:2) to give the title compound (10 mg, 8% yield) as an orange-yellow solid. Mp 140° C. ¹H NMR (300 MHz, CDCl₃) δ 6.80-8.50 (m, 13H), 3.90 (s, 3H). Mass spectrum (API-TIS) m/z 344 (MH⁺).

### Example 53

### 2-(2-Methoxy-5-((10-oxo(9-anthrylidene))methyl)phenoxy)-1-(4-((nitrooxy)methyl)piperidyl)ethan-1-one

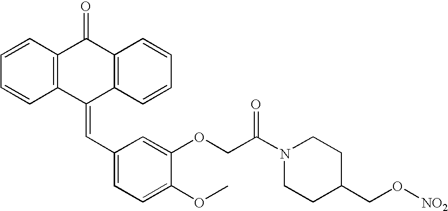

### 53a. 2-(2-Methoxy-5-((10-oxo(9-anthrylidene))methyl)phenoxy)acetic acid

A mixture of the product of Example 50a (0.7 g, 2.1 mmol), bromoacetic acid (1.1 g, 7.9 mmol) and potassium hydroxide (1.6 g, 28.5 mmol) in anhydrous DMSO (20 mL)/CH₂Cl₂ (5 mL) was stirred at room temperature for 2 hours. The residue after evaporation of the solvent was dissolved in water, washed with EtOAc, acidified with 6N hydrochloric acid and extracted with EtOAc. The combined organic layer was dried over Na₂SO₄. The residue after evaporation was chromatographed on silica gel eluting with EtOAc:Hexane (1:1) to MeOH:CH₂Cl₂ (1:3) to give the title compound (0.6 g, 73% yield) as a yellow solid. Mp 227-230° C. ¹H NMR (300 MHz, d₆-DMSO) δ 6.75-8.35 (m, 12H), 4.12 (s, 2H), 3.71 (s, 3H). ¹³C NMR (75 MHz, d₆-DMSO) δ 183.5, 148.9, 147.8, 140.1, 136.0, 134.5, 133.1, 131.5, 131.1, 129.7, 128.8, 128.4, 128.0, 127.6, 126.2, 123.6, 121.9, 113.8, 111.7, 55.5. Mass spectrum (API-TIS) m/z 387(MH⁺), 385 (M−H).

### 53b. 2-(2-Methoxy-5-((10-oxo(9-anthrylidene))methyl)phenoxy)-1-(4-((nitrooxy)methyl)piperidyl)ethan-1-one

A mixture of nitrooxy(4-piperidylmethyl)hydrogen nitrate (prepared as described in U.S. application No. 2004/0024057, Example 19a, 0.23 g, 1 mmol) and N,N-dimethylaminopyridine (DMAP, 125 mg, 1 mmol) in CH₂Cl₂ (5 mL) at 0° C., was treated with the product of Example 53a (0.2 g, 0.5 mmol) and 1-(3-(dimethylamino)propyl)-3-ethylcarbodiimide hydrochloride (0.12 g, 0.62 mmmol). The reaction mixture was stirred at 0° C. to 4° C. for 4 hours. The reaction mixture was diluted with CH₂Cl₂, washed with water, 1% hydrochloric acid, satd. NaCl and dried over Na₂SO₄. The residue after filtration and evaporation was chromatographed on silica gel eluting with EtOAc:MeOH:CH₂Cl₂ (1:0.1:2) to give the title compound (30 mg, 11% yield) as a yellow solid. Mp 63-65° C. ¹H NMR (300 MHz, CDCl₃) δ 6.80-8.32 (m, 12H), 4.56 (s, 2H), 4.22-4.40 (bs, 2H), 3.89 (s, 3H), 2.90-3.08 (m, 1H), 2.50-2.70 (m, 1H), 1.50-2.10 (m, 4H), 1.08-1.30 (m, 3H). ¹³C NMR (75 MHz, CDCl₃) δ 179.6, 160.6, 144.4, 142.1, 135.4, 131.2, 127.6, 127.3, 125.6, 125.4, 125.1, 124.4, 124.0, 123.0, 122.4, 121.7, 118.7, 117.8, 109.9, 106.6, 63.1, 50.7, 39.5, 36.5, 29.1, 24.0, 23.0. Mass spectrum (API-TIS) m/z 529 (MH⁺), 546 (MNH₄)⁺.

### Example 54

### 2-((4-((2,4-Dioxo(1,3-thiazolidin-5-yl))methyl)phenoxy)methyl)-2,5,7,8-tetramethylchroman-6-yl 2-(((N-(2-methyl-2-(nitrosothio)propyl)-N-benzylcarbamoyl)methyl)cyclopentyl)acetate

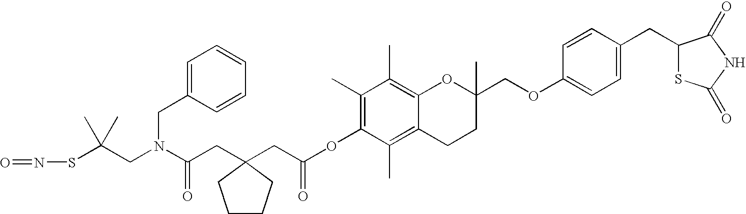

### 54a. di-1,1-Dimethyl-2-(benzylamino)ethyl disulfide

A mixture of 2-((1,1-dimethyl-2-oxoethyl)disulfanyl)-2-methylpropanal(prepared as described by Roy et al J. Org. Chem. 59, 7019-7026, 1994, 10.31 g, 50 mmol) and benzylamine (10.71 g, 100 mmol) in CHCL₃ (150 mL) was refluxed 2 hours and allowed to cool to room temperature. The solvent was evaporated and the residue dissolved in MeOH (100 mL) and sodium borohydride (6 g, 158 mmol) added in portions with ice cooling. The reaction mixture was warmed to room temperature and water (300 mL) added. The aqueous phase was extracted with EtOAc and ether, the combined extracts were dried over Na₂SO₄, filtered and evaporated to give the title compound (18.45 g, 95%). ¹H NMR (300 MHz, CDCl₃) δ 7.20-7.35 (m, 10H), 3.81 (s, 4H), 2.57 (s, 4H), 1.60 (s, 2H), 1.28 (s, 12H).

### 54b. 2-Methyl-1-(benzylamino)propane-2-thiol

A solution of the product of Example 54a (13.2 g, 34.1 mmol) in ether (70 mL) was treated with liquid mmonia (100 mL) followed by addition of sodium to give a permanent blue colour (approx 2 g). The blue solution was stirred for 1 hour and ammonium chloride (5 g) added. The excess ammonia was allowed to evaporate and water added. The aqueous phase was extracted with ether and the combined organic phase washed with satd. NaCl, dried with Na₂SO₄, filtered and evaporated to give the title compound (12.8 g, 96% yield). ¹H NMR (300 MHz, d₆-DMSO) δ 9.57 (br s, 2H), 7.71-7.79 (m, 2H), 7.51-7.58 (m, 3H), 4.30 (s, 2H), 3.06 (s, 2H), 1.48 (s, 6H).

### 54c. 2-((((2-((4-((2,4-Dioxo(1,3-thiazolidin-5-yl))methyl)phenoxy)methyl)-2,5,7,8-tetramethylchroman-6-yl)oxycarbonyl)methyl)cyclopentyl)acetic acid

A mixture of the product of Example 54b (50 mg, 0.11 mmol), 4-dimethylaminopyridine (14 mg, 0.11 mmol) and 3,3-tetramethyleneglutaric anhydride (19 mg, 0.11 mmol) in CH₂Cl₂ (1 mL) was stirred at room temperature for 24 hours. The reaction mixture was diluted with more CH₂Cl₂ washed with 2N HCl and dried with Na₂SO₄. Filtration and evaporation gave the title compound which was used in the next step without further purification (69 mg, 100% yield). Mass spectrum (API-TIS) m/z 627 (MNH₄⁺).

### 54d. 2-((4-((2,4-dioxo(1,3-thiazolidin-5-yl))methyl)phenoxy)methyl)-2,5,7,8-tetramethylchroman-6-yl 2-(((N-(2-methyl-2-(nitrosothio)propyl)-N-benzylcarbamoyl)methyl)cyclopentyl)acetate

A mixture of the product of Example 54b (23 mg, 0.12 mmol), the product of Example 54c (69 mg, 0.11 mmol), 4-dimethylaminopyridine (14 mg, 0.11 mmol), triethylamine (17.5 μL, 12.6 mg, 0.11 mmol) and benzotriazol-1-yloxytris(dimethylamino)phosphonium hexafluorophosphate (25 mg, 0.056 mmol) in CH₂Cl₂ (2 mL) was stirred at room temperature overnight. The reaction mixture was diluted with more CH₂Cl₂, washed with water, dried over Na₂SO₄, filtered and evaporated. The residue was chromatographed on silica gel, eluting with EtOAc:Hexane 1:2, to give the title product.(31 mg, 71% yield). ¹H NMR (300 MHz, CDCl₃) δ 7.02-7.35 (m, 7H), 6.86 (d, J=8.5 Hz, 2H), 4.88 (s, 2H), 4.46 (dd, J=9.6 and 3.8 Hz, 1H), 3.92 (dd, J=31.4 and 9.1 Hz, 2H), 3.60 (s, 1H), 3.45 (dd, J=14.7 and 3.9 Hz, 1H), 3.05-3.13 (m, 3H), 2.71 (s, 2H), 2.61 (t, J=6.4 Hz, 2H), 2.07 (s, 3H), 1.98 (s, 3H), 1.92 (s, 3H), 1.82 (s, 1H), 1.50-2.20 (m, 12H), 1.40 (s, 6H), 1.35-1.45 (m, 4H). Mass spectrum (API-TIS) m/z 788 (MH⁺).

### Example 55

### (7-Methyl(4-hydro-1,2,4-triazolo(1,5-a)pyrimidin-5-yl))(2-methyl-2(nitrosothio)propyl)amine

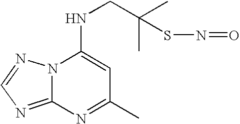

### 55a. 2-Mercapto-2-methyl-1-propylamine

To a suspension of 2-mercapto-2-methyl-1-propylamine hydrochloride (8 g, 56.7 mmol) in ether (100 mL) was added triethylamine (20 mL, 143.5 mmol). The reaction mixture was stirred overnight at room temperature, filtered and the filtrate evaporated to give the product as a volatile solid (3.95 g, 91% yield). ¹H NMR (300 MHz, CDCl₃) δ 2.77 (s, 2H), 1.72 (s, 3H), 1.34 (s, 6H). ¹³C NMR (75 MHz, CDCl₃) δ 56.2, 46.9, 29.6.

### 55b. 2-Methyl-1-((7-methyl(4-hydro-1,2,4-triazolo(1,5-a)pyrimidin-5-yl))amino)propane-2-thiol

To a solution of 7-chloro-5-methyl-7a-hydro-1,2,4-triazolo(1,5-a)pyrimidine (prepared as described in U.S. Pat. No. 5,869,486, 2.15 g, 12.8 mmol) in ethanol (20 mL) was added triethylamine (1.3 g, 12.8 mmol) and the product of Example 54a (1.88 g, 17.9 mmol). The reaction mixture was stirred at 80° C. for 36 hours, cooled to room temperature, evaporated, dissolved in CH₂Cl₂, washed with water, dried with Na₂SO₄, filtered and evaporated. The residue was chromatographed on silica gel, eluting with CH₂Cl₂:MeOH (1:9), to give the title compound (1.9 g, 63% yield). Mp 137-139° C. ¹H NMR (300 MHz, CDCl₃) δ 8.32 (s, 1H), 6.66 (t, J=6.4 Hz, 1H), 6.06 (s, 1H), 3.49 (d, J=6.4 Hz, 2H), 2.59 (s, 3H), 1.90 (s, 1H), 1.52 (s, 6H). ¹³C NMR (75 MHz, CDCl₃) δ164.9, 155.4, 154.6, 147.2, 88.0, 55.3, 44.8, 30.0, 25.4. Mass spectrum (API-TIS) m/z 237 (M⁺). Anal. calcd for C₁₀H₁₅N₅S: C, 50.61; H, 6.37; N, 29.51, Found: C, 50.42; H, 6.38; N, 29.22.

### 55c. (7-Methyl(4-hydro-1,2,4-triazolo(1,5-a)pyrimidin-5-yl))(2-methyl-2-(nitrosothio)propyl)amine

The product of Example 55b (170 mg, 0.72 mmol) in CH₂Cl₂ (3 mL) was added dropwise to tert-butyl nitrite (90% solution, 92 μL, 80 mg, 0.78 mmol) in CH₂Cl₂ (1 mL). The reaction mixture was stirred at room temperature for 40 minutes in the dark, the solvent evaporated and the residue chromatographed (CH₂Cl₂:MeOH 9;1) to give the title compound (135 mg, 71% yield). ¹H NMR (300 MHz, CDCl₃) δ 8.32 (s, 1H), 6.75 (t, J=6.6Hz, 1H), 6.15 (s, 1H), 4.27 (d, J=6.6 Hz, 2H), 2.61 (s, 3H), 2.07 (s, 6H). ¹³C NMR (75 MHz, CDCl₃) δ 165.0, 155.3, 154.5, 147.2, 87.9, 56.2, 52.6, 26.8, 25.4. Mass spectrum (API-TIS) m/z 267 (MH⁺). Anal. calcd for C₁₀H₁₄N₆OS: C, 45.10; H, 5.30; N, 31.56, Found: C, 44.97; H, 5.28; N, 31.80.

### Example 56

### 2-(2-(Nitrosothio)adamantan-2-yl)ethyl 1-(7-methyl-4-hydro-1,2,4-triazolo(1,5-a)pyrimidin-5-yl)piperidine-4-carboxylate

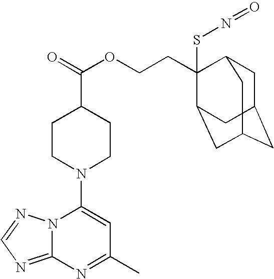

### 56a. 1-(5-Methyl-7a-hydro-1,2,4-triazolo(1,5-a)pyrimidin-7-yl)piperidine-4-carboxylic acid

A mixture of 7-chloro-5-methyl-7a-hydro-1,2,4-triazolo(1,5-a)pyrimidine (prepared as described in U.S. Pat. No. 5,869,486, 1.68 g, 10 mmol), triethylamine (4.2 mL, 3 g, 30 mmol) and isonipecotic acid (1.29 g, 10 mmol) was dissolved in water (20 mL) and heated at 80° C. for 2 h. The solvent was removed by azeotropic distillation with CH₃CN to give the title compound as the triethylamine salt which was used without further purification.

### 56b. 2(2,4,6-Trimethoxyphenylmethylthioadamant-2-yl)acetic acid

A suspension of 2-(2-sulfanyladamantan-2-yl)acetic acid (prepared as described in U.S. application No. 2003/0203915, Example 12b, 2.5 g, 11 mmol) in CH₂Cl₂ (90 mL) was cooled to 0° C. Trifluoroacetic acid (17.9 mL, 232 mmol) was added dropwise over a period of 3 minutes then the product of Example 56a (2.19 g, 11 mmol) in CH₂Cl₂ (45 mL) was added dropwise at 0° C. The reaction mixture was stirred for 2 hours at 0° C., the solvent evaporated and the solid was dissolved in CH₂Cl₂. The organic phase was washed with water, dried with Na₂SO₄, filtered and evaporated. The solid was dissolved in CH₂Cl₂ (20 mL) and stirred at room temperature for 15 minutes. The insoluble material was filtered and the residue after evaporation was chromatographed on silica gel, eluting with EtOAc:Hexane (1:1) to give the title compound (1.35g, 30% yield). Mp 157-159° C. ¹H NMR (300 MHz, CDCl₃) δ 10.45 (br s, 1H), 6.11 (s, 2H), 3.82 (s, 6H), 3.80 (s, 3H), 3.67 (s, 2H), 3.13 (s, 2H), 2.59 (d, J=12.5, 2H), 2.07 (d, J=17.8, 2H), 1.89 (m, 4H), 1.75 (m, 4H), 1.62 (m, 2H). ¹³C NMR (75 MHz, CDCl₃) δ 171.9, 160.9, 158.8, 104.1, 90.6, 55.9, 55.8, 55.4, 40.6, 39.0, 34.3, 32.9, 32.8, 27.3, 27.1, 19.1. Mass spectrum (API-TIS) m/z 407 (MH⁺). Anal. calcd for C₂₂H₃₀SO₅: C, 64.00; H, 7.44. Found: C, 64.39; H, 7.53.

### 56c. 2-(2-((2,4,6-Trimethoxyphenyl)methylthio)adamantan-2-yl)ethan-1-ol

A solution of the product of Example 56b (7.5 g, 19 mmol) in THF (75 mL) was treated carefully in portions with lithium aluminum hydride (0.9 g, 24 mmol). The reaction mixture was stirred at 70° C. for 2 hours, cooled to room temperature and quenched carefully with water then satd sodium bicarbonate solution. The aqueous phase was extracted with EtOAc and the organic phase was dried with Na₂SO₄, filtered and evaporated to give the title compound (7 g, 97% yield). ¹H NMR (300 MHz, CDCl₃) δ 6.10 (s, 2H), 3.89 (t, J=5.5 Hz, 2H), 3.84 (s, 6H), 3.81 (s, 3H), 3.70 (s, 2H), 2.70 (d, J=12.0 Hz, 2H), 2.30 (t, J=5.5 Hz, 2H), 2.06 (d, J=13.1 Hz, 2H), 1.95 (br s, 2H), 1.89 (br s, 2H), 1.54-1.75 (m, 7H).

### 56d. 2-(2-((2,4,6-Trimethoxyphenyl)methylthio)adamantan-2-yl)ethyl 1-(7-methyl-4-hydro-1,2,4-triazolo(1,5-a)pyrimidin-5-yl)piperidine-4-carboxylate

A mixture of the product of Example 56a (2.65 g, 7.4 mmol), the product of Example 56c (3.94 g, 10 mmol) and 4-dimethylaminopyridine (0.25 g, 2 mmol) in DMF (60 mL) was treated with 1-(3-(dimethylamino)propyl)-3-ethylcarbodiimide hydrochloride (2.42 g, 12.7 mmol). The reaction mixture was stirred overnight at room temperature, the solvent removed by vacuum distillation, the residue suspended in EtOAc and washed several times with water. The organic phase was dried with Na₂SO₄, filtered and evaporated. The residue was chromatographed on silica gel, eluting with EtOAc:MeOH 9:1 to give the title compound (3 g, 64% yield). ¹H NMR (300 MHz, CDCl₃) δ 8.21 (s, 1H), 6.15 (s, 1H), 6.07 (s, 2H), 4.54 (t, J=6.1 Hz, 2H), 4.08 (dt, J=22.1 and 3.2 Hz, 2H), 3.82 (s, 6H), 3.80 (s, 3H), 3.60 (s, 2H), 3.27-3.38 (m, 2H), 2.60-2.71 (m, 3H), 2.59 (s, 3H), 2.29 (t, J=6.2 Hz, 2H), 1.68-2.20 (m, 14H), 1.56 (d, J=12 8 Hz, 2H). Mass spectrum (API-TIS) m/z 636 (MH⁺).

### 56e. 2-(2-Sulfanyladamantan-2-yl)ethyl 1-(7-methyl-4-hydro-1,2,4-triazolo(1,5-a)pyrimidin-5-yl)piperidine-4-carboxylate

A mixture of the product of Example 56d (2.7 g, 4.3 mmol), phenol (0.5 g, 5.3 mmol), anisole (0.5 mL, 4.8 mmol) and water (1 mL) was treated with trifluoroacetic acid (40 mL). The reaction mixture was stirred at room temperature for 50 minutes, the volatile material was evaporated and the residue neutralised with sodium bicarbonate solution and extracted with EtOAc. The organic phase was washed with satd. NaCl, dried over Na₂SO₄, filtered and evaporated. The residue was chromatographed on silica gel, eluting with EtOAc:methanol (9:1) to give the title compound (1.3 g, 67% yield). Mp 157-159° C. ¹H NMR (300 MHz, CDCl₃) δ 8.20 (s, 1H), 6.02 (s, 1H), 4.22-4.38 (m, 4H), 3.21 (t, J=10.9 Hz, 2H), 2.50-2.62 (m, 1H), 2.48 (s, 3H), 2.34 (d, J=12.6Hz, 2H), 2.17 (t, J=7.2Hz, 2H), 1.50-2.10 (m, 17H). ¹³C NMR (75 MHz, CDCl₃) δ 174.2, 165.1, 157.6, 154.5, 150.5, 95.0, 62.6, 55.8, 48.0, 40.9, 39.9, 39.3, 38.6, 34.4, 33.6, 28.0, 27.8, 27.1, 25.5. Mass spectrum (API-TIS) m/z 456 (MH⁺). Anal. calcd for C₂₄H₃₂NO₂S: C, 63.41; H, 7.10; N, 15.41. Found: C, 63.35; H, 7.19; N, 15.08.

### 56f. 2-(2-(Nitrosothio)adamantan-2-yl)ethyl 1-(7-methyl-4-hydro-1,2,4-triazolo(1,5-a)pyrimidin-5-yl)piperidine-4-carboxylate

A solution of the product of Example 56e (154 mg, 0.34 mmol) in CH₂Cl₂ (2 mL) was added dropwise to a solution of tert-butyl nitrite (225 μL of a 90% solution, 174 mg, 1.69 mmol) in CH₂Cl₂. The reaction mixture was stirred at room temperature for 1 hour in the dark, the solvent evaporated and the residue chromatographed (EtOAc:acetone 4:1) to give the title compound. ¹H NMR (300 MHz, CDCl₃) δ 8.24 (s, 3H), 6.10 (s, 1H), 4.20-4.36 (m, 4H), 3.22 (t, J=10.6 Hz, 2H), 3.03 (t, J=7.2 Hz, 2H), 2.52 (s, 3H), 2.50-2.65 (m, 2H), 1.60-2.11 (m, 15H). ¹³C NMR (75 MHz, CDCl₃) δ 173.7, 164.8, 154.0, 150.1, 94.7, 67.6, 61.5, 47.6, 40.5, 38.8, 35.6, 35.5, 33.8, 33.1, 27.4, 27.3, 27.1, 25.1. Mass spectrum (API-TIS) m/z 485 (MH⁺).

### Example 57

### (1S,11S,14S,15S,10R)-15-Methyl-5-phenylcarbonyloxytetracyclo(8.7.0.0<2,7>0.0<11,15>)heptadeca-2,4,6-trien-14-yl(1S,2S,5R,6R)-6-hydroxy-4,8-dioxabicyclo(3.3.0)oct-2-yl butane-1,4-dioate

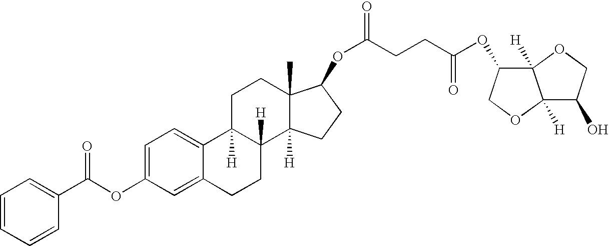

To the product of Example 14a (380 mg, 0.797 mmol) in THF (10 mL) at room temperature was added isosorbide (Aldrich, Wis.; 122 mg, 0.827 mmol, 1.05 eq) followed by the addition of a catalytic amount of DMAP (2mg) and EDAC (168 mg, 0.877 mmol, 1.1 eq). The reaction mixture was stirred overnight, diluted with CH₂Cl₂ (100 mL), and washed with H₂O and then brine. The organic layer was dried over Na₂SO₄, filtered and the solvent was removed to give a yellow oil. The product was chromatographed on silica gel eluting with EtOAc/Hexanes *2:3, 4:1 and 100:0) to give the title compound (160 mg, 33%) as a white solid. Mp 136-137° C. ¹H NMR (300 MHz, CDCl₃) δ 8.19 (m, 2H), 7.63 (m, 1H), 7.51 (m, 2H), 7.33 (m, 1H), 6.96 (m, 2H), 5.27 (d, J=3.0 Hz, 1H), 4.71 (dd, J=7.7, 8.9 Hz, 1H), 4.64 (t, J=4.9 Hz, 1H), 4.49 (d, J=4.4 Hz, 1H), 4.32 (m, 1H), 4.03 (m, 2H), 3.73 (AB part of ABX, Δν_{AB}=125.9 Hz, J_{AB}=9.5 Hz, J_{AX}=6.0 Hz, J_{BX}=6.0 Hz, 2H), 2.89 (m, 2H), 2.66 (br s, 4H), 2.40-2.15 (m, 4H), 1.88 (m, 2H), 1.77 (m, 1H), 1.64-1.25 (m, 7H), 0.84 (s, 3H). Mass spectrum (API-TIS) m/z 605 (MH⁺), 622 (MNH₄⁺).

### Example 58

### (1S,11S,14S,15S,10R)-14-Hydroxy-15-Methyltetracyclo(8.7.0.0<2,7>0.0<11,15>)heptadeca-2,4,6-trien-5-yl(1S,2S,5R,6R)-6-hydroxy-4,8-dioxabicyclo(3.3.0)oct-2-yl butane-1,4-dioate

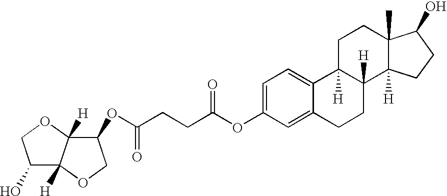

### 58a. 3-(((1S,2S,5S,6R)-6-(Hydroxy)-4,8-dioxabicyclo(3.3.0)oct-2-yl)oxycarbonyl)propanoic acid

Isosorbide (Aldrich, Wis.; 4.17 g, 28.53 mmol), succinic anhydride (Aldrich, Wis., US; 2.38 g, 23.78 mmol, 0.83 eq), and DMAP (2.91 g, 23.78 mmol, 0.83 eq) were slurried in THF (30 mL), and refluxed overnight. The reaction mixture was diluted with EtOAc, washed twice with 3N HCl, and then finally brine. The organic layer was dried over Na₂SO₄, filtered, and the solvent was removed in vacuo to give the title compound (4.8 g, 82%) as a thick pale yellow oil. ¹H NMR (300 MHz, CDCl₃) δ 8.2 (br s, 1H), 5.21 (m, 2H), 4.85 (m, 1H), 4.48 (m, 1H), 4.33 (m, 1H), 4.05-3.72 (m, 4H), 2.66 (m, 4H). Mass spectrum (API-TIS) m/z 247 (MH⁺), 264 (MNH₄⁺).

### 58b. (1S,11S,14S,15S,10R)-14-Hydroxy-15-methyltetracyclo(8.7.0.0<2,7>0.0<11,15>)heptadeca-2,4,6-trien-5-yl(1S,2S,5R,6R)-6-hydroxy-4,8-dioxabicyclo(3.3.0)oct-2-yl butane-1,4-dioate

To estradiol (Steraloids, R.I., US; 590 mg, 2.17 mmol) and the product of Example 58a (800 mg, 3.25 mmol, 1.5 eq) in CH₂Cl₂ (20 mL) was added a catalytic amount of DMAP (10 mg) followed by the addition of EDAC (642 mg, 3.25 mmol, 1.5 eq). The reaction mixture was stirred at ambient temperature for 1.5 hours, diluted with CH₂Cl₂, washed twice with H₂O and finally brine. The organic layer was dried over Na₂SO₄, filtered, and the solvent was removed in vacuo. The product was chromatographed on silica gel eluting with EtOAc/Hexanes (1:4, 1:1) to give the title compound (104 mg, 10%) as a white solid. Mp 79-80° C. ¹H NMR (300 MHz, CDCl₃) δ 7.27 (m, 1H), 6.84 (m, 1H), 6.79 (m, 1H), 5.21 (m, 2H), 4.84 (t, J=4.9 Hz, 1H), 4.47 (d, J=4.3 Hz, 1H), 4.29 (m, 1H), 4.03-3.56 (m, 4H), 2.89-2.63 (m, 6H), 2.38-1.17 (m, 15H), 0.76 (s, 3H). Mass spectrum (API-TIS) m/z 501 (MH⁺), 518 (MNH₄⁺).

### Example 59

### Suppression of Proliferation of Human Coronary Artery Smooth Muscle Cells (CASMC)

Vascular Smooth Muscle Cell (SMC) Antiproliferation Assay

The cells used in this assay were human coronary artery smooth muscle cells (CASMC) supplied by Clonetics Corp. (San Diego, Calif.). They were maintained in SmGM-2 growth medium (Clonetics Corp.), which consisted of modified MCDB 131 medium supplemented with 5% (v/v) fetal bovine serum (FBS), 0.5 ng/mL human recombinant epidermal growth factor (EGF), 2 ng/mL human recombinant fibroblast growth factor (FGF), 5 μg/mL bovine insulin, 50 μg/mL gentamicin sulfate, and 50 ng/mL amphotericin B under humidified 95% air-5% CO₂ at 37° C. Cells were used for experiments up to about 17 cumulative population doublings (i.e., passage 9); at this age they still stained positive for smooth muscle actin, a protein marker for smooth muscle cells.

For the SMC antiproliferation assay, the cells were seeded at 3×10⁴ viable cells in 2 mL of SmGM-2 medium per well of a Corning 24 tissue culture well plate (Corning, N.Y.). Stock solutions of the test compounds were prepared just prior to addition to the cells by dissolving in DMSO at a concentration of 1000 times the highest concentration to be assayed. This stock solution was diluted, as required, with DMSO to lower concentrations. On the same day the cells were seeded, but after they had attached and spread out (about 3 hours), each test compound in varying concentrations (2 μL of the diluted stock solutions) was added to four replicate wells (n=4) for each concentration. Control cultures received 2 μL of DMSO per well (n=4). On the following morning, the cultures were examined microscopically and their condition recorded. On the third day after test compound addition (˜68 hours), the cultures were examined microscopically again and the viable cells counted with an hemacytometer following trypsinization with 0.25% trypsin-1 mM EDTA. Trypan Blue dye exclusion was used to discriminate between viable and dead cells. The results were usually presented as % of the control viable cell count (mean±SEM) and were used to determine the IC₅₀ for the inhibition of proliferation of vascular smooth muscle cells. The IC₅₀ for some the nitric oxide donors is given in Table 1.

TABLE 1

| | |
|---|---|
| Nitrosated and/or | Non-nitrosated and/or |
| Nitrosylated Compound | Non-nitrosylated Compound |

| Example # | IC 50 μM | Example # | IC 50 μM |
|---|---|---|---|
| 15 | >>80 | 57 | >80 |
| 17c | Non-inhibitory | 17b | cytostatic |
| 18 | 9 | 49 | 4 |
| 25 | 4 | 30 | 4 |
| 28i | 13 | 28h | 14 |
| 32b | 8 | 58 | 12 |
| 35 b | Non-inhibitory | 35a | >>80 |
| 36b | 5 | 45 | 8 |
| 37 | 1.4-2 | 46 | 5-12 |
| 41 | 10 | 47 | >>80 |
| 42 | 9 | 48 | 11 |
| 51 | 10 | 52 | 0.6 |

Table 1 shows that the nitrosated (i.e. nitrate) and/or nitrosylated (i.e. nitrosothiol) compound inhibits the proliferation of vascular smooth muscle cells while the correspond non-nitrosated (i.e. alcohol) and/or non-nitrosylated (i.e. sulfhydryl) derivative either had no inhibition, slight inhibition or had a much higher IC₅₀ for the inhibition of the proliferation of vascular smooth muscle cells. These results indicate that the inhibition of the proliferation of vascular smooth muscle cells was attributable to the presence of the NO moiety.

The disclosure of each patent, patent application and publication cited or described in the specification is hereby incorporated by reference herein in its entirety.

Although the invention has been set forth in detail, one skilled in the art will appreciate that numerous changes and modifications may be made without departing from the spirit and scope of the invention.

What is claimed is:

1. An estradiol compound comprising at least one NO group, or at least one NO and NO₂ group, a stereoisomer thereof, and/or a pharmaceutically acceptable salt thereof, wherein the at least one NO group, or the at least one NO and NO₂ group is linked to the estradiol compound, through an oxygen atom, a nitrogen atom or a sulfur atom.

2. A nitrosated and/or nitrosylated compound of Formula (I), a stereoisomer thereof, and/or a pharmaceutically acceptable salt thereof,
wherein the compound of Formula (I) is: 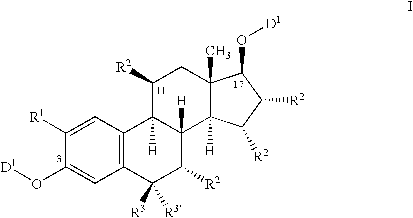
wherein:
R¹ is hydrogen, alkoxy, —O—(C(Rₑ)(R_{f}))ₕ—U—V or —(C(Rₑ)(R_{f}))ₕ—U—V;
R² at each occurrence is independently a hydrogen or —W′ₐ—U—V;
R³and R^{3′} are independently a hydrogen or —O-D¹;
R³and R^{3′} taken together are oxygen or ═N—O-D¹;
D¹ is a hydrogen, V or K;
V is —NO or —NO₂;
K is —W′ₐ-E_{b}-(C(Rₑ)(R_{f}))_{p′}-E_{c}-(C(Rₑ)(R_{f}))ₓ—W′_{d}—(C(Rₑ)(R_{f}))_{y}—W′ᵢ-Eⱼ-W′_{g}—(C(Rₑ)(R_{f}))_{z}—U—V;
a, b, c, d, g, i and j are each independently an integer from 0 to 3;
p′, x, y and z are each independently an integer from 0 to 10;
W′ at each occurrence is independently —C(O)—, —C(S)—, -T″-, —(C(Rₑ)(R_{f}))ₕ—, an alkyl group, an aryl group, a heterocyclic ring, an arylheterocyclic ring, or —(CH₂CH₂O)_{q′}—;
E at each occurrence is independently -T″-, an alkyl group, an aryl group, —(C(Rₑ)(R_{f}))ₕ—, a heterocyclic ring, an arylheterocyclic ring, or —(CH₂CH₂O)_{q′}—;
T″ at each occurrence is independently a covalent bond, a carbonyl, an oxygen, —S(O)ₒ— or —N(Rₐ)Rᵢ;
h is an integer form 1 to 10;
q′is an integer from 1 to 5;
Rₑ and R_{f} are each independently a hydrogen, an alkyl, a cycloalkoxy, a halogen, a l0 hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, W′ₕ, —(CH₂)ₒ—U—V, or —(C(R_{g})(Rₕ))ₖ—U—V, or Rₑ and R_{f} taken together with the carbons to which they are attached form a carbonyl, a methanthial, a heterocyclic ring, a cycloalkyl group, an aryl group, an oxime, a hydrazone or a bridged cycloalkyl group;
R_{g} and Rₕ at each occurrence are independently Rₑ;
k is an integer from 1 to 3;
U at each occurrence is independently a covalent bond, a carbonyl, an oxygen, —S(O)ₒ— or —N(Rₐ)Rᵢ;
o is an integer from 0 to 2;
Rₐ is a lone pair of electrons, a hydrogen or an alkyl group;
Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U—V)(Rₑ)(R_{f}), a bond to an adjacent atom creating a double bond to that atom, —(N₂O₂—)⁻·M⁺, wherein M⁺ is an organic or inorganic cation; and with the proviso that the compounds of Formula (I) must contain at least one NO group, or at least one NO₂ group wherein the at least one NO group or the at least one NO and NO₂ group is linked to the compound of Formula (I) through an oxygen atom, a nitrogen atom or a sulfur atom.

3. The compound of claim 2, wherein the compound of Formula (I) is a nitrosated estradiol compound, a nitrosylated estradiol compound, a nitrosated and/or nitrsylated estradiol compound.

4. A composition comprising the compound of claim 2 and a pharmaceutically acceptable carrier.

5. A method for treating a cardiovascular disease or disorder in a patient in need thereof comprising administering a therapeutically effective amount of the composition of claim 4.

6. The method of claim 5, wherein the cardiovascular disease or disorder is restenosis, coronary artery disease, atherosclerosis, atherogenesis, cerebrovascular disease, angina, ischemic disease, congestive heart failure, pulmonary edema associated with acute myocardial infarction, aneurysm, thrombosis, hypertension, platelet adhesion, platelet aggregation, smooth muscle cell proliferation, a vascular or non-vascular complication associated with the use of a medical device, a wound associated with the use of a medical device, pulmonary thromboembolism, cerebral thromboembolism, thrombophlebitis, thrombocytopenia or a bleeding disorder.

7. The method of claim 6, wherein the cardiovascular disease or disorder is restenosis or atherosclerosis.

8. A method for treating an autoimmune disease, a pathological condition resulting from abnormal cell proliferation, polycyctic kidney disease, or an inflammatory disease, for preserving an organ and/or a tissue, or for inhibiting wound contraction in a patient in need thereof comprising administering a therapeutically effective amount of the composition of claim 4.

9. The method of claim 8, wherein the pathological condition resulting from abnormal cell proliferation is a cancer, a Karposi's sarcoma, a cholangiocarcinoma, a choriocarcinoma, a neoblastoma, a Wilm's tumor, Hodgkin's disease, a melanoma, multiple myelomas, a chronic lymphocytic leukemia or an acute or chronic granulocytic lymphoma.

10. The method of claim 8, wherein the inflammatory disease is rheumatoid arthritis, an inflammatory skin disease, multiple sclerosis, a surgical adhesion, tuberculosis, a graft rejection, an inflammatory lung disease, an inflammatory bowel disease, an inflammatory disease that affects or causes obstruction of a body passageway, an inflammation of the eye, an inflammation of the nose, an inflammation of the throat or a neovascular disease of the eye.

11. The method of claim 5 or 8, wherein the compound is administered intravenously, orally, bucally, parenterally, by an inhalation spray or by topical application.

12. The method of claim 5 or 8, wherein the composition is administered via local administration.

13. The method of claim 12, wherein the local administration of the compound is via a suture, a vascular implant, a stent, a heart valve, a drug pump, a drug delivery catheter, an infusion catheter, a drug delivery guidewire or an implantable medical device.

14. A composition comprising the compound of claim 2 and at least one therapeutic agent and/or at least one nitric oxide donor compound.

15. The composition of claim 14, wherein the therapeutic agent is a antithrombogenic agent, a thrombolytic agent, a fibrinolytic agent, a vasospasm inhibitor, a potassium channel blocker, a calcium channel blocker, an antihypertensive agent, an antimicrobial agent, an antibiotic, a platelet reducing agent, an antimitotic agent, an antiproliferative agent, a microtubule inhibitor, an antisecretory agent, a remodelling inhibitor, an antisense nucleotide, an anti-cancer chemotherapeutic agent, a steroid, a non-steroidal antiinflammatory agent, a selective COX-2 inhibitor, an immunosuppressive agent, a growth factor antagonist or antibody, a dopamine agonist, a radiotherapeutic agent, a heavy metal functioning as a radioplaque agent, a biologic agent, an aldosterone antagonist, an alpha-adrenergic receptor antagonist, an angiotensin II antagonist, a β-adrenergic agonist, an anti-hyperlipidemic drug, an angiotensin converting enzyme (ACE) inhibitor, an antioxidant, a β-adrenergic antagonist, an endothelin antagonist, a neutral endopeptidase inhibitor, a renin inhibitior, a free radical scavenger, an iron chelator, a sex hormone, an antipolymerase, an antiviral agent, a photodynamic therapy agent, an antibody targeted therapy agent, a gene therapy agent, or a mixture of two or more thereof.

16. The composition of claim 14, wherein the nitric oxide donor compound is an S-nitrosothiol, a compound that comprises at least one ON—O— group, ON—N— group, O₂N—O— group, O₂N—N— group or O₂N—S— group, a N-oxo-N-nitrosoamine, L-arginine, L-homoarginine, N-hydroxy-L-homoarginine, N-hydroxydebrisoquine, N-hydroxypentamidine, N-hydroxy-L-arginine, nitrosated L-arginine, nitrosylated L-arginine, nitrosated N-hydroxy-L-arginine, nitrosylated N-hydroxy-L-arginine, nitrosated L-homoarginine, a N-hydroxyguanidine compound, an amidoxime, a ketoxime, an aldoxime compound, citrulline, ornithine, glutamine, lysine, an arginase inhibitor, a nitric oxide mediator or a NONOate.

17. The composition of claim 4 bound to a matrix, wherein the matrix is a natural polymer, a synthetic polymer, a natural fiber, a synthetic fiber, or a combination of two or more thereof.

18. The composition of claim 17, further comprising at least one nitric oxide donor compound and/or at least one therapeutic agent.

19. The composition of claim 15, wherein the composition coats all or a portion of the surface of a medical device or forms all or part of the medical device.

20. The medical device of claim 18, wherein the medical device is a balloon, a catheter tip, a stent, a catheter, a prosthetic heart valve, a synthetic vessel graft, an arteriovenous shunt, a heart valve, a suture, a vascular implant, a drug pump, a drug delivery catheter, plastic tubing, a dialysis bag, a lead, a pacemaker, an implantable pulse generator, an implantable cardiac defibrillator, a cardioverter defibrillator, a defibrillator, a spinal stimulator, a brain stimulator, a sacral nerve stimulator, a chemical sensor or a membrane surface.
